(19) United States
(12) Patent Application Publication
(10) Pub. No.: US 2004/0006208 A1
(43) Pub. Date: Jan. 8, 2004

(54) Co-crystal structure of monoclonal antibody 5C8 and CD154, and use thereof in drug design

(76) Inventors: Michael Karpusas, Upper Darby, PA (US); Yen-Ming Hsu, Lexington, MA (US); Frederick R. Taylor, Milton, MA (US); Zhongli Zheng, Lexington, MA (US)

Correspondence Address: FISH & NEAVE, 1251 AVENUE OF THE AMERICAS, 50TH FLOOR, NEW YORK, NY, 10020-1105, US

(21) Appl. No.: 10/378,567

(22) Filed: Feb. 28, 2003

Related U.S. Application Data
(63) Continuation of application No. PCT/US01/27352, filed on Aug. 31, 2001.
(60) Provisional application No. 60/229,933, filed on Sep. 1, 2000.
(60) Provisional application No. 60/276,452, filed on Mar. 16, 2001.

(51) Int. Cl. G06F 19/00; G01N 33/48; G01N 33/50; C07K 14/74; C07K 16/28

(52) U.S. Cl. 530/350; 530/388.22; 702/19

(57) ABSTRACT

The present invention relates to compositions and crystals of CD154 (CD40L) in complex with an anti-CD154 antibody. In addition, this invention relates to the high resolution structure of a CD154/anti-CD154 antibody complex as obtained by X-ray crystallography. Specifically, this structure provides binding sites defined by the structure coordinates determined herein. This invention also relates to a computer (machine) comprising a machine-readable data storage medium comprising a data storage material encoded with machine-readable data comprising the structure coordinates provided by this invention. The computer has instructions to process said machine-readable data into a three-dimensional representation of a molecular complex of CD154/anti-CD154 antibody based on the structure coordinates provided by this invention. This invention also relates to methods using the structure coordinates of an CD154/anti-CD154 antibody complex to solve the structure of similar or homologous molecular complexes, as well as methods using the structure coordinates of an CD154/anti-CD154 antibody complex to design chemical entities or compounds, including agonists or antagonists of CD154, that specifically bind CD154 and function as CD40:CD154 binding interrupters, as well as to design variants of monoclonal antibody 5c8, or humanized monoclonal antibody 5c8, or antigen binding fragments thereof, having improved properties (such as increased or decreased binding affinity for CD154). This invention also relates to compositions comprising said chemical entities, compounds or variants of monoclonal antibody 5c8 or humanized monoclonal antibody 5c8. The invention further relates to uses of said chemical entities, compounds or variants of monoclonal antibody 5c8 or humanized monoclonal antibody 5c8 to treat a subject having one or more conditions associated with inappropriate or abnormal CD154 induced activation.

### TECHNICAL FIELD OF THE INVENTION

The present invention relates to the field of crystallography and computer-assisted analysis of proteins and polypeptides. The present invention further relates to the field of computational drug design.

### BACKGROUND OF THE INVENTION

Data establishing that T cell activation requires both T cell receptor (“TCR”) mediated signals and simultaneously delivered costimulatory signals have accumulated over the past twenty years. For example, antibody production by B lymphocytes in response to protein antigens requires a specific, costimulatory interaction with T lymphocytes. This B cell/T cell interaction is mediated through several receptor-ligand binding events in addition to engagement of the TCR. See, e.g., Noelle et al. Immunology Today 13: 431-433 (1992). See also Hollenbaugh et al. EMBO J. 11: 4313-4321 (1992). These additional binding events include the binding of CD40 on B cells to CD154 (CD40L, and also known as gp39, T-BAM, 5c8 antigen, CD40CR and TRAP) on T cells. Human CD40 is a 50 kilodalton cell surface protein expressed on mature B cells, as well as macrophages, dendritic cells, fibroblasts and activated endothelial cells. CD40 belongs to a class of receptors involved in cell signaling and in programmed cell death, including Fas/CD95 and the tumor necrosis factor (TNF) alpha receptor. Human CD154, a 32 kilodalton type II membrane glycoprotein having homology to TNF alpha, is a member of the TNF family of receptors and is transiently expressed primarily on activated T cells. CD40:CD154 binding has been shown to be required for T cell-dependent antibody responses. In particular, CD40:CD154 binding provides anti-apoptotic and/or lymphokine stimulatory signals. See, e.g., Karpusas et al. Structure 3, 1031-1039 (1995) and Karpusas et al. Structure 3, 1446 (1995), U.S. patent application Ser. No. 09/180,209 and PCT patent application WO 97/00895, the disclosures of which are hereby incorporated by reference.

The importance of CD40:CD154 binding in promoting T cell dependent biological responses is underscored by the development of X-linked hyper-IgM syndrome (X-HIGM) in humans lacking functional CD154. These individuals have normal or high IgM levels, but fail to produce IgG, IgA or IgE antibodies. Affected individuals suffer from recurrent, sometimes severe, bacterial infection (most commonly Streptococcus pneumoniae, Pneumocystis carinii and Hemophilus influenzae) and certain unusual parasitic infections, as well as an increased incidence of lymphomas and abdominal cancers. These clinical manifestations of disease can be managed through intravenous immunoglobulin replacement therapy.

The effects of X-HIGM are simulated in animals rendered nullizygous for the gene encoding CD154 (knockout animals). Studies with nullizygotes have confirmed that, while B cells can produce IgM in the absence of CD40:CD154 binding, they are unable to undergo isotype switching, or to survive normally and undergo affinity maturation. In the absence of a functional CD40:CD154 interaction, spleen and lymph node germinal centers do not develop properly, and the development of memory B cells is impaired. These defects contribute to a severe reduction in or absence of a secondary (mature) antibody response.

Individuals with X-HIGM and CD154 nullizygotes also have defects in cellular immunity. These defects are manifested by an increased incidence of Pneumocystis carinii, Histoplasma capsulatum, Cryptococcus neoformans infection, as well as chronic Giardia lambli infection. Murine nullizygotes are deficient in their ability to fight Leishmania infection. Many of these cell-mediated defects are reversible by administration of IL-12 or IFN-gamma. These data substantiate the view that CD40:CD154 binding promotes the development of Type I T-helper cell responses. Further support is derived from the observation that macrophage activation is defective in CD154-deficient settings, and that administration of anti-CD154 antibodies to mice diminished their ability to clear Pneumocystis infection. Blockade of CD40:CD154 binding appears to reduce the ability of macrophages to produce nitric oxide, which mediates many of the macrophages' pro-inflammatory activities. It should be noted, however, that mammals (including humans) who lack functional CD154 do not develop significant incidences of viral infection.

A number of preclinical studies, including those described in co-pending, commonly assigned PCT patent applications published as WO98/30241, WO98/30240, WO98/52606, WO98/58669 and WO99/45958, describe the promise of agents capable of interrupting CD40:CD154 binding as immunomodulating agents. In murine systems, antibodies to CD154 block primary and secondary immune responses to exogenous antigens, both in vitro and in vivo. Antibodies to CD154 cause a reduction in germinal centers in mice and monkeys, consistent with data on CD154 immunodeficiency. Administration of three doses of anti-CD154 antibody to lupus-prone mice, age three months, substantially reduced titers against double-stranded DNA and nucleosomes, delayed the development of severe nephritis, and reduced mortality. Moreover, administration of anti-CD154 antibodies to mice age five to seven months with severe nephritis was shown to stabilize or even reverse renal disease. Anti-CD154 antibodies given concomitantly with small resting allogeneic lymphocytes permitted unlimited survival of mouse pancreatic islet allografts. In other animal models, interference with CD40:CD154 binding has been demonstrated to reduce symptoms of autoimmune disease (e.g., multiple sclerosis, rheumatoid arthritis, inflammatory bowel disease), graft rejection (e.g., cardiac allograft, graft-versus-host disease), and mercuric chloride induced glomerulonephritis, which is mediated by both humoral and cellular mechanisms.

Such studies with anti-CD154 antibodies demonstrate the role of CD154 as a critical target for modulating immune responses.

Currently, the most effective of the available CD40:CD154 binding interrupters are anti-CD154 antibodies. Antibodies, however, may not, in all cases, be the most effective CD40:CD154 binding interrupters for use as a human therapeutic agent. Further development of novel agents that are more effective in interrupting CD40:CD154 interactions and serve as improved human therapeutic agents is hampered by the lack of structural information of CD154 and an agent known to bind specifically to CD154. That information is provided for the first time by the present invention.

### SUMMARY OF THE INVENTION

Applicants have solved the above-identified problem by providing compositions, which can be crystallizable, and crystals of CD154(CD40L) in complex with an antibody that specifically binds CD154 (an anti-CD154 antibody) and methods for using such compositions and crystals.

This invention also provides the structure coordinates of CD154 in complex with an antibody that specifically binds CD154.

This invention also provides methods for determining at least a portion of the three-dimensional structures of molecular complexes which contain at least some structurally similar features to a CD154/anti-CD154 antibody complex.

This invention also provides methods for designing chemical entities, compounds, such as agonists and antagonists of CD154, and variants of the 5c8 monoclonal antibody, or an antigen binding fragment thereof, that specifically bind CD154 and, accordingly, act as CD40:CD154 binding interruptors. This invention further relates to compositions comprising the chemical entities, the compounds, such as agonists and antagonists of CD154, and the variants of the 5c8 monoclonal antibody, or antigen binding fragments thereof, that specifically bind CD154 and that are rationally designed by means of the structure coordinates of a CD154/anti-CD154 antibody complex. The invention further relates to use of the above-identified chemical entities, compounds, such as agonists and antagonists of CD154, and variants of the 5c8 monoclonal antibody, or antigen binding fragments thereof, to treat conditions associated with inappropriate or abnormal CD154 activation in a subject.

This invention also provides a computer, which comprises a storage medium comprising a data storage material, for producing three-dimensional representations of molecular complexes comprising binding sites defined by structure coordinates of CD154 and an anti-CD154 antibody and methods for using these three-dimensional representations to design: 1) chemical entities and compounds that associate with CD154 or anti-CD154 antibody, 2) compounds, such as potential agonists or antagonists of CD154; specifically, or 3) variants of anti-CD154 antibodies (such as variants of 5c8 mAb) with improved properties, such as those that bind with higher or lower affinity to CD154 as compared to the non-variant, parent anti-CD154 antibody (such as 5c8 mAb), by using computational means to perform a fitting operation between chemical entities, compounds, such as agonists and antagonists of CD154, and variants of the 5c8 monoclonal antibody, or an antigen binding fragment thereof, and a binding site. This invention also provides the chemical entities, the compounds, such as agonists and antagonists of CD154, and the variants of the 5c8 monoclonal antibody, or an antigen binding fragment thereof and compositions comprising them.

The foregoing and other objects, features and advantages of the present invention, as well as the invention itself, will be more fully understood from the following description of preferred embodiments.

### BRIEF DESCRIPTION OF THE DRAWINGS

The patent or application file contains at least one drawing executed in color. Copies of this patent or patent application publication with color drawing(s) will be provided by the Patent Office upon request and payment of the necessary fee.

FIG. 1 depicts a ribbon diagram of a complex comprising a trimer of the extracellular domain of human CD154 and three Fab fragments of humanized monoclonal antibody 5c8 (“hu5c8 mAb”). Each Fab fragment of humanized 5c8 mAb binds to a monomer of CD154. This figure provides a view along the 3-fold axis. The three CD154 monomers, located in the center of the Figure, are colored yellow, green and dark blue. The three Fab heavy chains (“H chains”), located in the foreground relative to the Fab light chain, are colored grey, dark grey and magenta and the three Fab light chains (“L chains”), located in the background relative to the Fab heavy chain, are colored dark blue, orange and turquoise.

FIG. 2 depicts a ribbon diagram of a complex comprising a trimer of the extracellular domain of human CD154 and three Fab fragments of humanized 5c8 mAb. Each Fab fragment of humanized 5c8 mAb binds to a monomer of CD154. This figure provides a view that is perpendicular to the 3-fold axis. The 3-fold axis runs from top to bottom of the diagram. The three CD154 monomers, located in the center of the Figure, are colored yellow, green and dark blue. The three Fab heavy chains are colored grey, dark grey and magenta; and the three Fab light chains are colored dark blue, orange and turquoise. Only two of the three Fab fragments of hu5c8 mAb are displayed; the third Fab fragment has been omitted for clarity.

FIG. 3 depicts a stereo view of a representative portion of the final 2Fo-Fc electron density map. The map is contoured at 1.2σ and superimposed on corresponding atoms from the final refined model.

FIG. 4 lists the atomic structure coordinates for the extracellular domain of human CD154 in complex with the Fab fragment of humanized 5c8 mAb, as derived by X-ray diffraction from crystals of that complex in protein data bank (PDB) format.

FIG. 5 shows a diagram of a system used to carry out the instructions encoded by the storage media of FIGS. 6 and 7.

FIG. 6 shows a cross-section of a magnetic storage medium.

FIG. 7 shows a cross section of an optically-readable data storage medium.

FIG. 8 shows the amino acid sequence of human CD154 (the fragment in brackets was crystallized) and shows the amino acid sequence of humanized 5c8 mAb heavy and light chains (the fragments in brackets were visible in the crystal structure; whereas the actual molecule crystallized could be a few residues longer (heavy chain) or was presumably the whole sequence (light chain)). Residues of the CDR loops of the hu5c8 mAb heavy and light chains are underscored.

FIG. 9 lists the atomic structure coordinates for the uncomplexed Fab fragment of humanized 5c8 mAb, as derived by X-ray diffraction from crystals of that Fab fragment in protein data bank (PDB) format.

FIG. 10 shows a view of the CD154-5c8 mAb interface. The CD154 backbone is represented as a yellow ribbon and the H and L chains of 5c8 mAb are represented as blue and red ribbons. Side chains of residues involved in CD154-5c8 mAb contacts are shown. The thin lines indicate H-bonds.

FIG. 11 shows mutated residues and the antigenic epitope on CD154 for 5c8 mAb.

(A) The solvent accessible surface shown with a dotted, darker surface represents the antigenic epitope. The representation of the antigenic epitope is on the monomer on the right side of the Figure. The two monomers of CD154 shown are in space-filling representation and are colored blue (on the left side of the Figure) and grey (on the right side of the Figure) respectively.

(B) Space-filling representation of CD154 indicating the position of mutated residues. The effects of the mutations are color-coded according to the data for 5c8 mAb binding in Table 2 in Example 2: green (+), yellow (+/−), red (−)

### DETAILED DESCRIPTION OF THE INVENTION

The following discussion illustrates and exemplifies the variety of contexts and circumstances in which the invention can be practiced, as well as providing specific embodiments of the invention.

Throughout this specification and claims, the word “comprise,” or variations such as “comprises” or “comprising,” will be understood to imply the inclusion of a stated integer or group of integers but not the exclusion of any other integer or group of integers.

AMINO ACID ABBREVIATIONS

| | | | | |
|---|---|---|---|---|
| A | = | Ala | = | Alanine |
| V | = | Val | = | Valine |
| L | = | Leu | = | Leucine |
| I | = | Ile | = | Isoleucine |
| P | = | Pro | = | Proline |
| F | = | Phe | = | Phenylalanine |
| W | = | Trp | = | Tryptophan |
| M | = | Met | = | Methionine |
| G | = | Gly | = | Glycine |
| S | = | Ser | = | Serine |
| T | = | Thr | = | Threonine |
| C | = | Cys | = | Cysteine |
| Y | = | Tyr | = | Tyrosine |
| N | = | Asn | = | Asparagine |
| Q | = | Gln | = | Glutamine |
| D | = | Asp | = | Aspartic Acid |
| E | = | Glu | = | Glutamic Acid |
| K | = | Lys | = | Lysine |
| R | = | Arg | = | Arginine |
| H | = | His | = | Histidine |

Applicants have solved the three-dimensional structure of a CD154/anti-CD154 antibody complex using high resolution X-ray crystallography. Importantly, this has provided, for the first time, the information about the shape and structure of both the binding site of CD154 (specifically, human CD154) for an anti-CD154 antibody (specifically, monoclonal antibody 5c8) and the binding site of an anti-CD154 antibody (specifically, monoclonal antibody 5c8) for CD154.

Compositions and Crystals

According to a preferred embodiment, the compositions of this invention are crystallizable. Those compositions comprise a CD154 polypeptide in complex with an antibody that specifically binds CD154 (an anti-CD154 antibody), or an antigen binding fragment thereof.

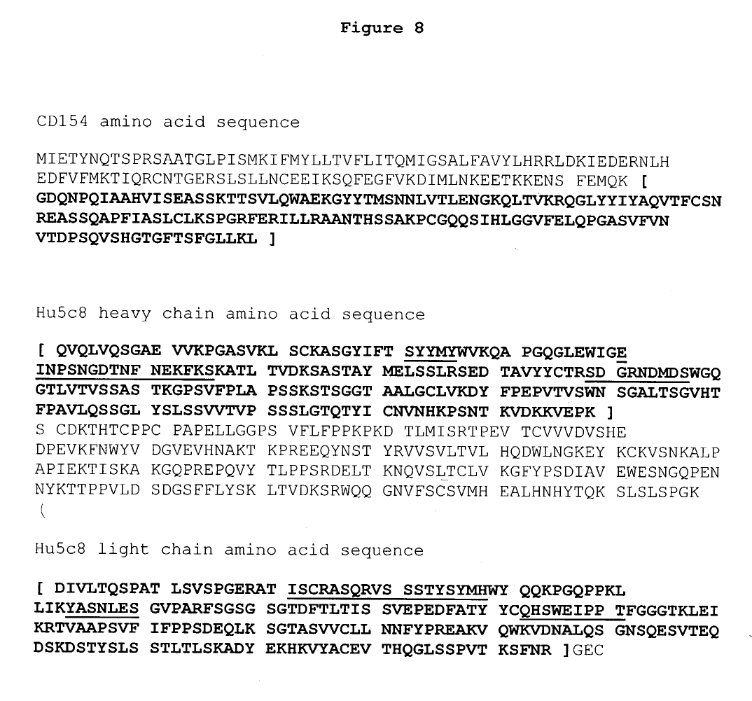

The CD154 polypeptide portion of the complex is any CD154 polypeptide capable of specifically binding to an anti-CD154 antibody, preferably an antibody that is capable of blocking the interaction between CD40 and CD154. In a preferred embodiment, the CD154 polypeptide comprises the extracellular domain, or a portion thereof, of CD154. In another preferred embodiment, the CD154 polypeptide comprises a polypeptide consisting of CD154 amino acid residues 116 to 261. See FIG. 8. In a preferred embodiment, the CD154 is human CD154. In another preferred embodiment, the crystallizable composition comprises a trimer of CD154 polypeptides and three anti-CD154 antibody molecules, or antigen binding fragments thereof. A CD154 polypeptide could be a fusion protein comprising CD154, or a portion thereof, and one or more other proteins or polypeptides. The fusion protein could also comprise CD154, or a portion thereof, and one or more epitope tags, such as a MYC tag.

The anti-CD154 antibody portion of the complex is an antibody, or an antigen binding fragment thereof, capable of specifically binding the epitope on CD154 that is specifically bound by an antibody, preferably an antibody capable of blocking the interaction between CD40 and CD154. Preferably, the anti-CD154 antibody is a monoclonal antibody. Examples include monoclonal antibody (“mAb”) 5c8 (produced by the hybridoma having ATCC Accession No. HB 10916), humanized 5c8 mAb, Fab′, (Fab)₂ and Fab fragments of 5c8 mAb or humanized 5c8 mAb. In a more preferred embodiment, the antibody, or an antigen binding fragment thereof, binds specifically to human CD154. Examples include 5c8 mAb, humanized 5c8 mAb, and Fab′, (Fab)₂ and Fab fragments of 5c8 mAb or humanized 5c8 mAb. An anti-CD154 antibody could be a fusion protein comprising an anti-CD154 antibody, or an antigen binding portion thereof, and one or more other proteins or polypeptides. The fusion protein could also comprise an anti-CD154 antibody, or an antigen binding portion thereof, and one or more epitope tags, such as a MYC tag.

In a preferred embodiment, the anti-CD154 antibody is a monoclonal antibody which specifically binds the 5c8 antigen, which is specifically bound by monoclonal antibody 5c8 (produced by the hybridoma having ATCC Accession No. HB 10916). 5c8 antigen is human CD154. A human CD154 DNA sequence and a human CD154 amino acid sequence were disclosed in Hollenbaugh et al., EMBO J., 11: 4313-4321 (1992).

An antibody that is capable of blocking the interaction between CD40 and CD154 is one that blocks the interaction of CD40, for example, cell surface CD40 (e.g., on B cells, dendritic cells, endothelial cells and other antigen presenting cells) with CD154, for example, CD154 expressed on the surface of activated T cells. CD40:CD154 binding interrupter compounds, such as anti-CD154 compounds, that are specifically contemplated include polyclonal antibodies and monoclonal antibodies, as well as antibody derivatives such as chimeric molecules, humanized molecules, molecules with altered (e.g., reduced) effector functions, bispecific molecules, and conjugates of antibodies. In a preferred embodiment, the antibody is 5c8 mAb (produced by the hybridoma having ATCC Accession Number HB 10916, deposited on Nov. 14, 1991), as described in U.S. Pat. No. 5,474,771, the disclosure of which is hereby incorporated by reference. In a highly preferred embodiment, the antibody is a humanized 5c8 mAb. Other known antibodies against CD154 include, for example, antibodies ImxM90, ImxM91 and ImxM92 (described in U.S. Pat. No. 5,961,974). Numerous additional anti-CD154 antibodies have been produced and characterized (see, e.g., PCT patent application WO96/23071 of Bristol-Myers Squibb, the specification of which is hereby incorporated by reference). The selection of an appropriate monoclonal antibody will depend on the animal species from which CD154 is derived and the species specificity of the anti-CD154 monoclonal antibody (for example, 5c8 mAb, produced by the hybridoma having ATCC Accession No. HB 10916 and raised against human CD154, specifically binds to human and some non-human primate CD154 molecules but not to mouse CD154). When the CD154 is mouse CD154 (known as gp39), an antibody that binds mouse CD154 should be used. An example of such an antibody is MR1 (see Noelle et al. (1992), Proc. Natl. Acad. Sci. USA 89: 6550).

The invention also includes anti-CD154 molecules of other types, such as complete Fab fragments, F(ab′)₂ compounds, V_{H} regions, F_{V} regions and single chain antibodies (see, e.g., PCT patent application WO96/23071) polypeptides.

Various forms of antibodies may also be produced using standard recombinant DNA techniques (Winter and Milstein, Nature 349: 293-99, 1991). For example, “chimeric” antibodies may be constructed, in which the antigen binding domain from a non-human animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81: 6851-55, 1984).

In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. Such antibodies are chimeras comprising mostly human immunoglobulin sequences into which the regions responsible for specific antigen binding have been inserted (see, e.g., PCT patent applications WO90/07861 and WO94/04679, the disclosures of which are incorporated hereby by reference). Animals are immunized with the desired antigen, the corresponding antibodies are isolated and the portions of the variable region sequences responsible for specific antigen binding are removed. The animal-derived antigen binding regions are then cloned into the appropriate position of the human antibody genes from which the antigen binding regions have been deleted. Humanized antibodies minimize the use of heterologous (inter-species) sequences in antibodies targeted for human therapies, and are less likely to elicit unwanted immune responses. Primatized antibodies can be produced similarly using primate (e.g., rhesus, baboon and chimpanzee) antibody genes.

Another embodiment of the invention includes the use of human antibodies, which can be produced in nonhuman animals, such as transgenic animals harboring one or more human immunoglobulin transgenes. Such animals may be used as a source for splenocytes for producing hybridomas, as described in U.S. Pat. No. 5,569,825.

Antibody fragments and univalent antibodies are also contemplated by this invention. Univalent antibodies comprise a heavy chain/light chain dimer bound to the Fc (or stem) region of a second heavy chain. “Fab region” refers to those portions of the chains which are roughly equivalent, or analogous, to the sequences which comprise the Y branch portions of the heavy chain and to the light chain in its entirety, and which collectively (in aggregates) have been shown to exhibit antibody activity. A Fab protein includes aggregates of one heavy and one light chain (commonly known as Fab′), as well as tetramers which correspond to the two branch segments of the antibody Y, (commonly known as F(ab)₂), whether any of the above are covalently or non-covalently aggregated, so long as the aggregation is capable of selectively reacting with a particular antigen or antigen family.

In addition, standard recombinant DNA techniques can be used to alter the binding affinities of recombinant antibodies with their antigens by altering amino acid residues in the vicinity of the antigen binding sites. The antigen binding affinity of a humanized antibody may be increased by mutagenesis based on molecular modeling (Queen et al., Proc. Natl. Acad. Sci. 86:10029-33, 1989; PCT patent application WO94/04679, which are hereby incorporated by reference). This may also be done utilizing phage display technology (see, e.g., Winter et al., Ann. Rev. Immunol. 12:433-455, 1994; and Schier et al., J. Mol. Biol. 255:28-43, 1996, which are hereby incorporated by reference).

Crystal Structures and Methods Using the Structure Coordinates that Define the Three-dimensional Structure of a CD154/Anti-CD154 Antibody Complex

The crystallizable compositions provided by this invention are amenable to X-ray crystallography. Therefore, this invention also encompasses crystals of the crystallizable compositions. This invention also provides the three dimensional structure as obtained by X-ray crystallography of a CD154/anti-CD154 antibody complex at high resolution, such as at 3.1 Åresolution. See Example 1. In a preferred embodiment, the CD154 polypeptide is the extracellular domain of human CD154 (for example, amino acids 116 to 261) and the anti-CD154 antibody, or an antigen binding fragment thereof, is the Fab fragment of humanized 5c8 mAb.

The three dimensional structures of other crystallizable compositions of this invention may also be determined by X-ray crystallography using X-ray crystallographic techniques routine in the art.

X-ray crystallography is a collection of techniques which allow the determination of the structure of a molecular entity. The techniques include crystallization of the entity, collection and processing of X-ray diffraction intensities, determination of phases (by, e.g., multiple isomorphous replacement, molecular replacement or difference Fourier techniques) and model building and refinement.

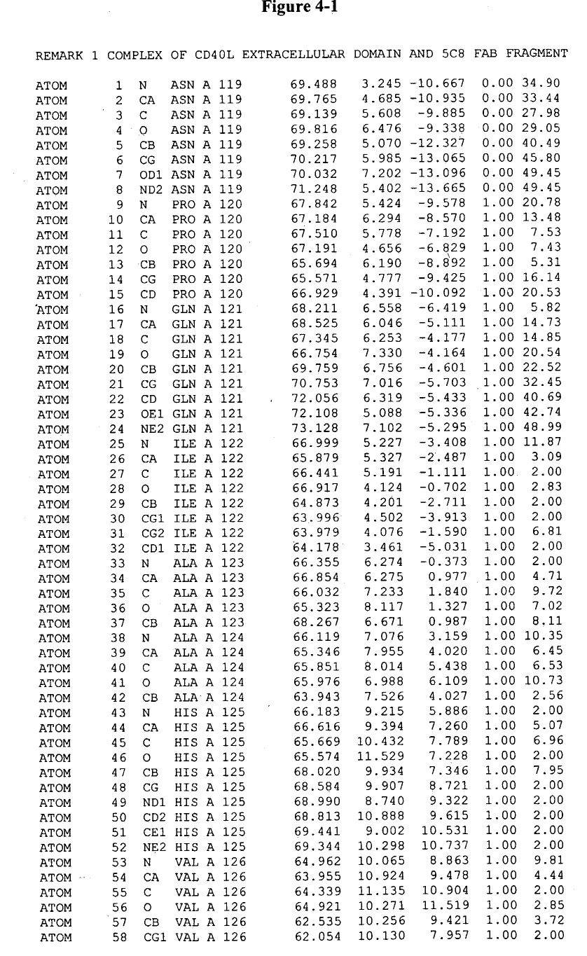

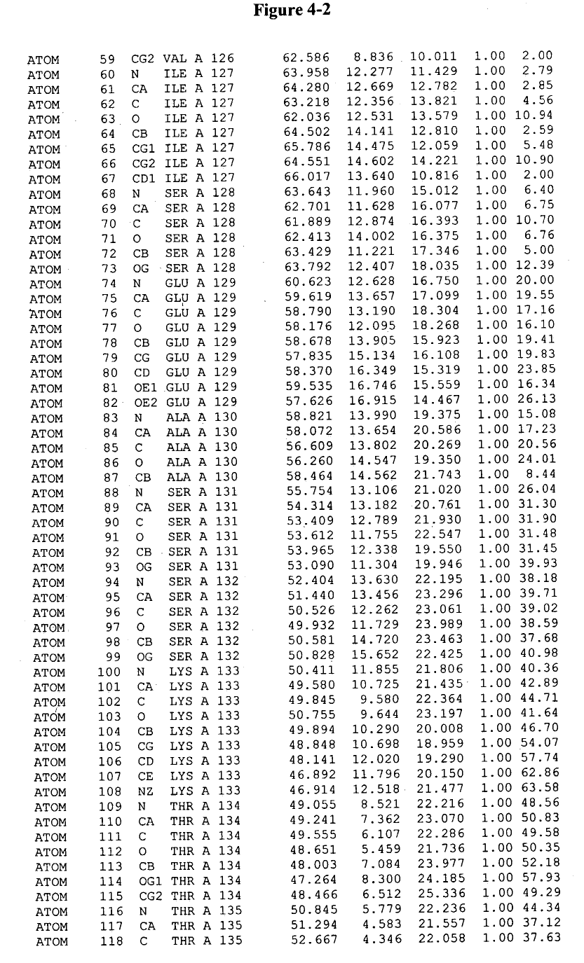

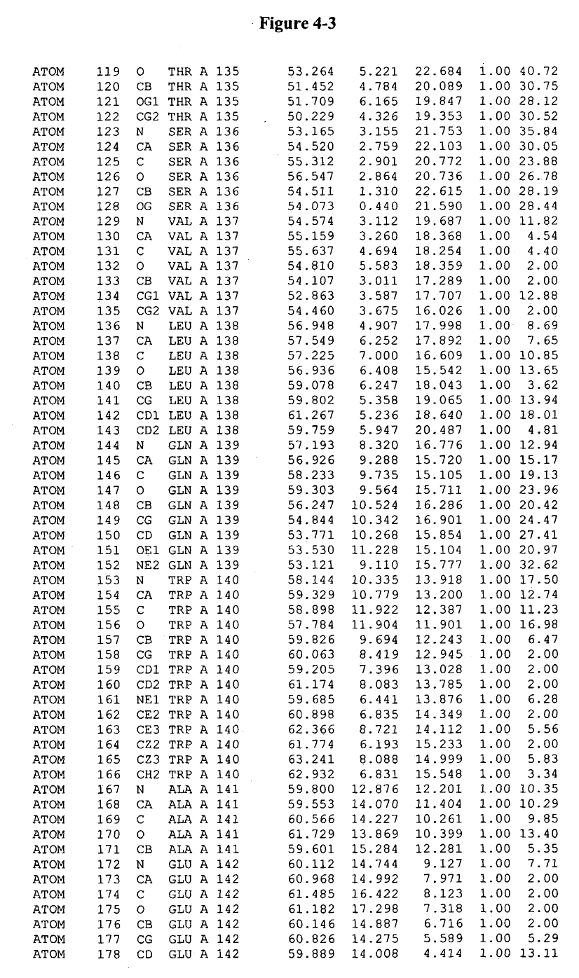

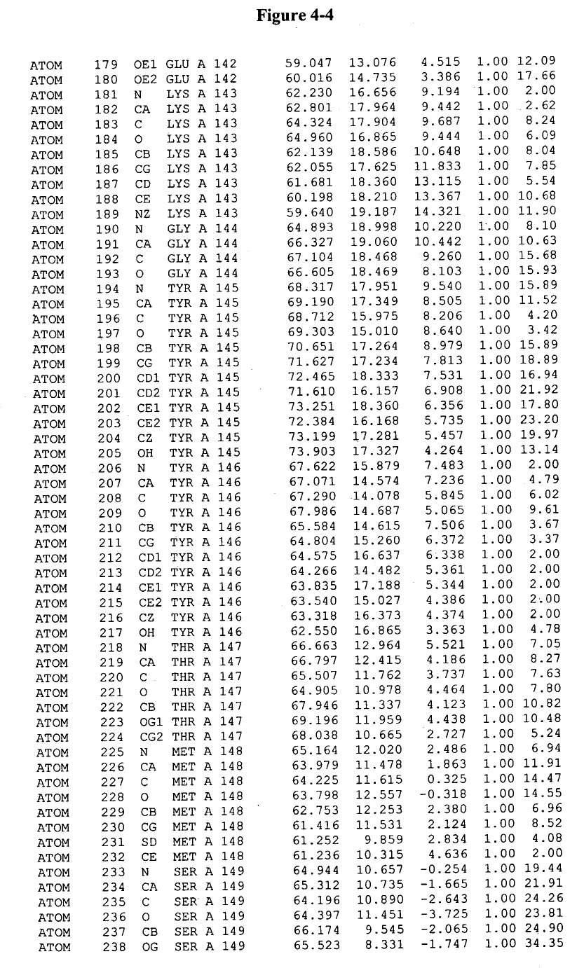

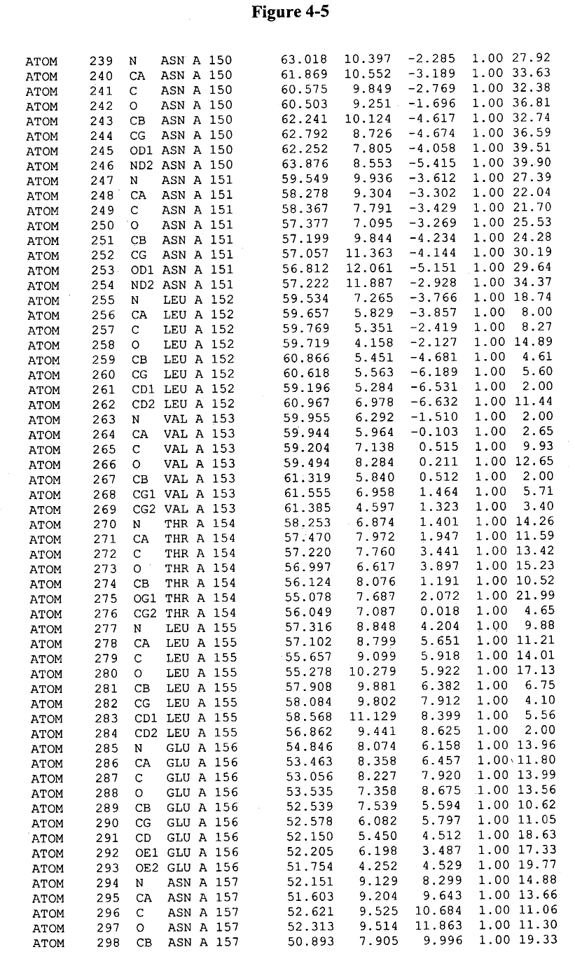

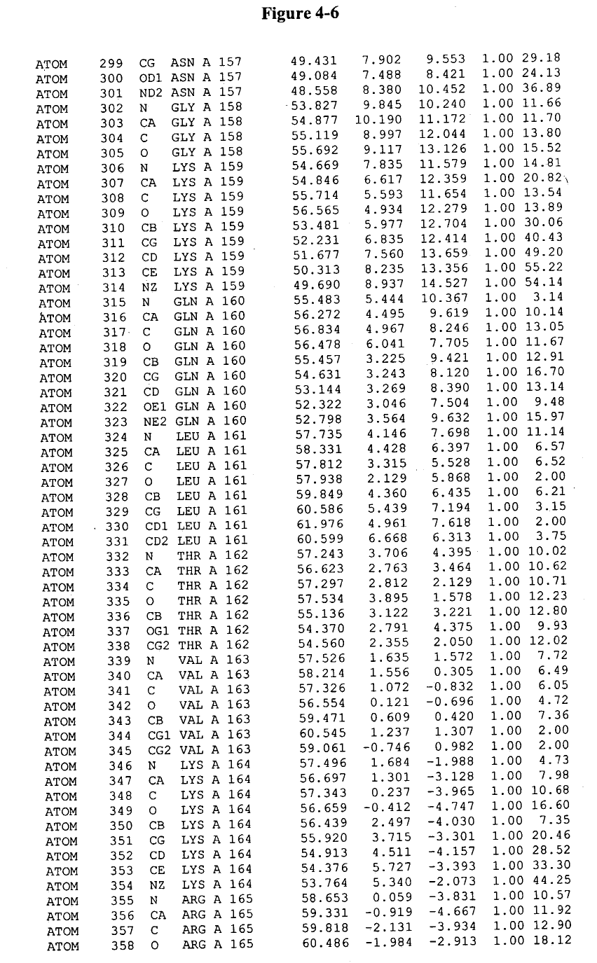

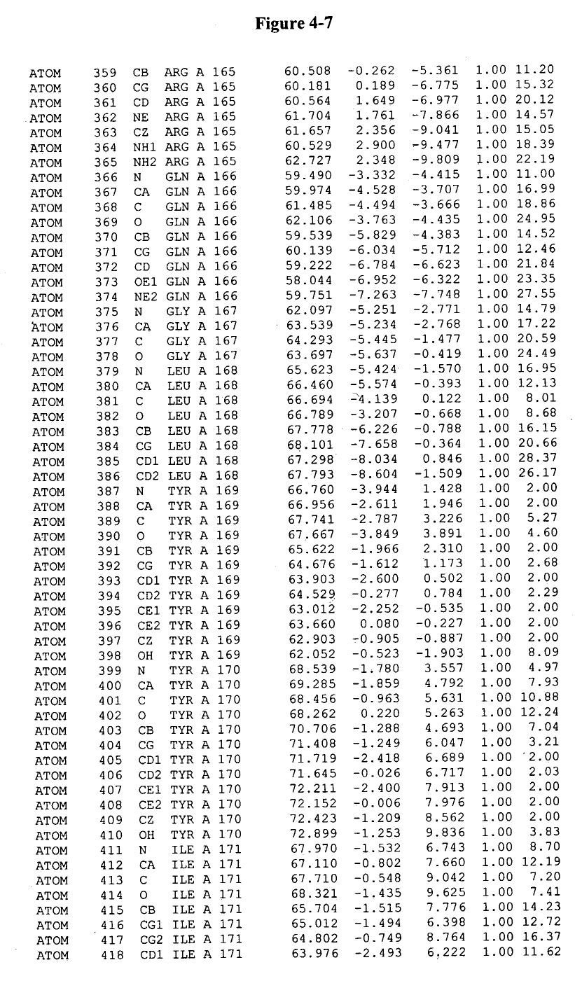

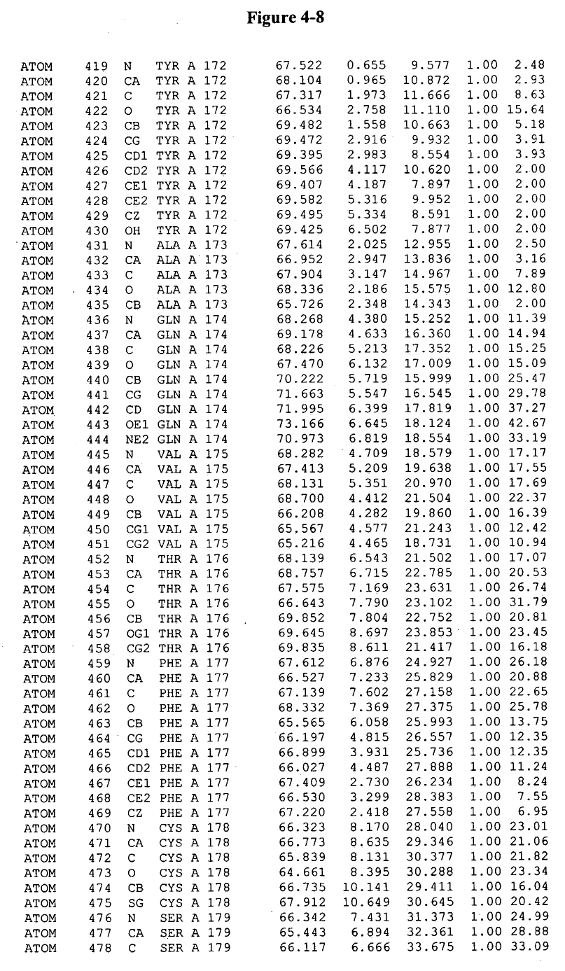

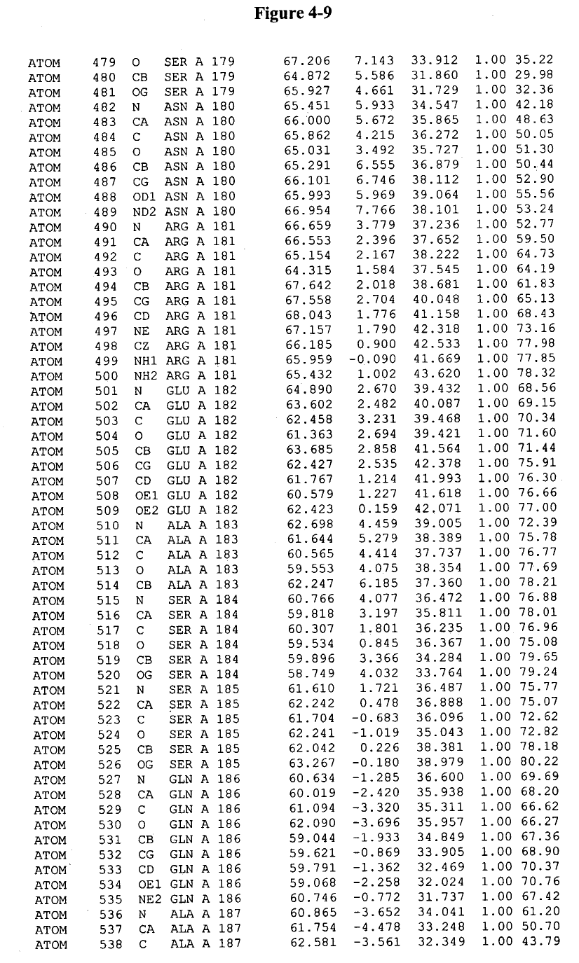

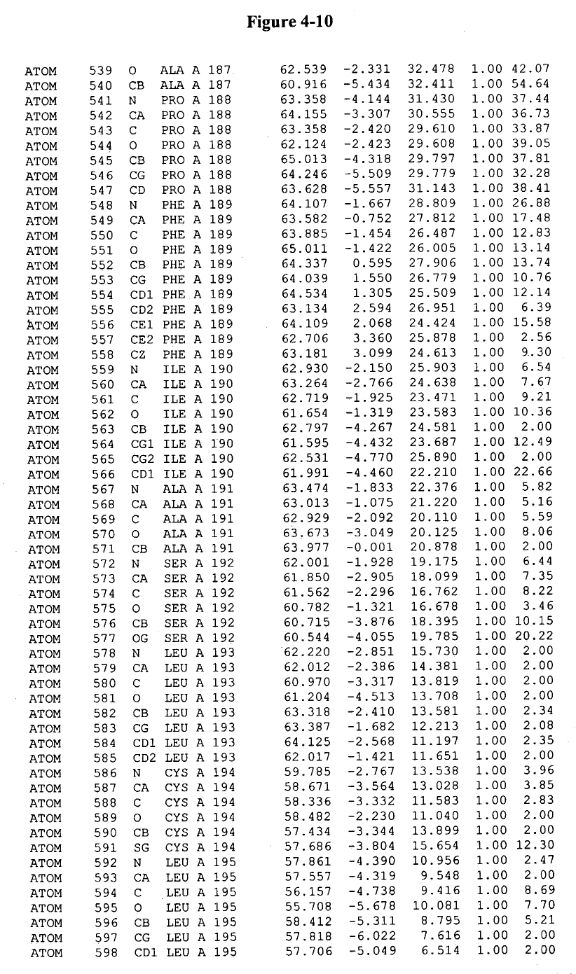

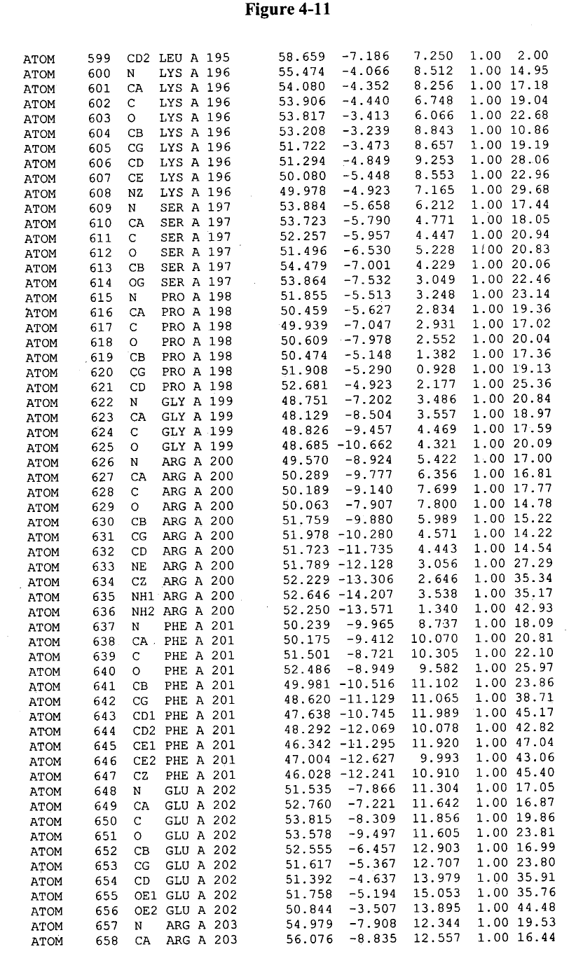

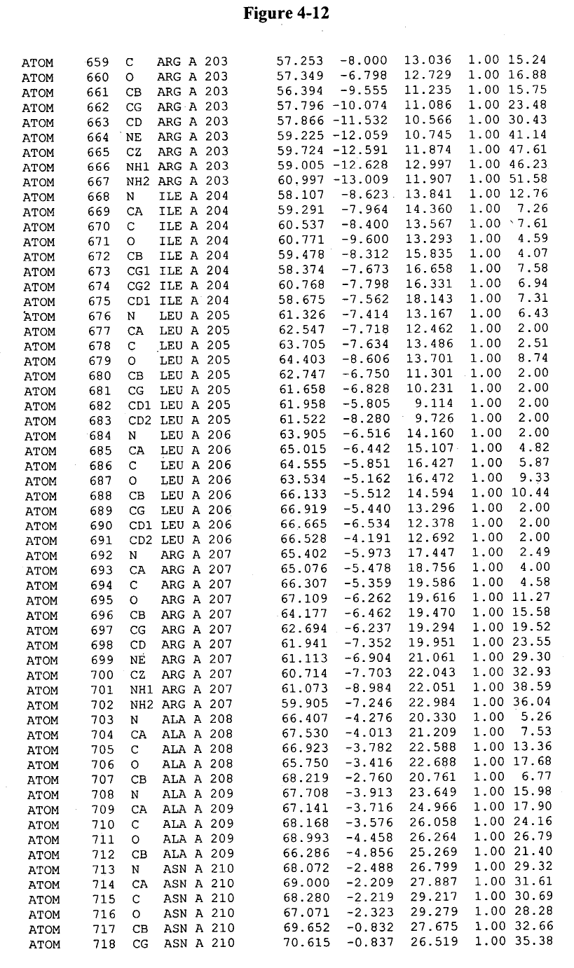

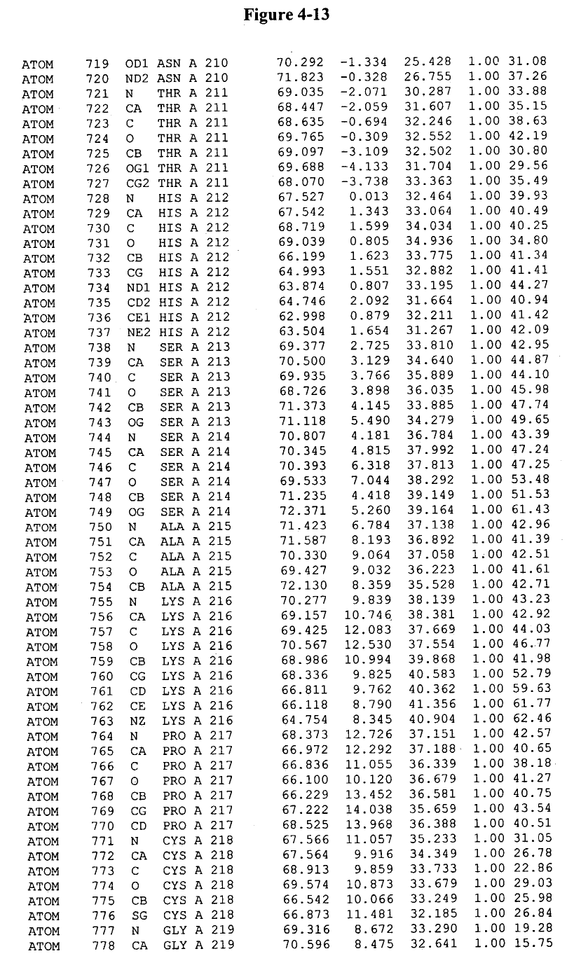

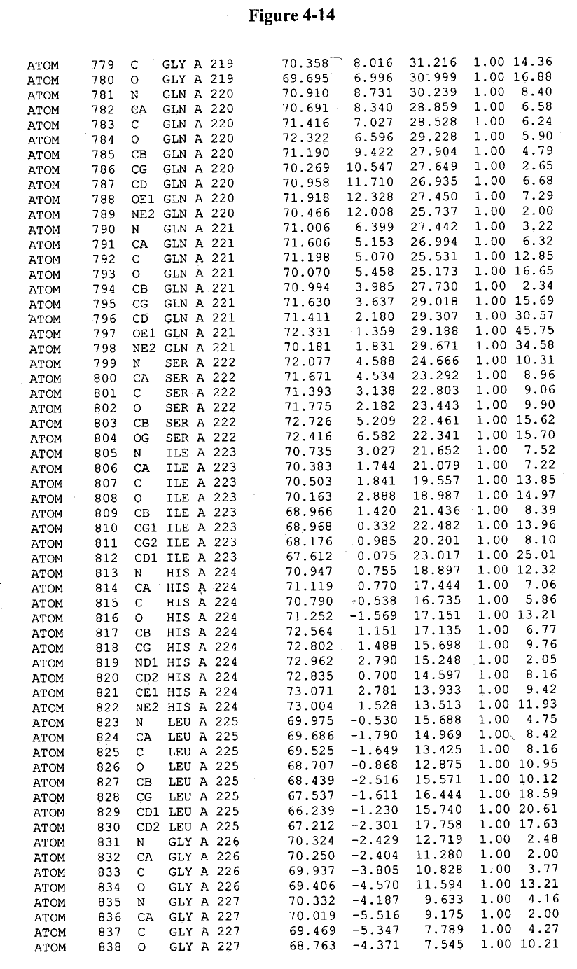

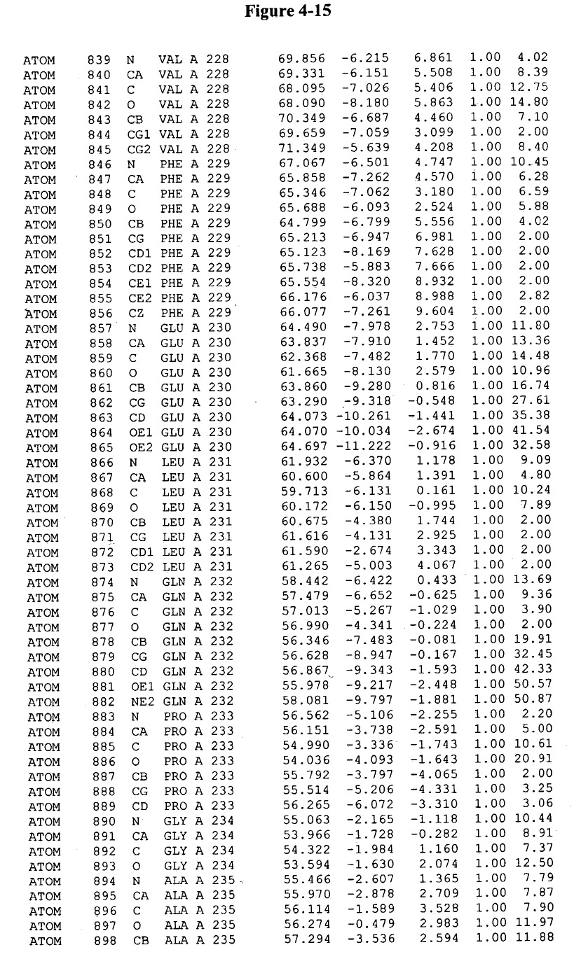

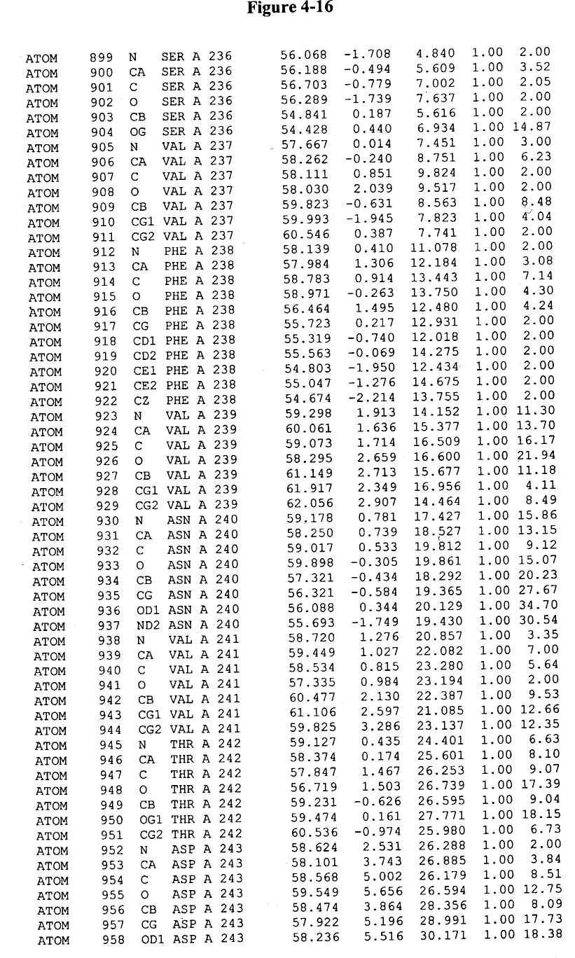

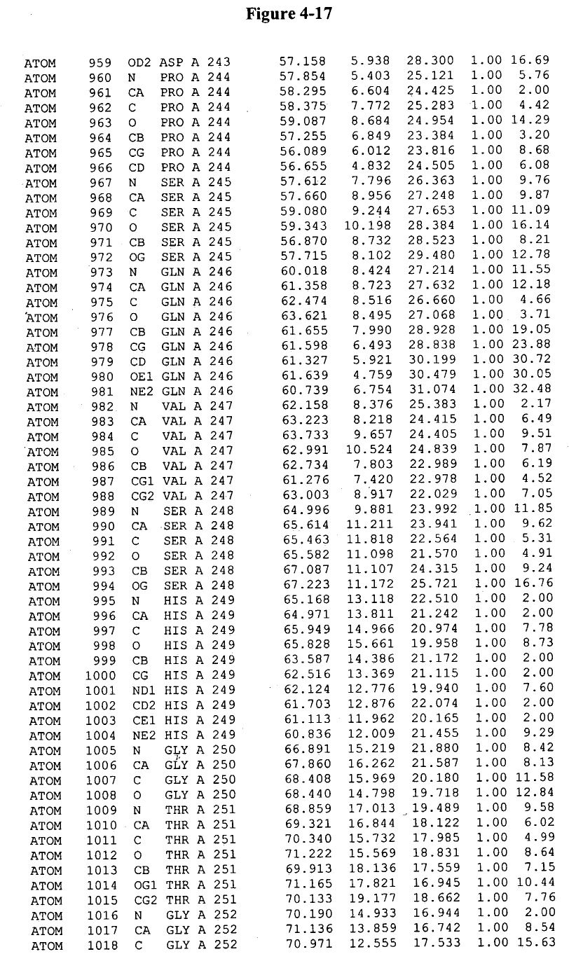

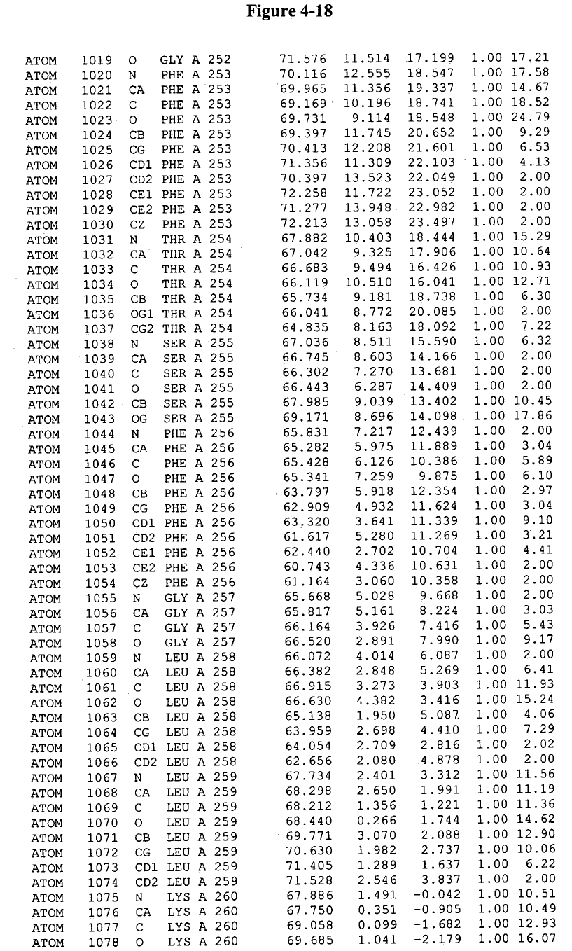

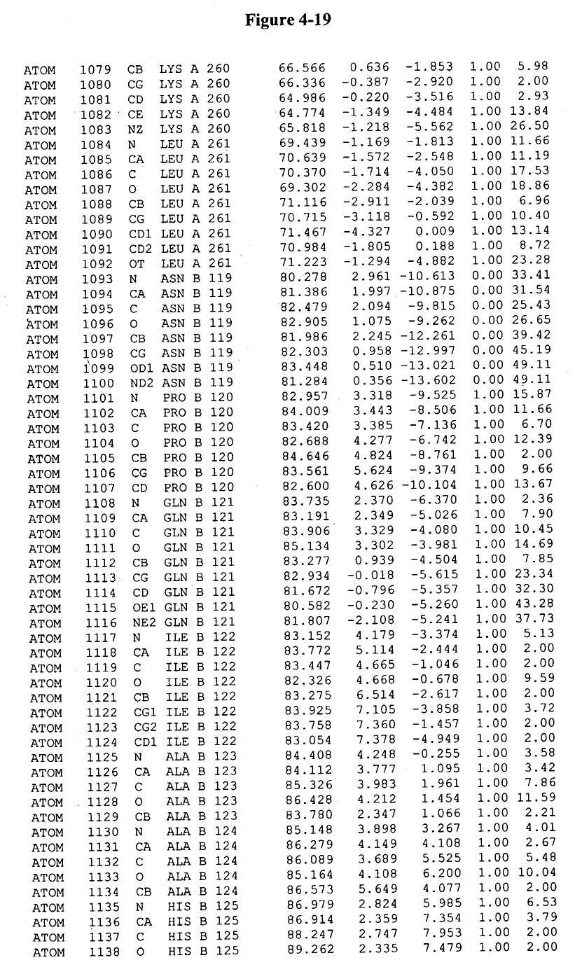

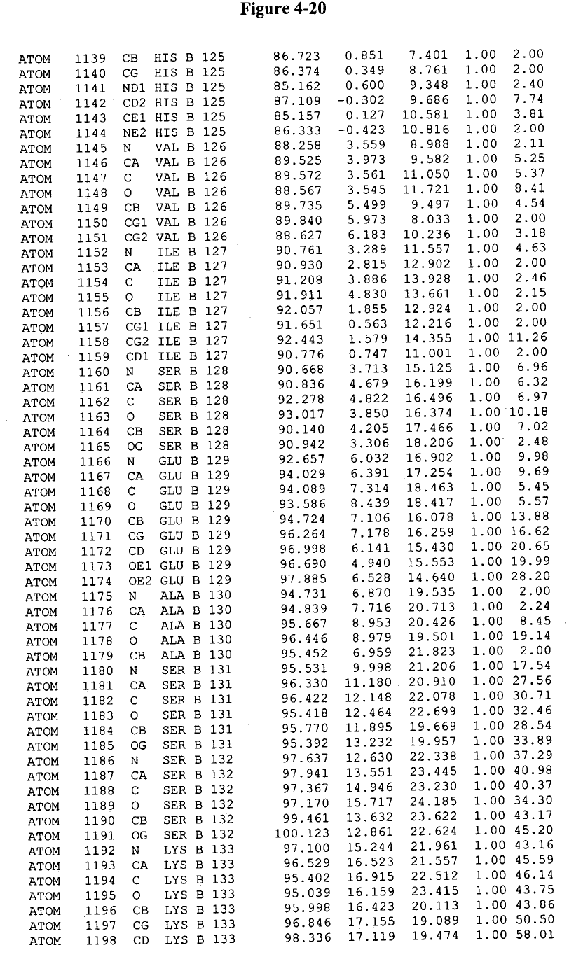

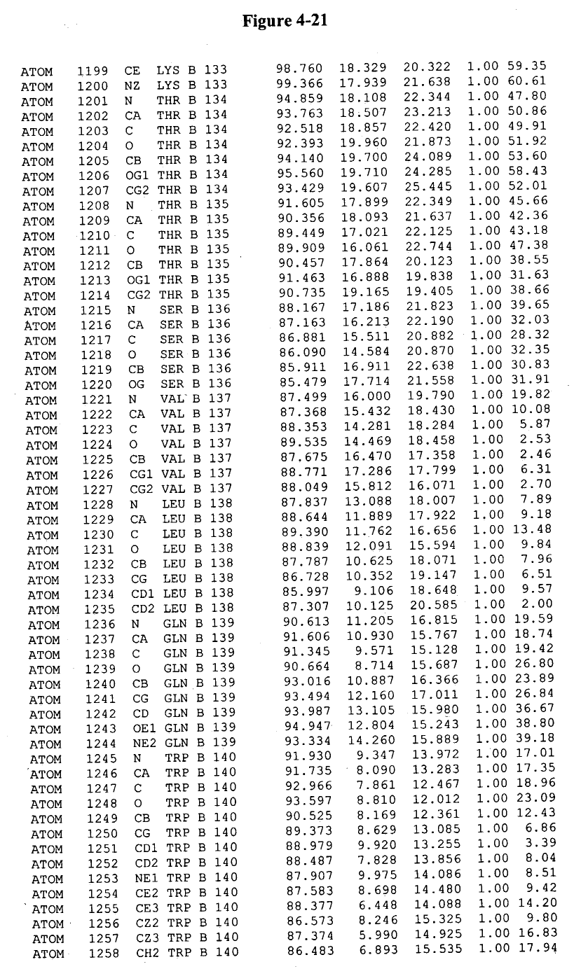

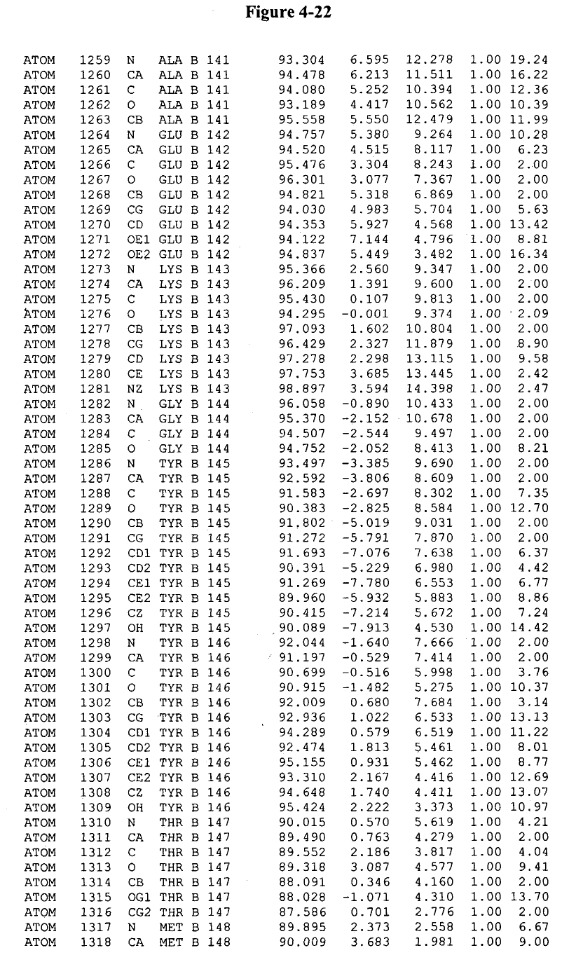

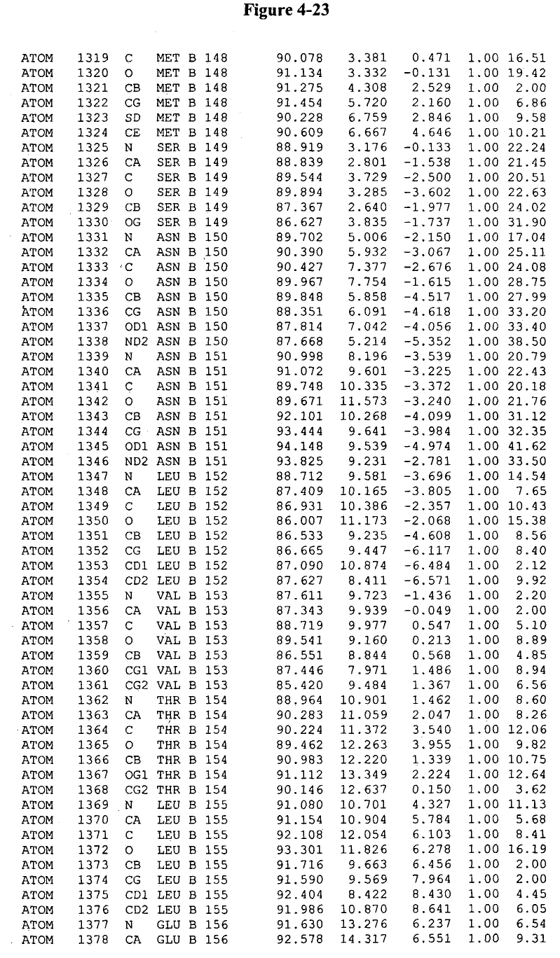

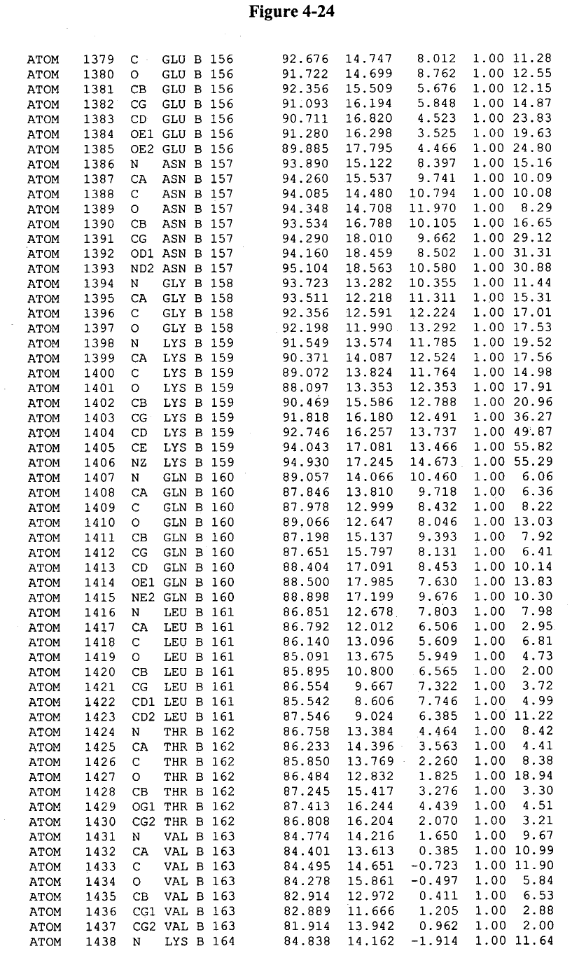

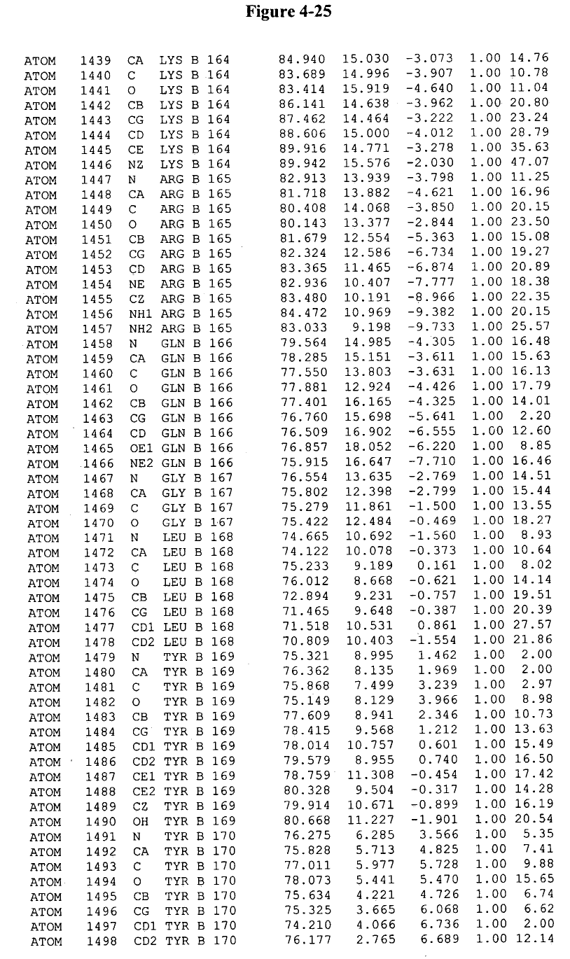

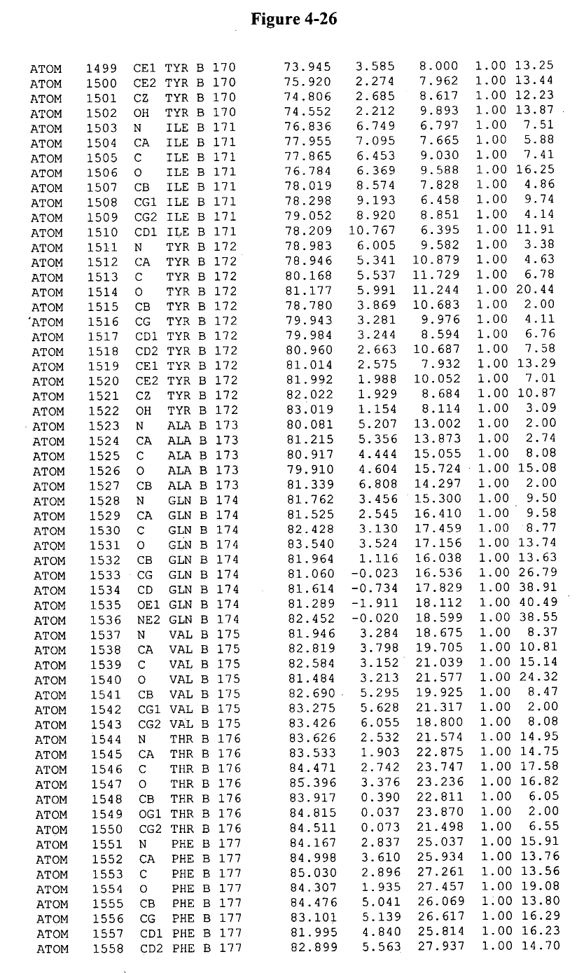

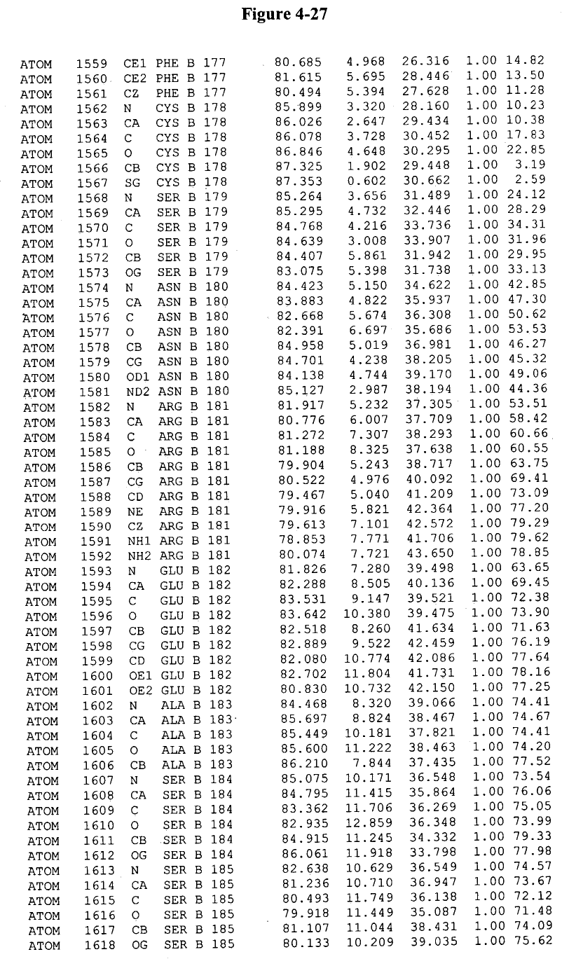

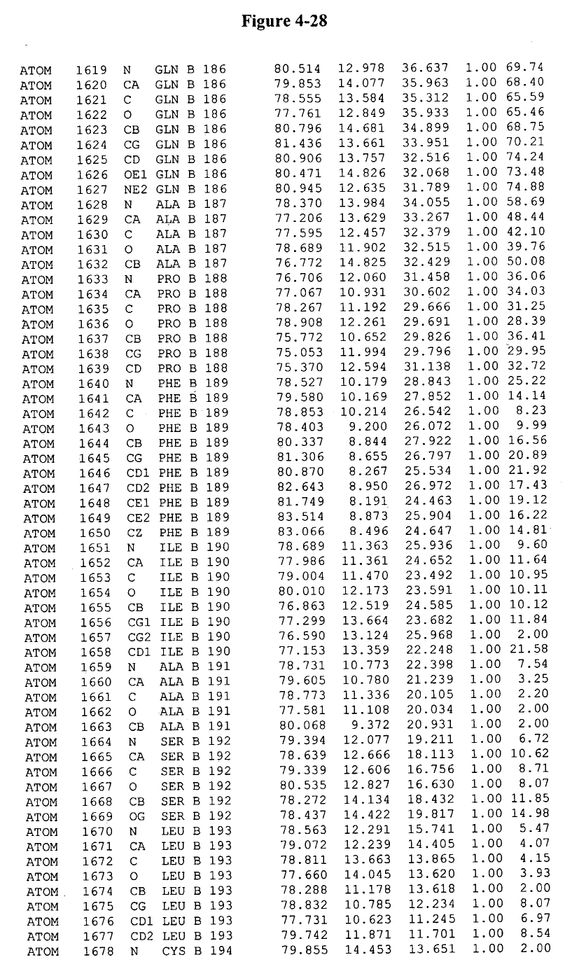

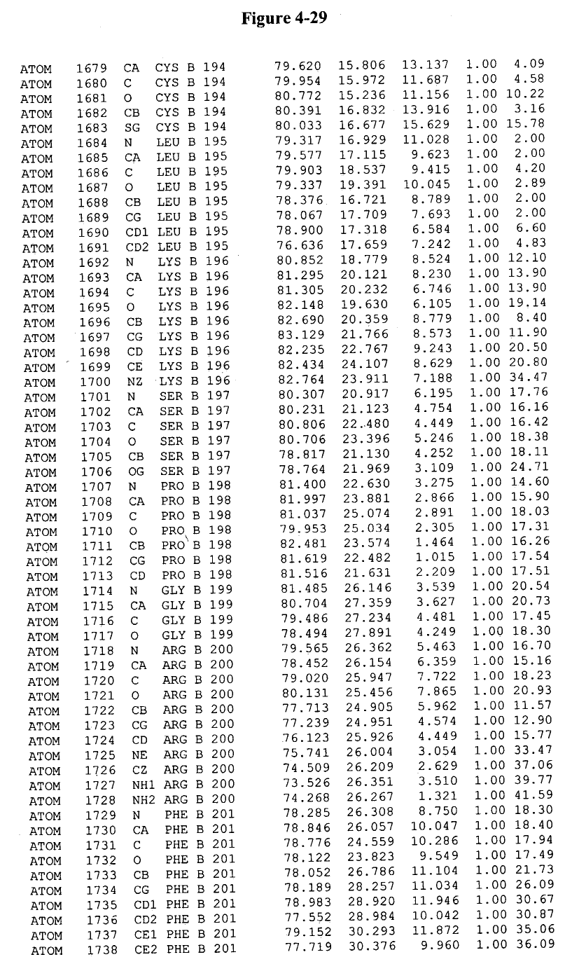

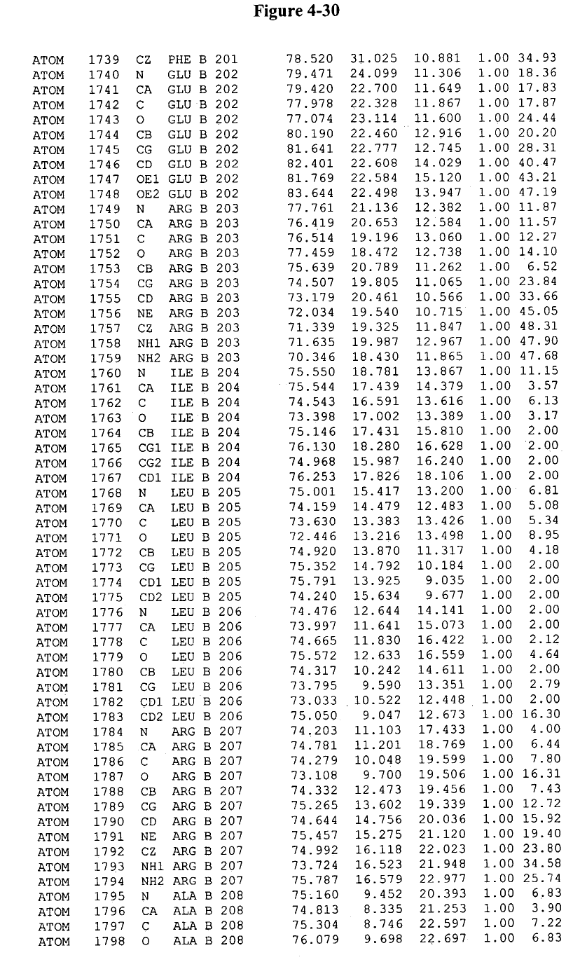

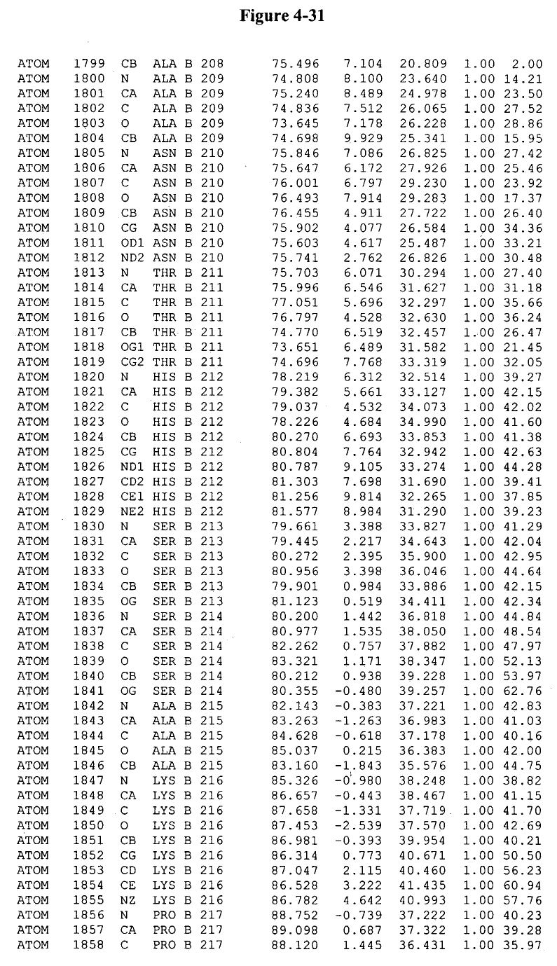

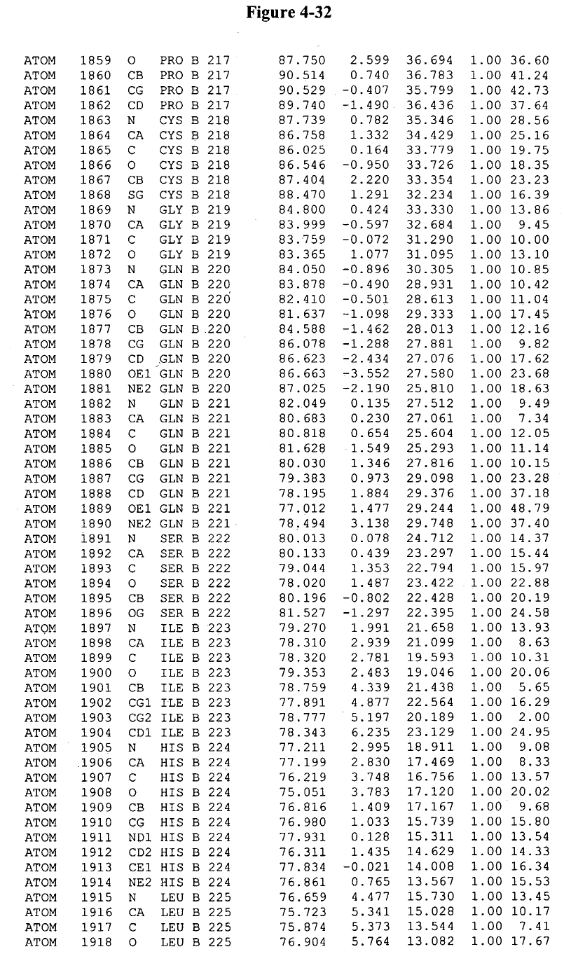

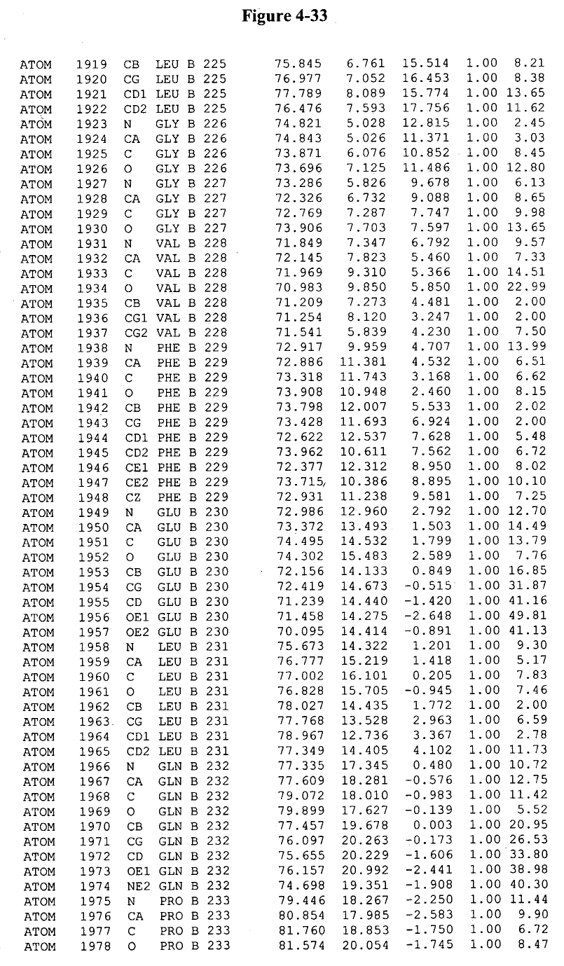

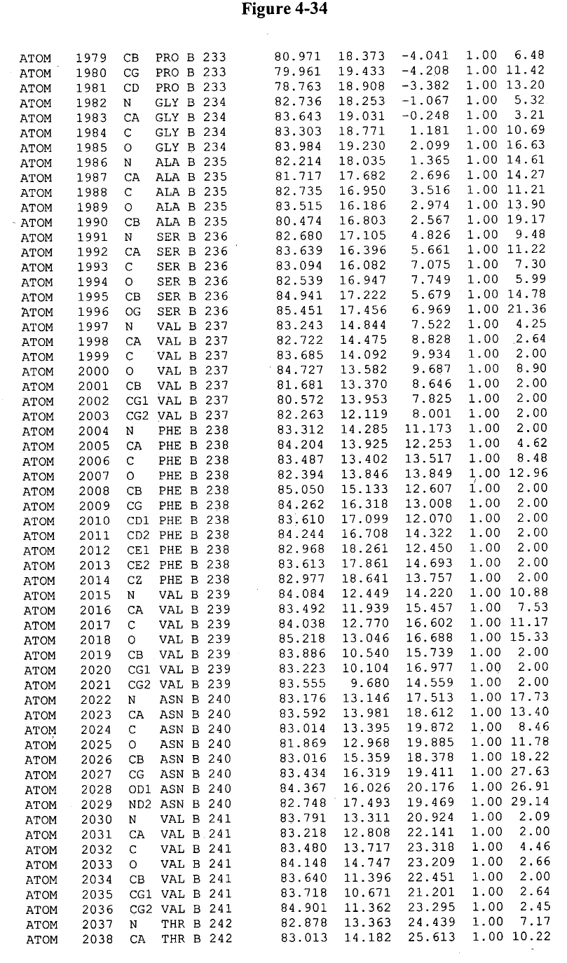

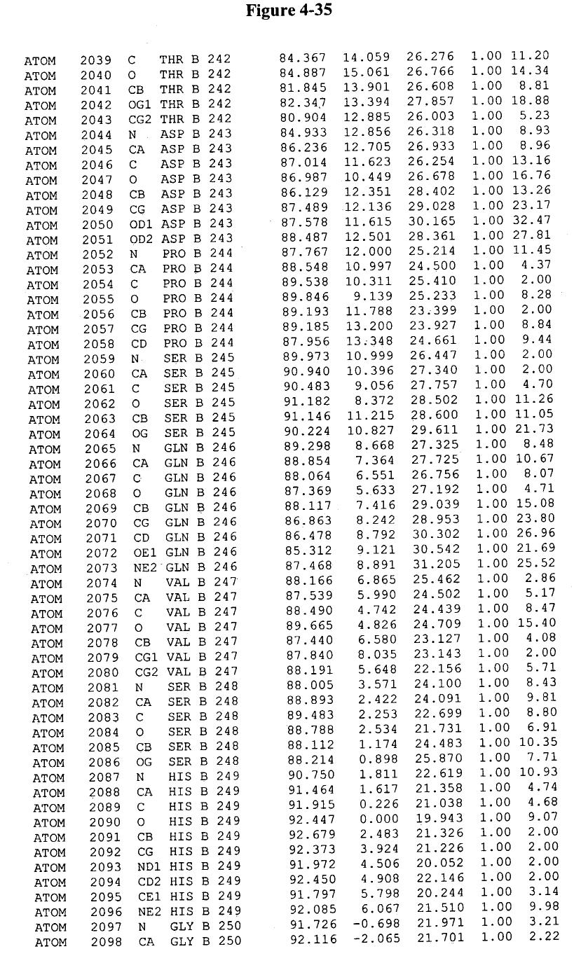

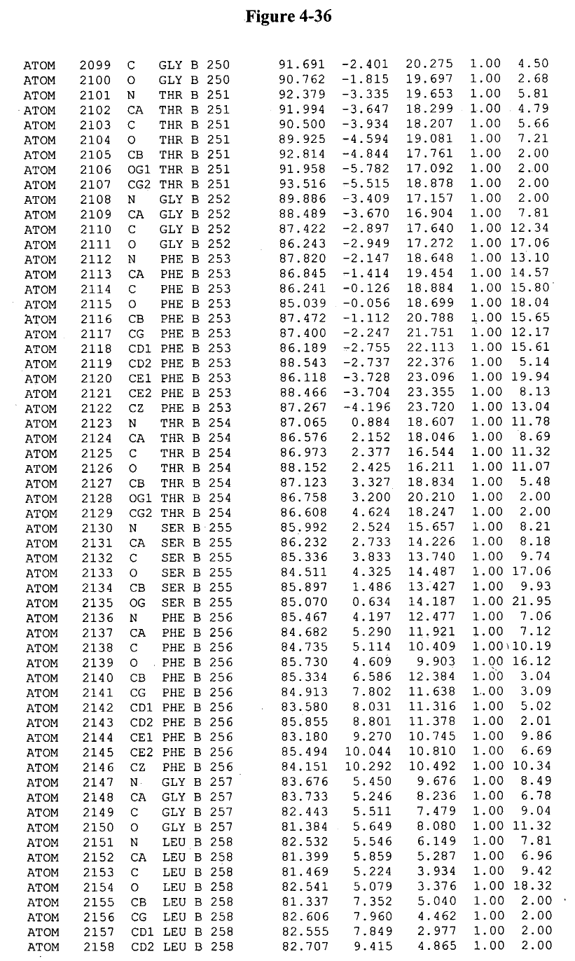

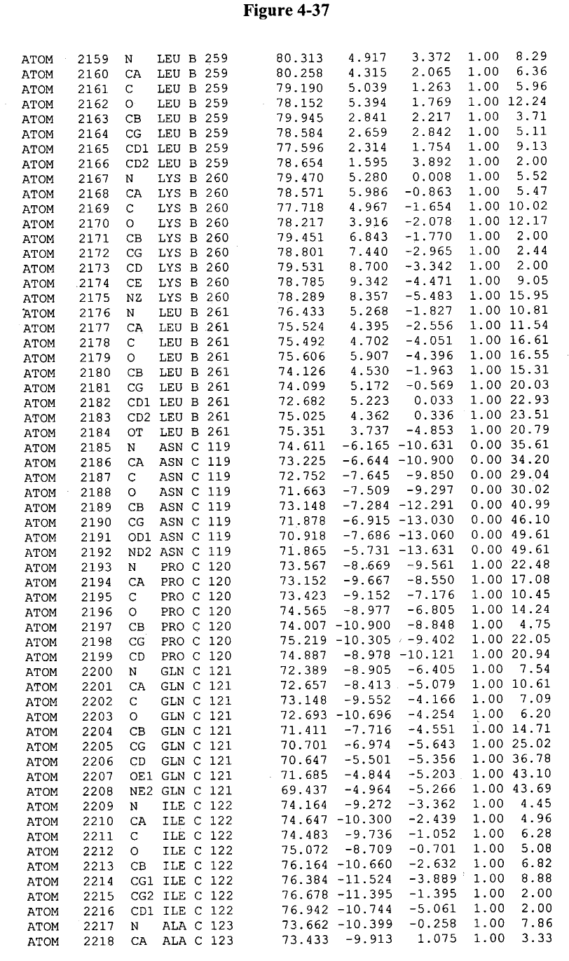

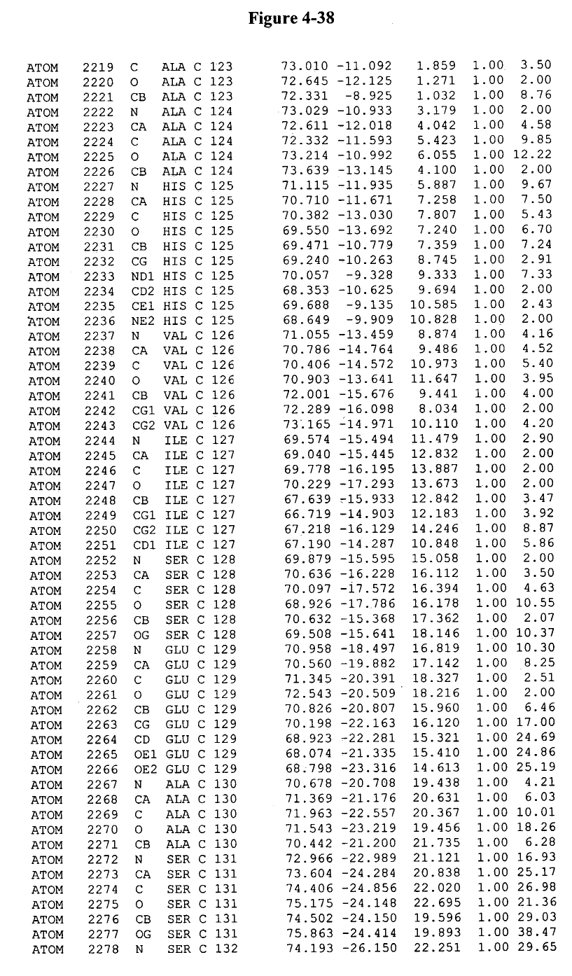

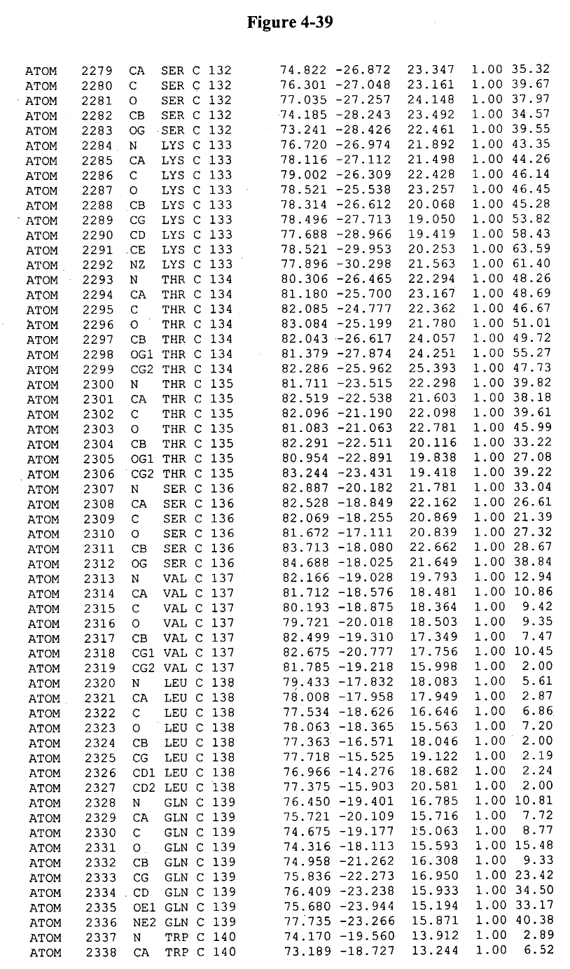

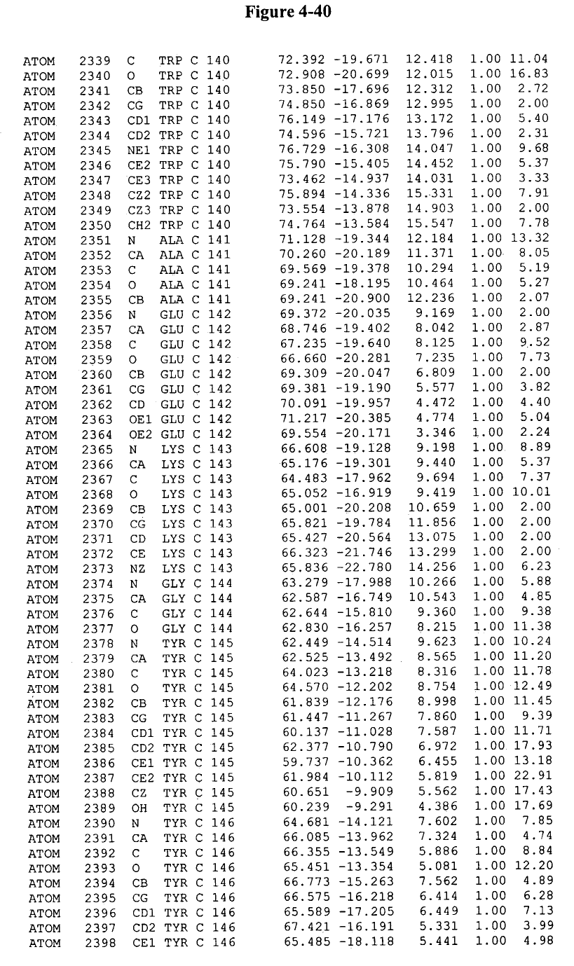

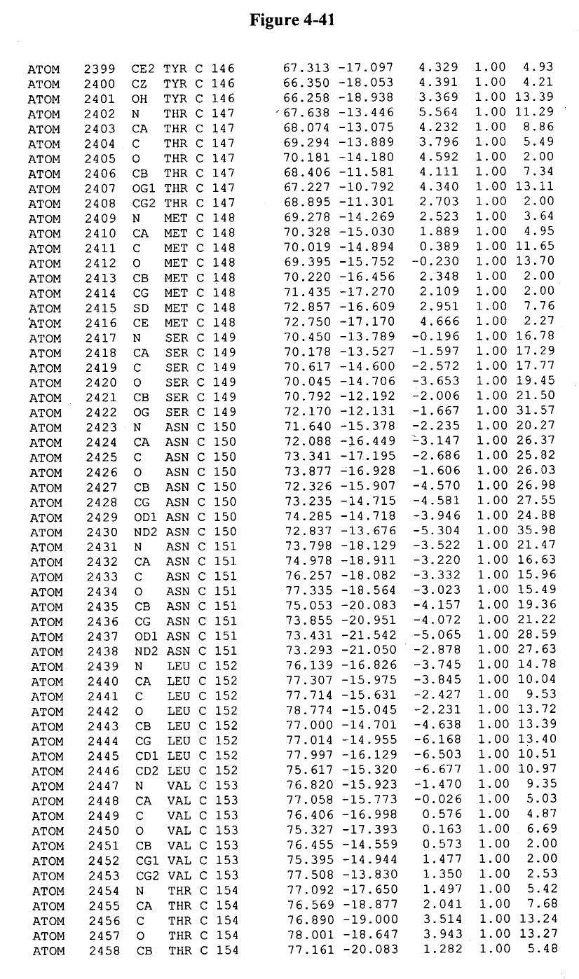

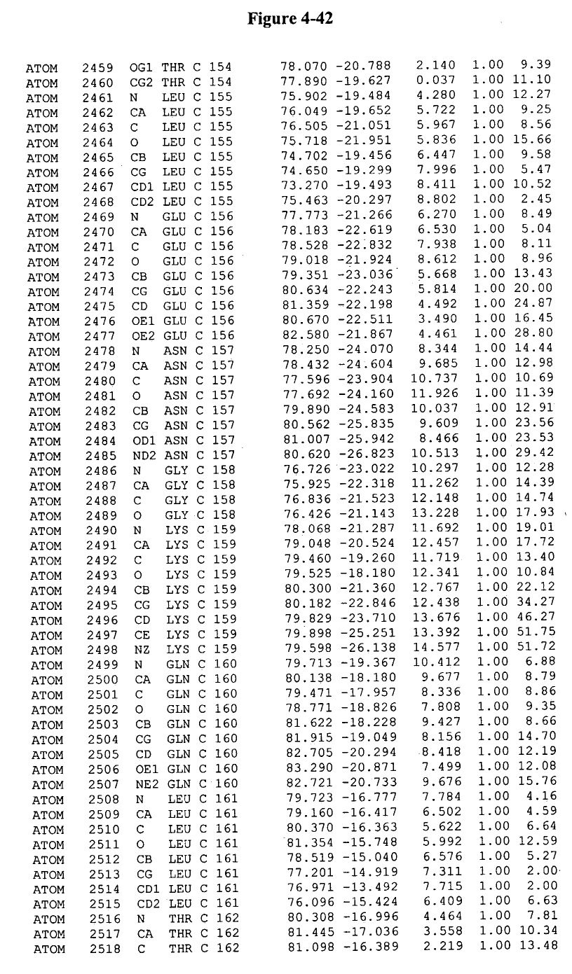

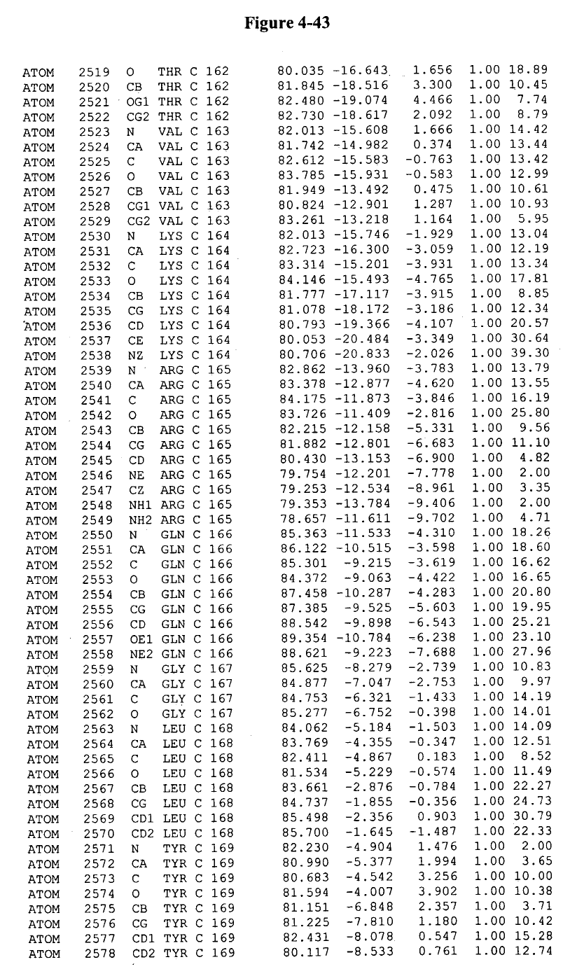

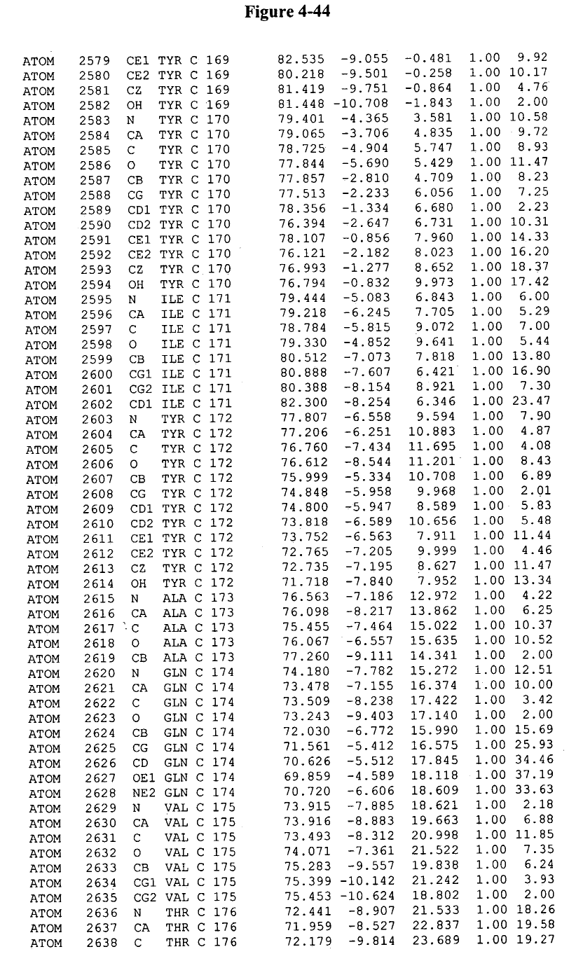

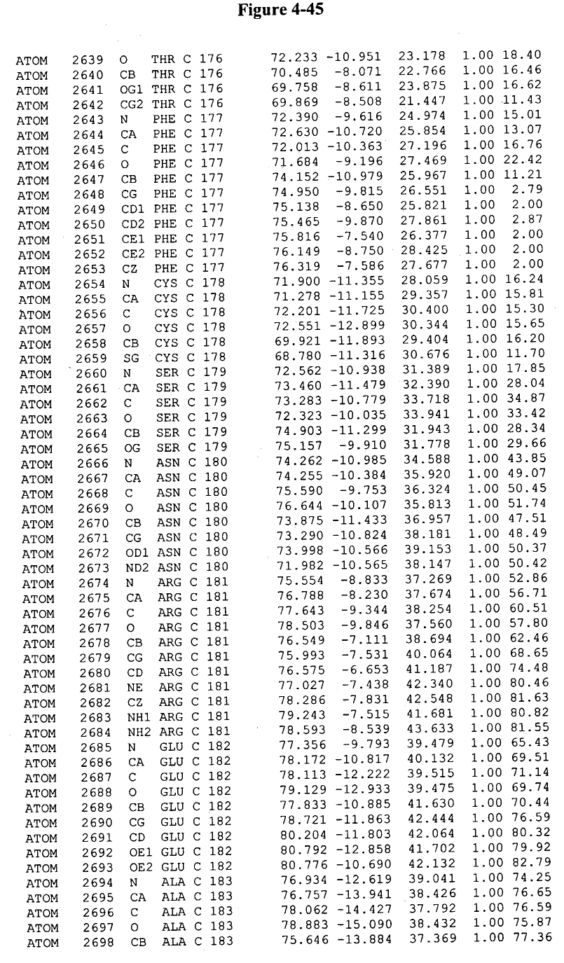

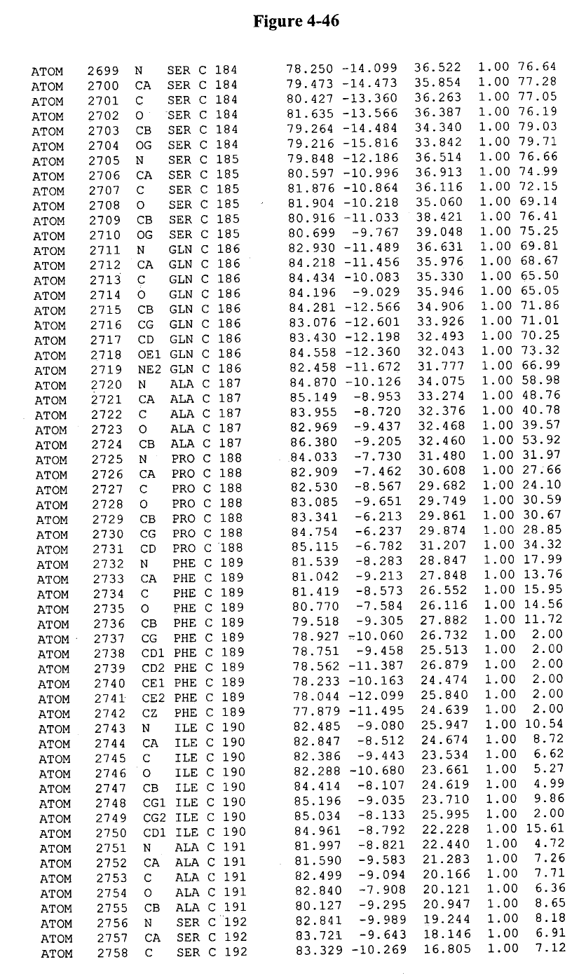

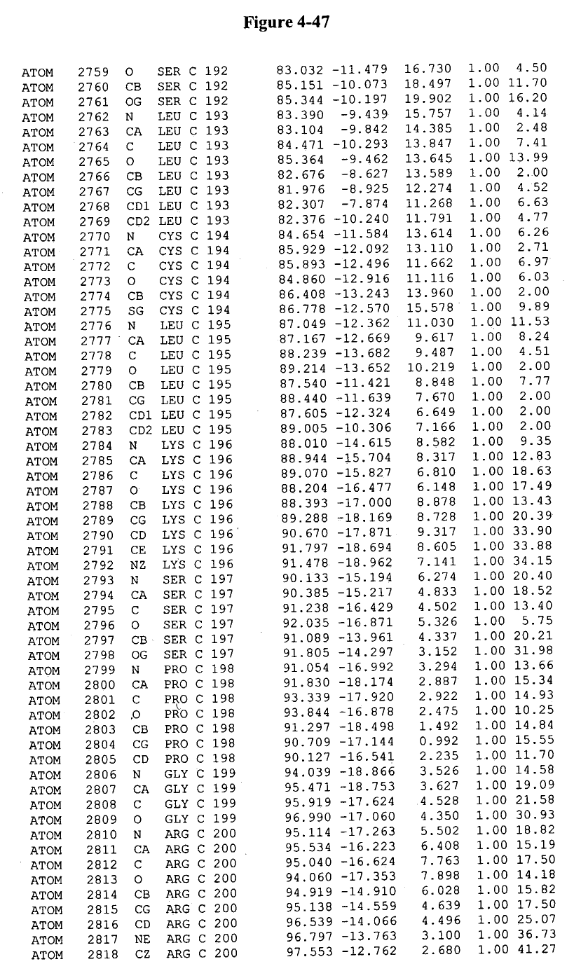

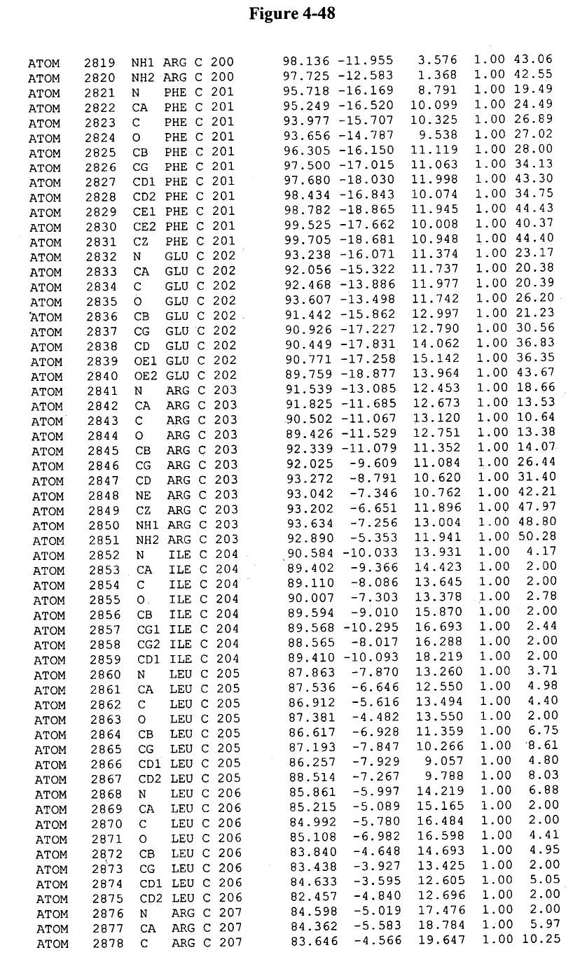

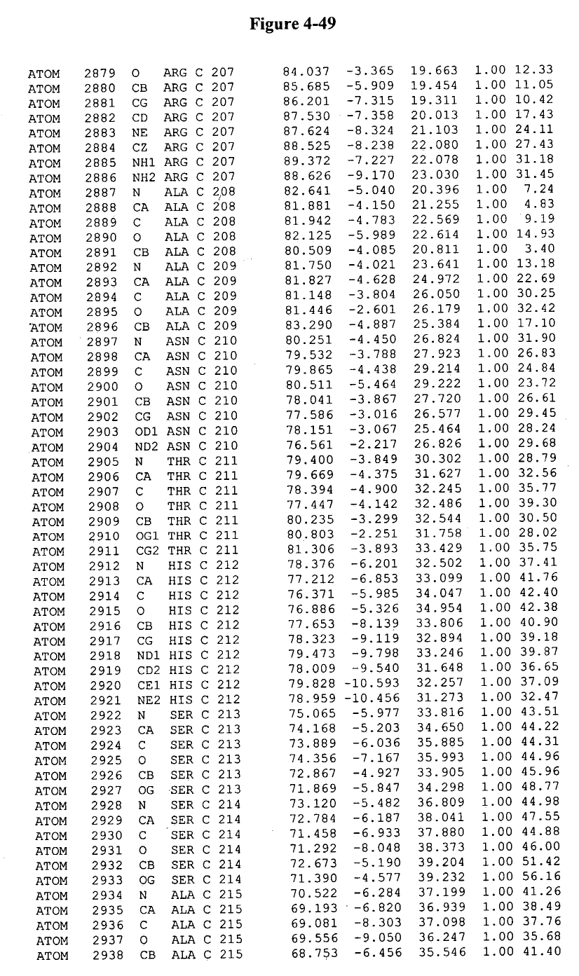

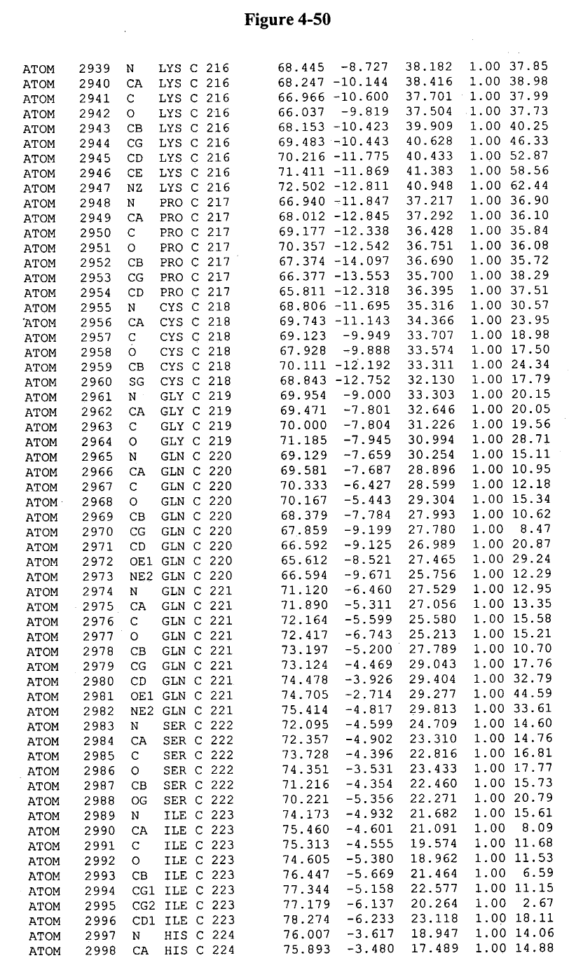

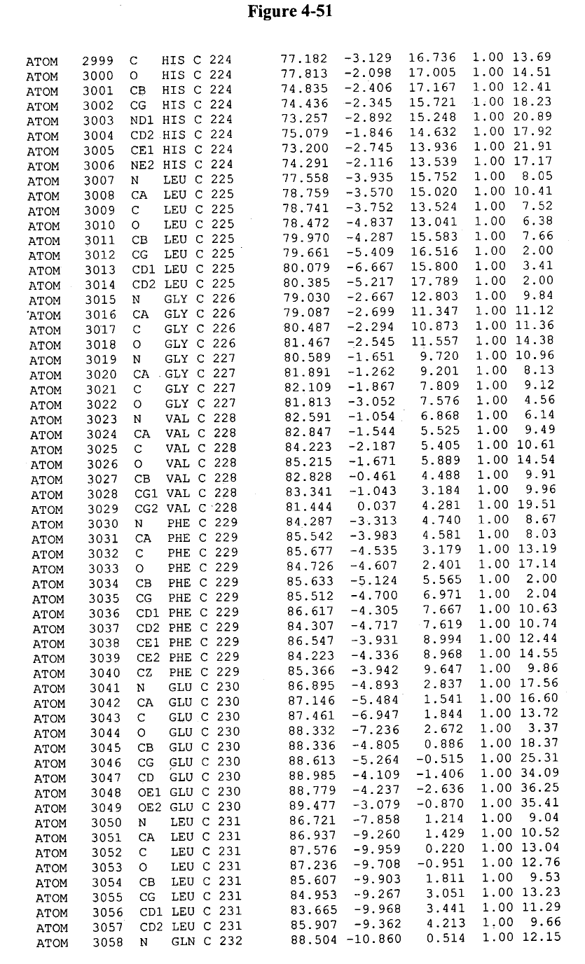

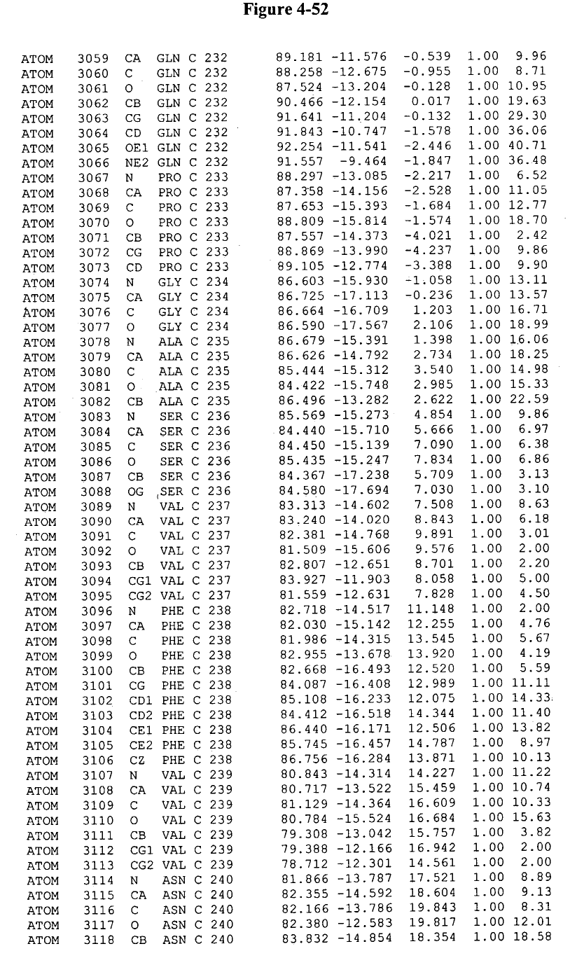

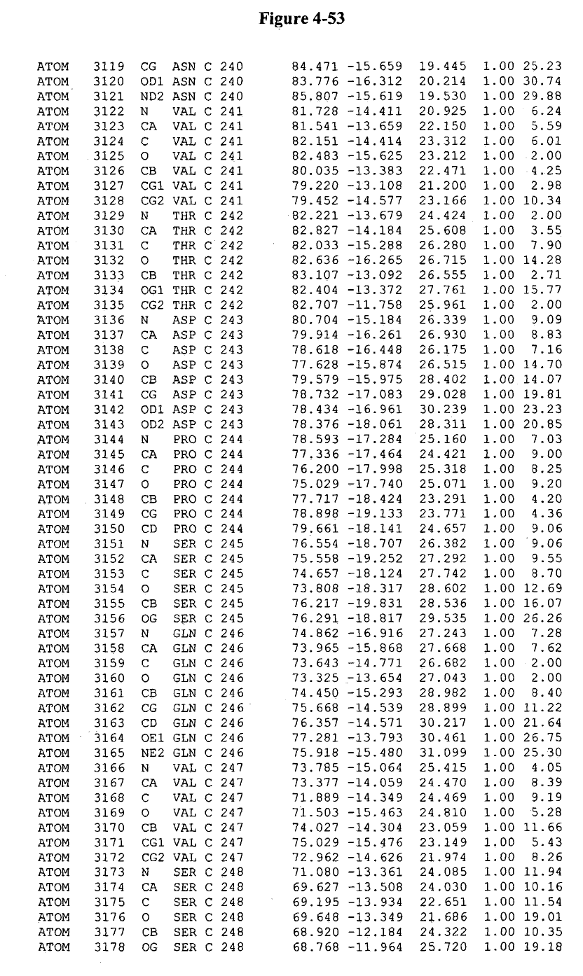

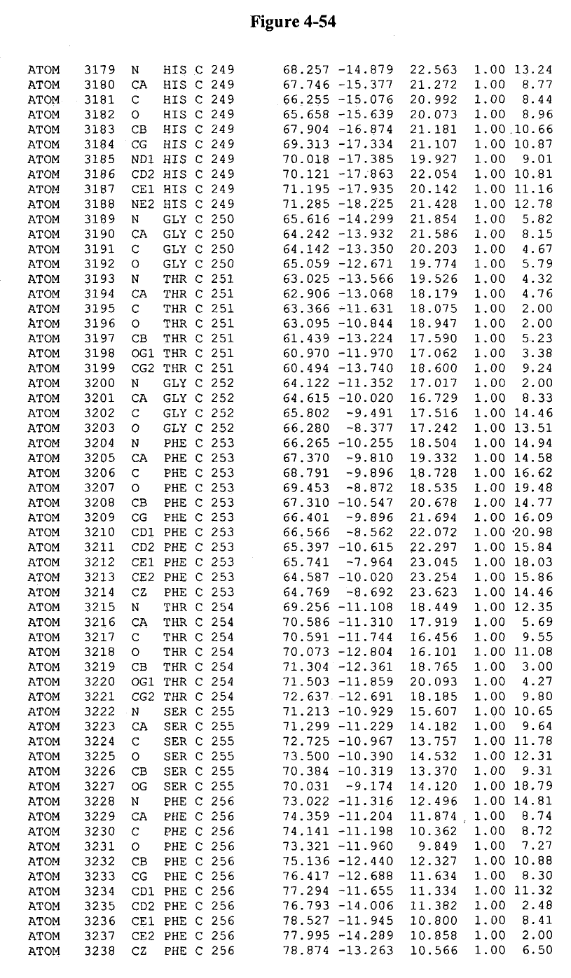

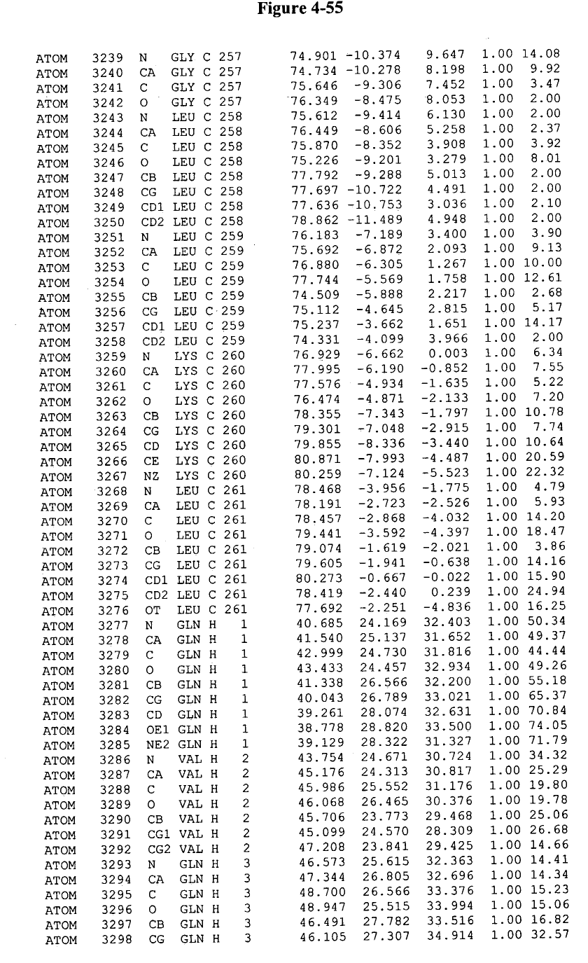

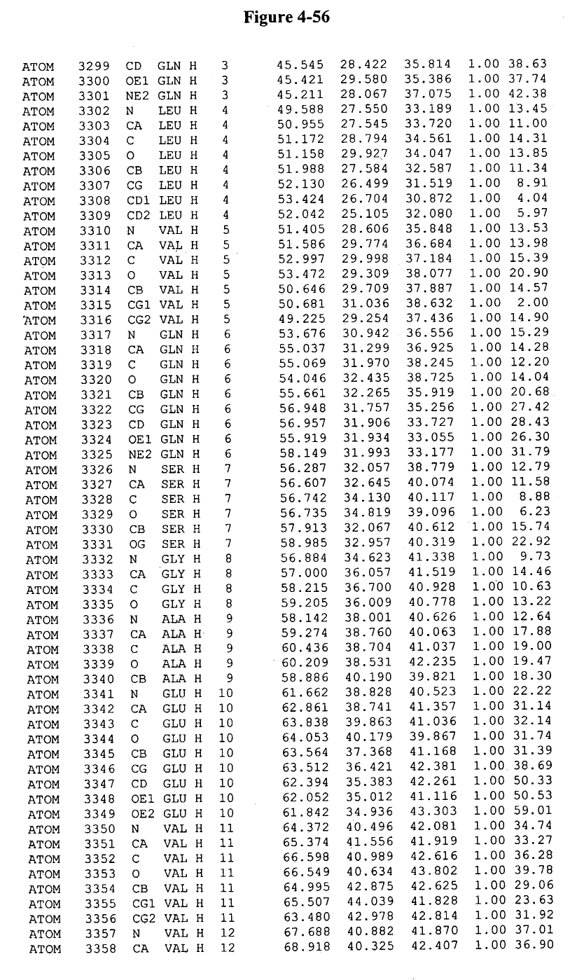

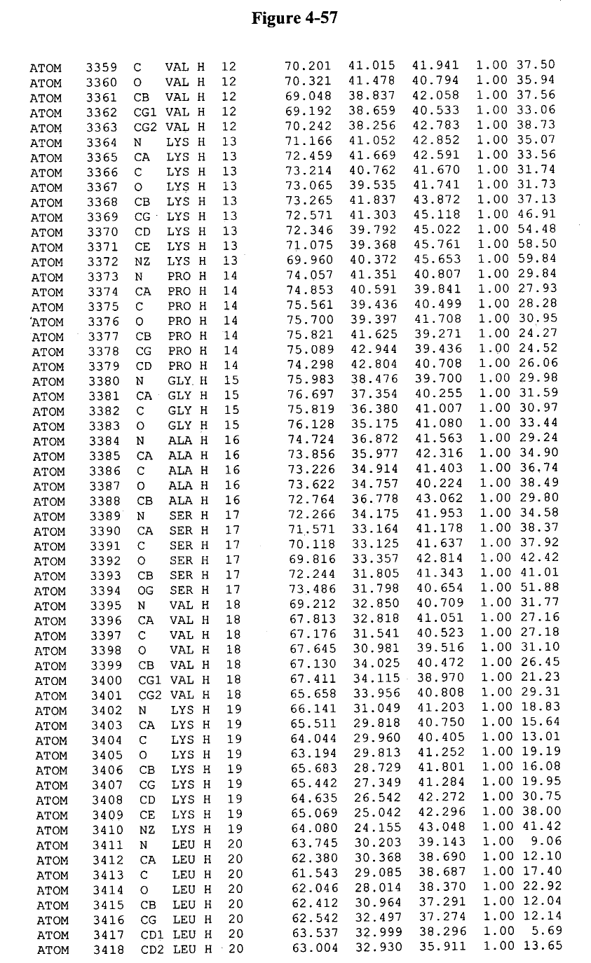

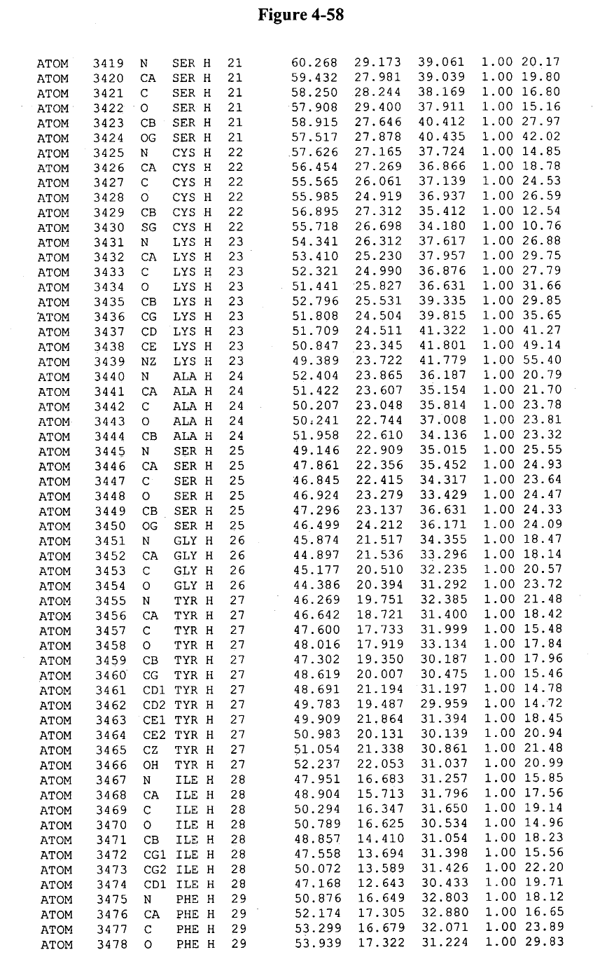

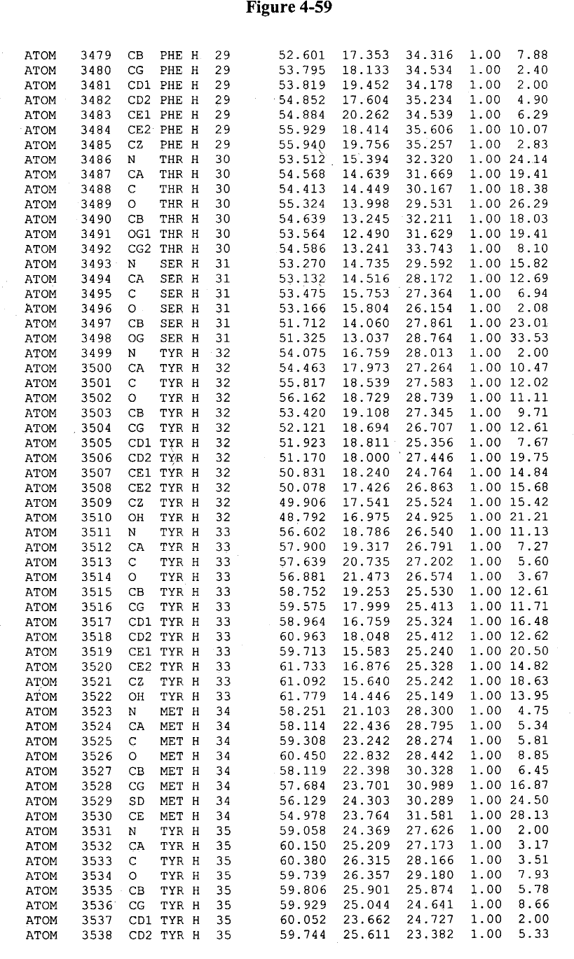

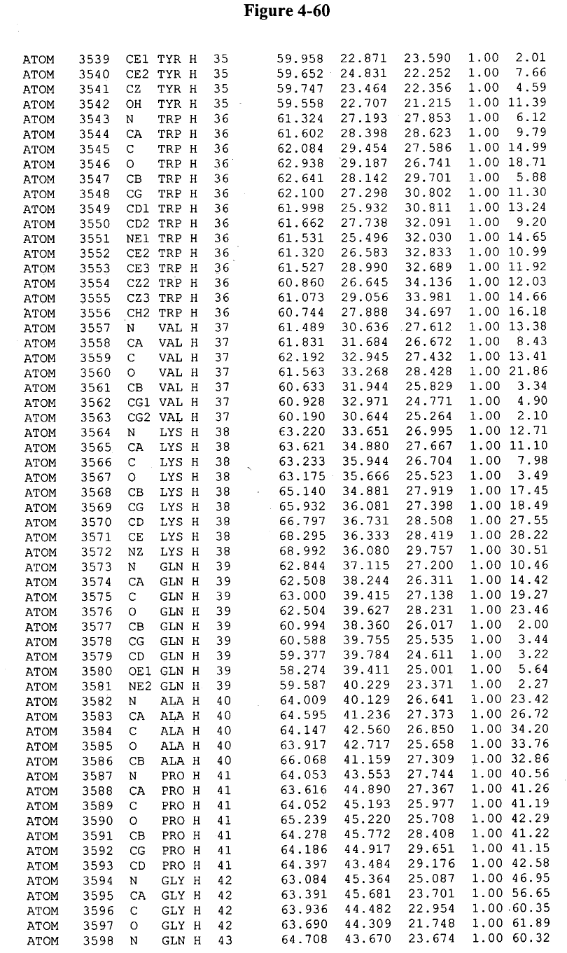

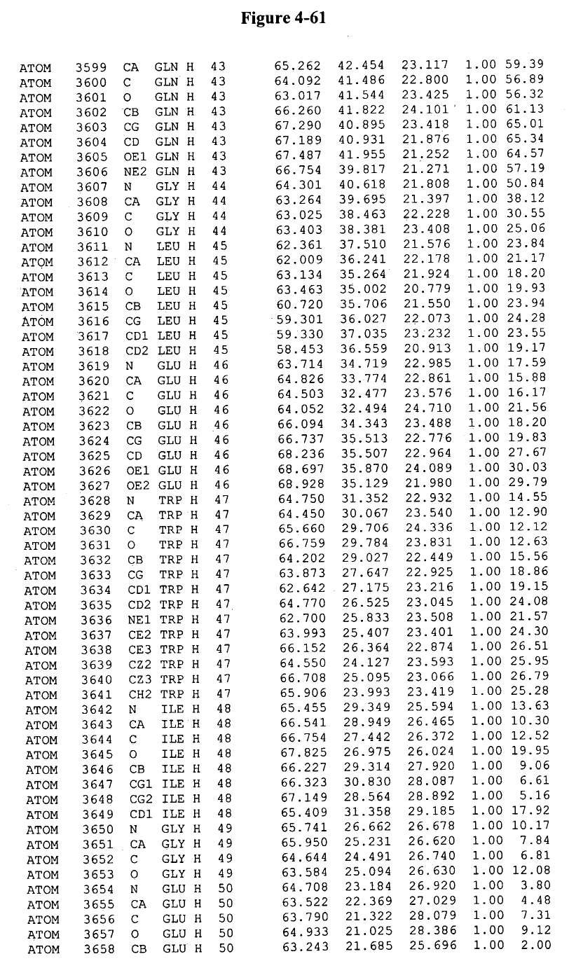

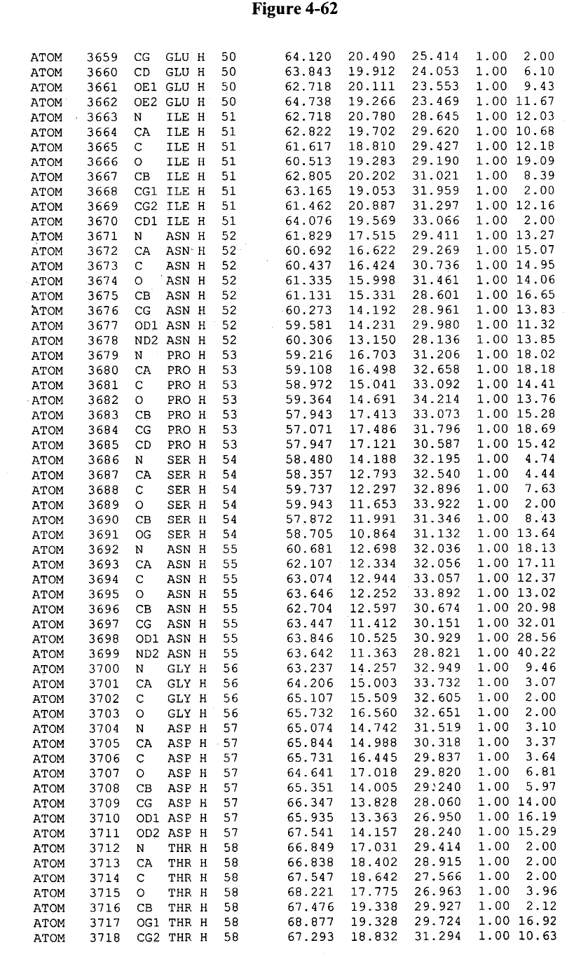

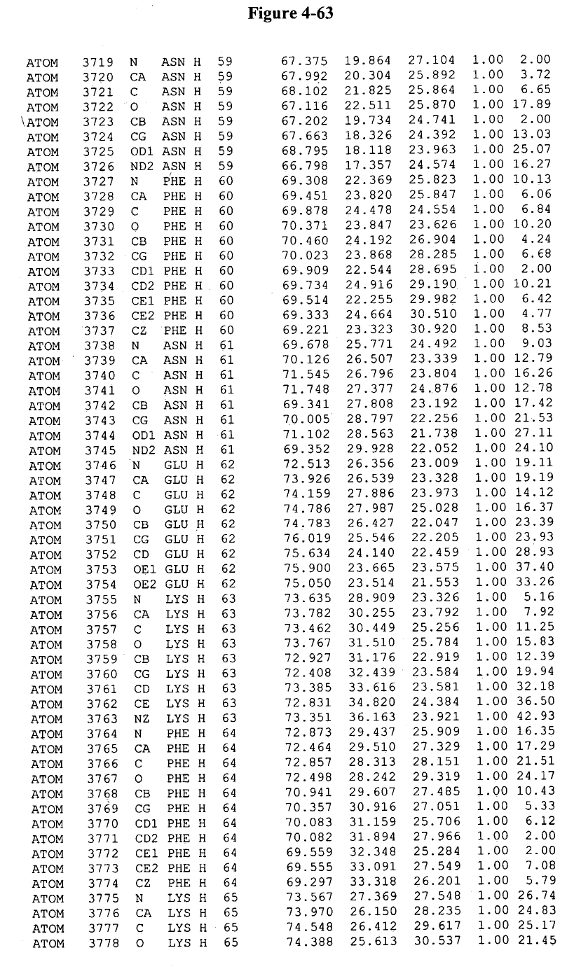

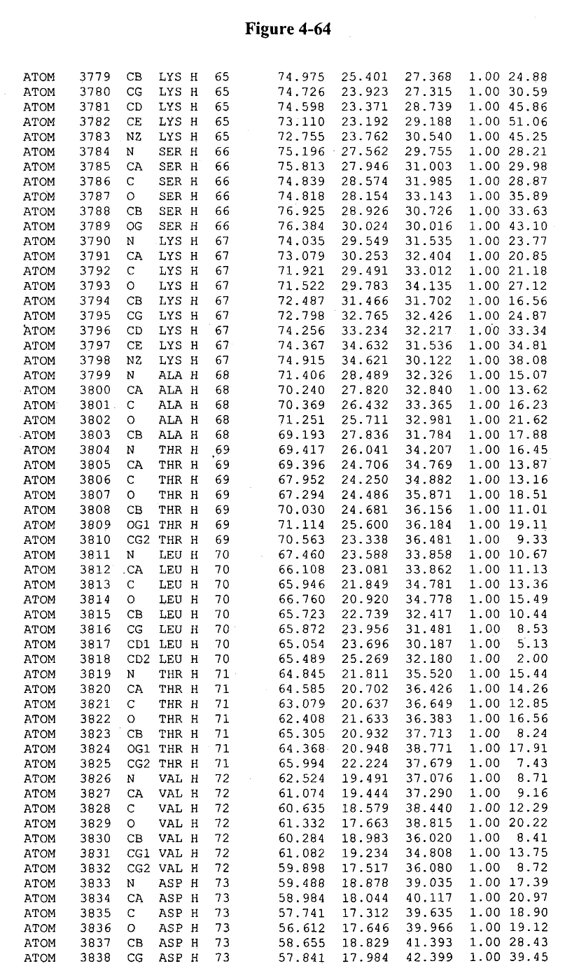

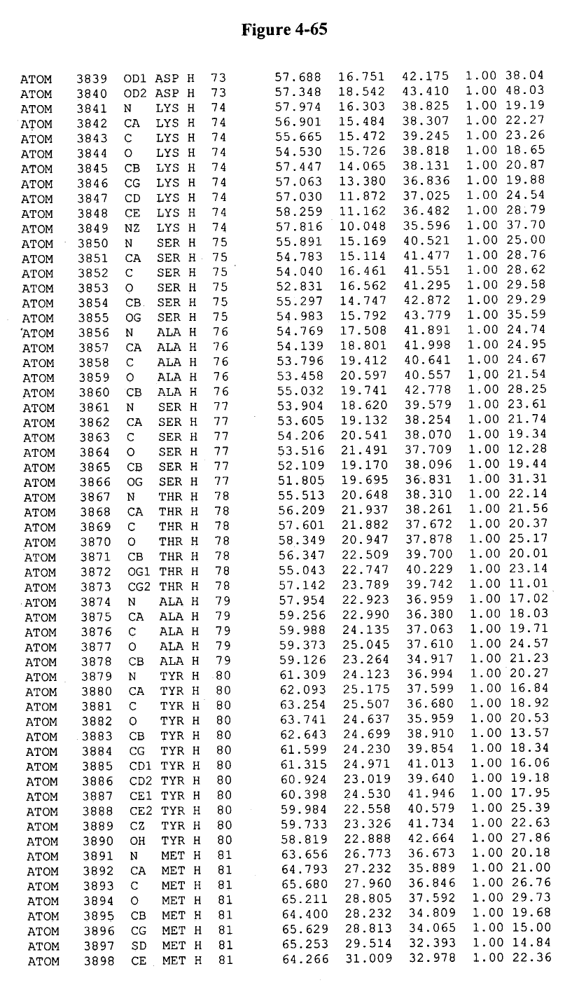

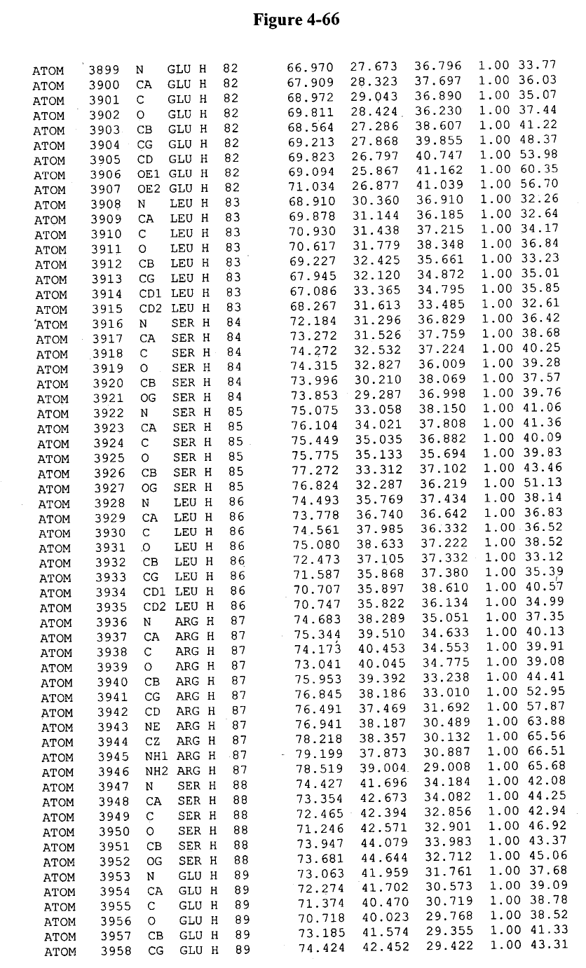

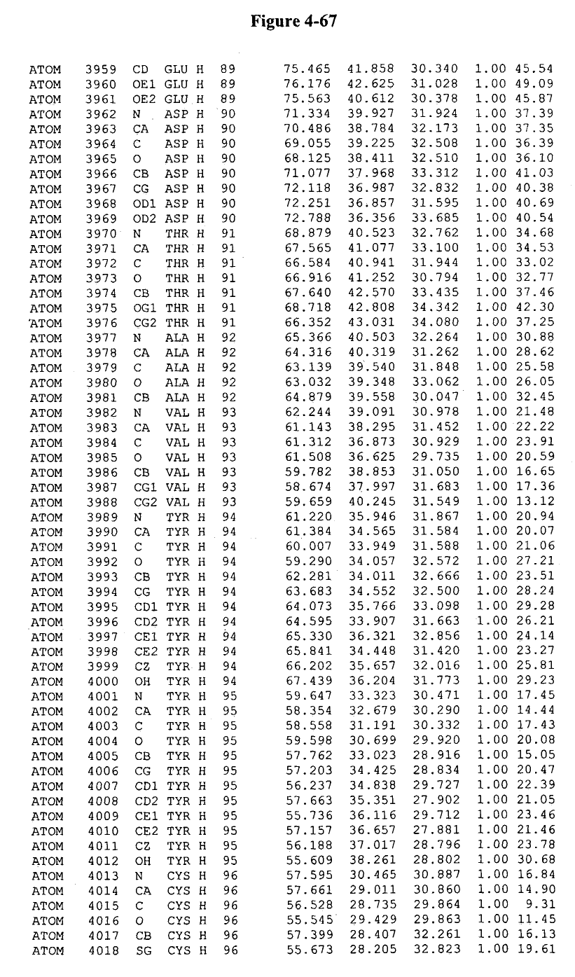

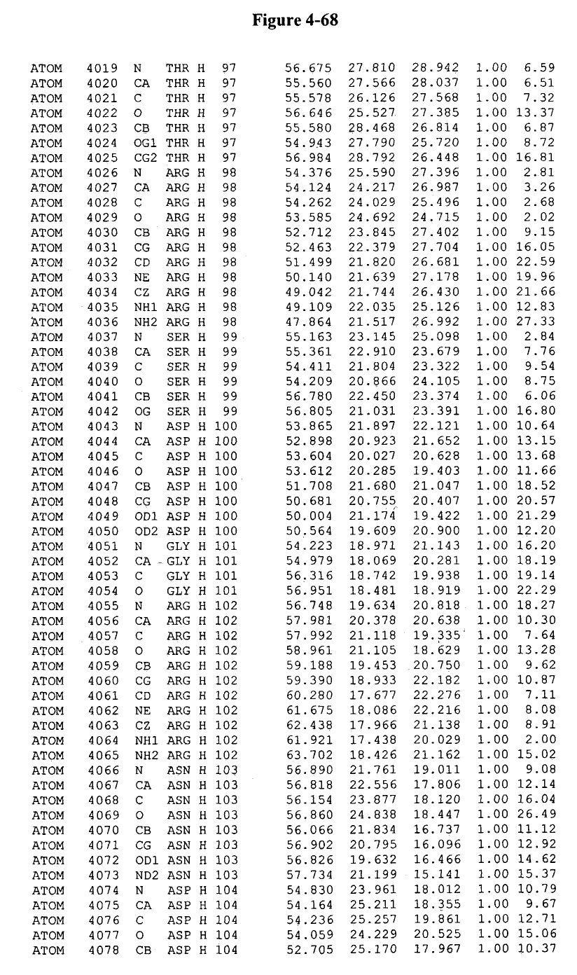

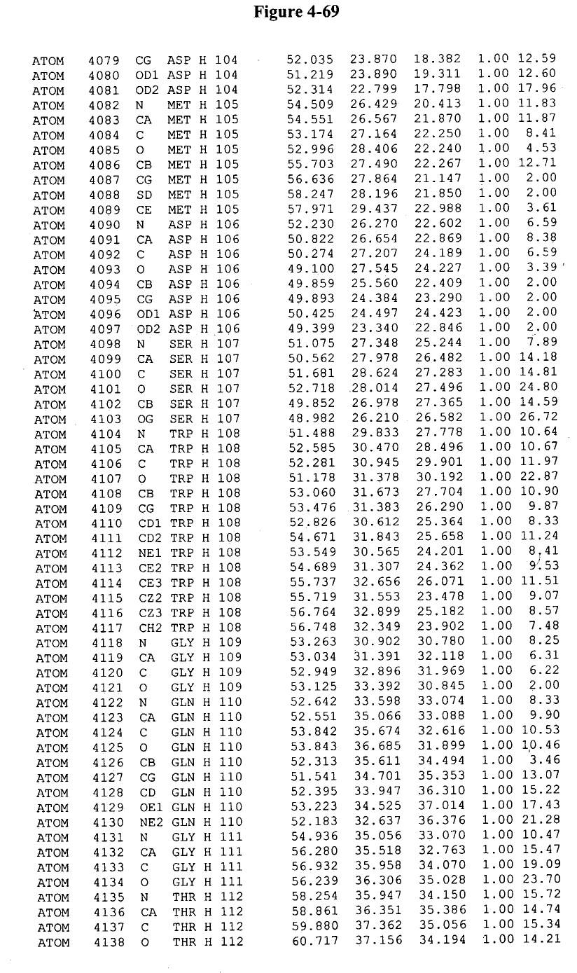

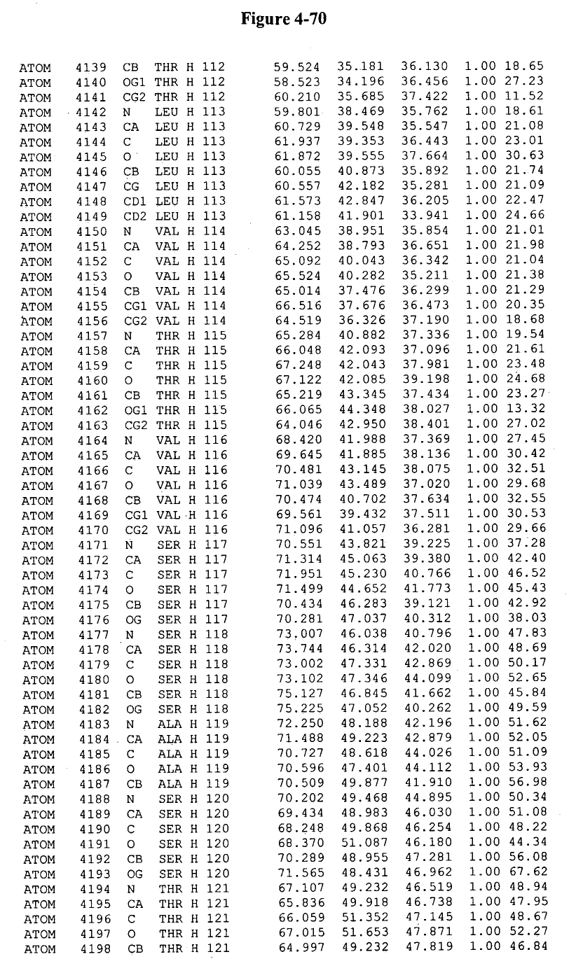

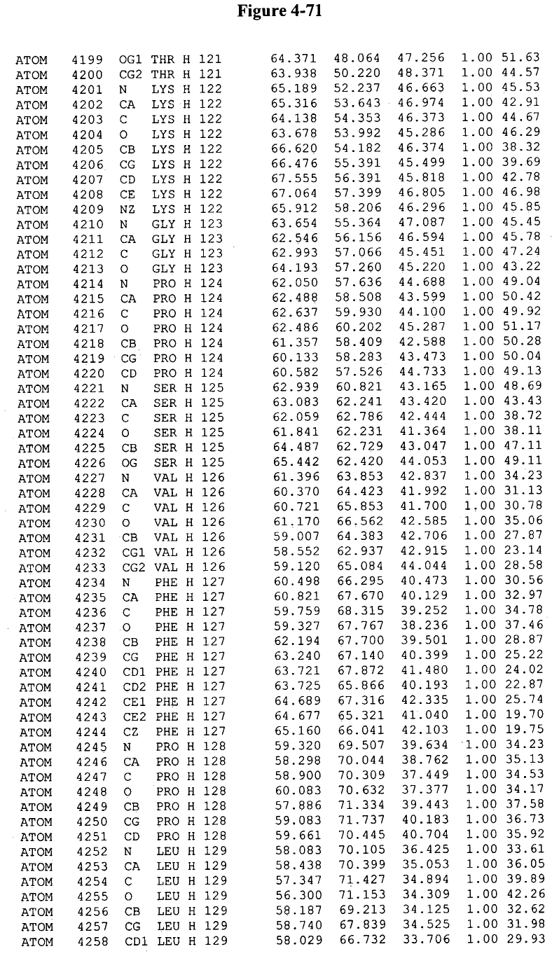

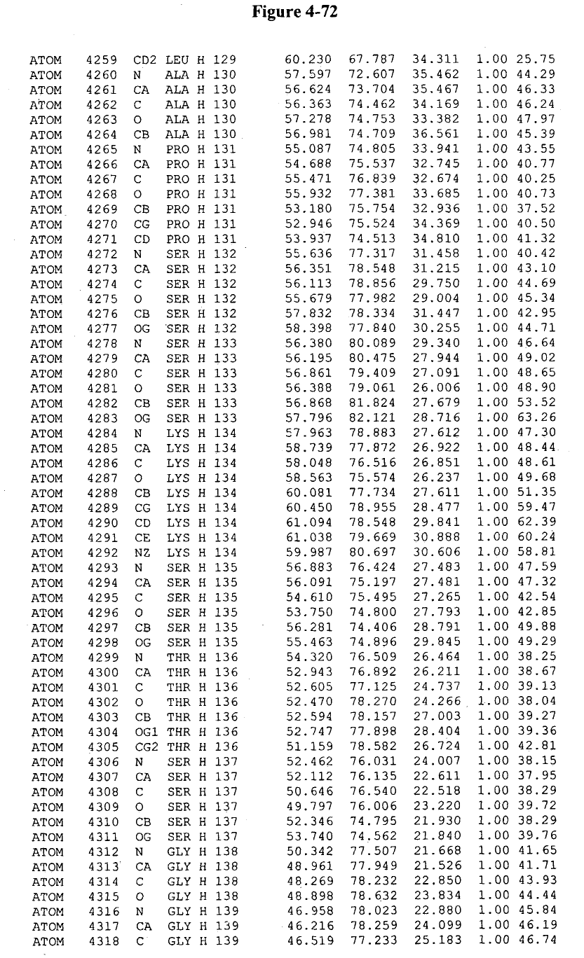

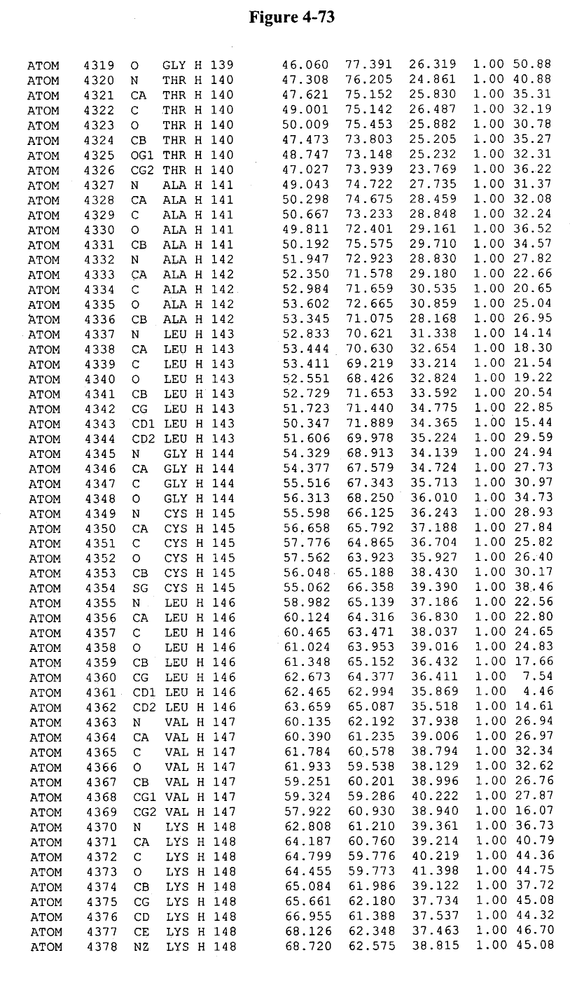

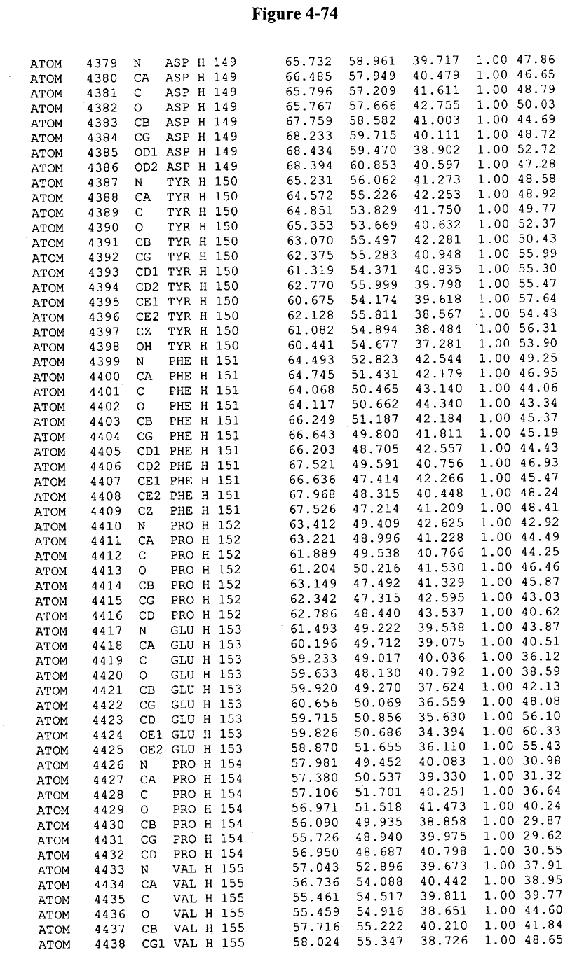

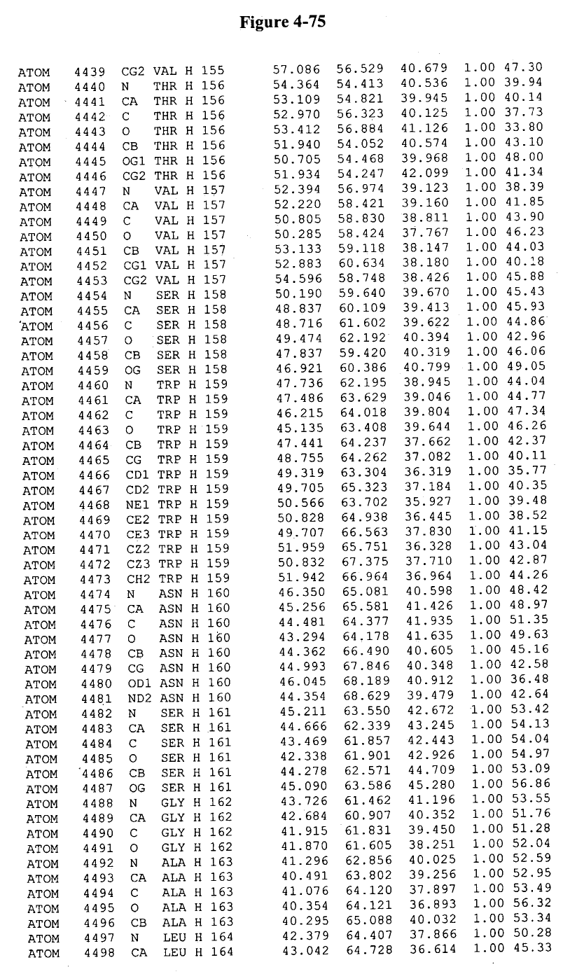

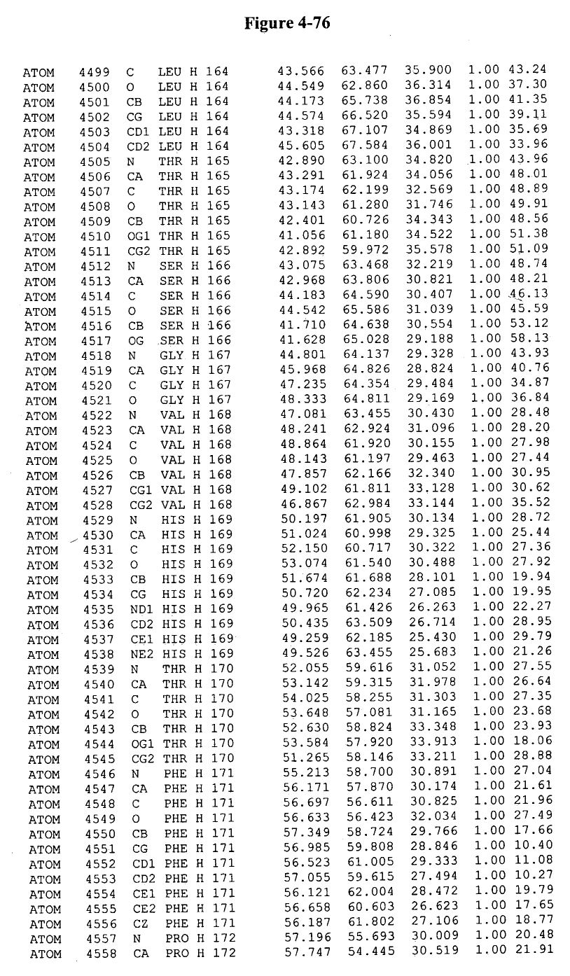

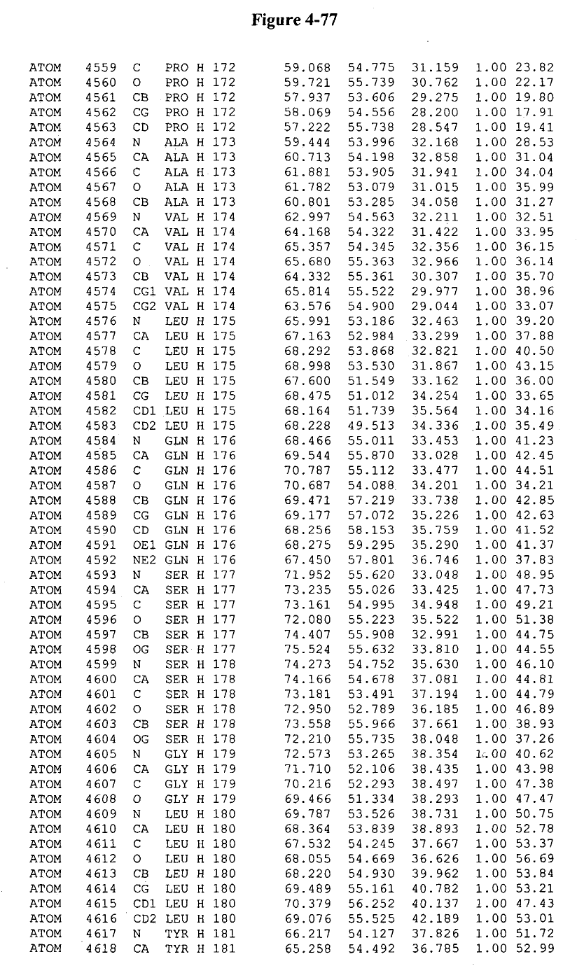

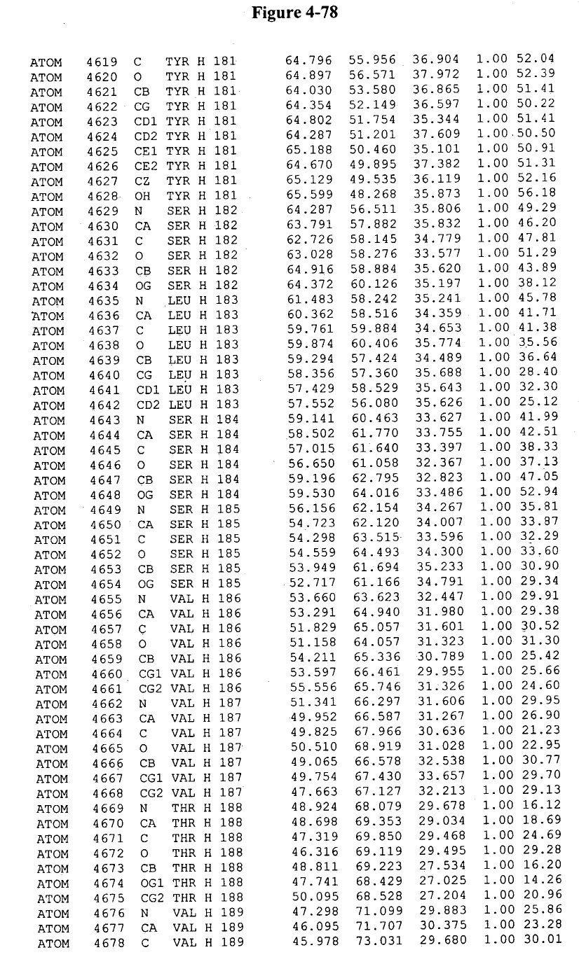

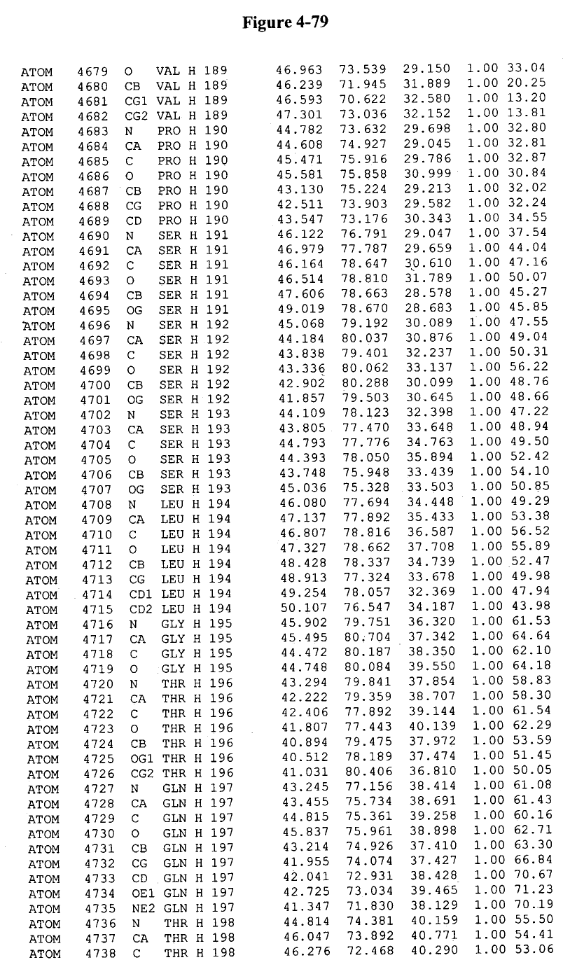

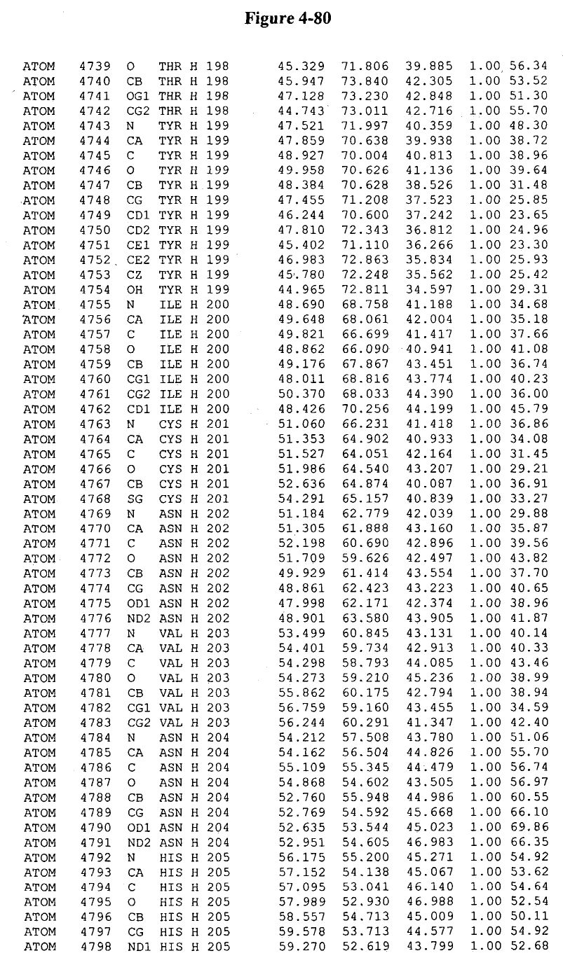

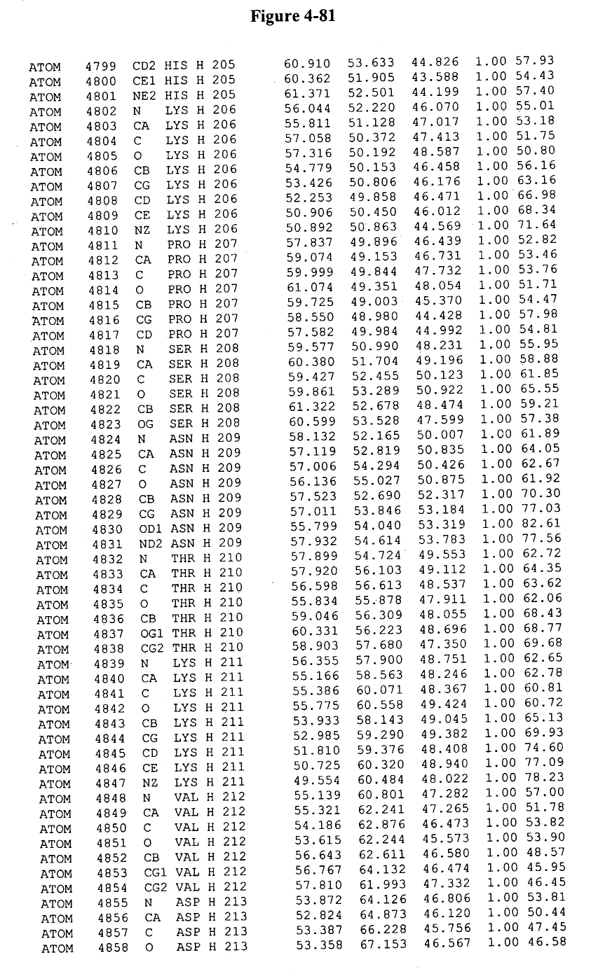

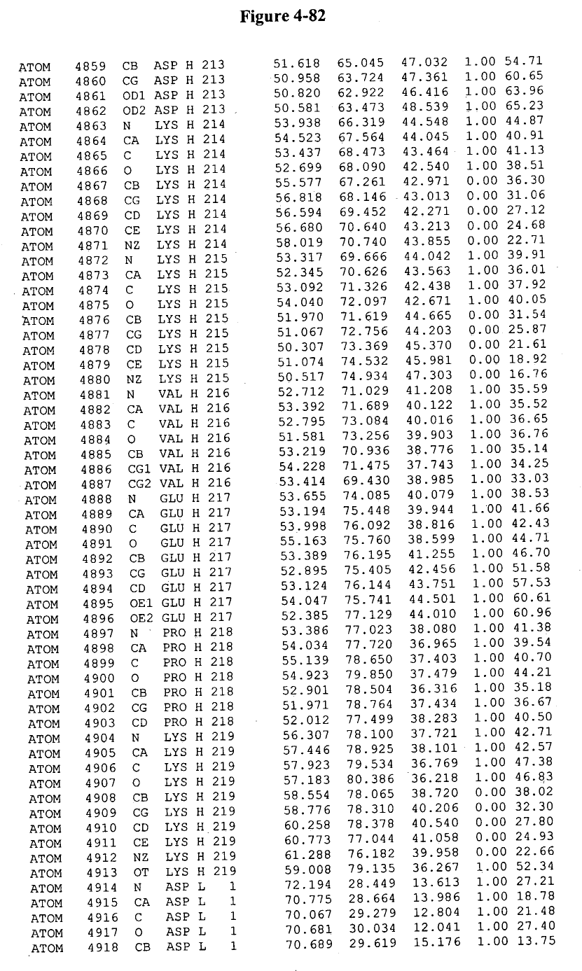

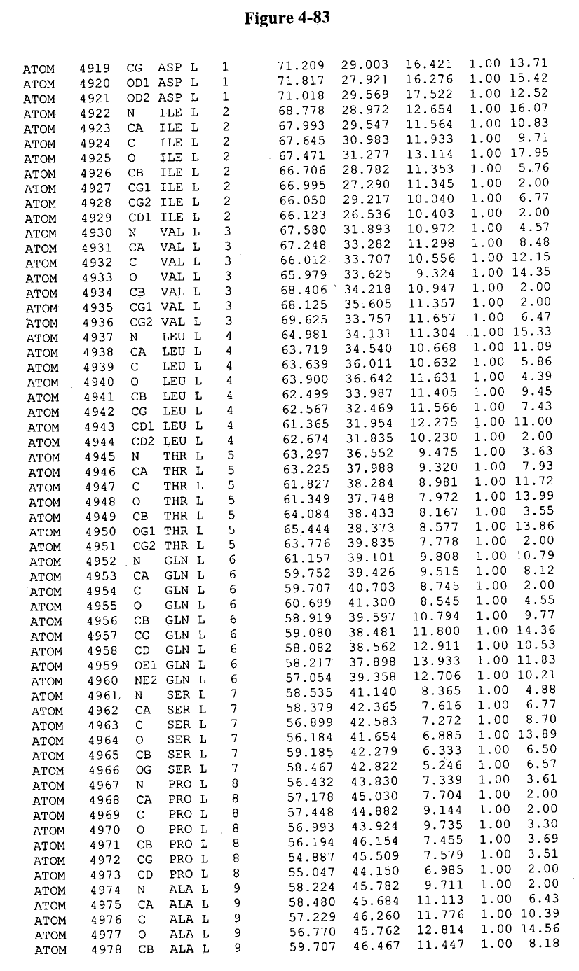

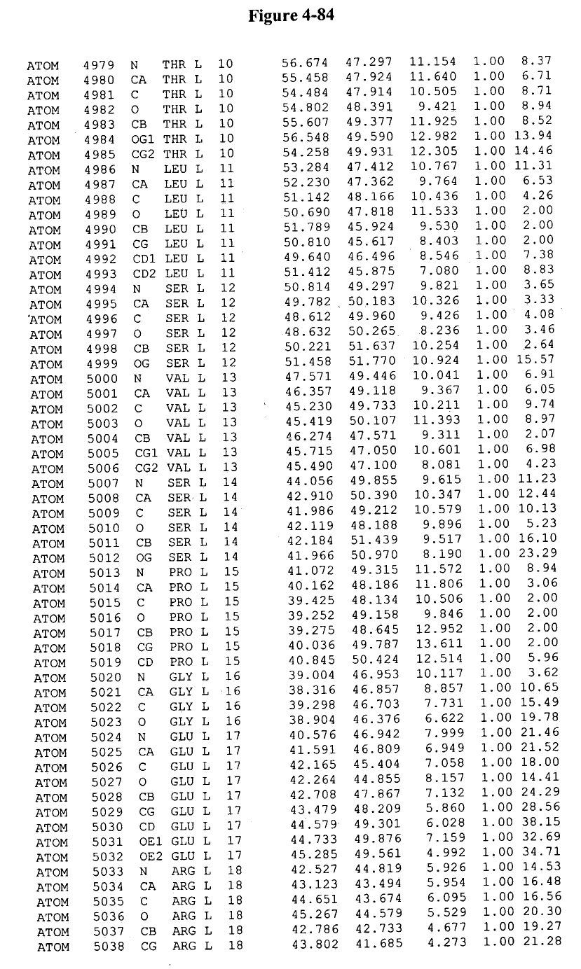

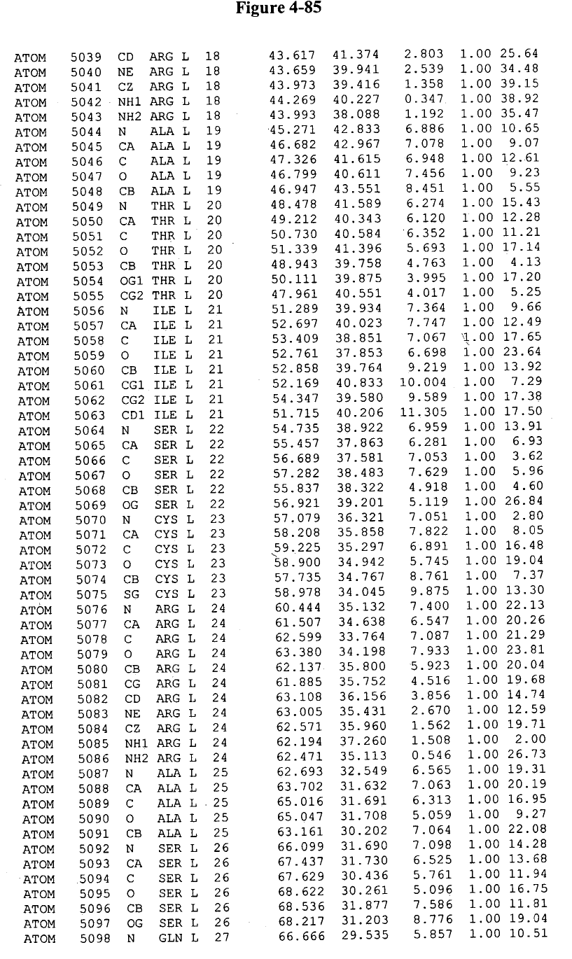

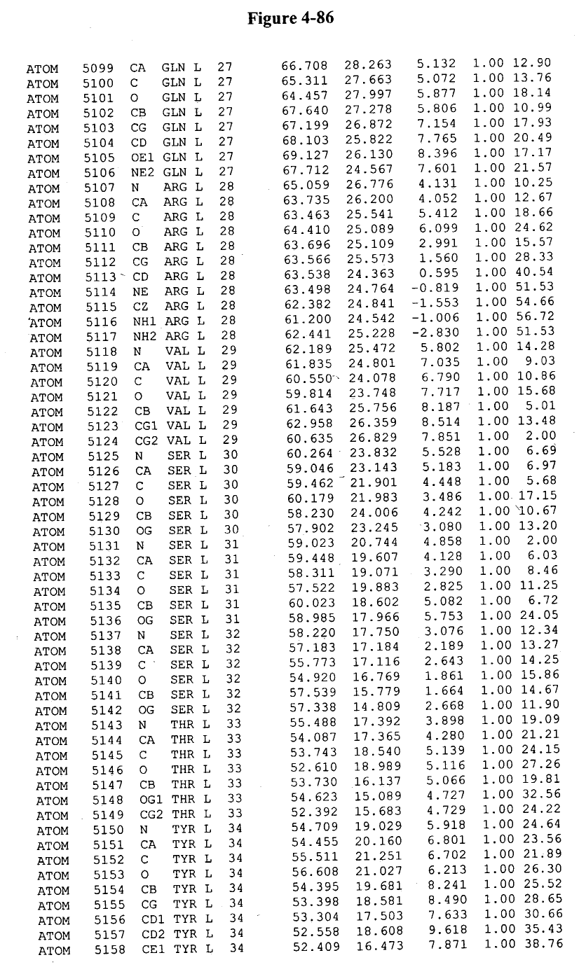

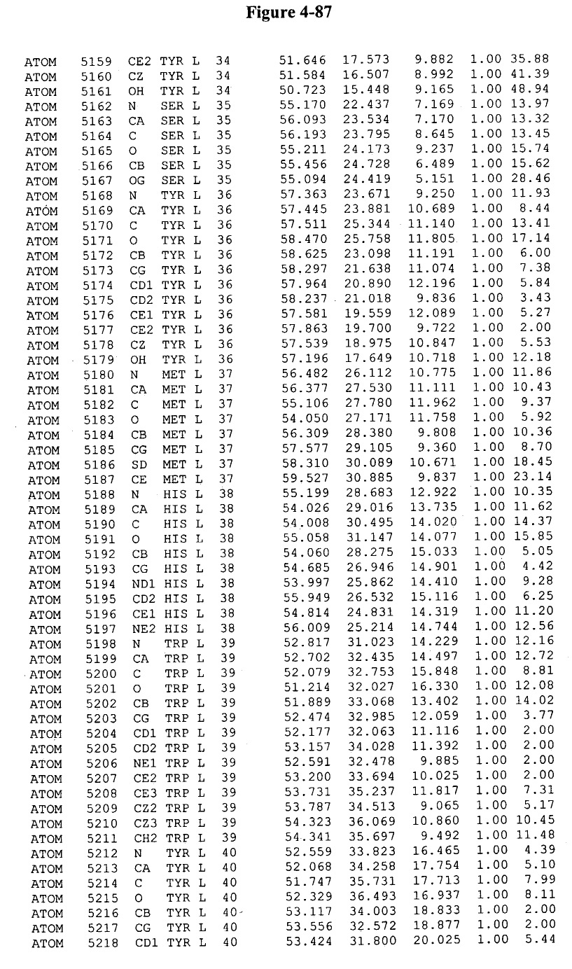

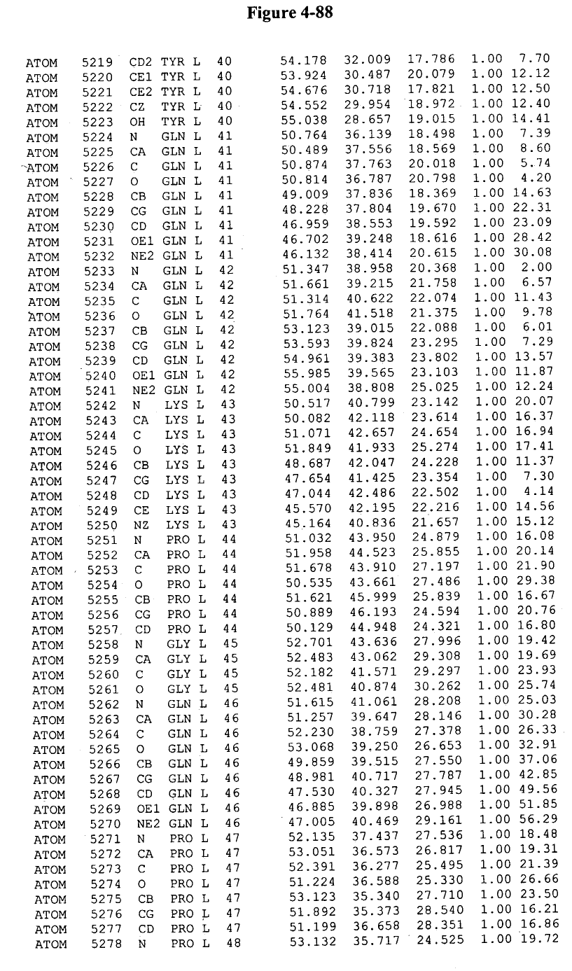

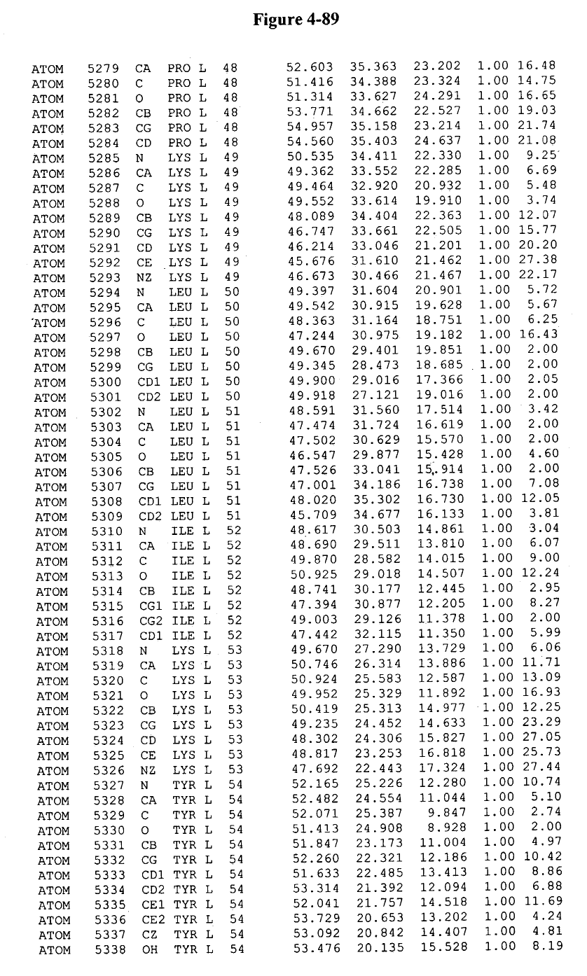

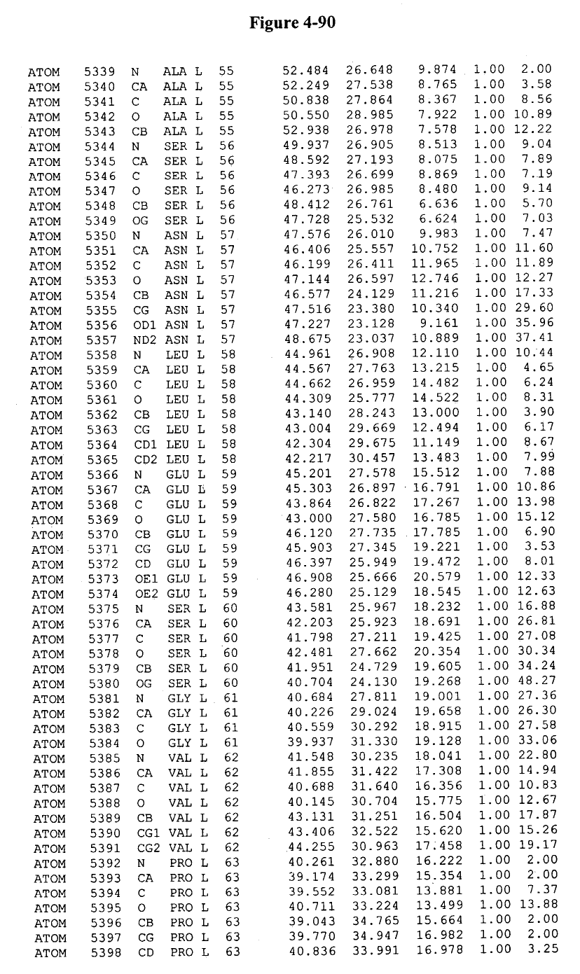

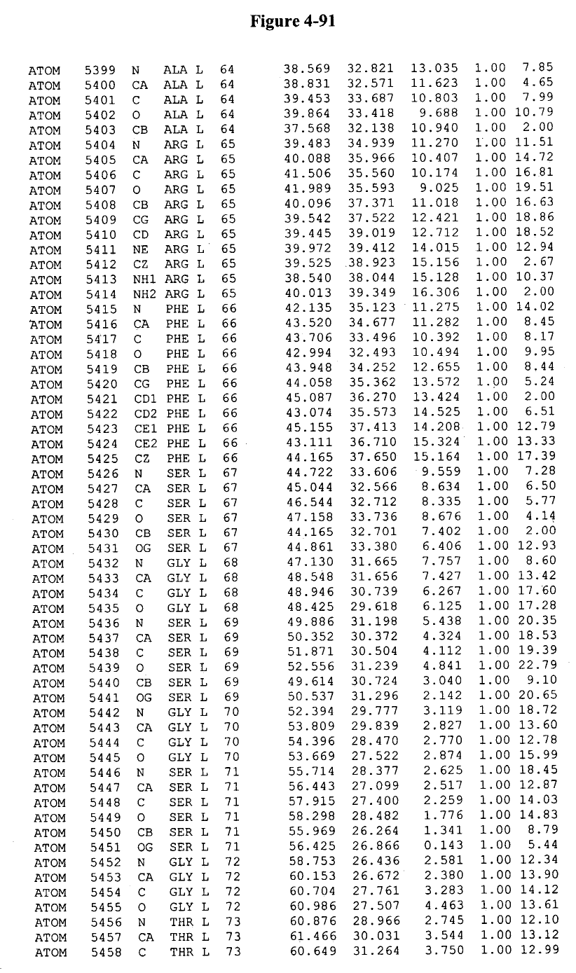

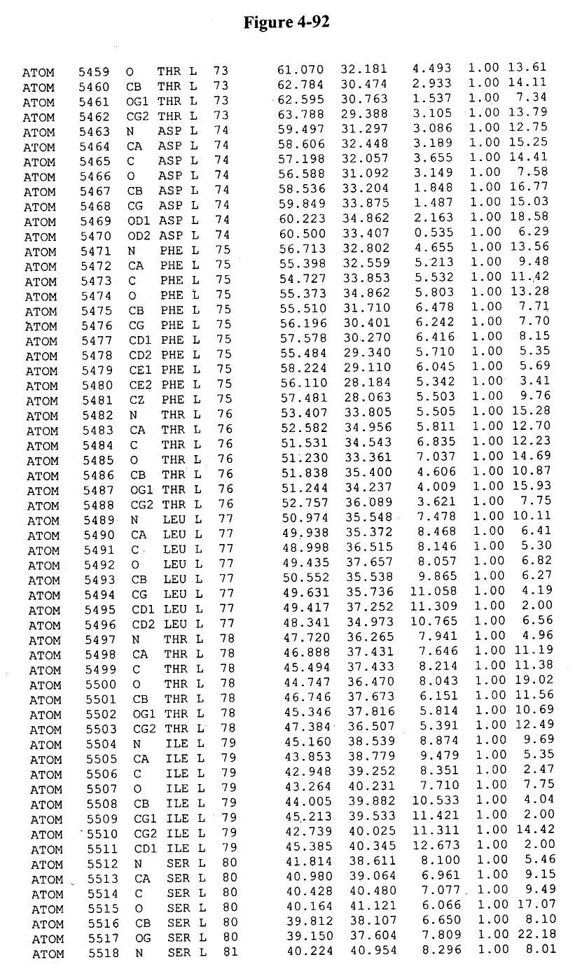

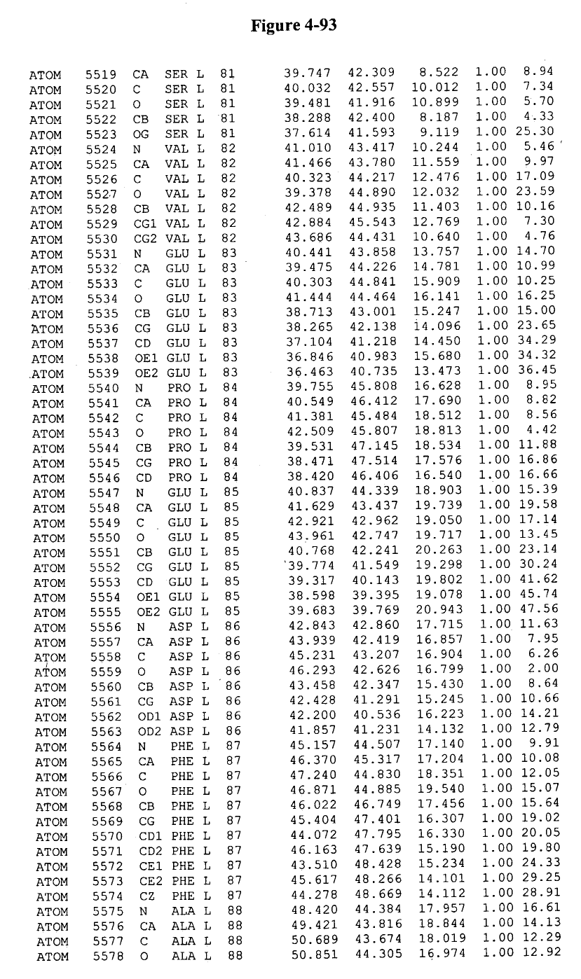

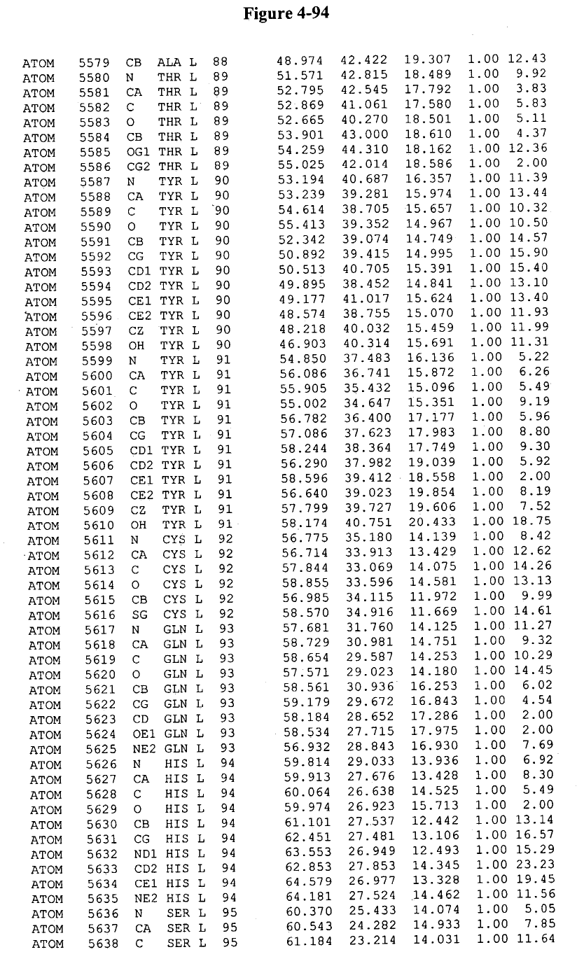

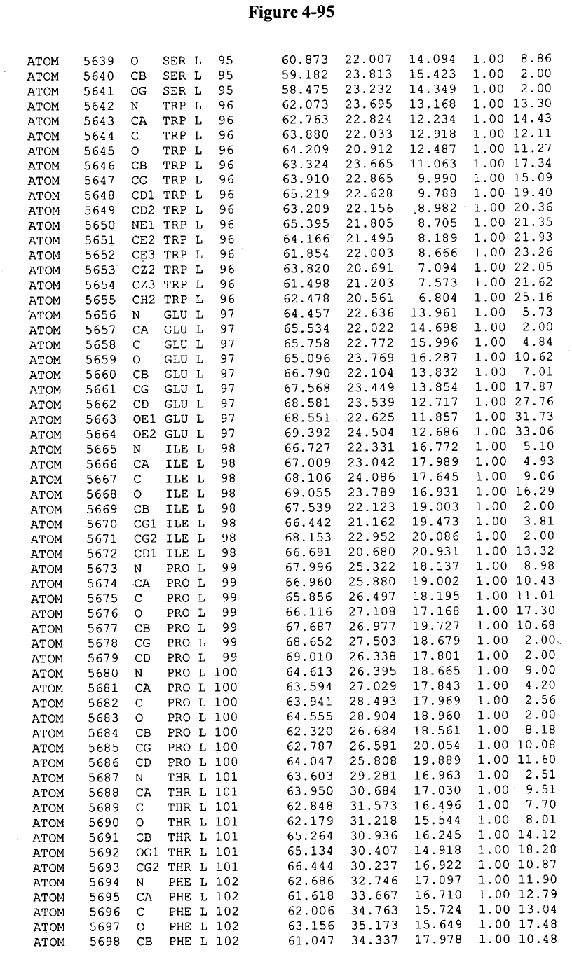

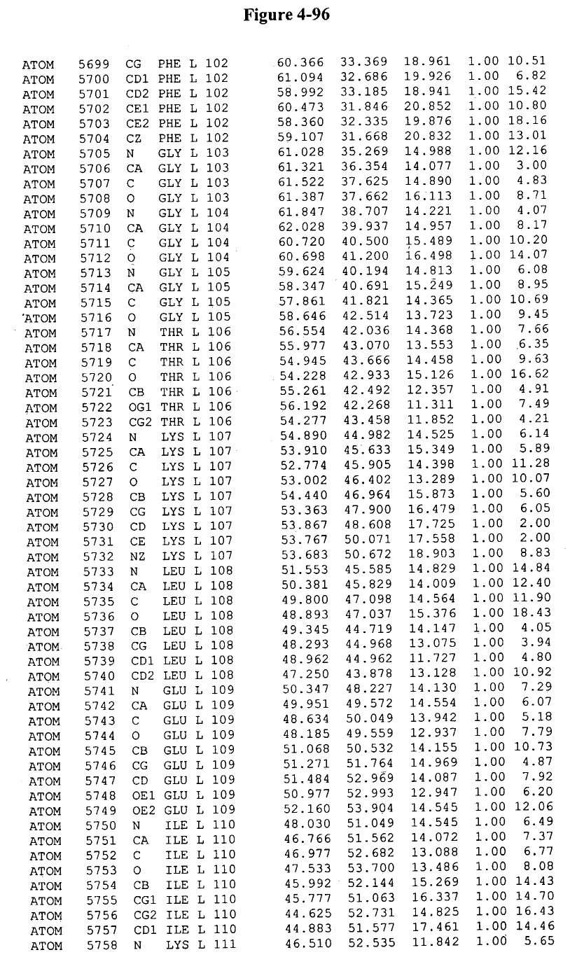

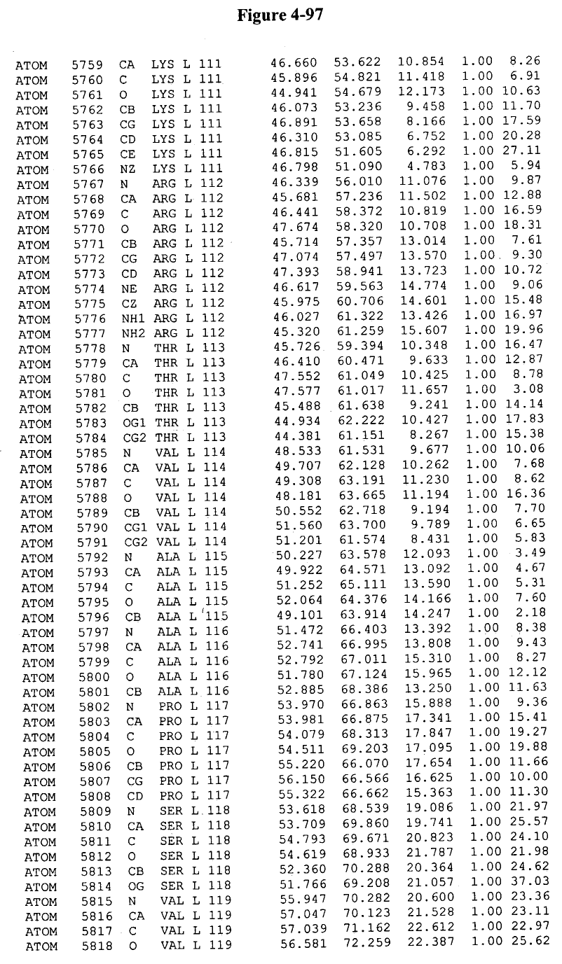

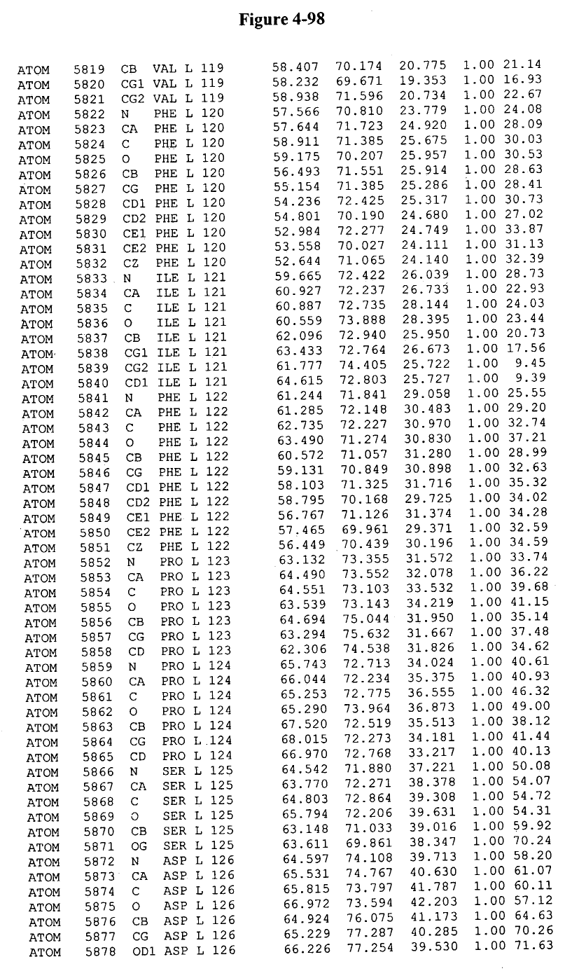

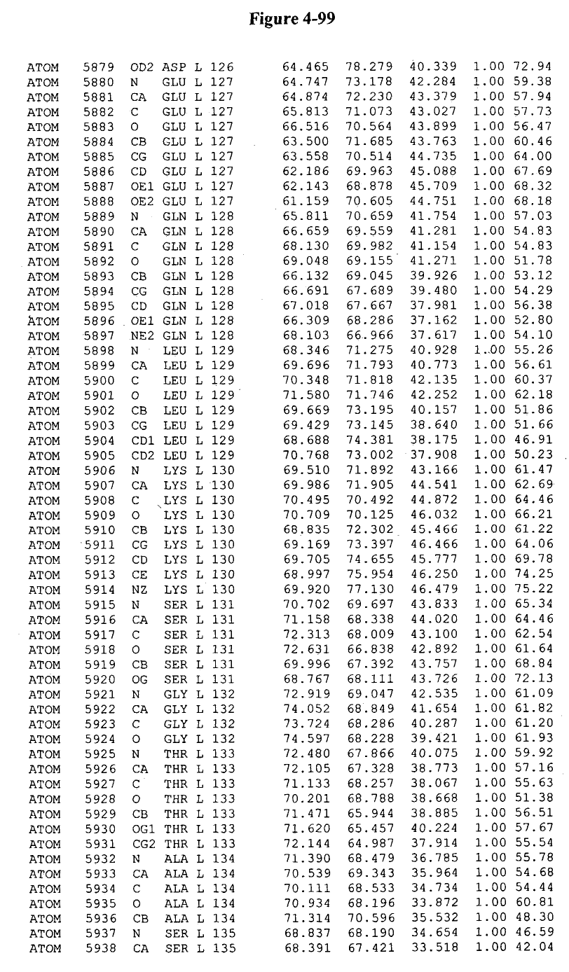

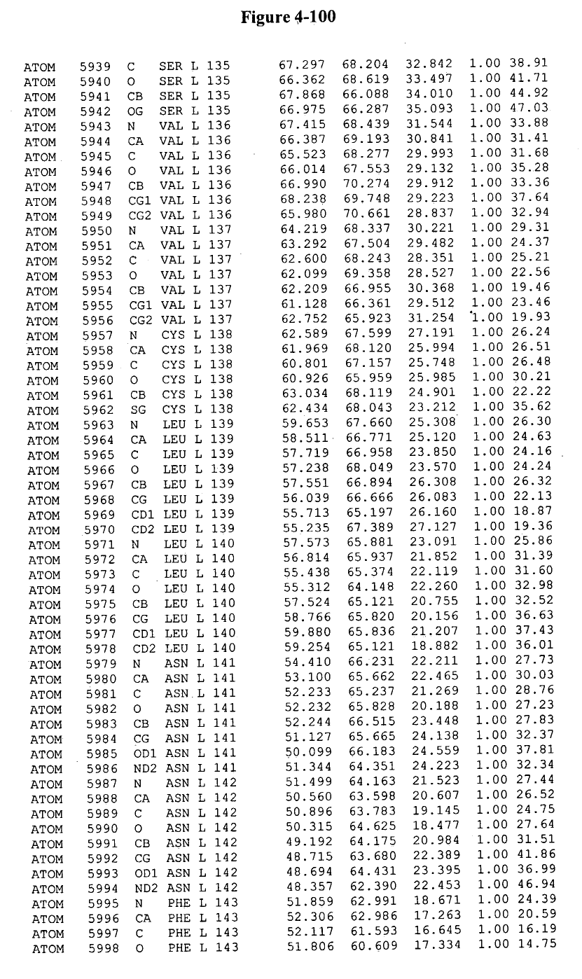

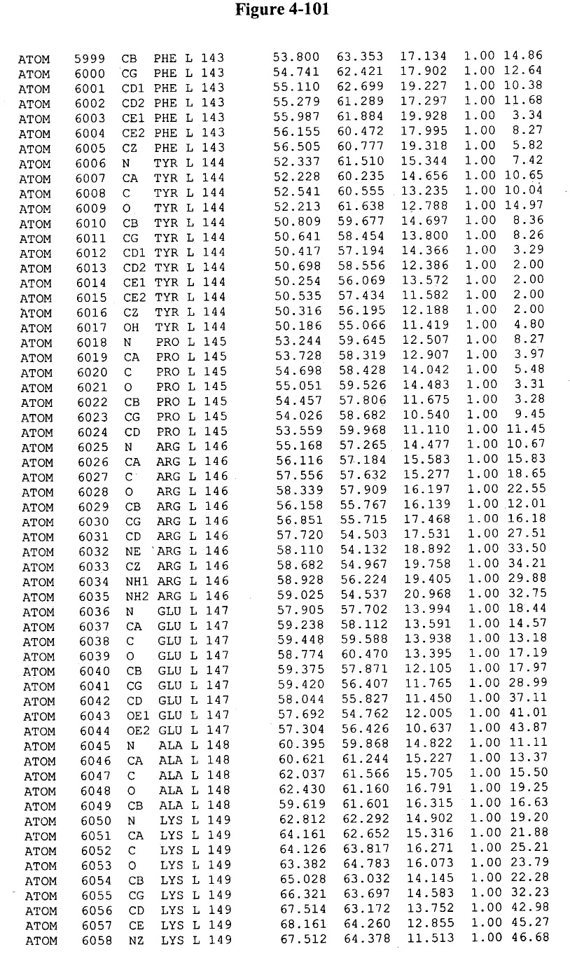

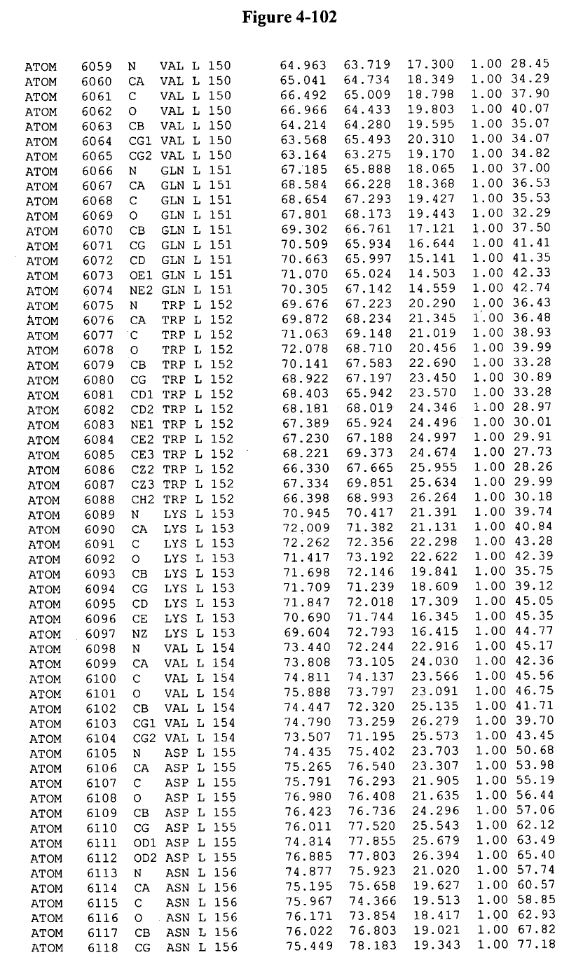

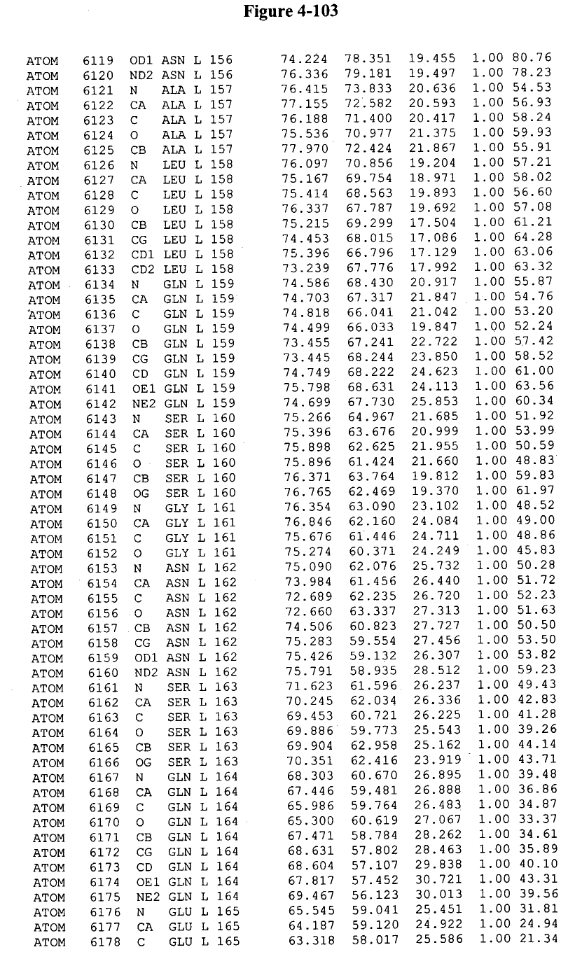

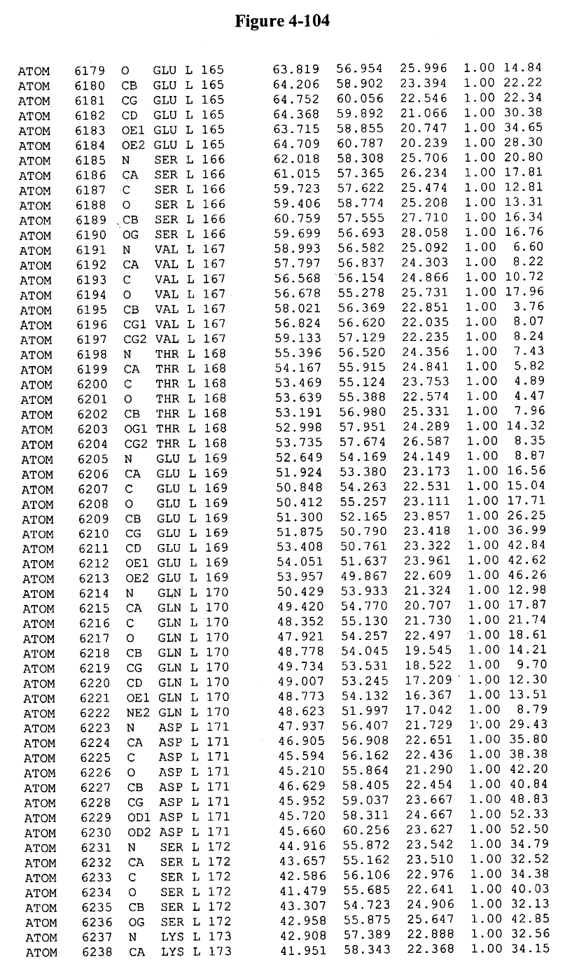

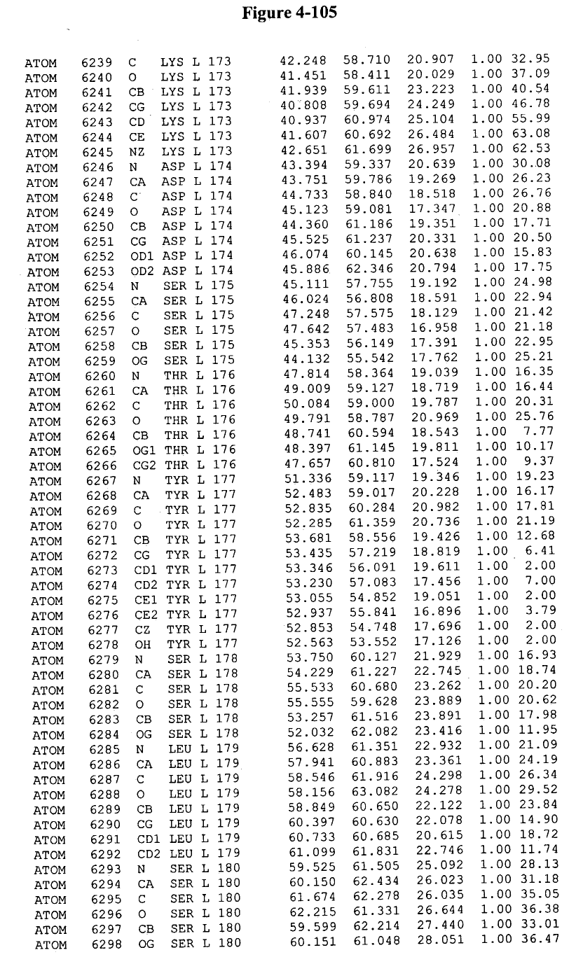

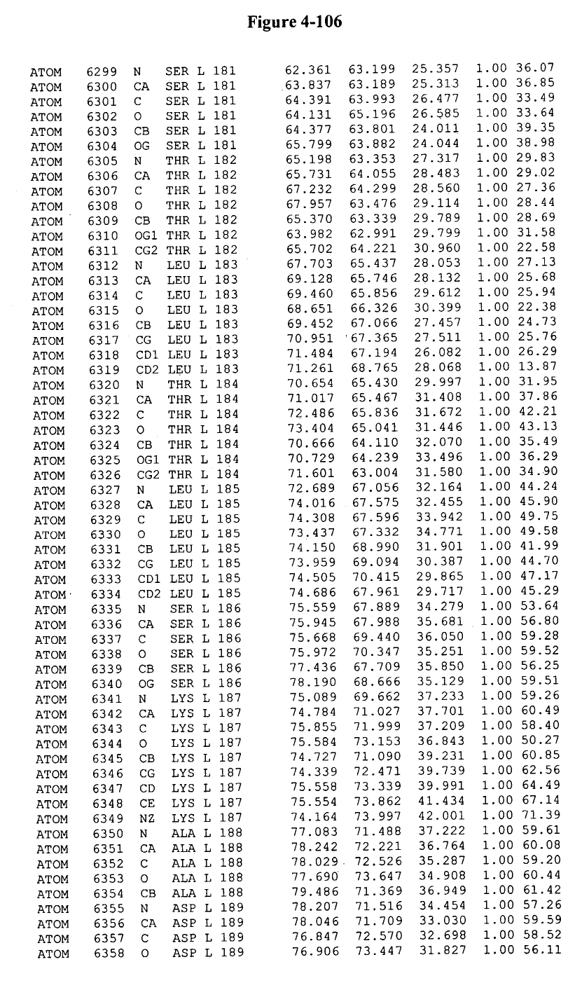

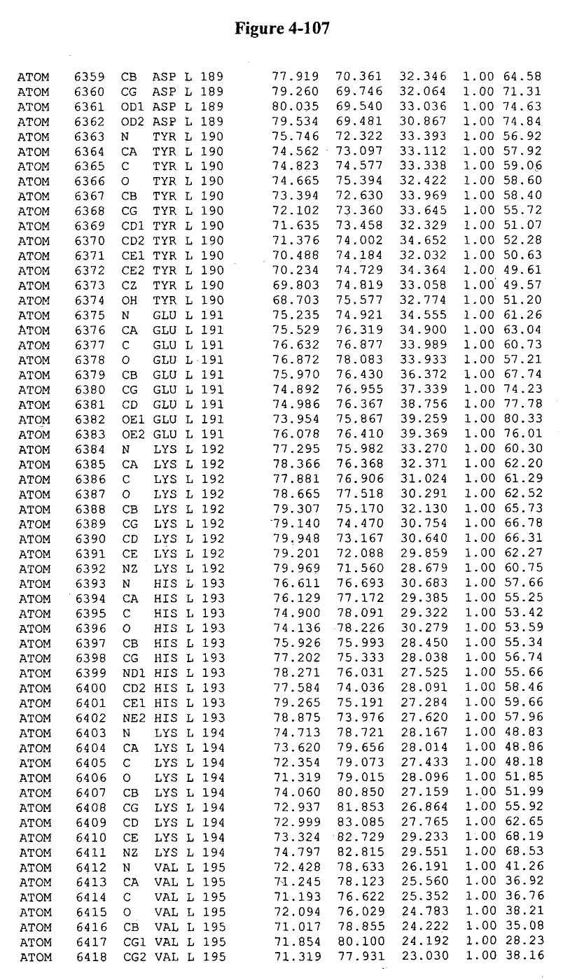

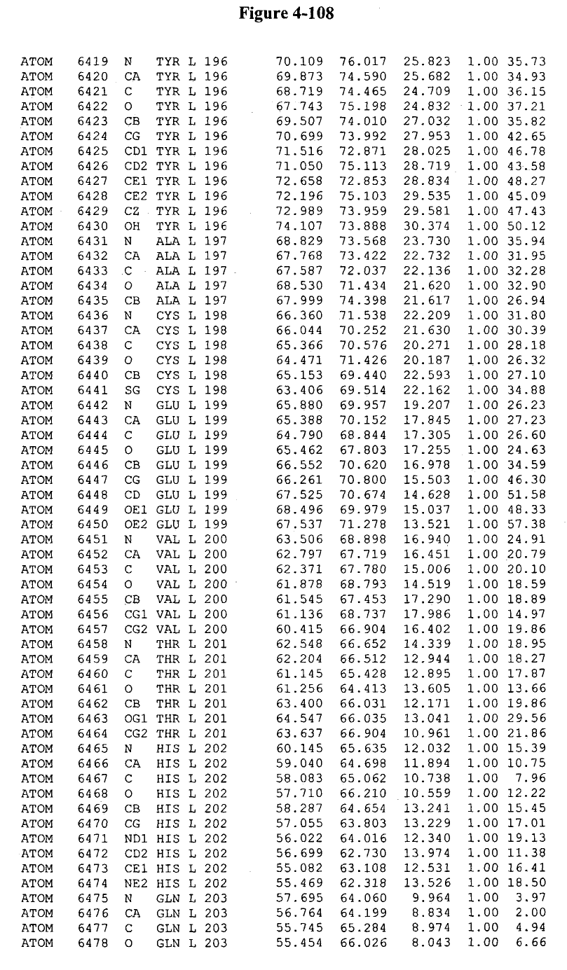

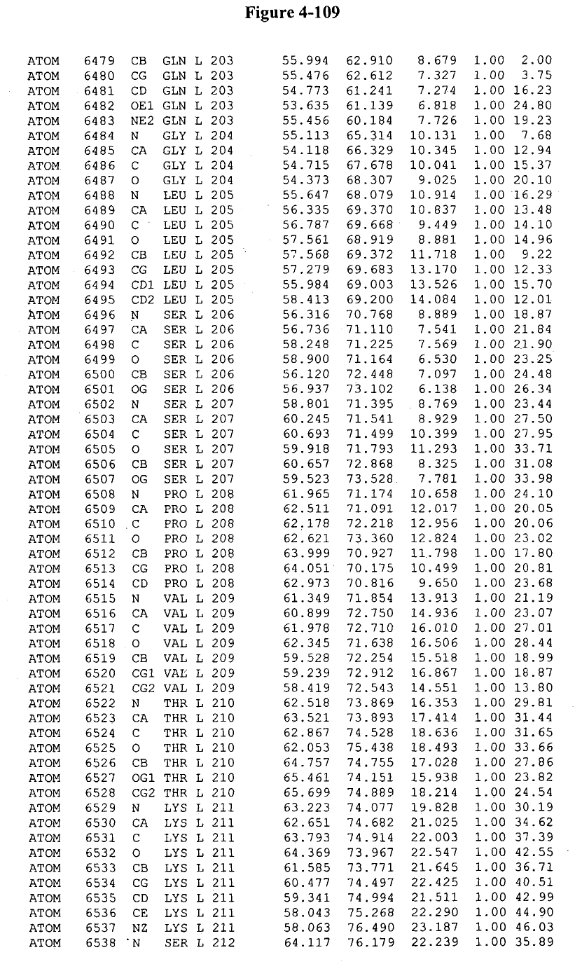

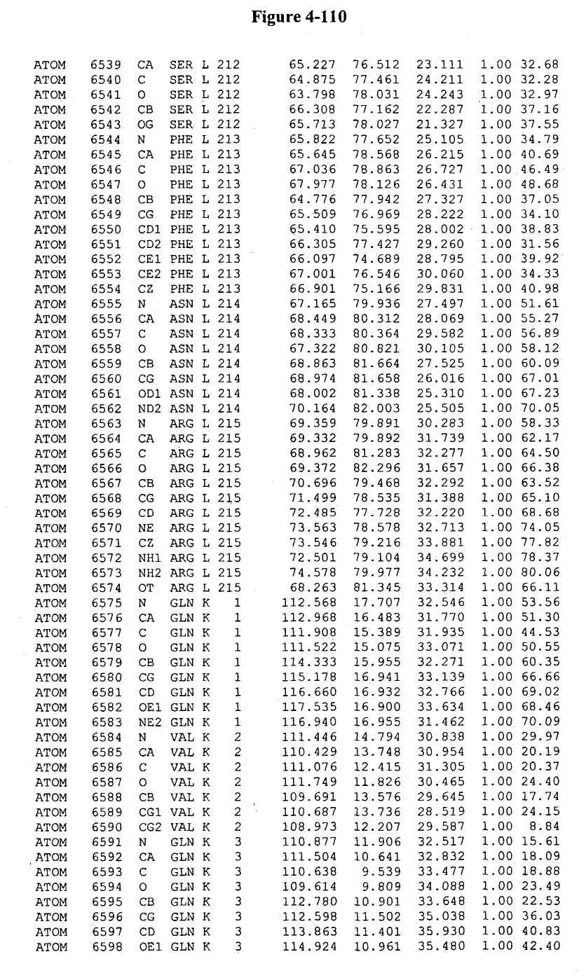

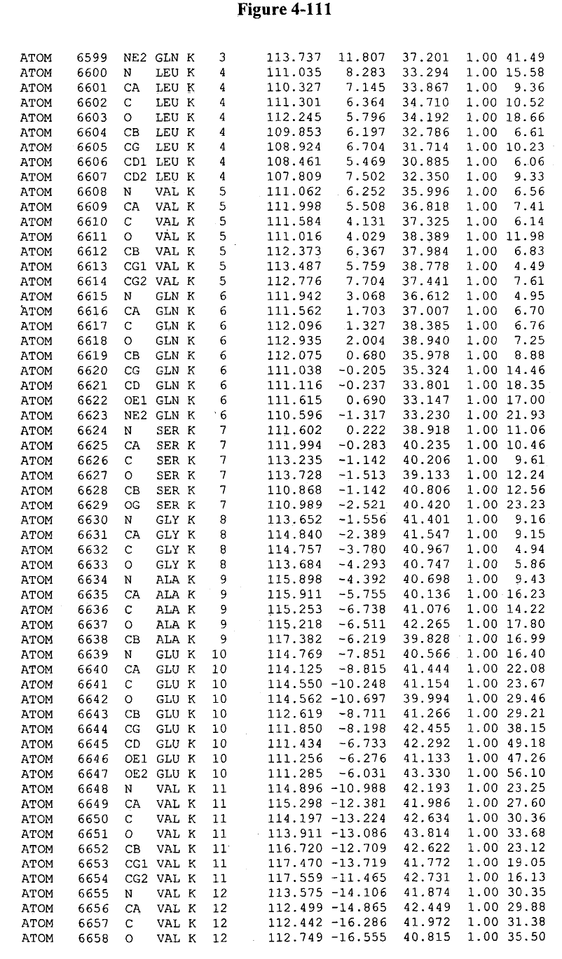

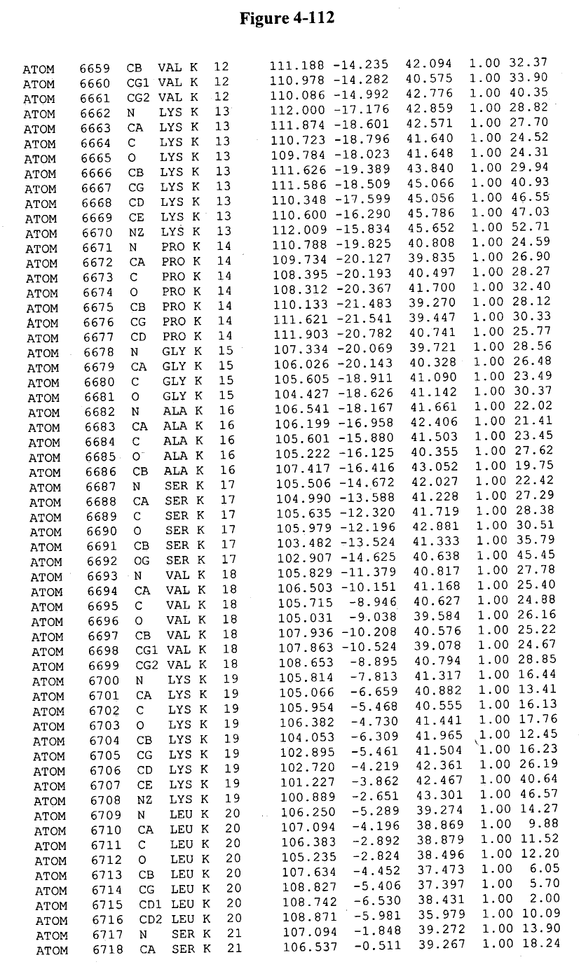

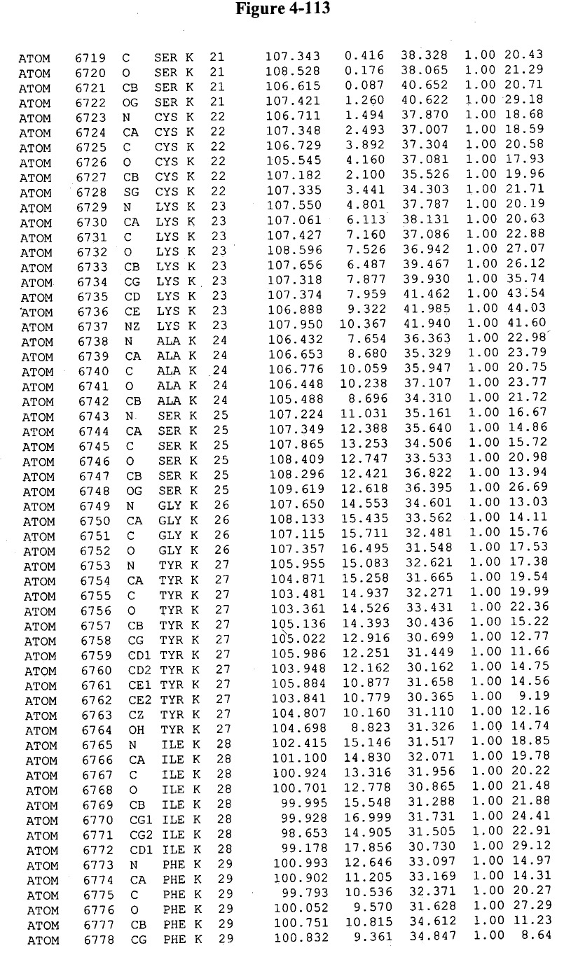

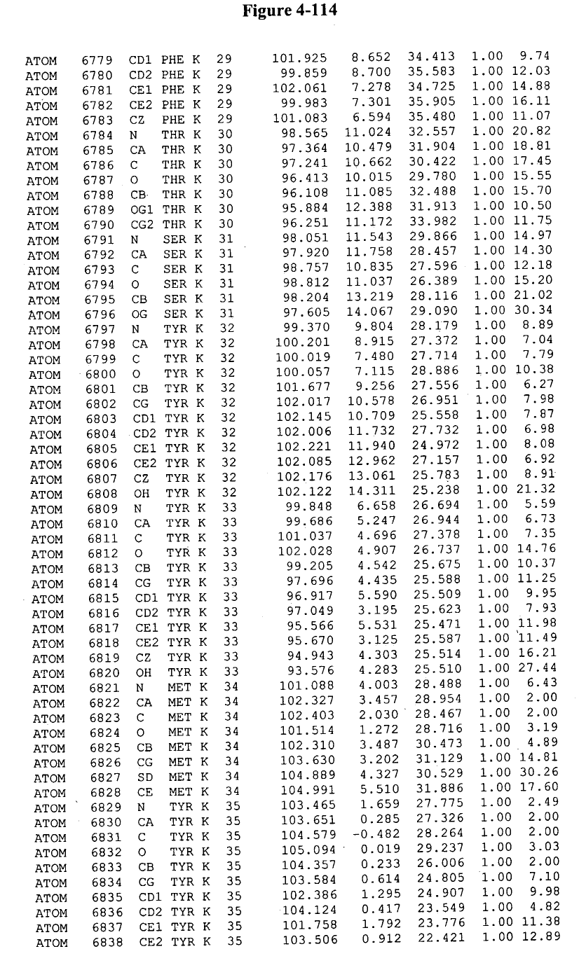

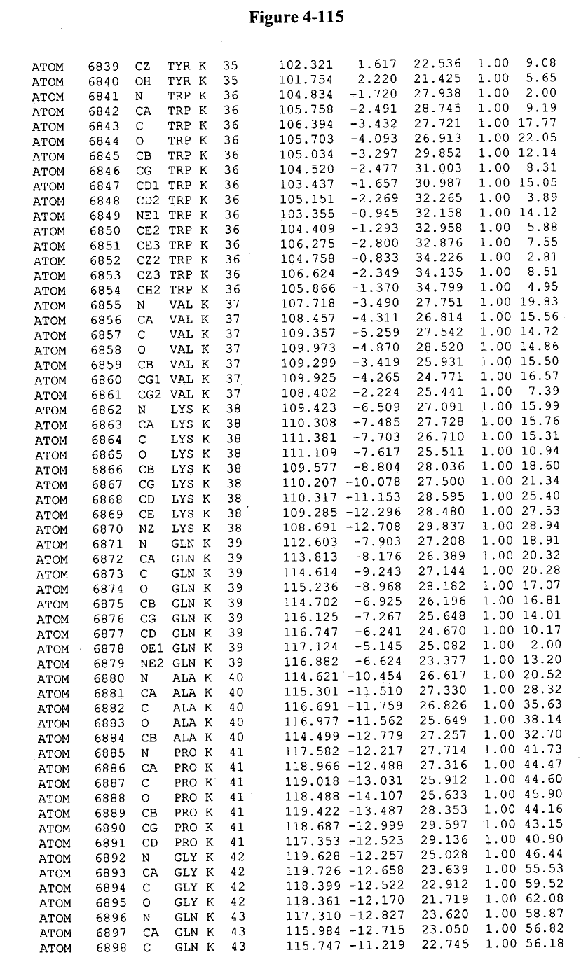

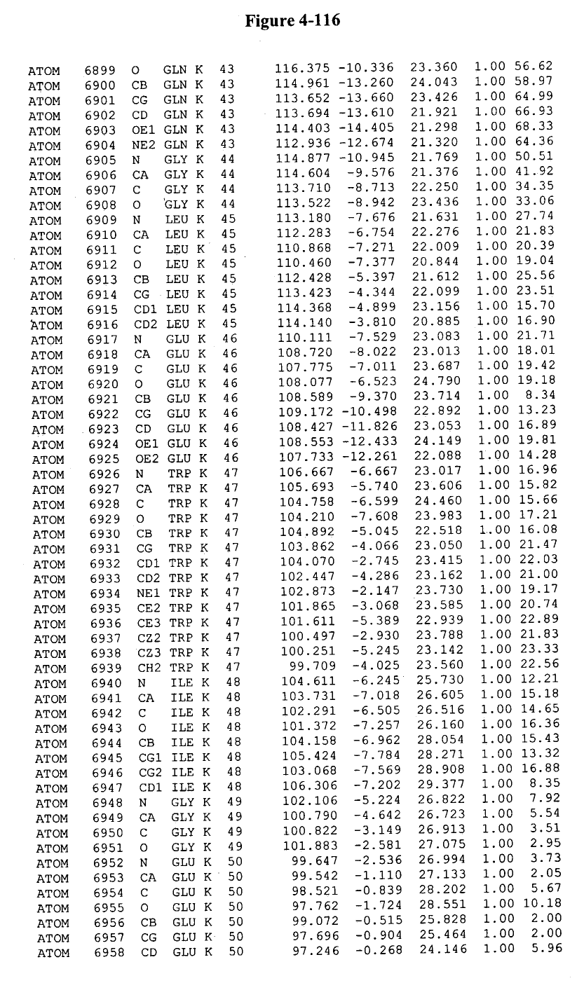

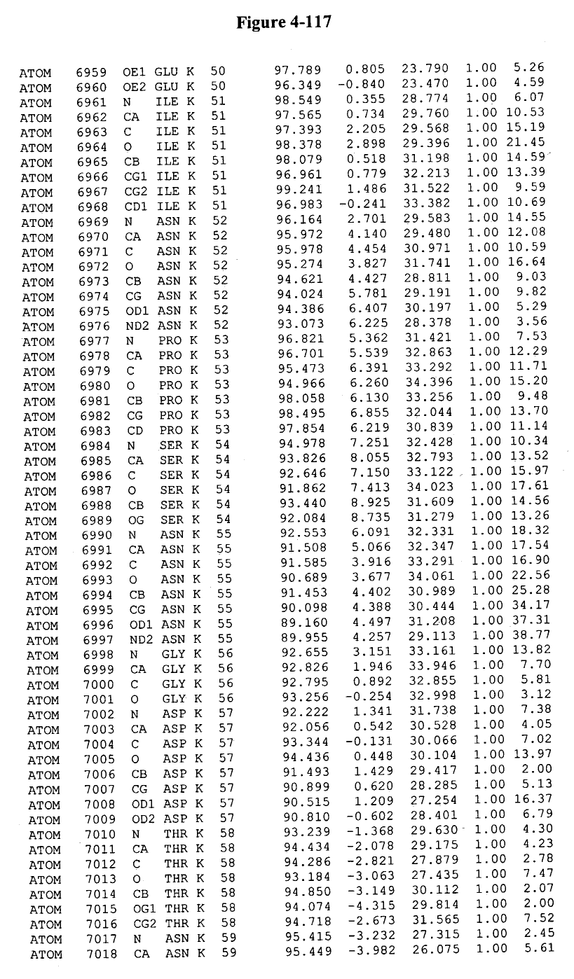

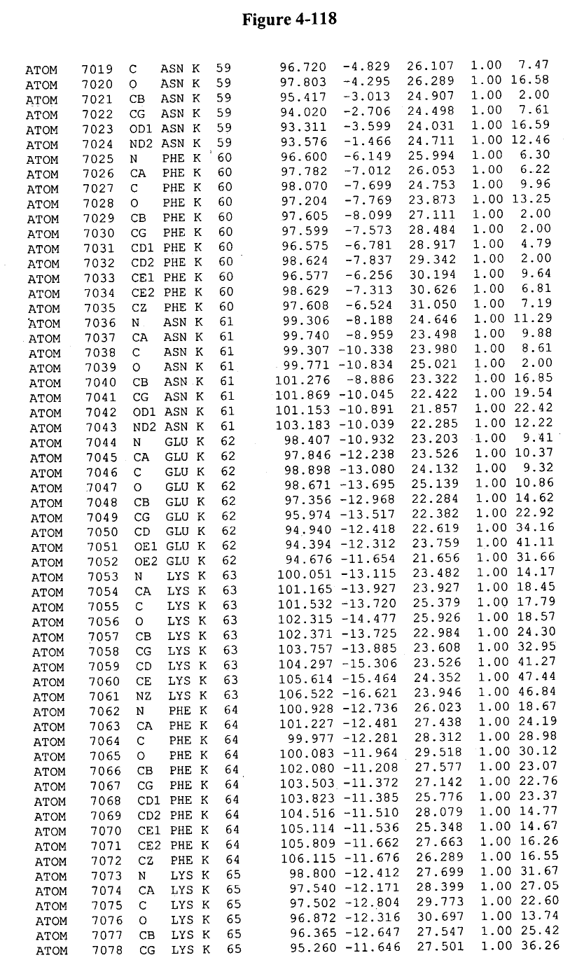

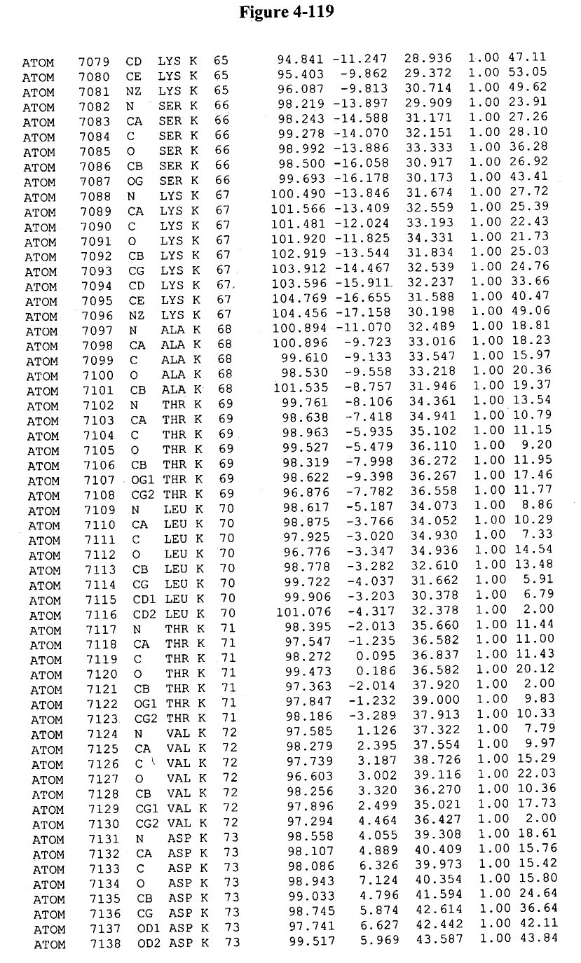

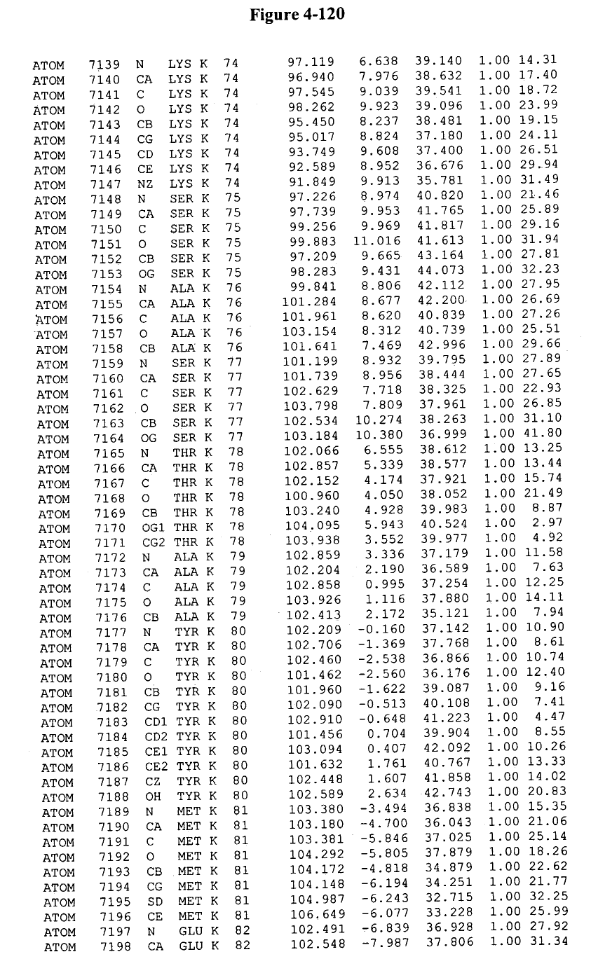

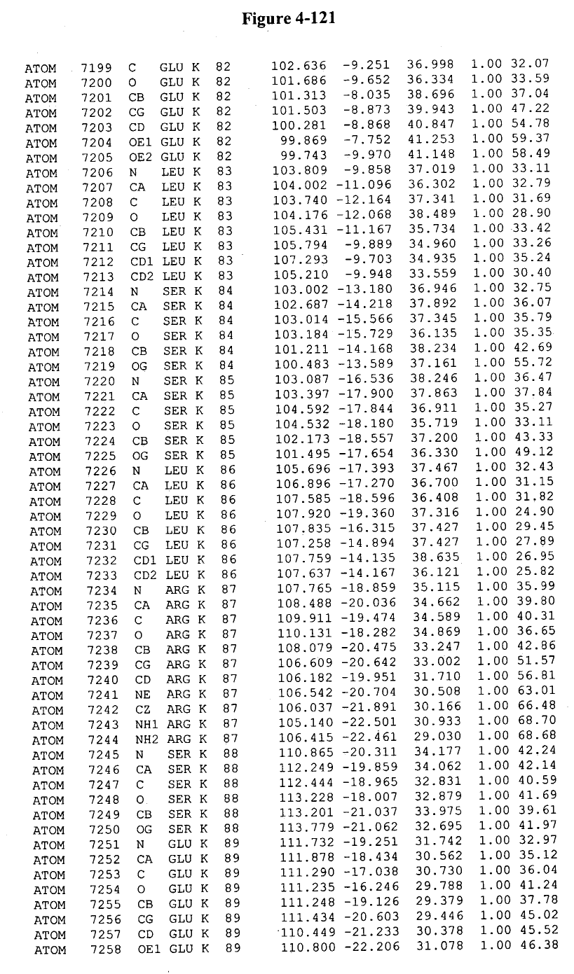

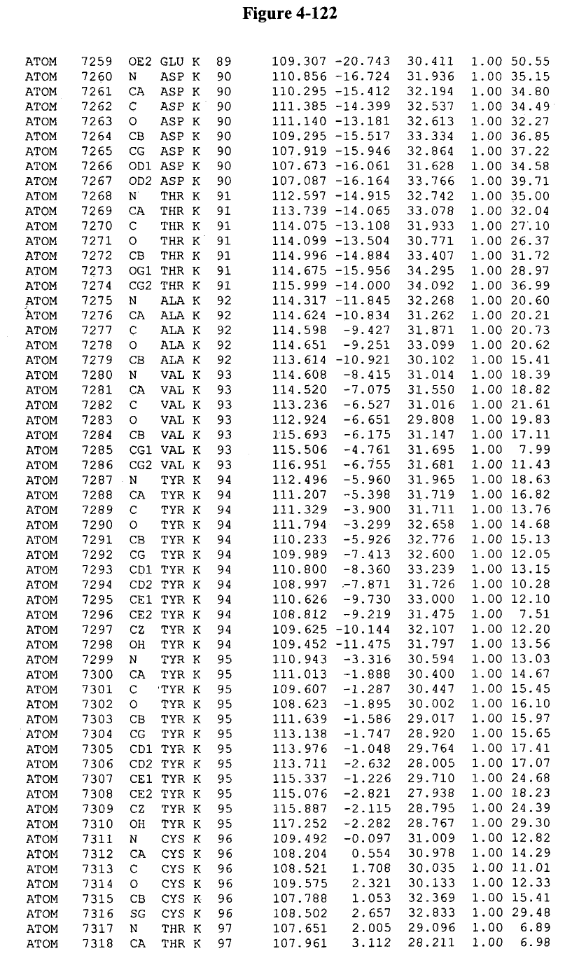

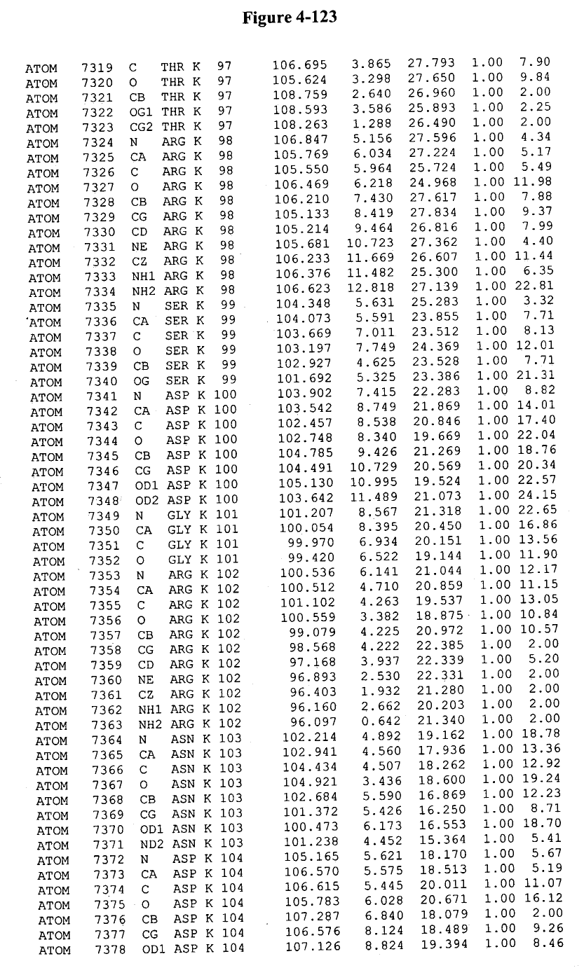

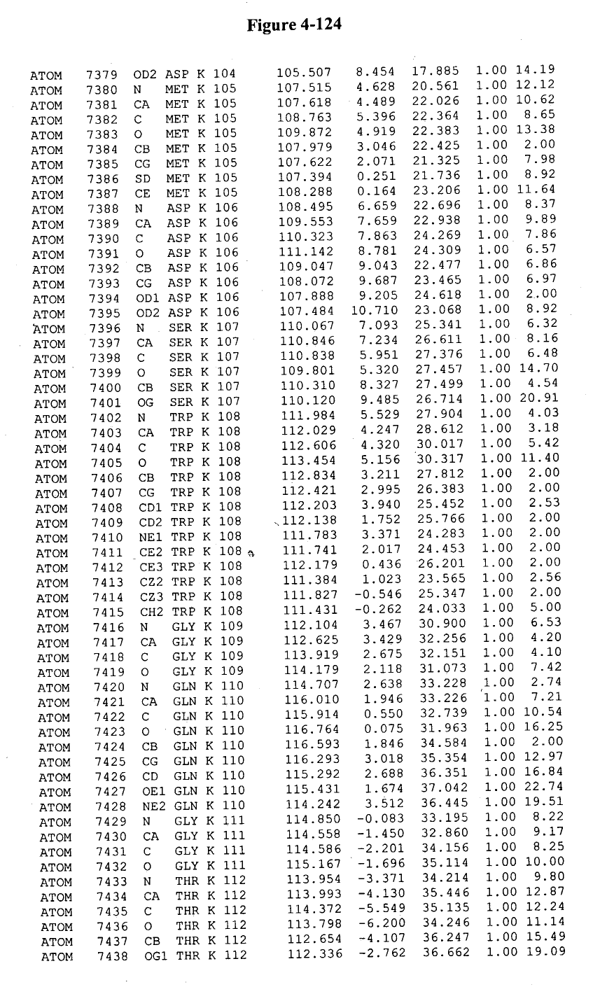

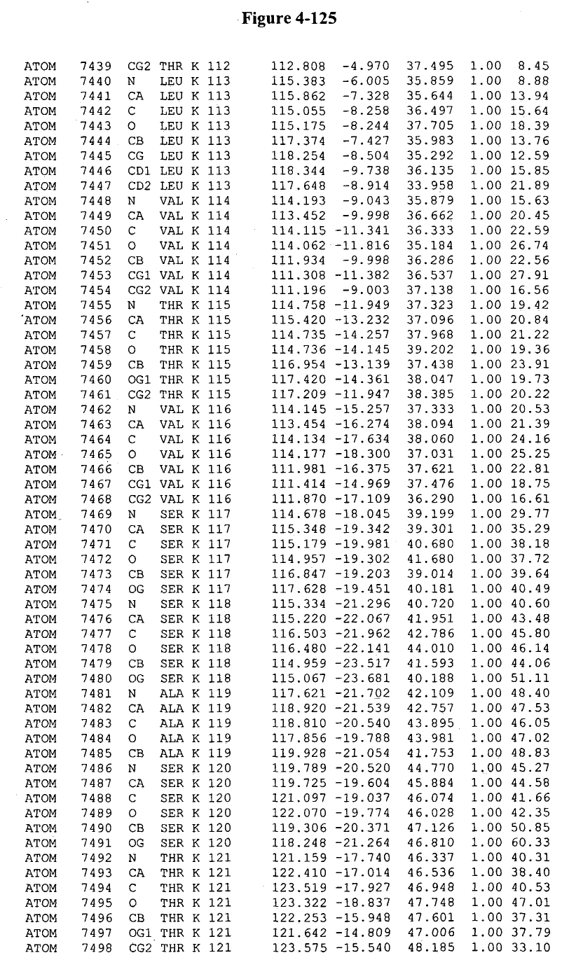

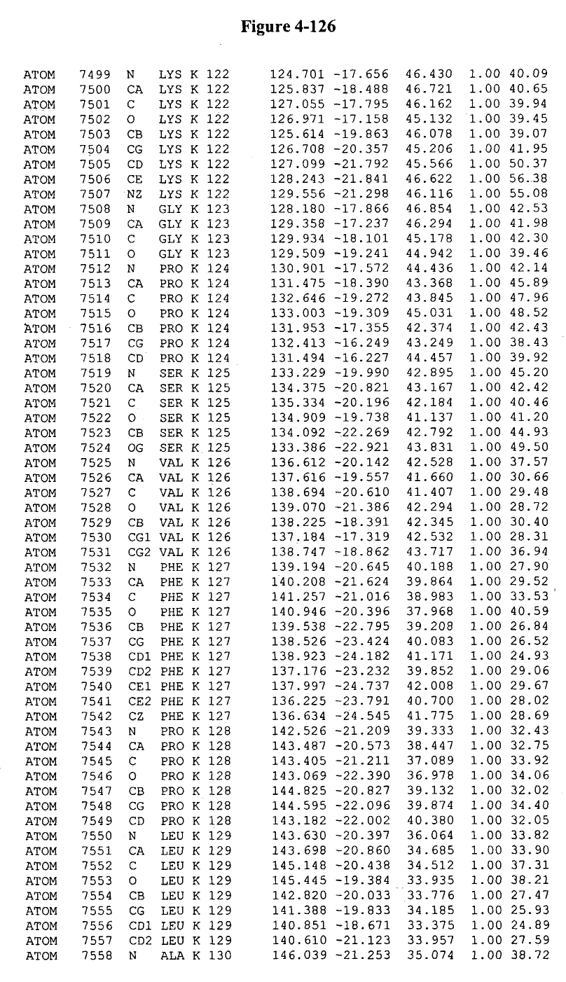

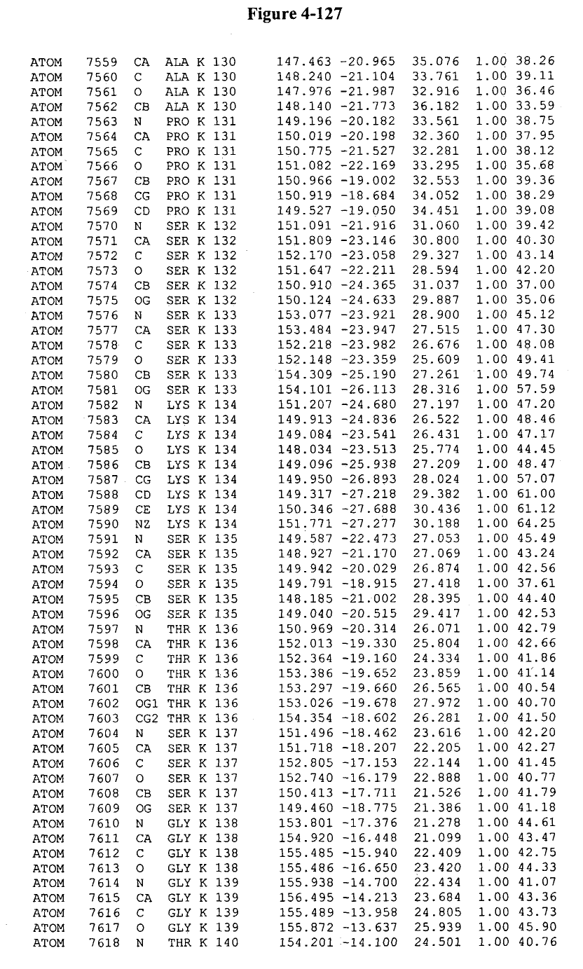

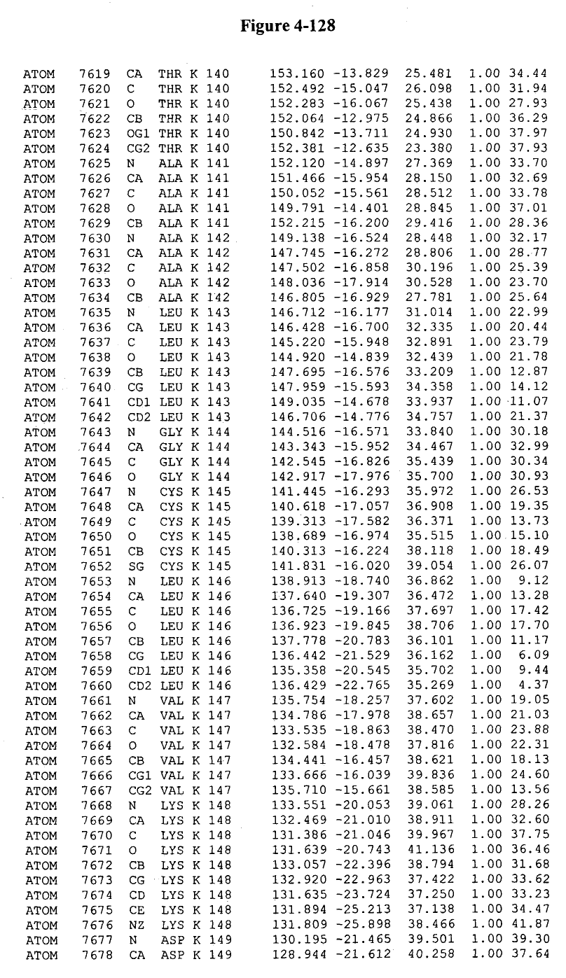

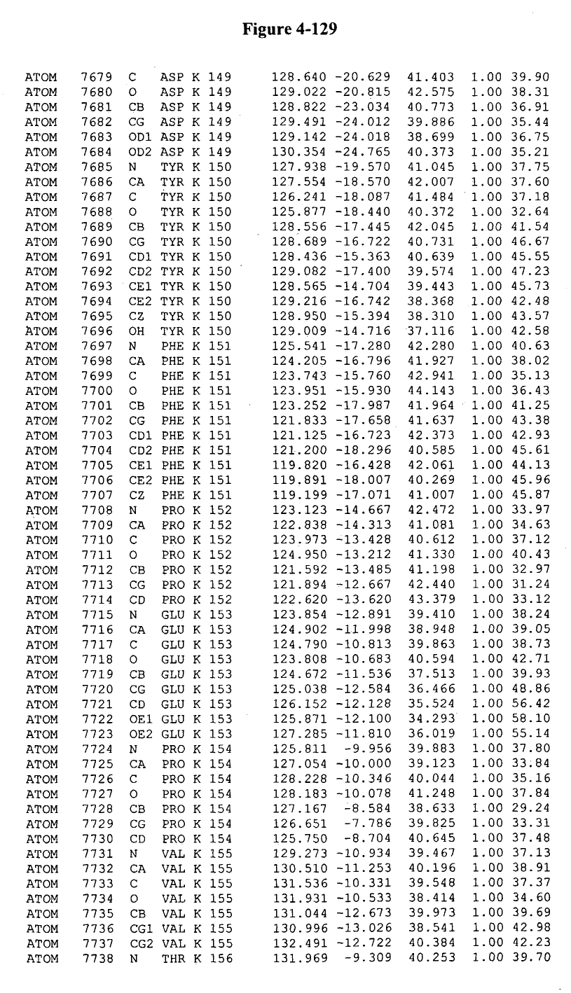

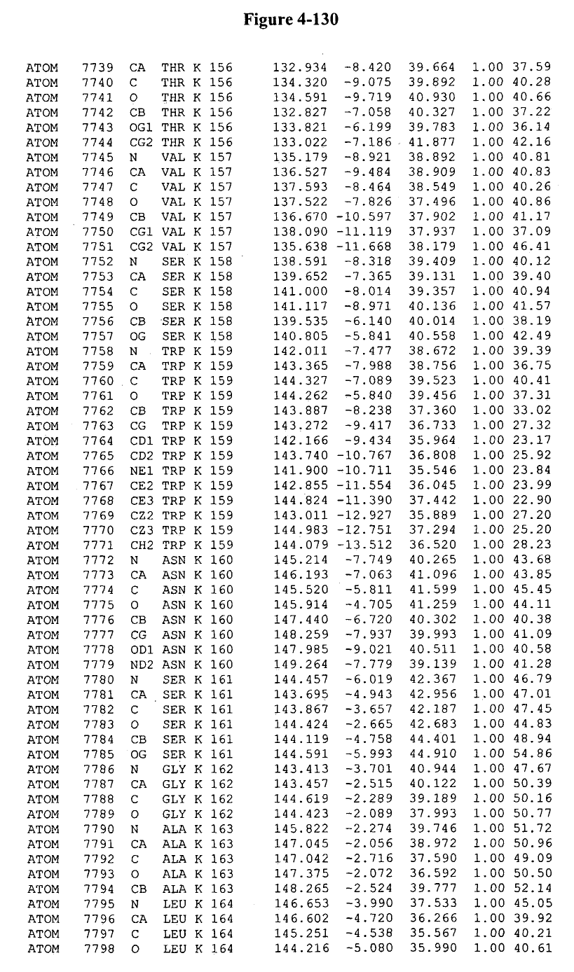

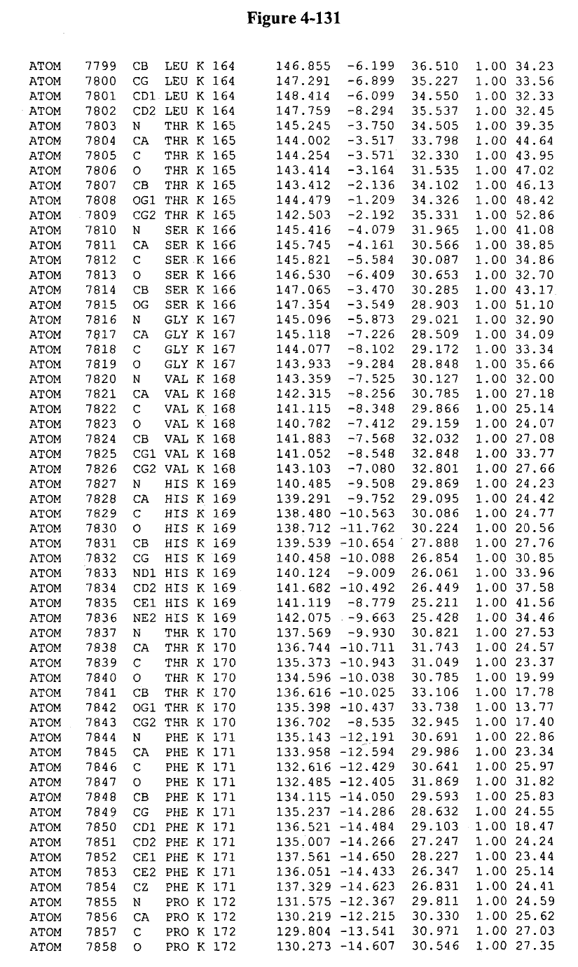

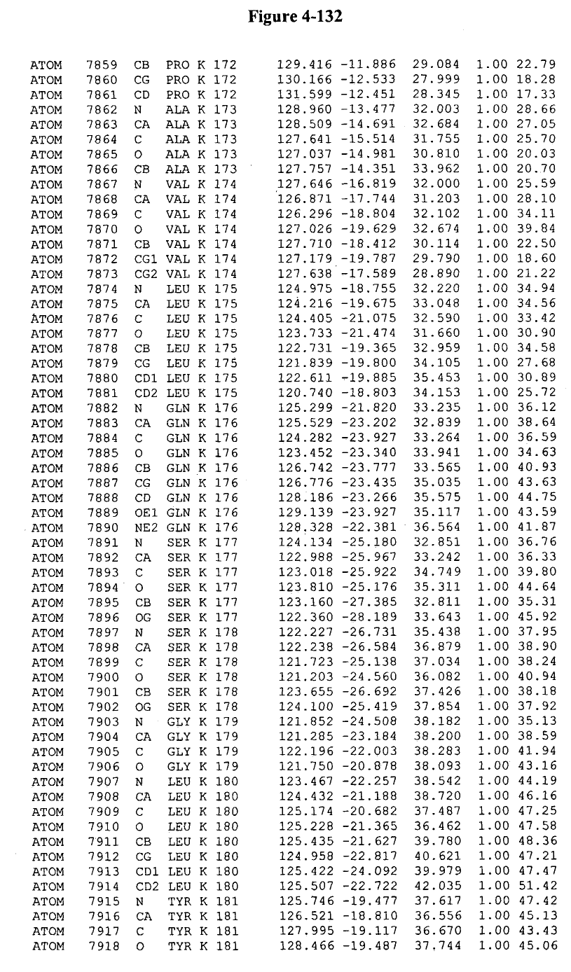

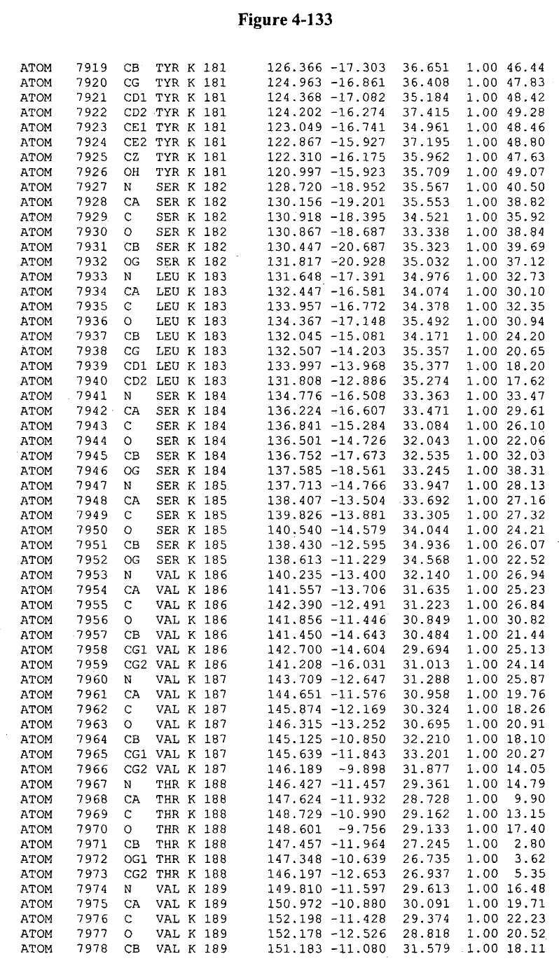

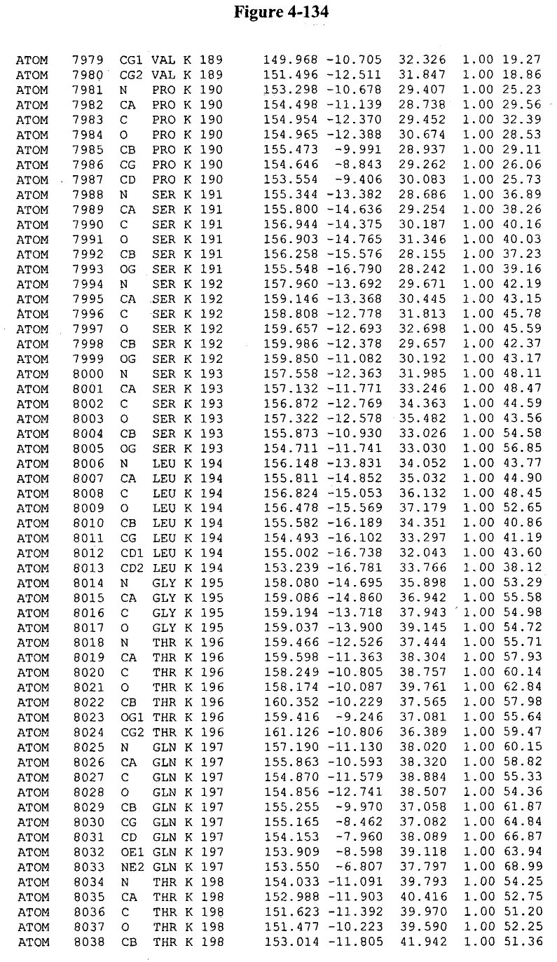

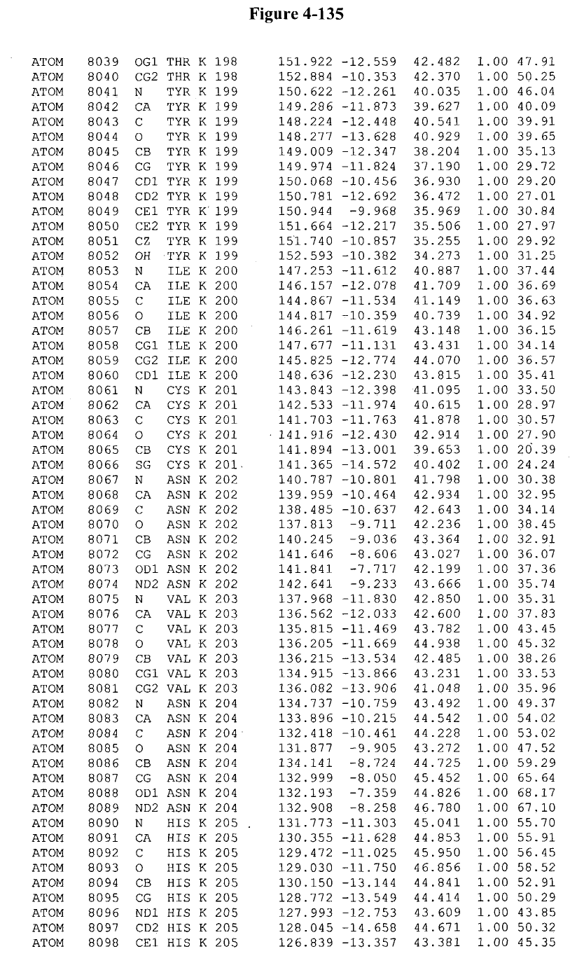

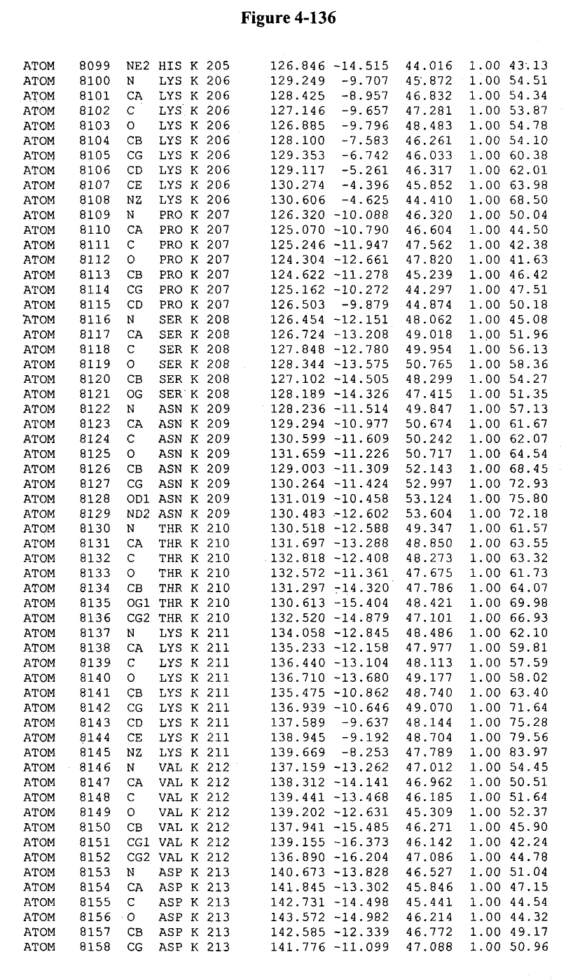

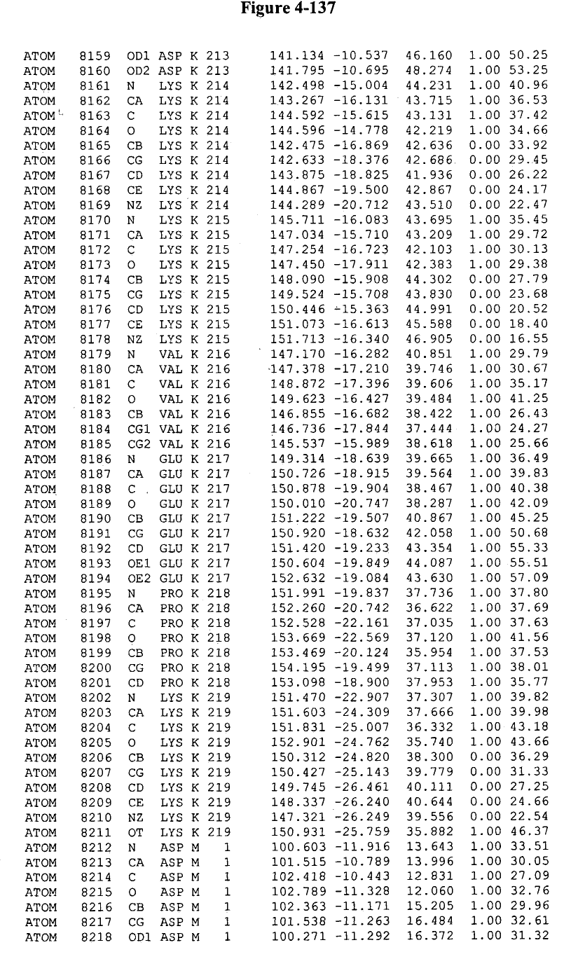

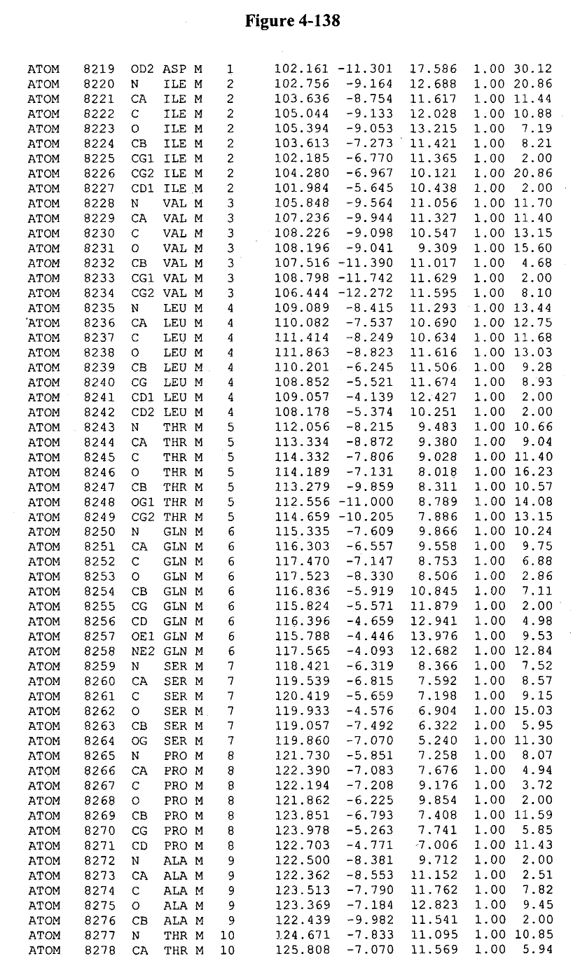

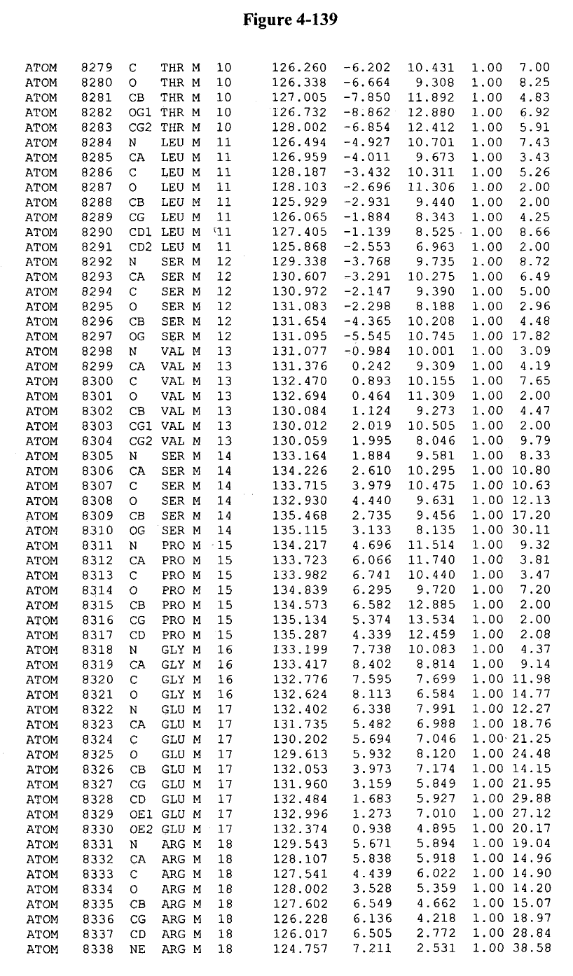

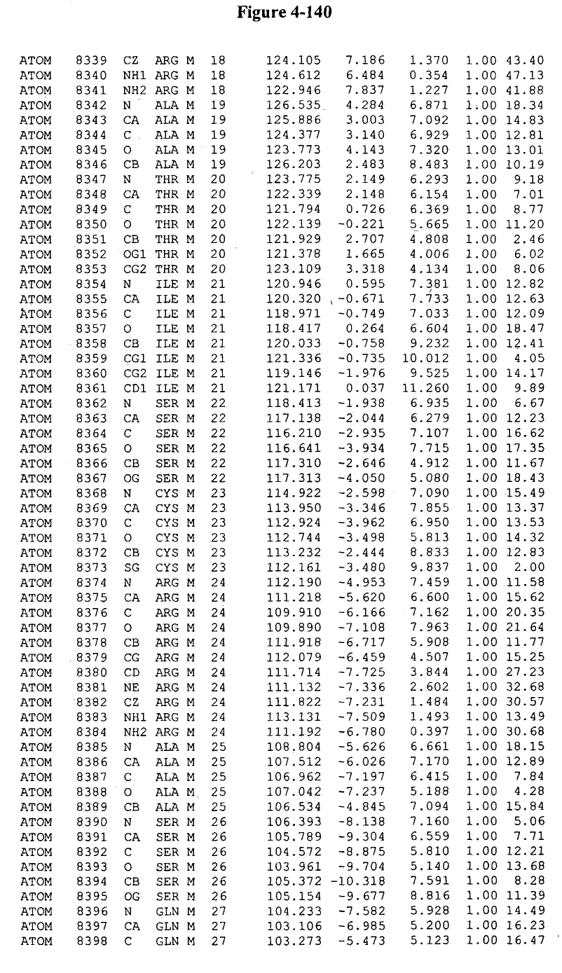

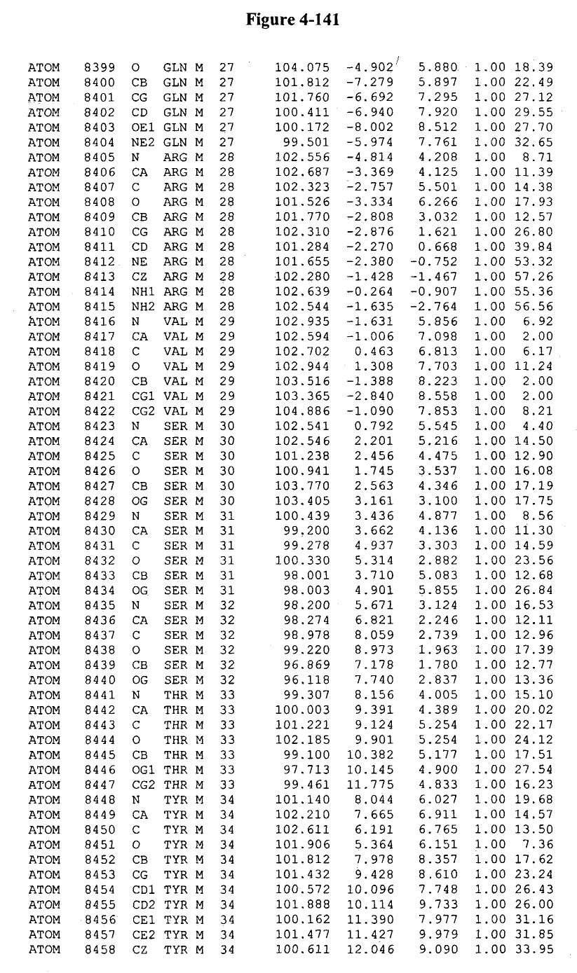

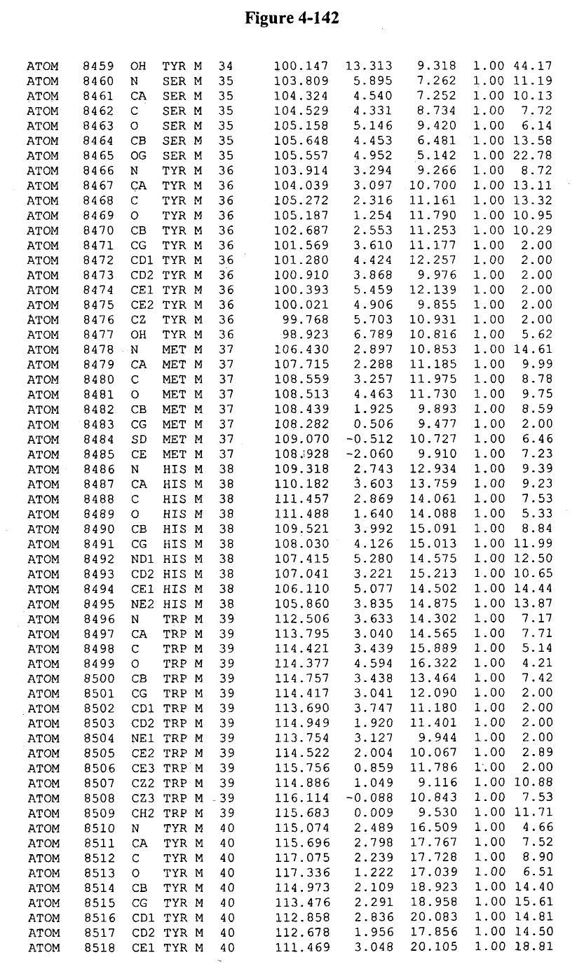

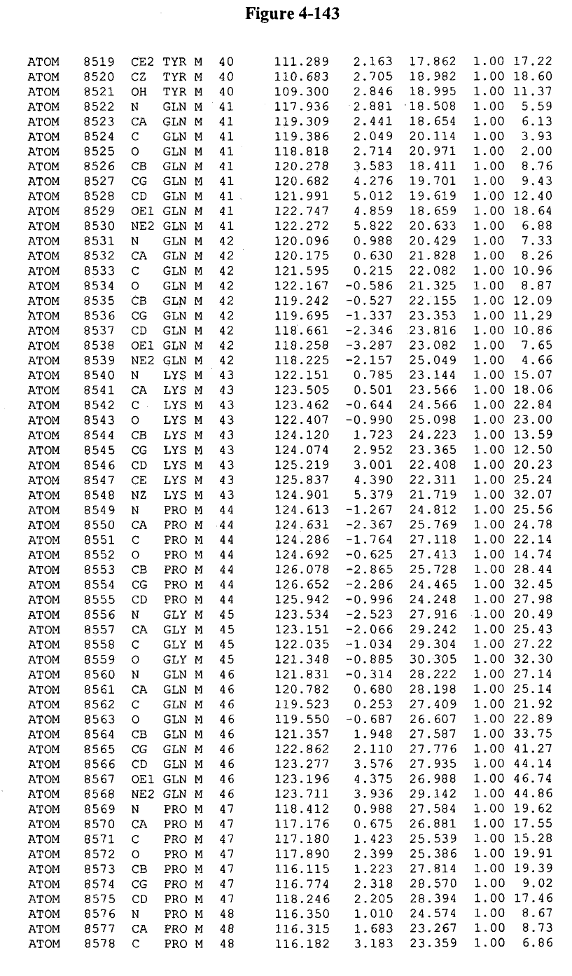

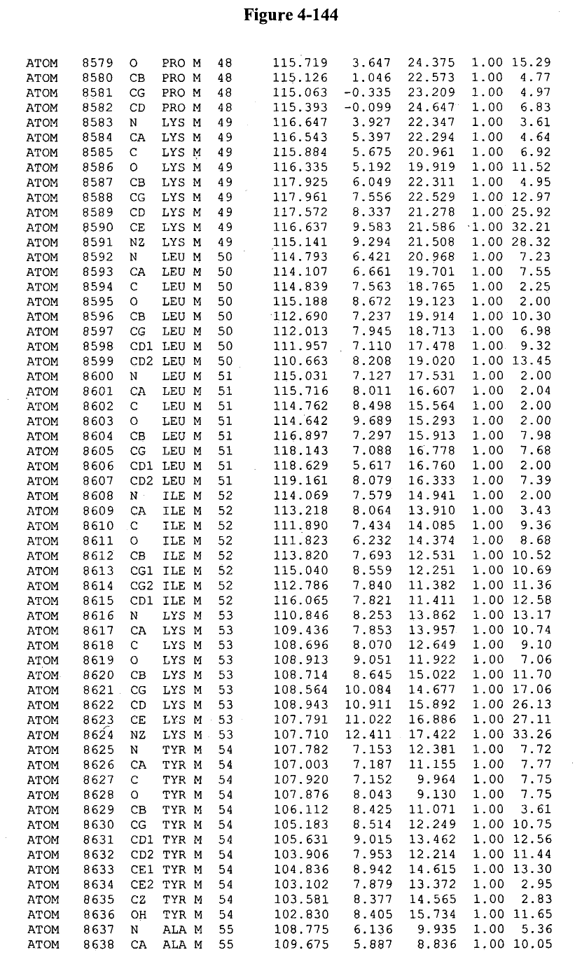

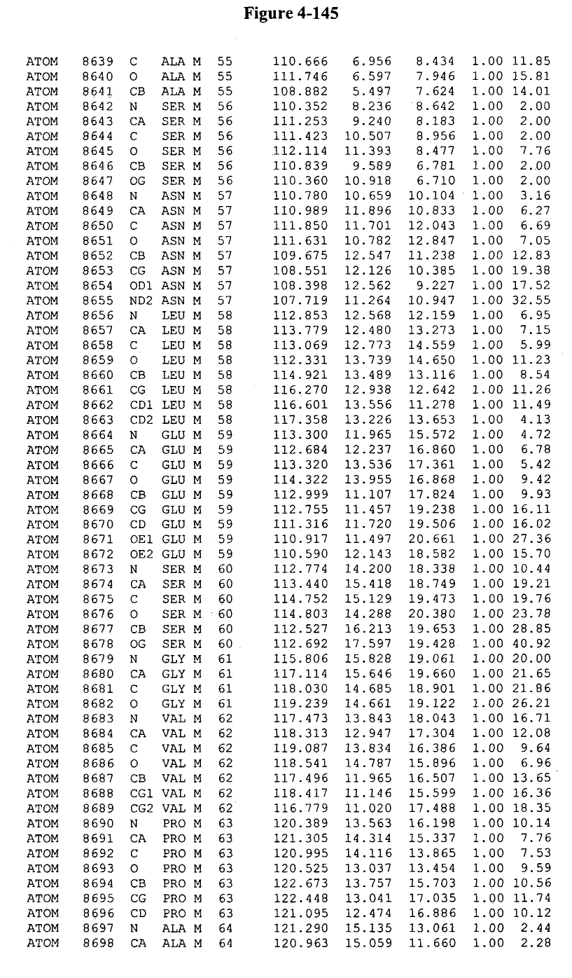

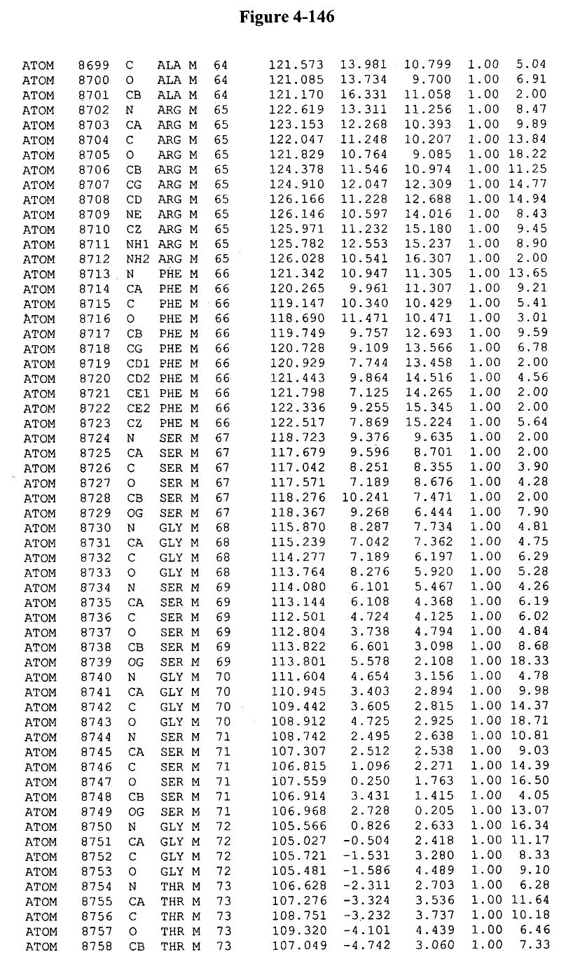

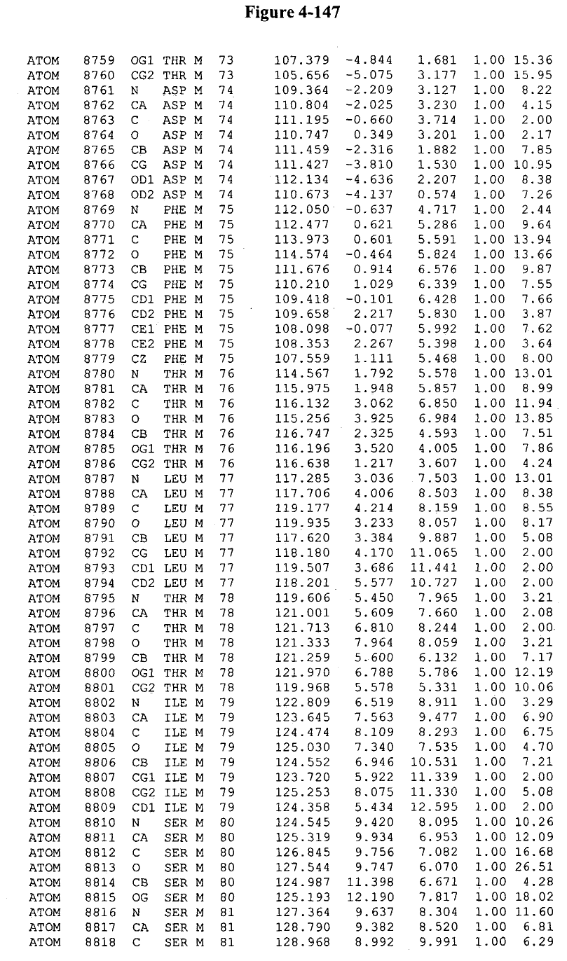

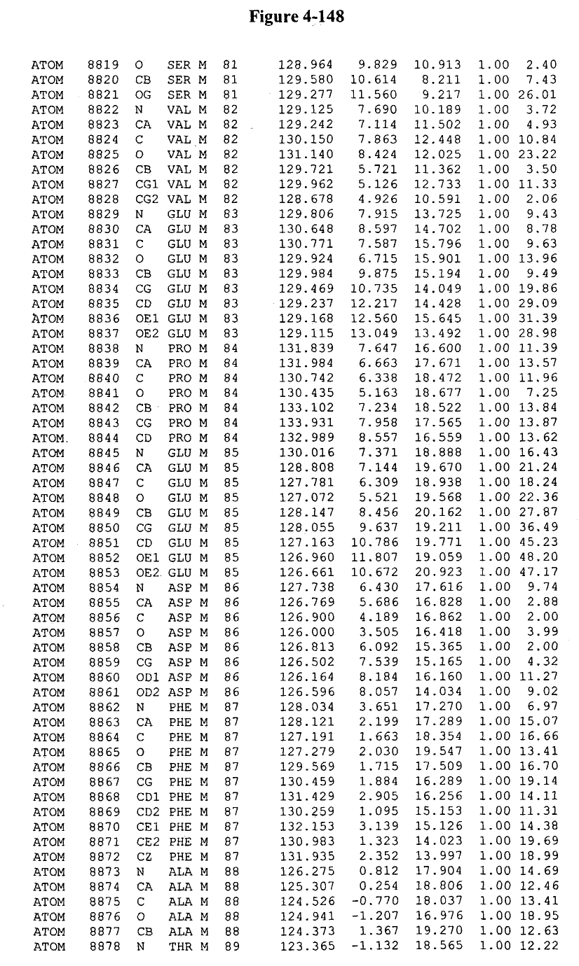

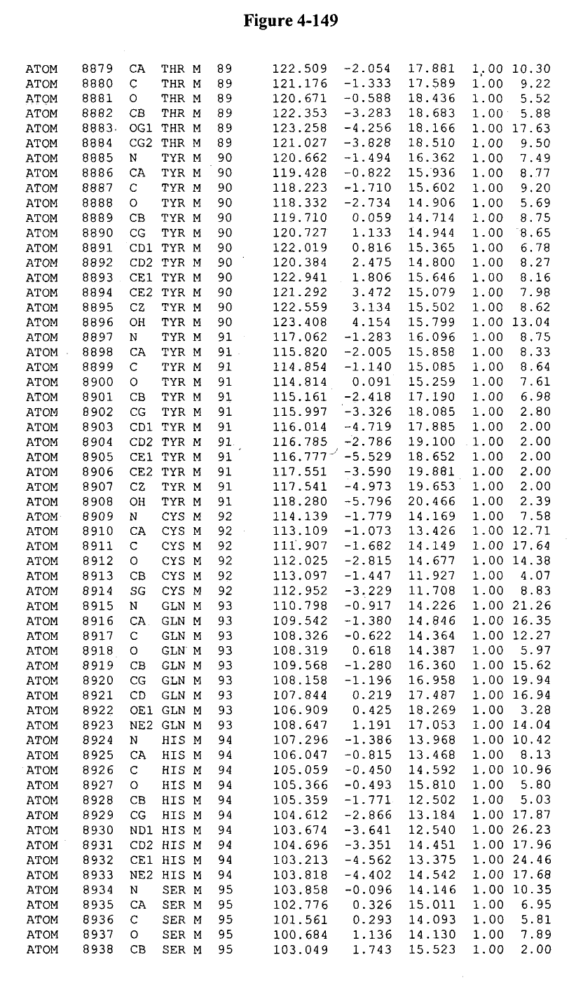

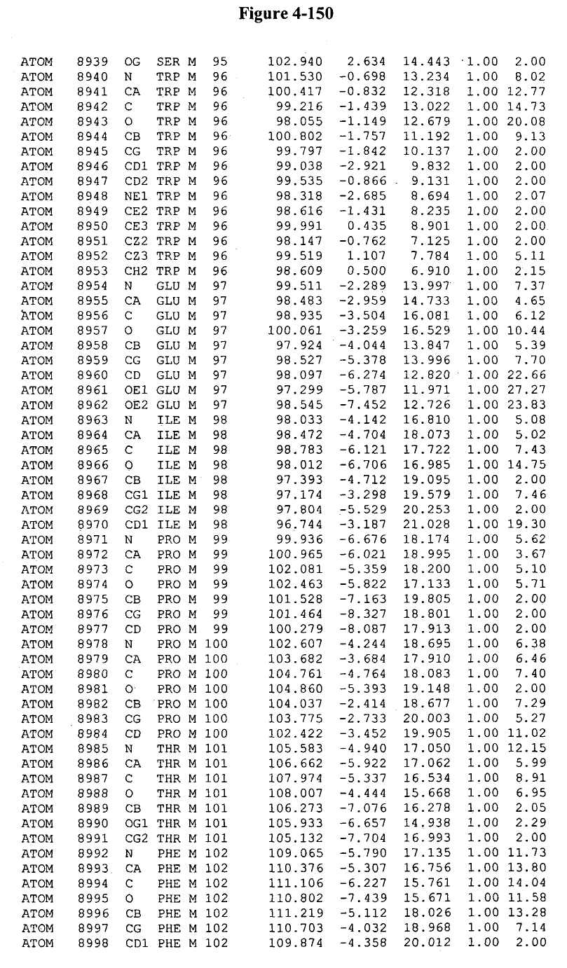

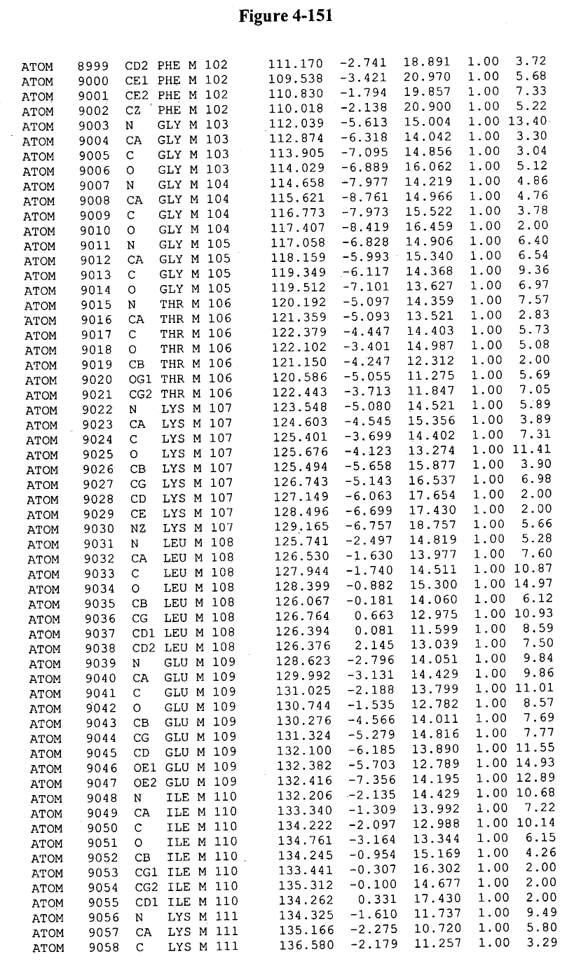

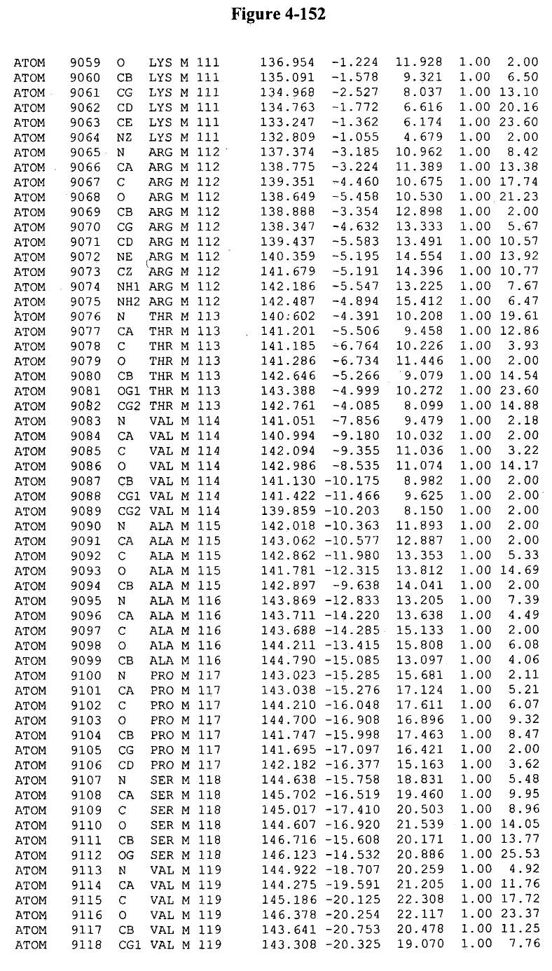

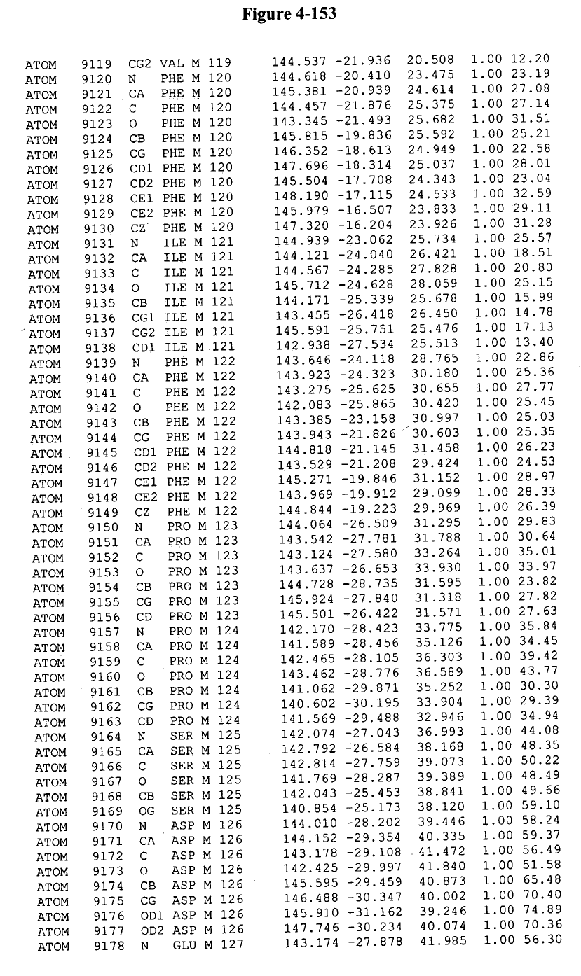

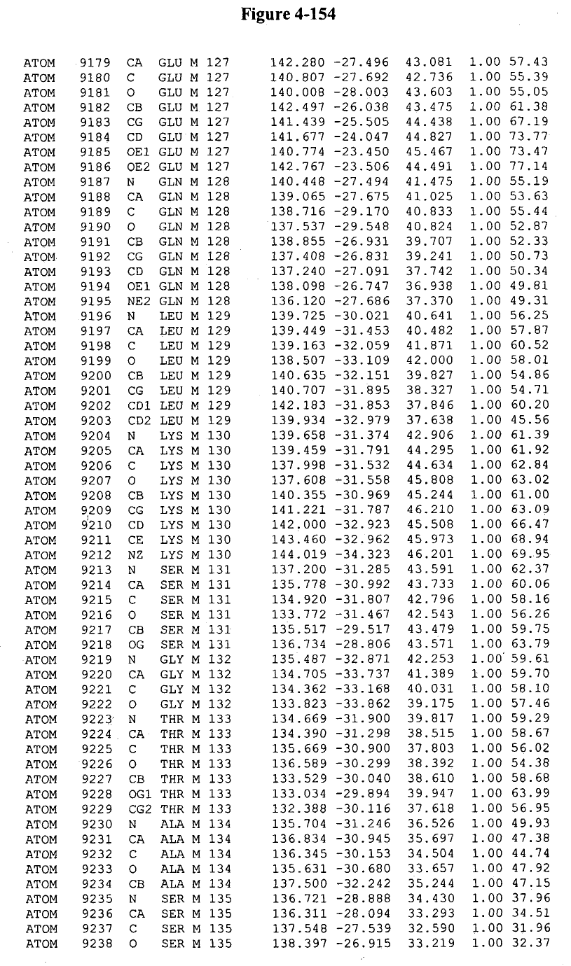

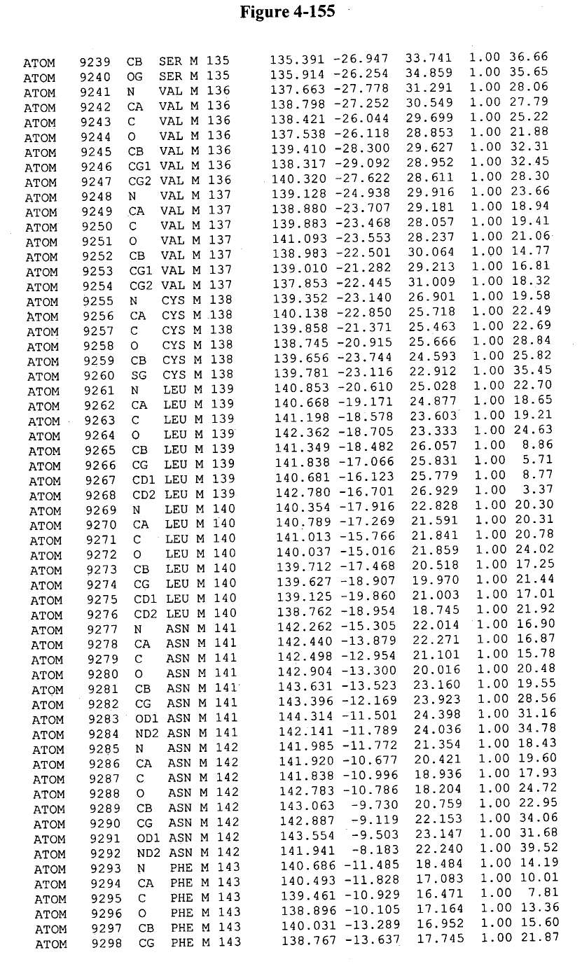

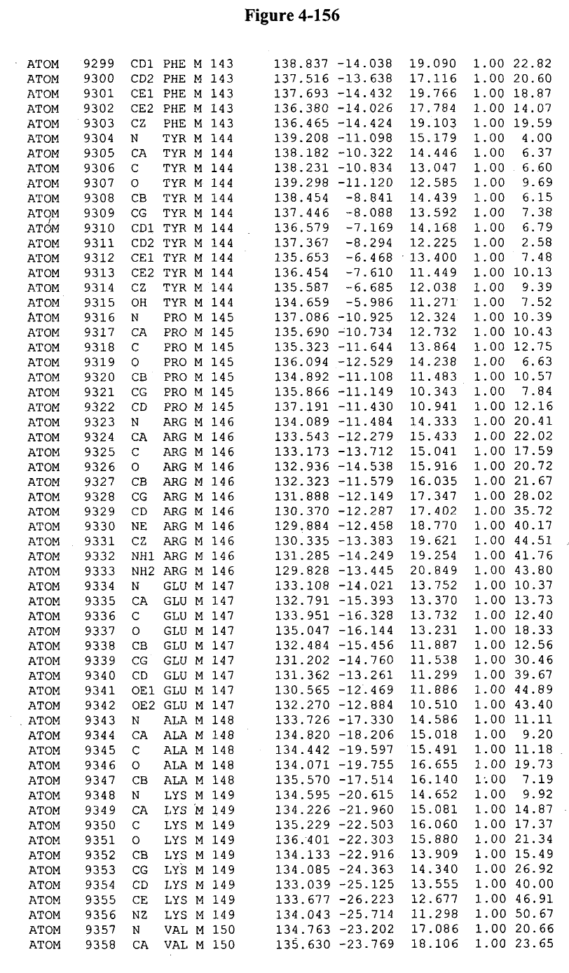

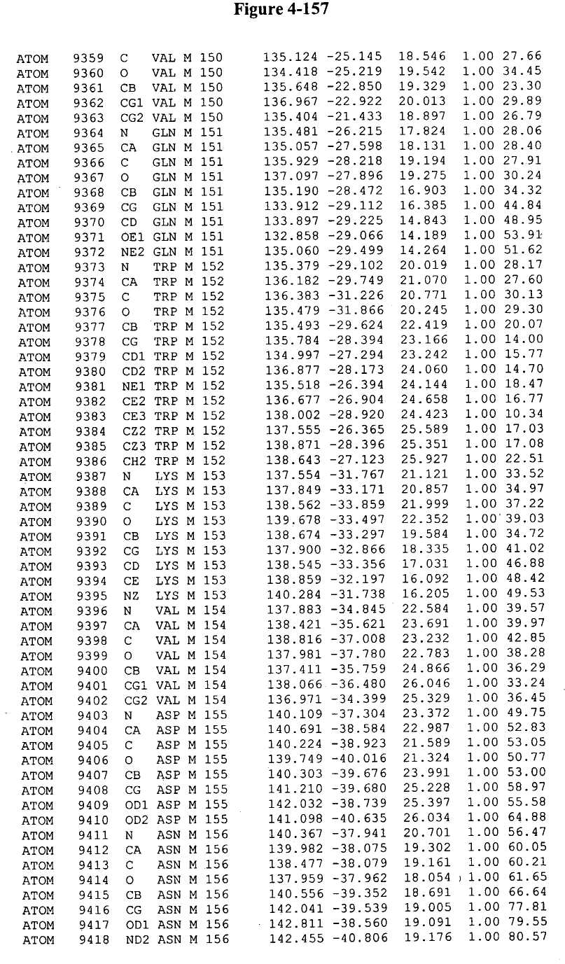

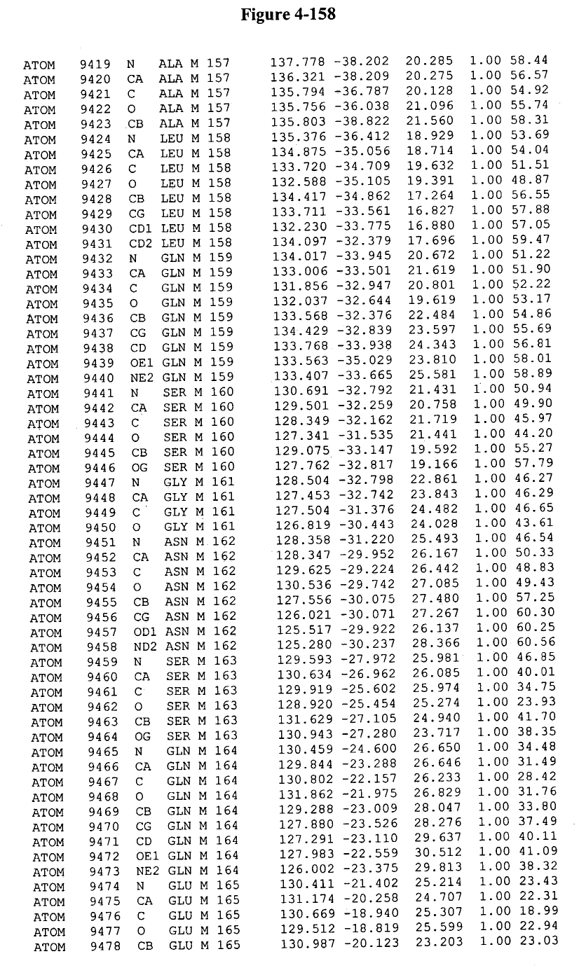

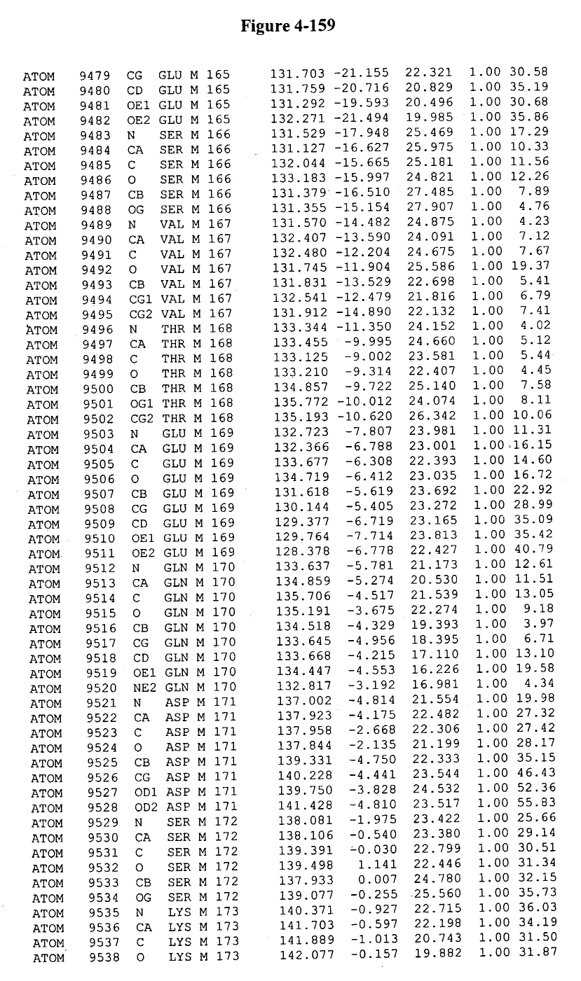

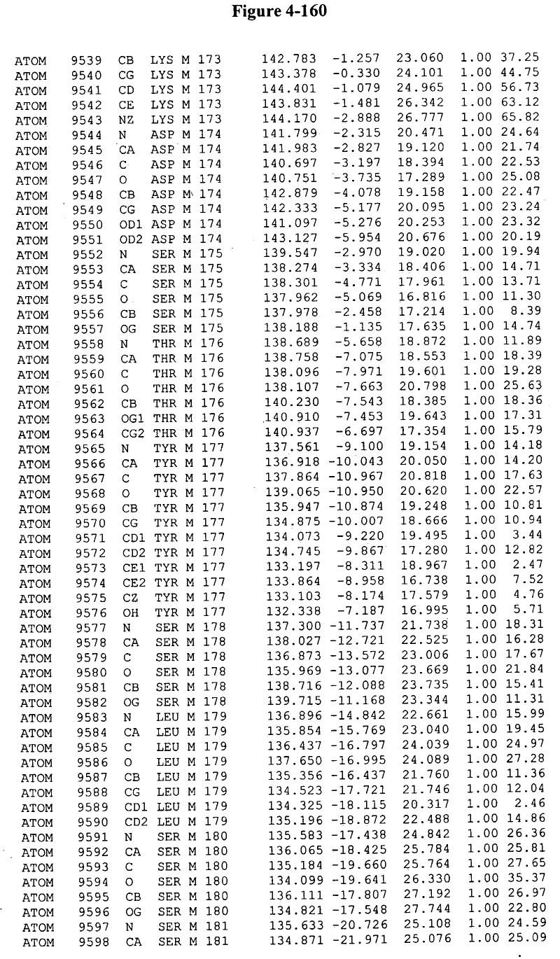

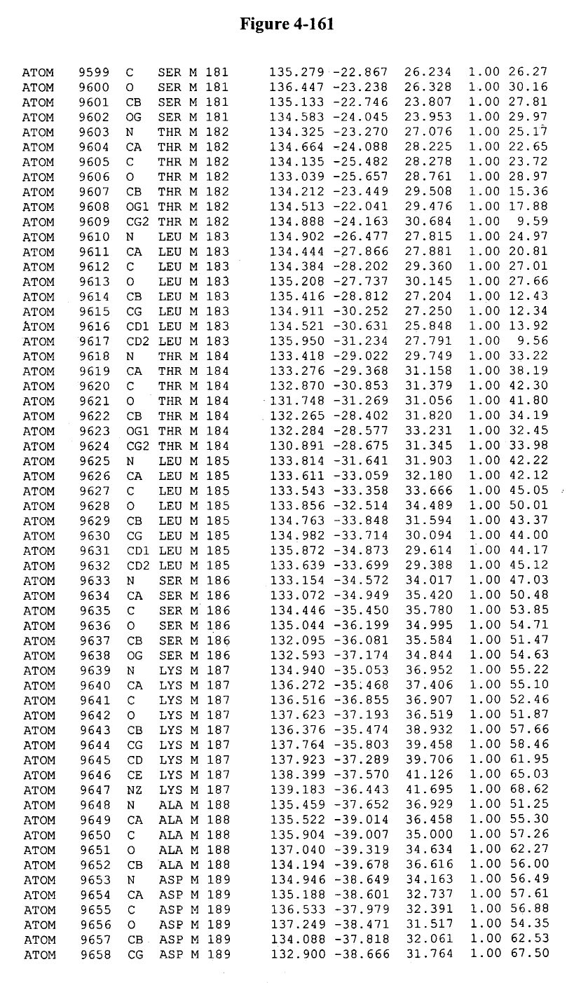

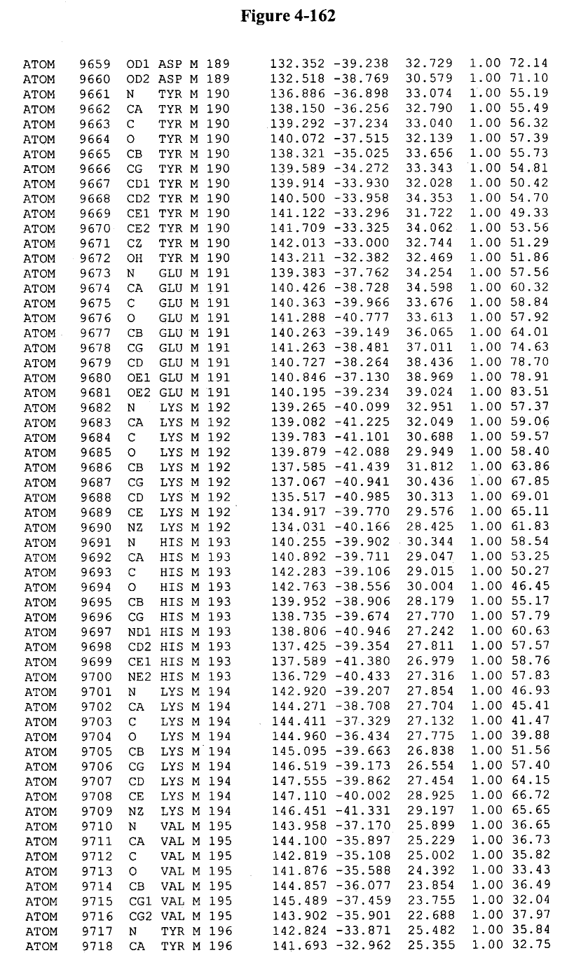

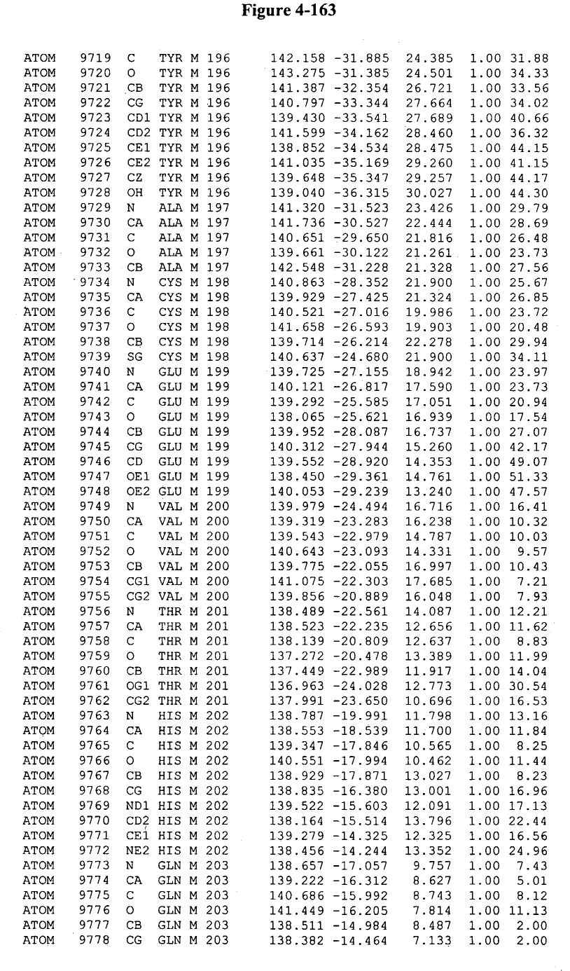

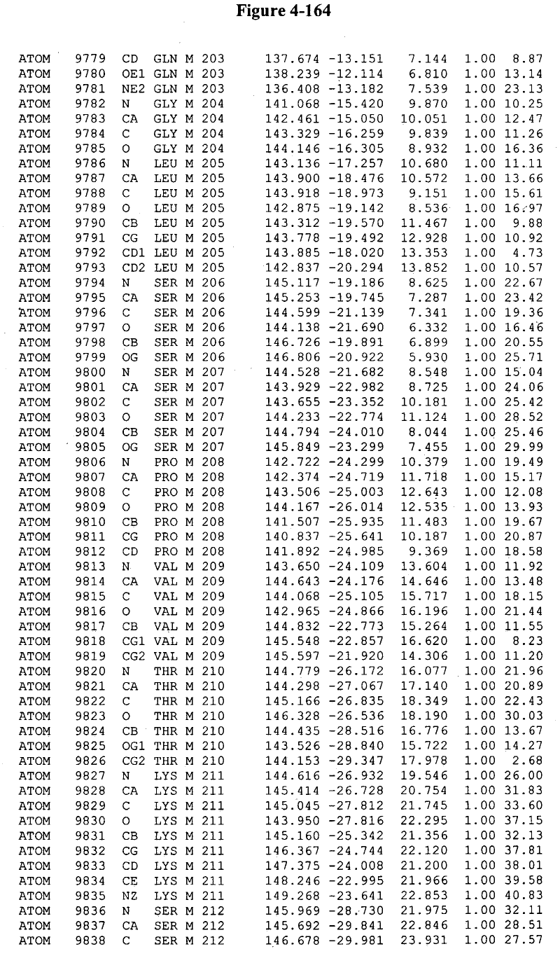

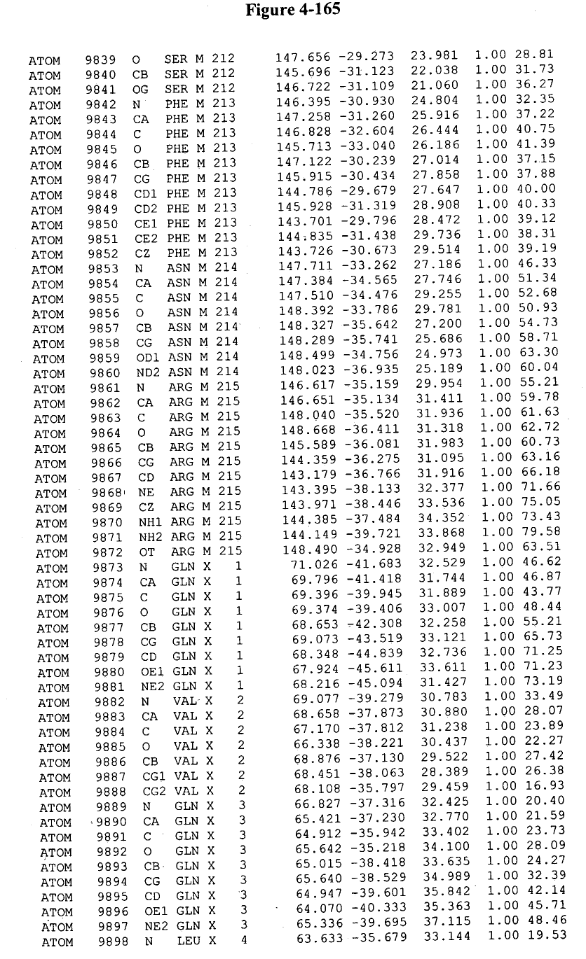

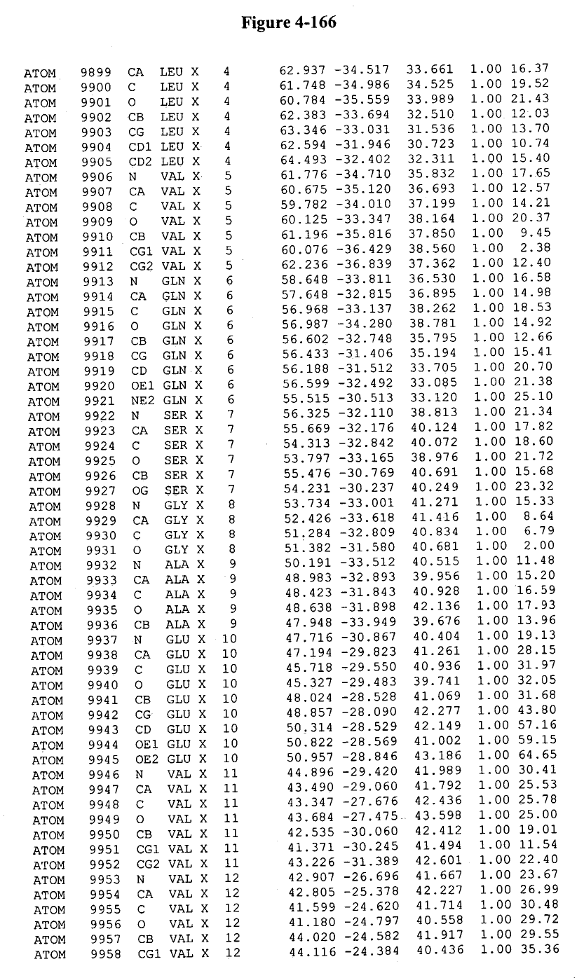

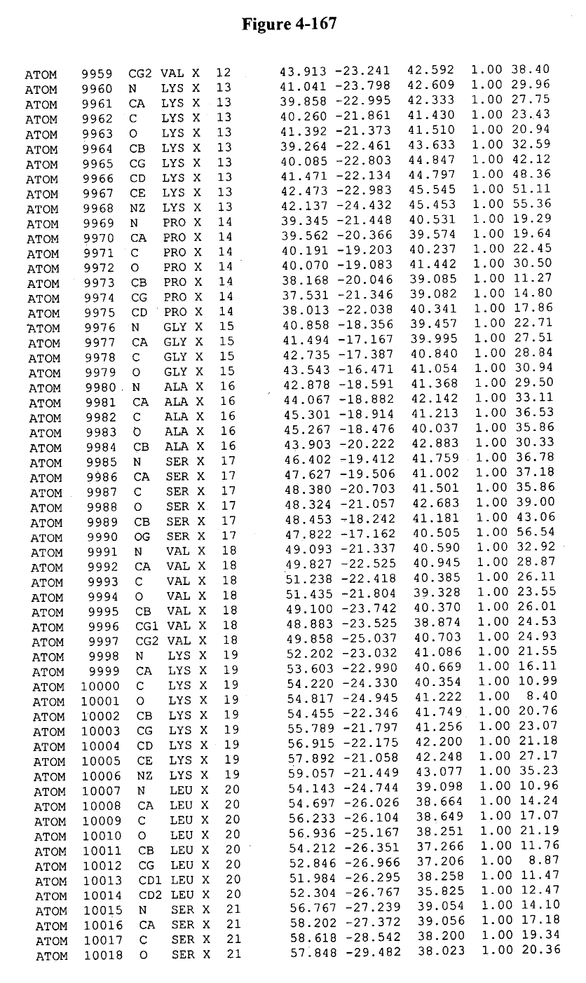

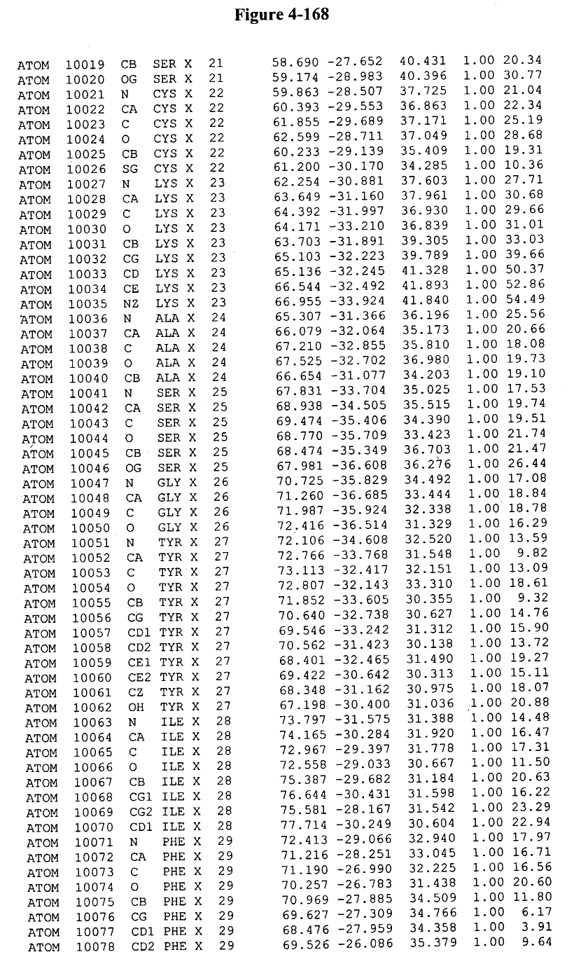

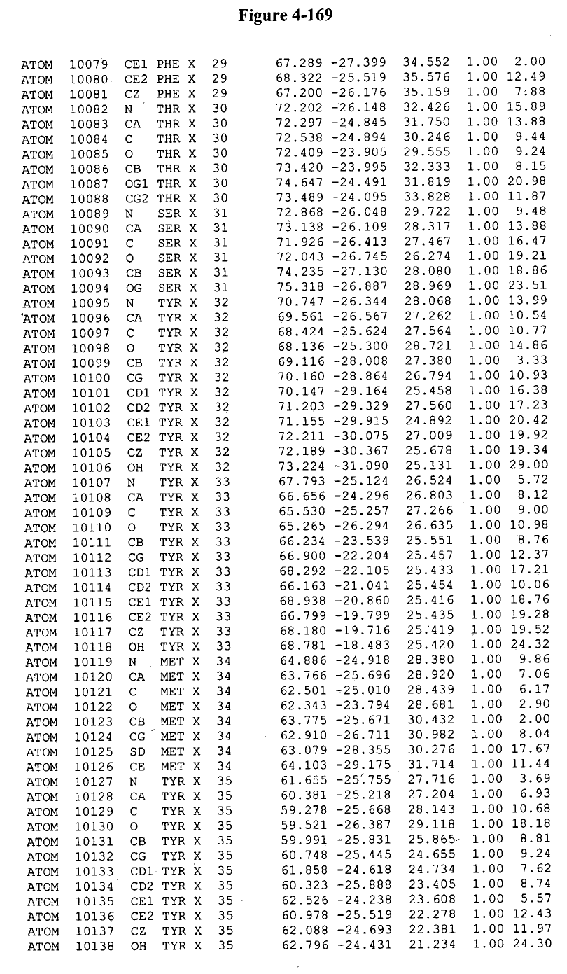

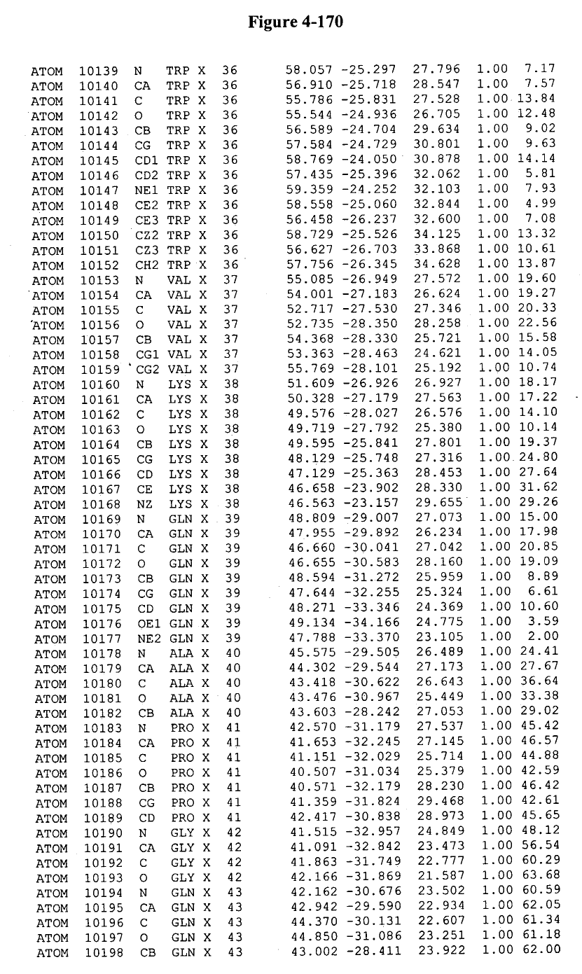

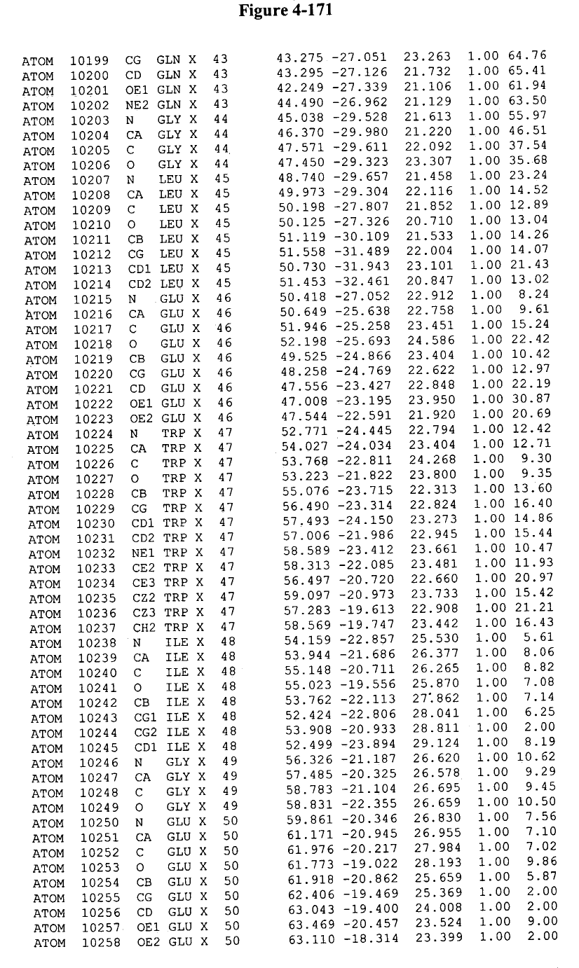

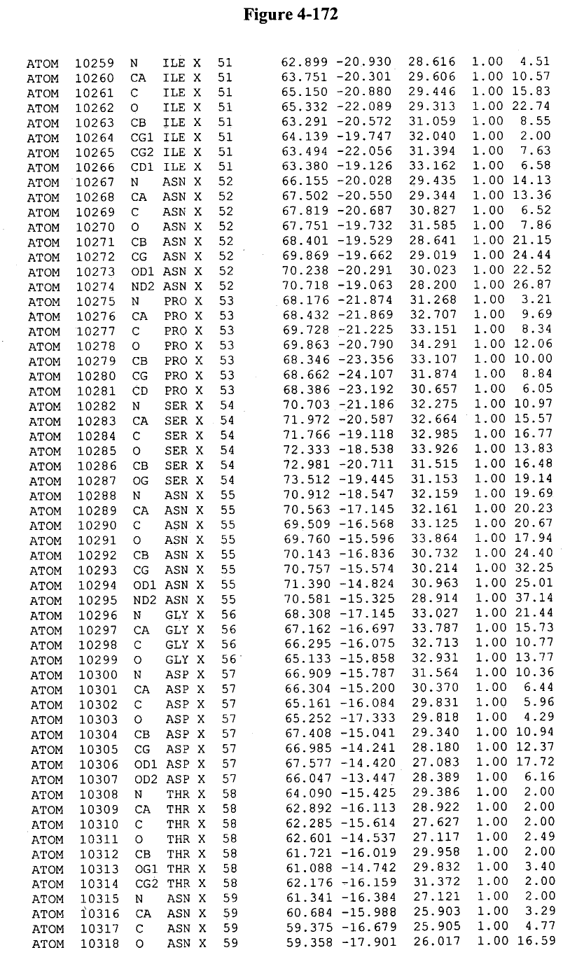

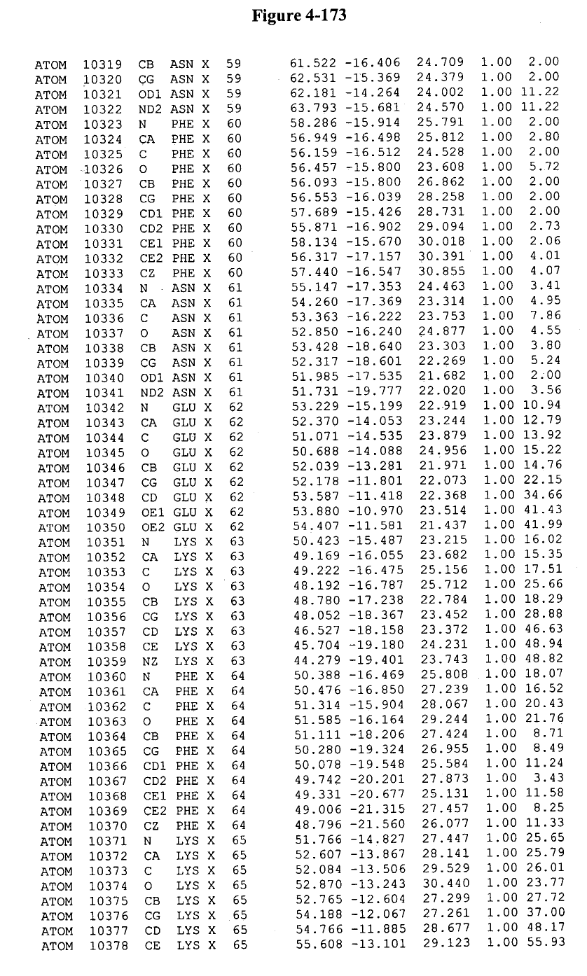

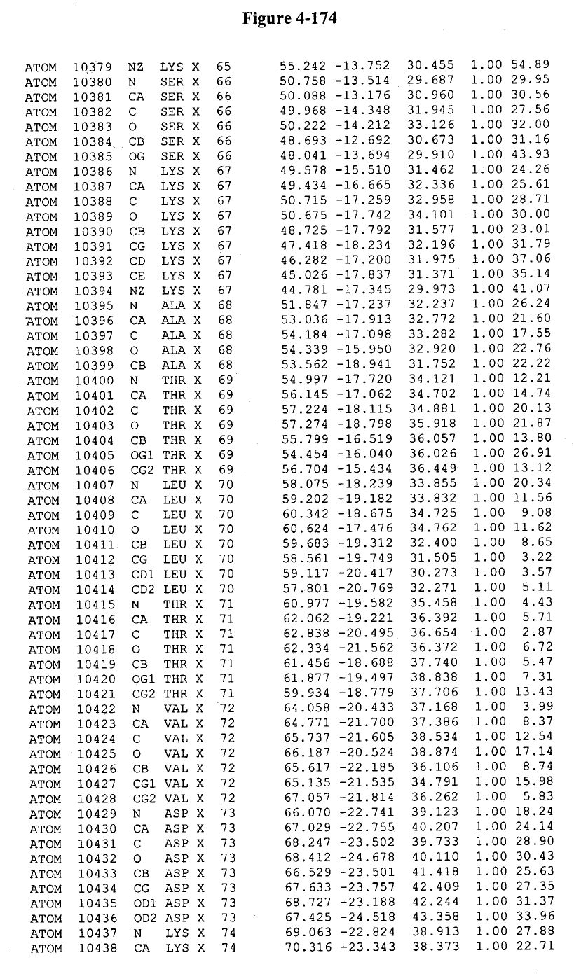

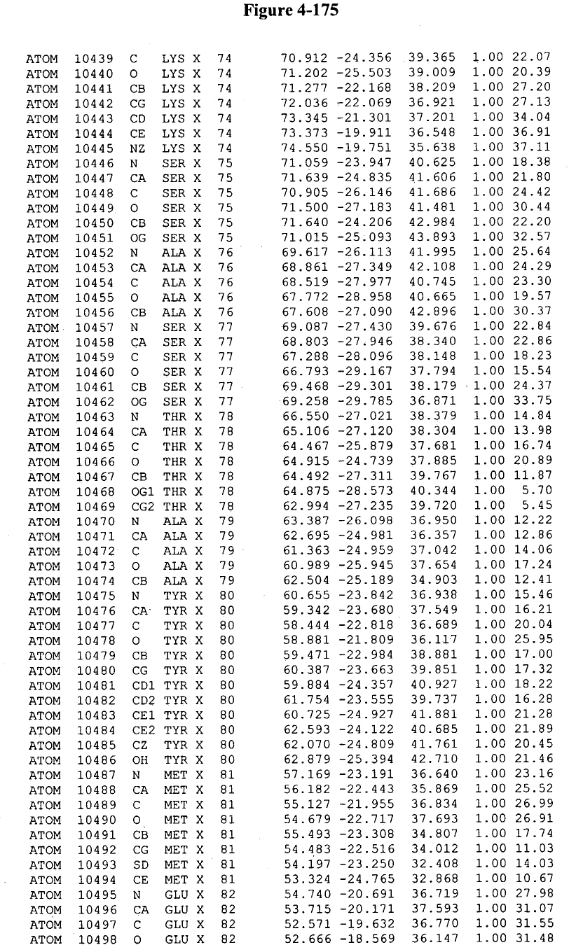

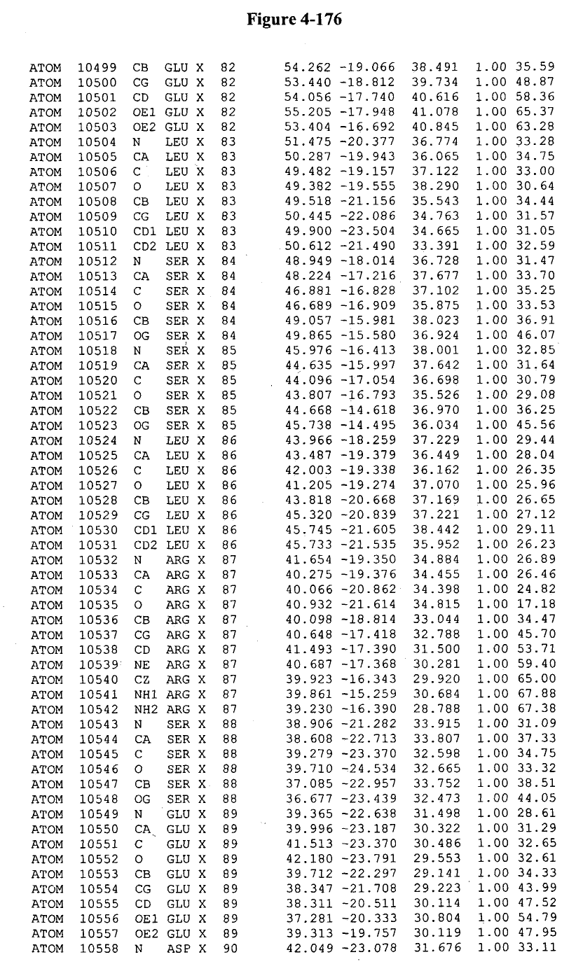

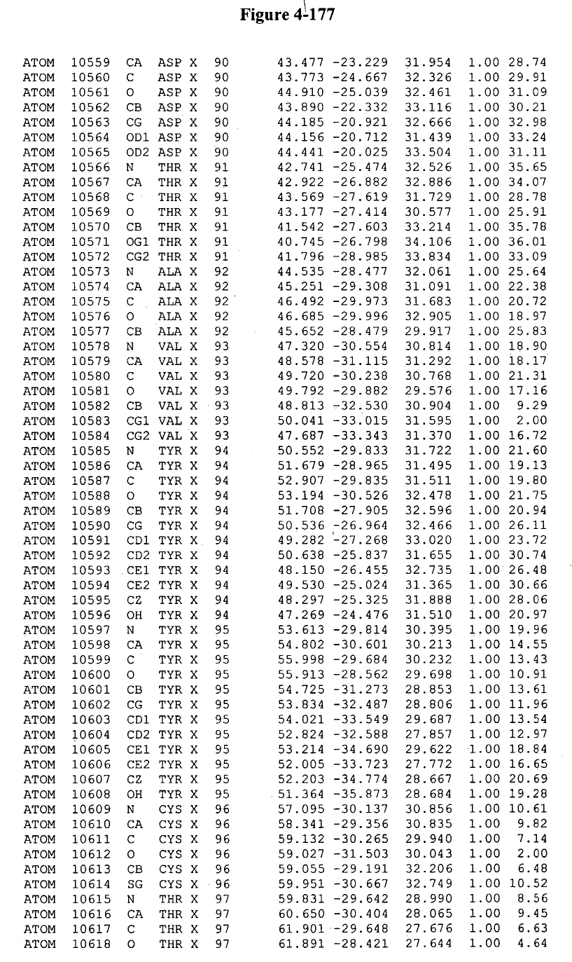

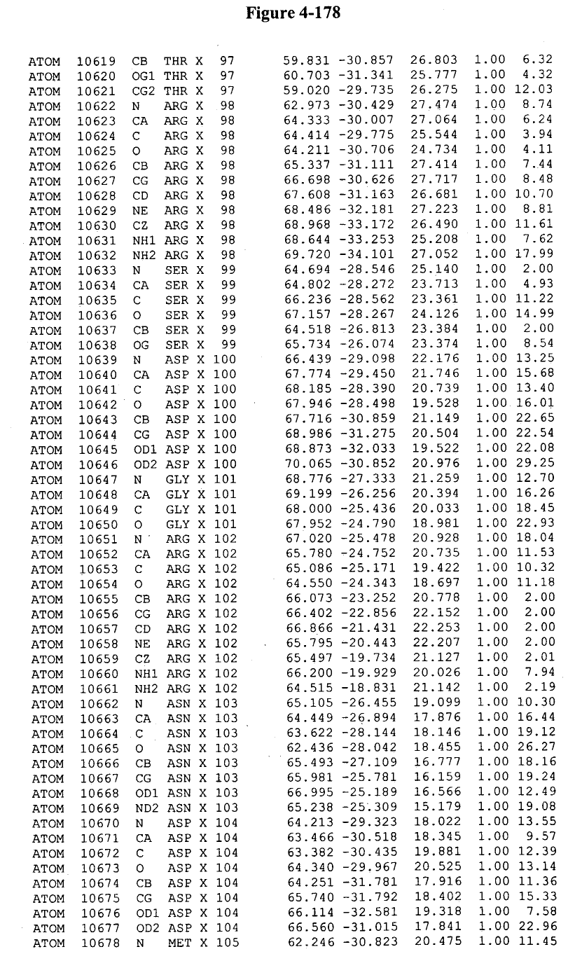

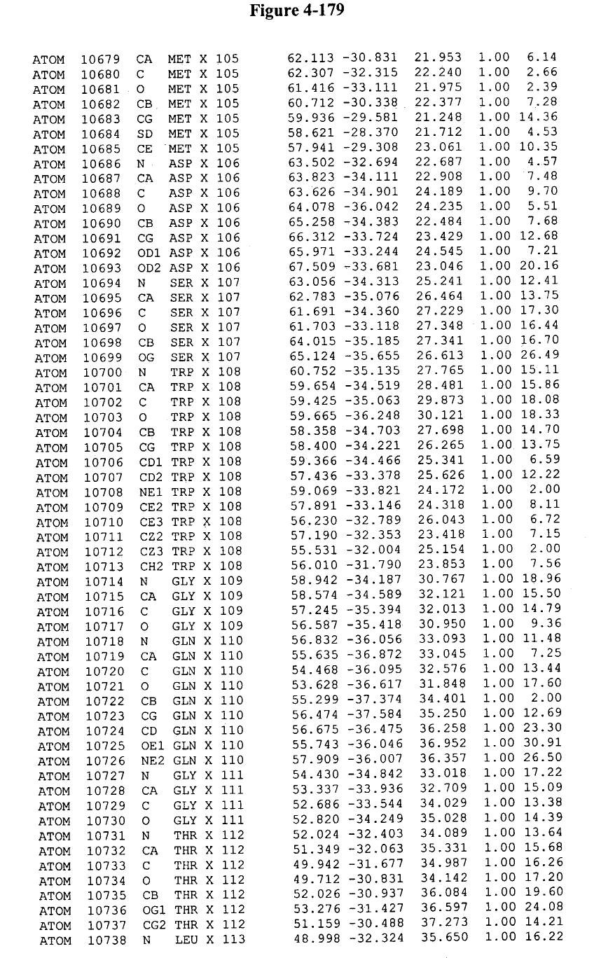

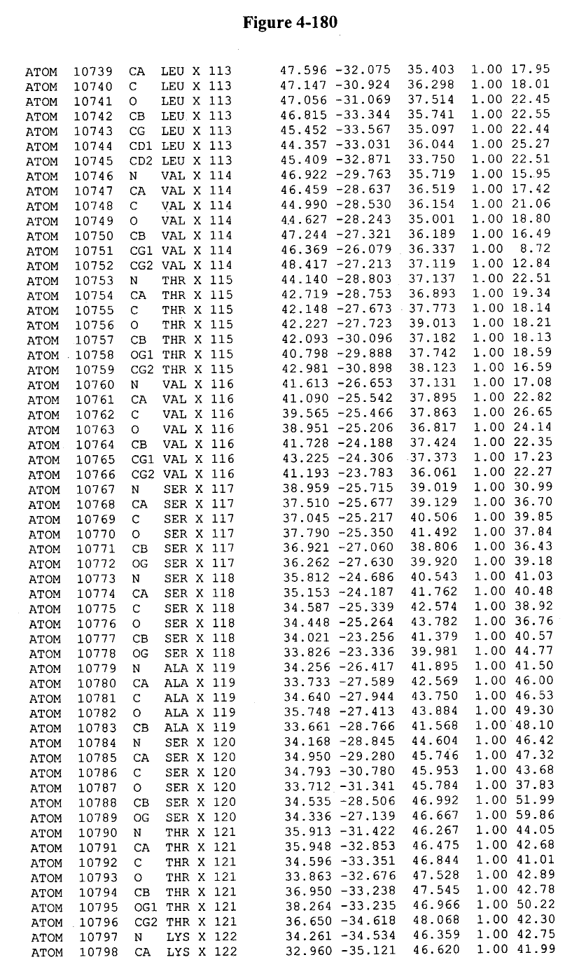

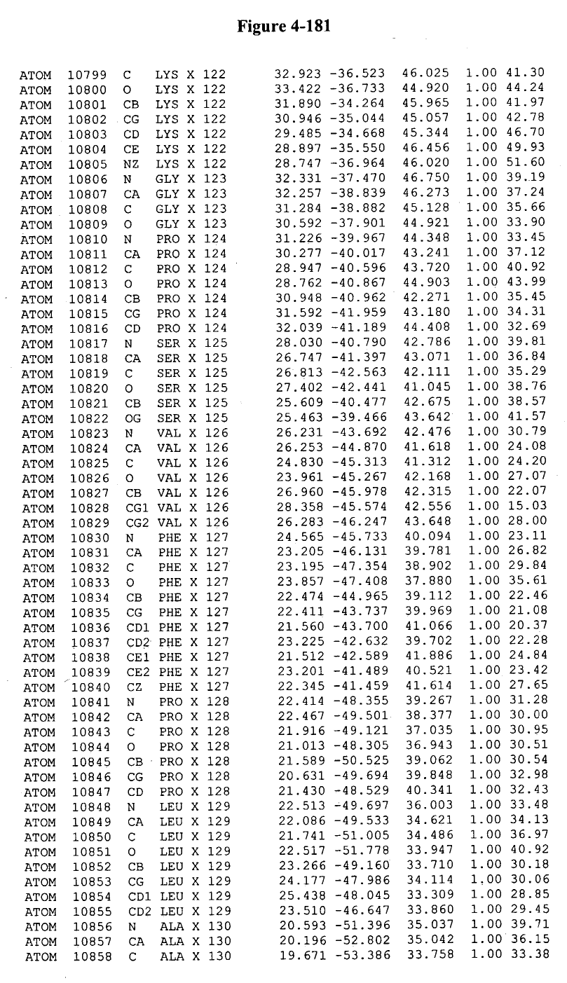

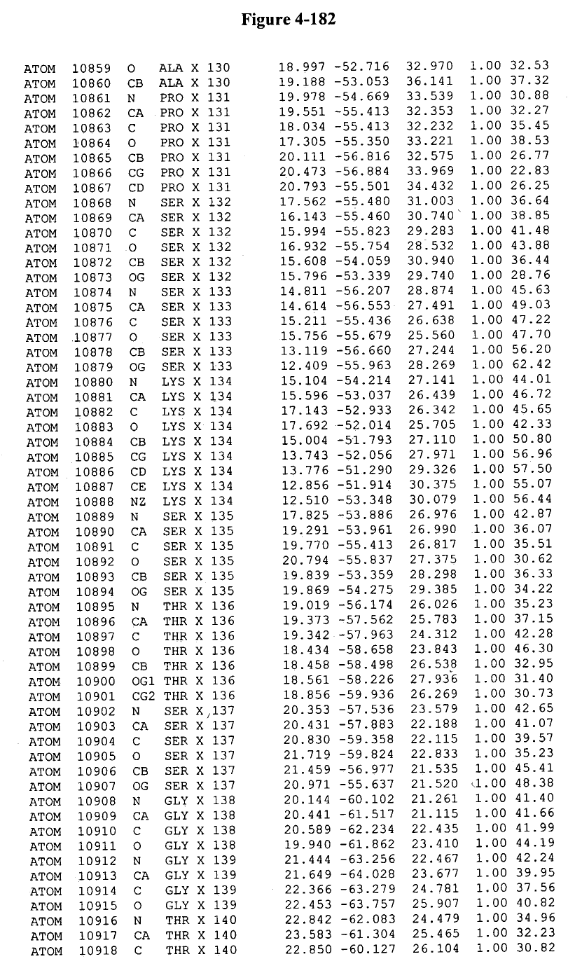

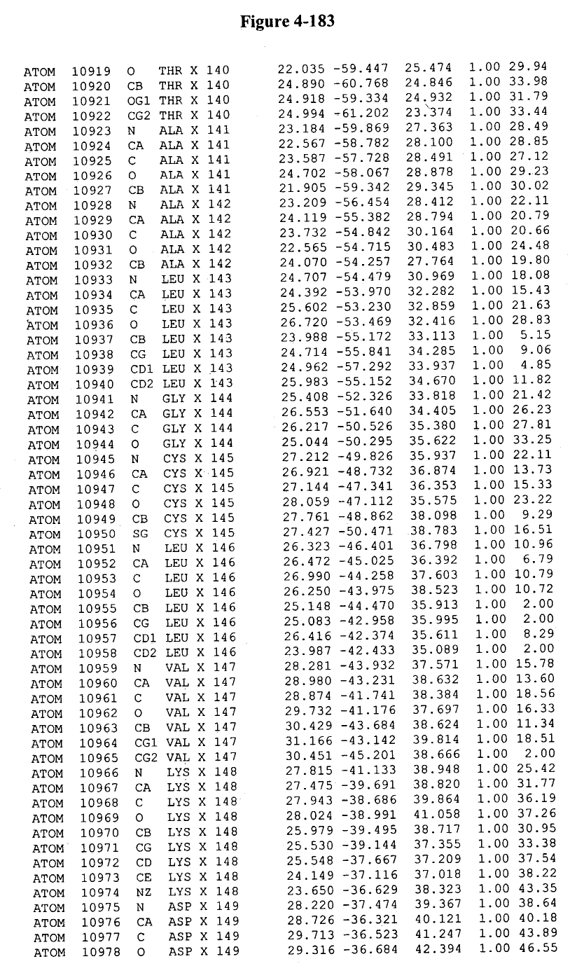

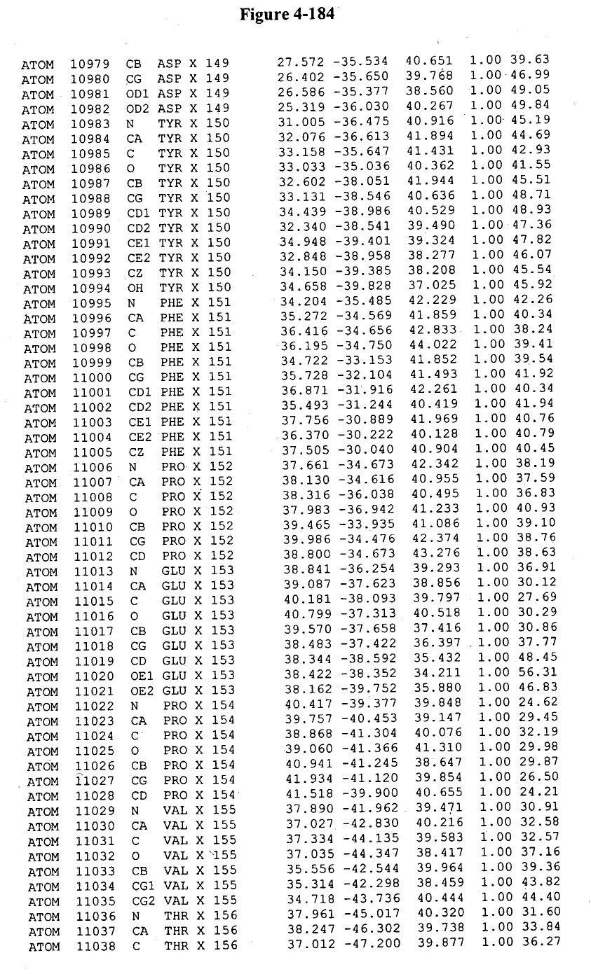

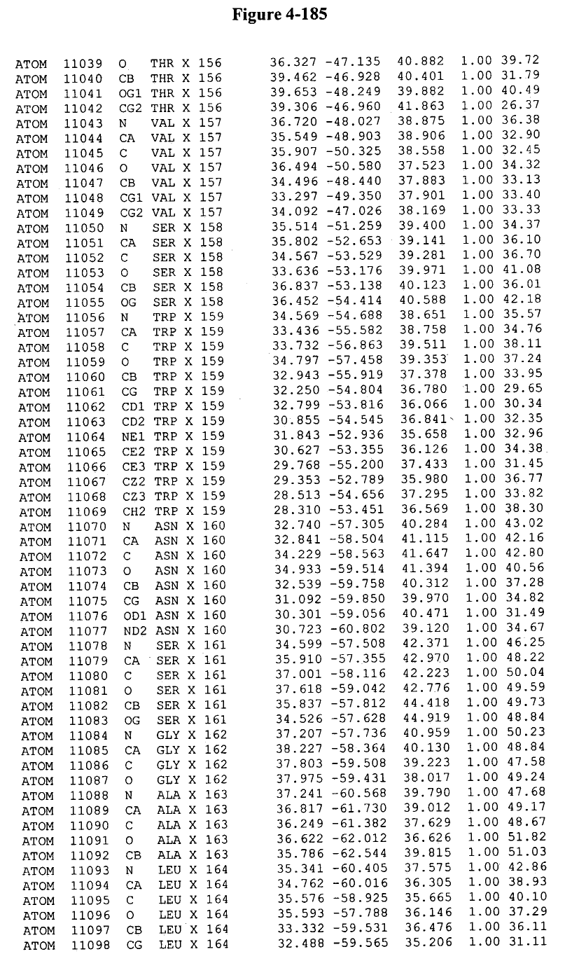

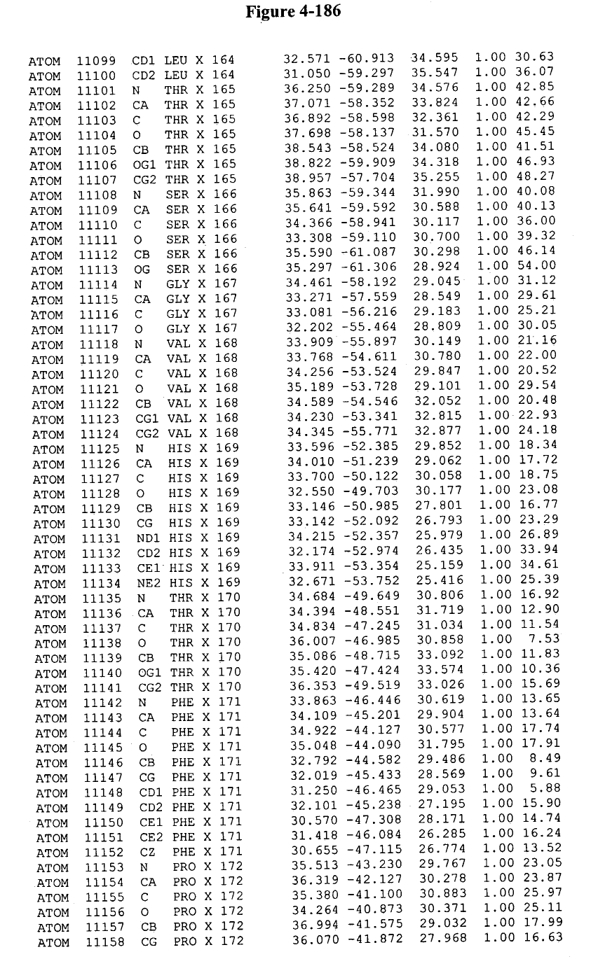

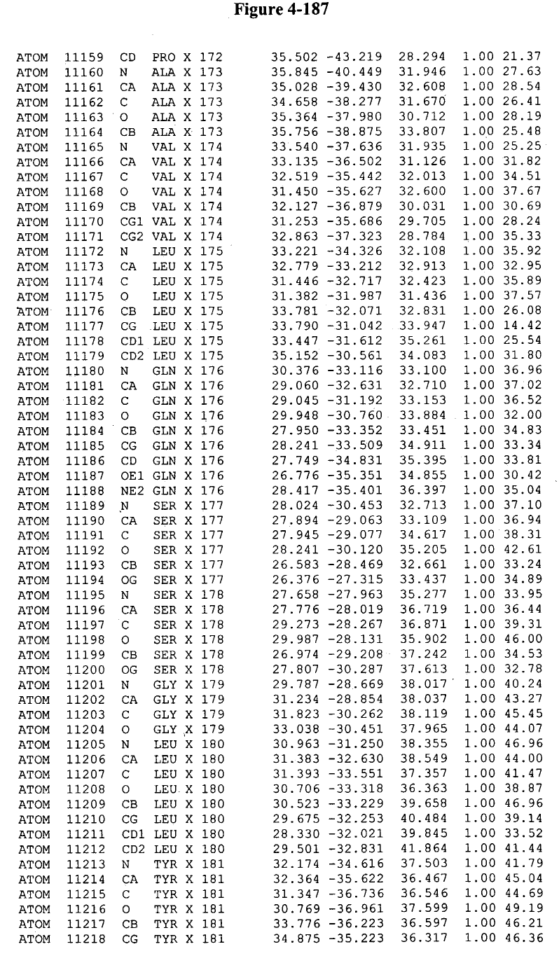

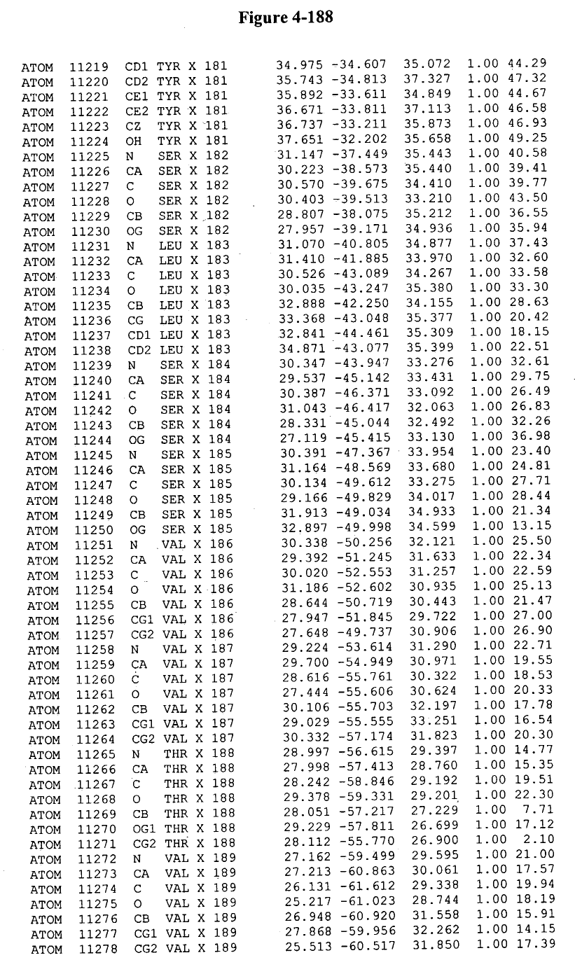

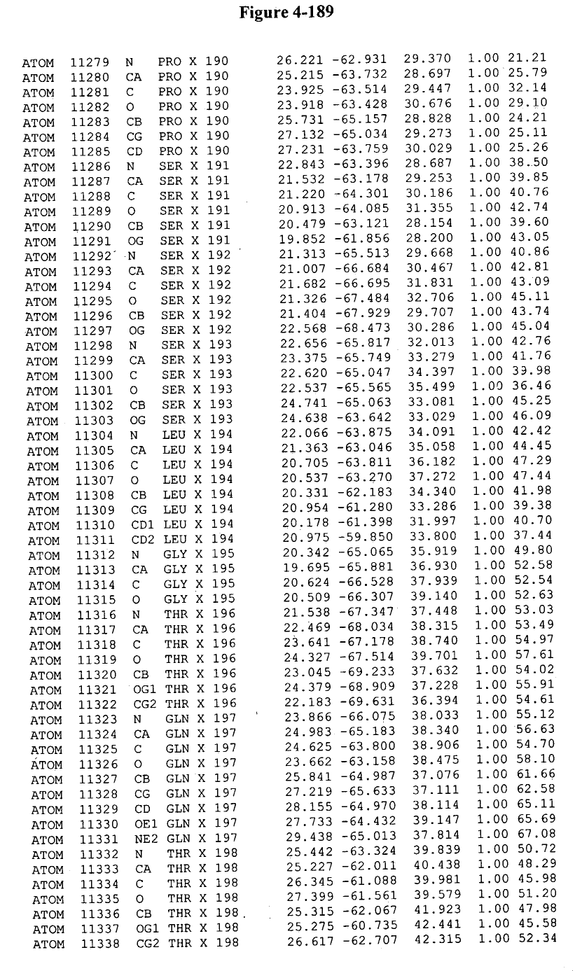

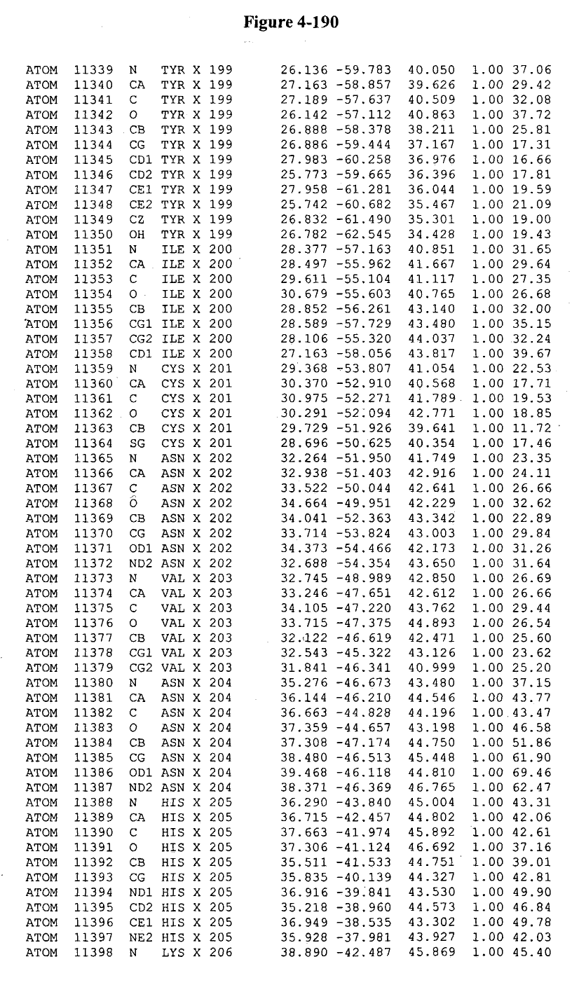

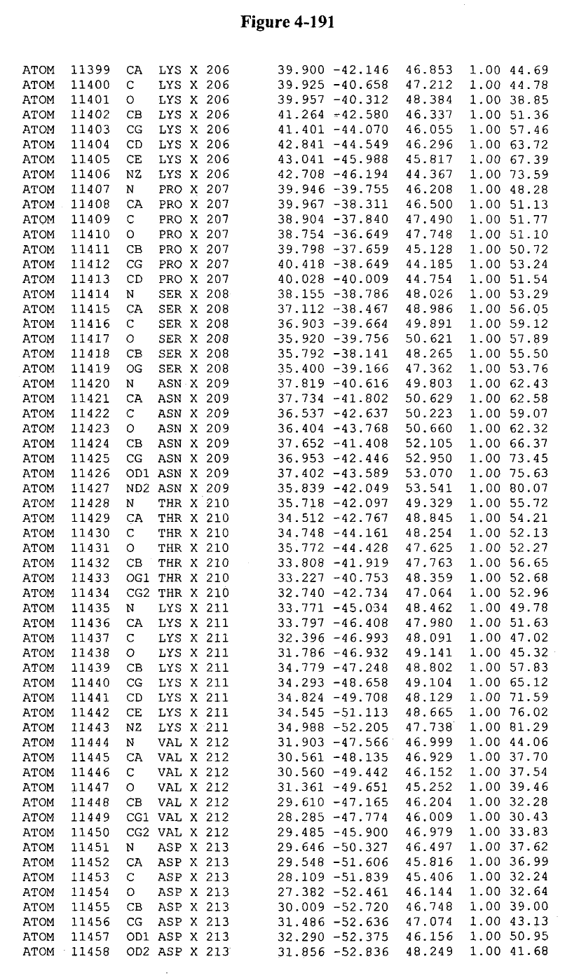

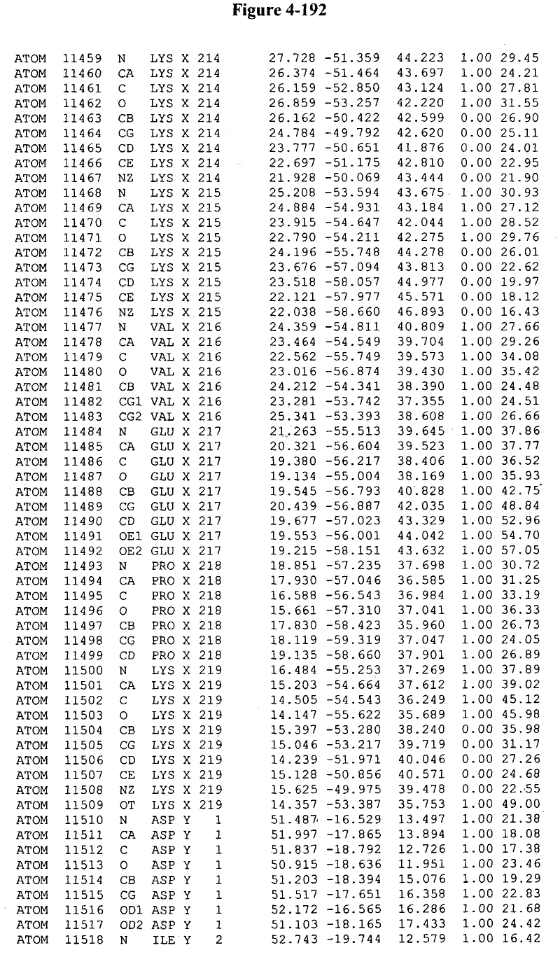

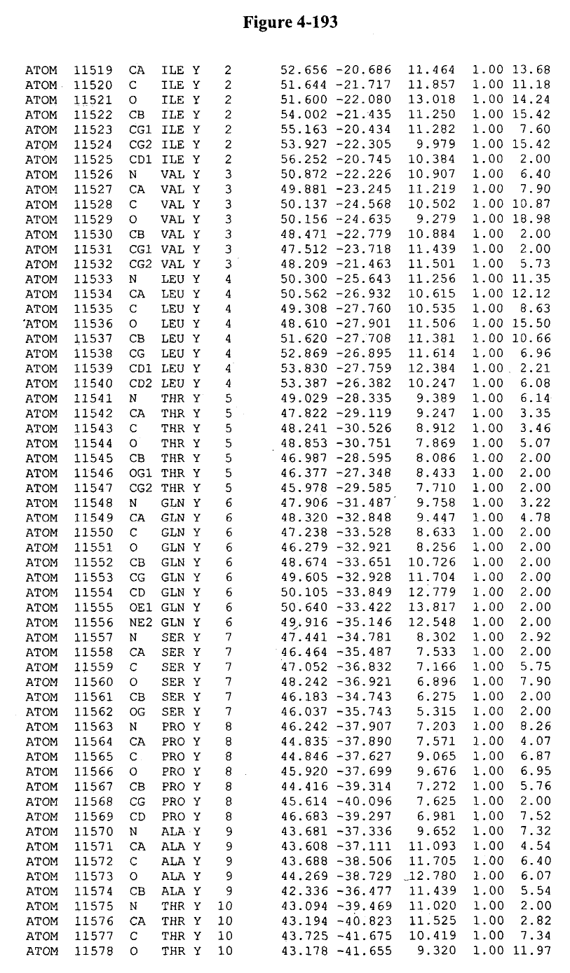

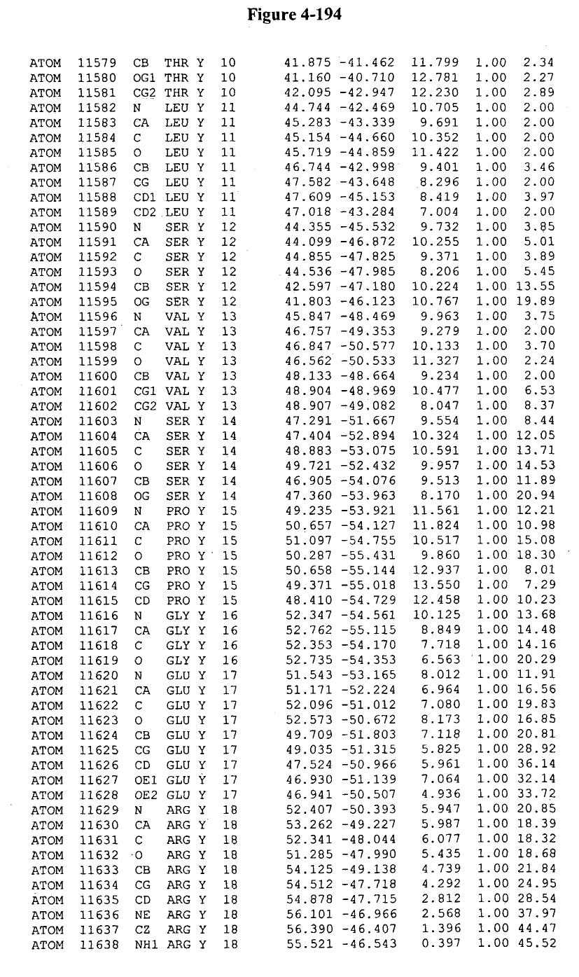

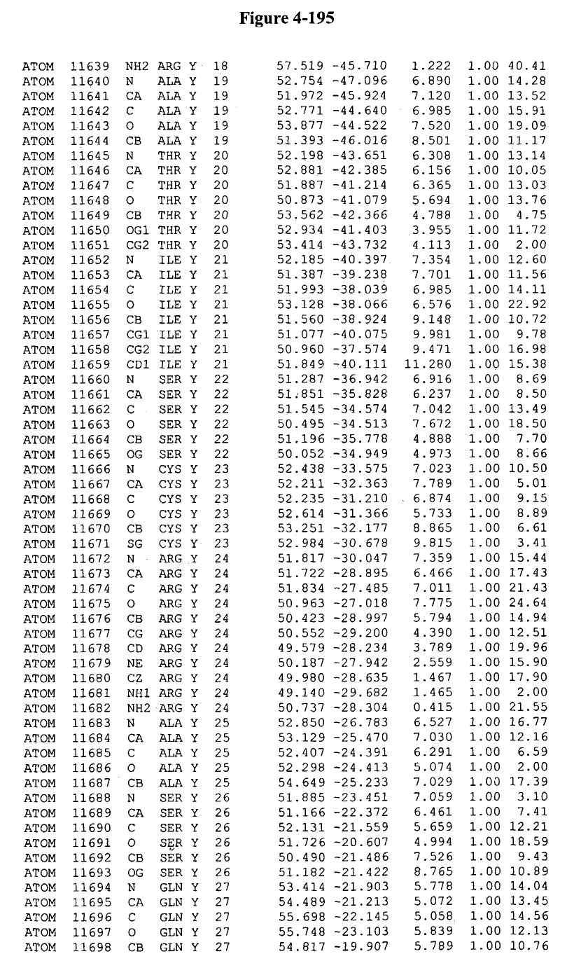

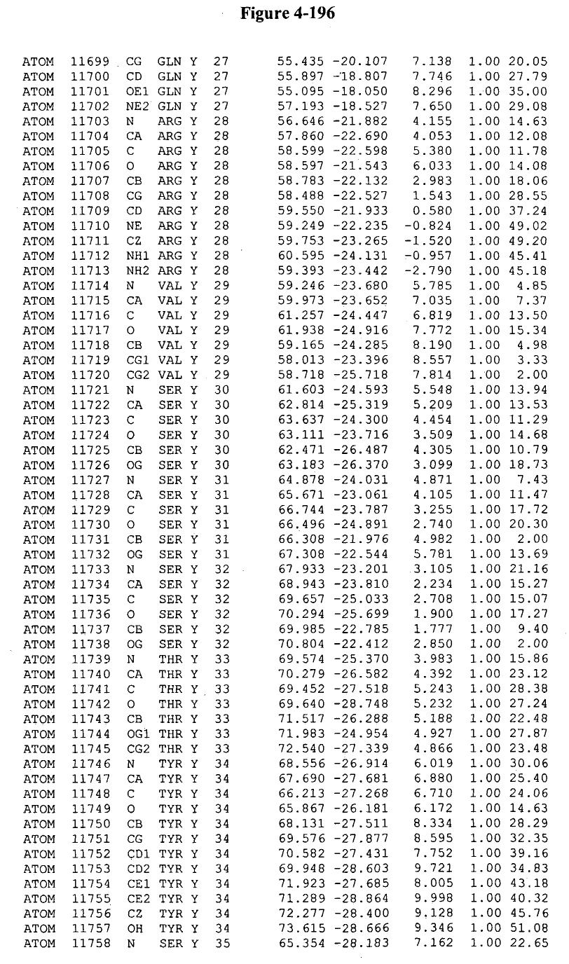

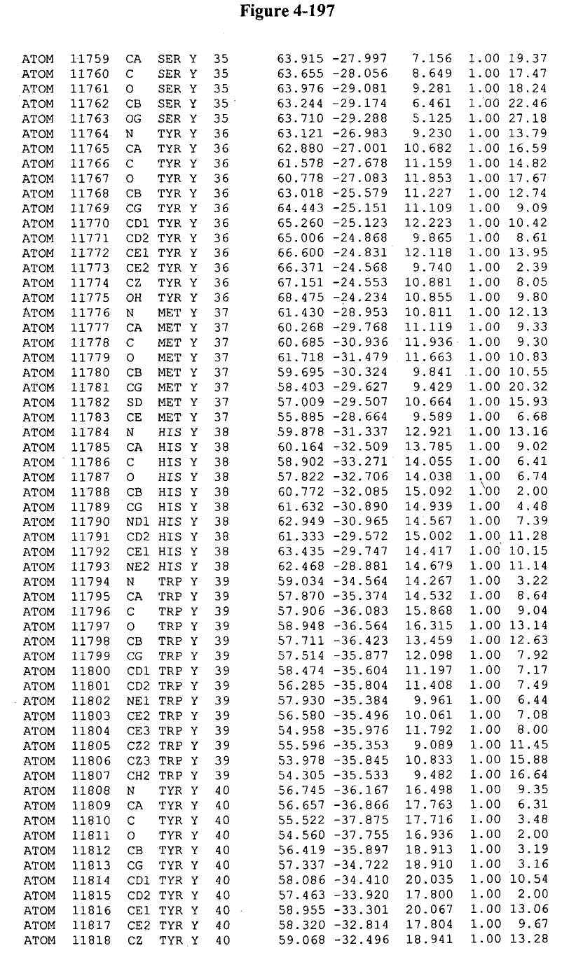

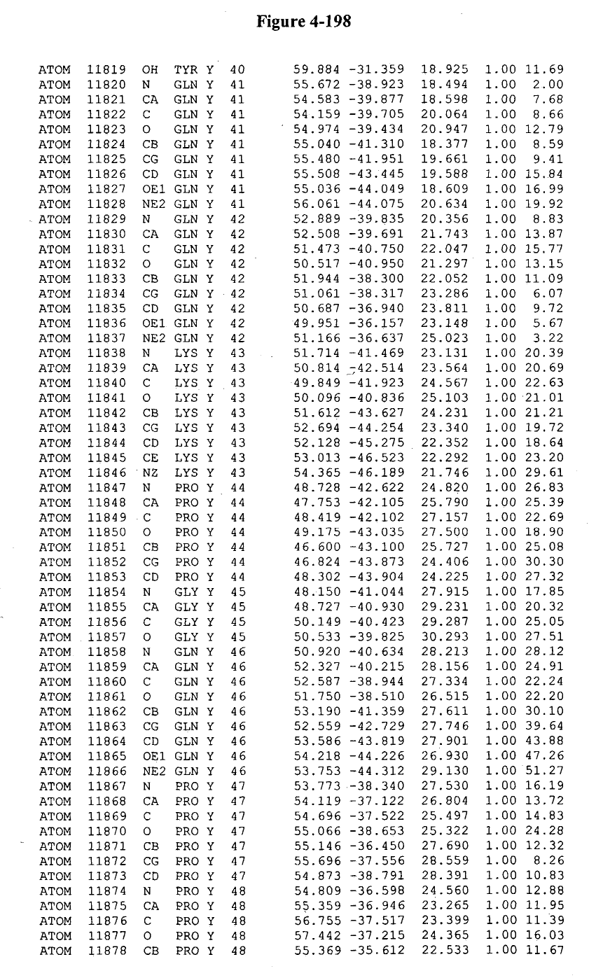

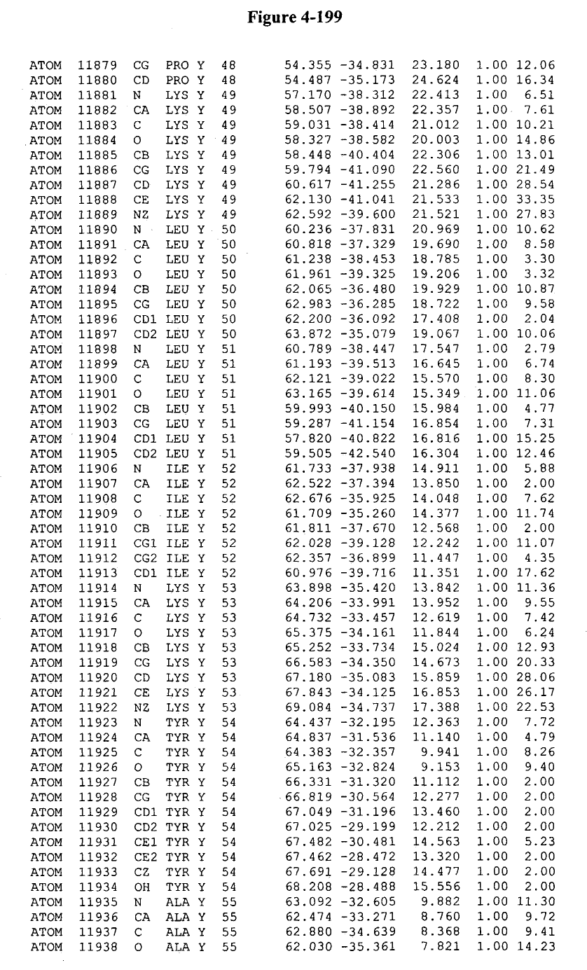

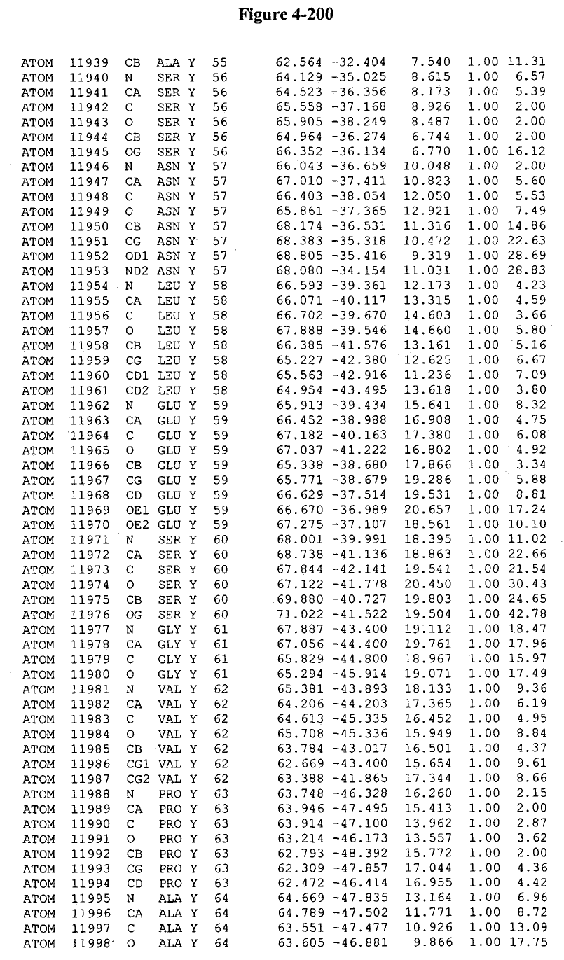

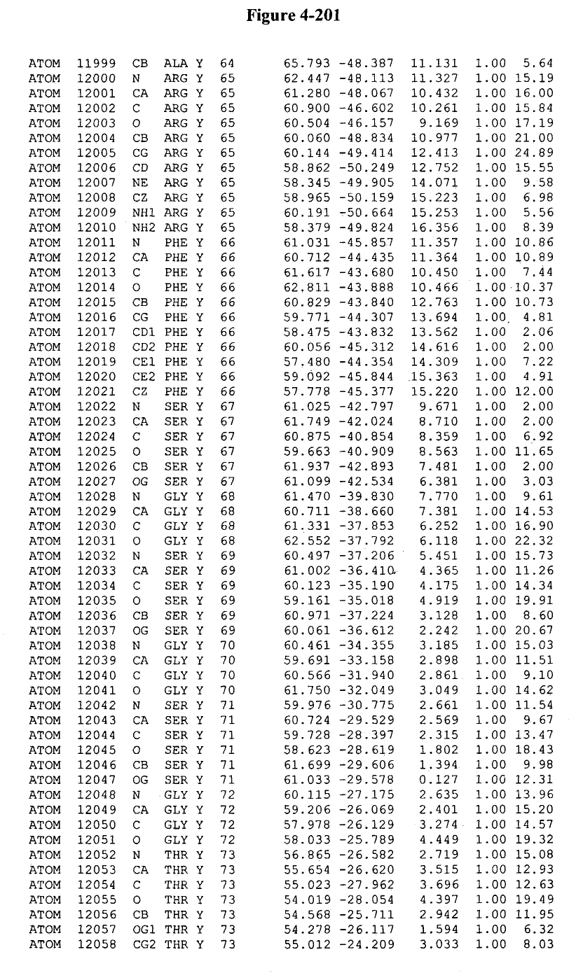

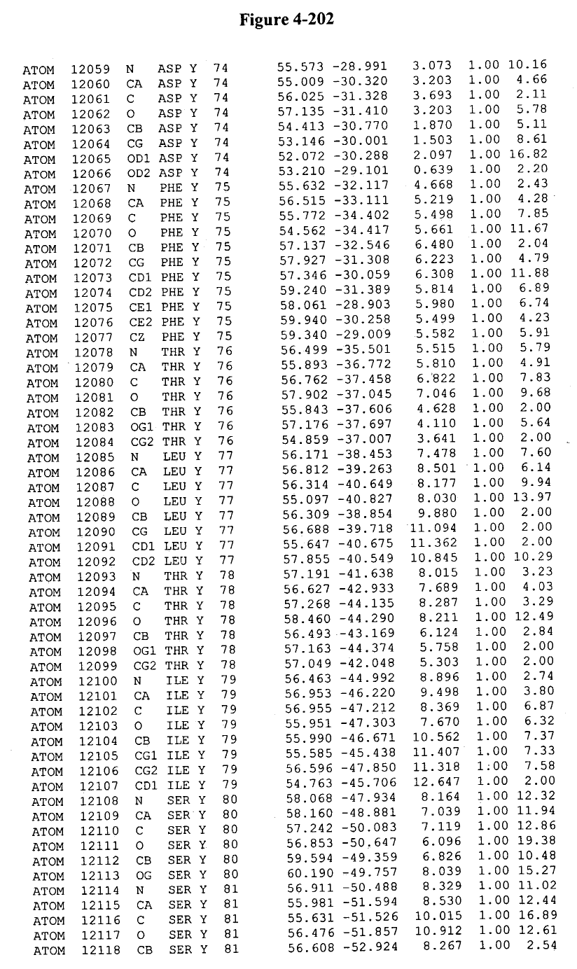

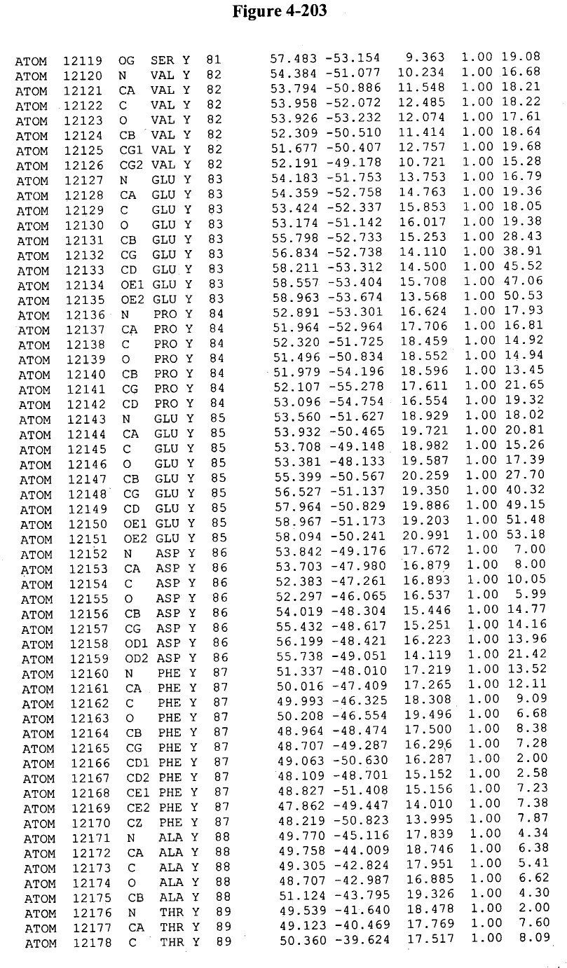

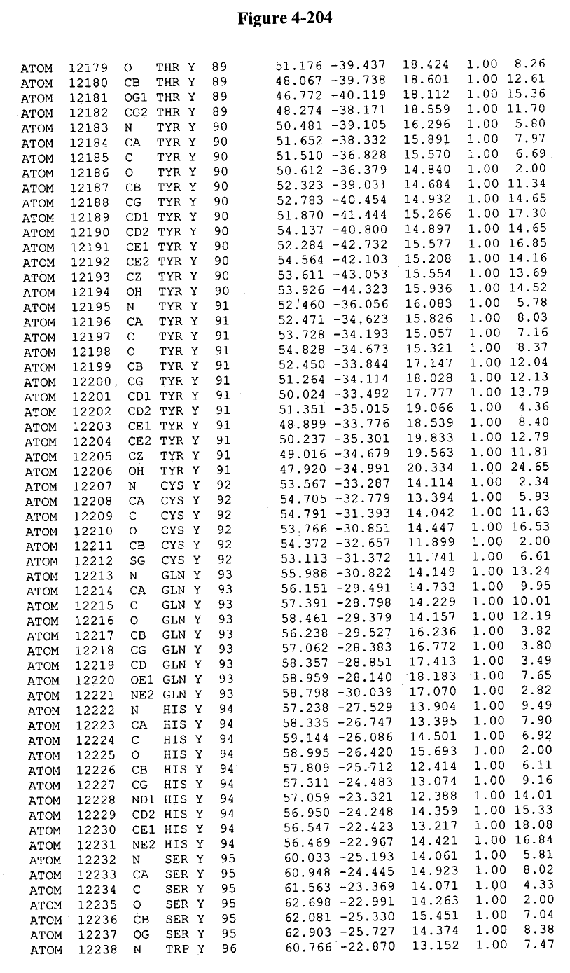

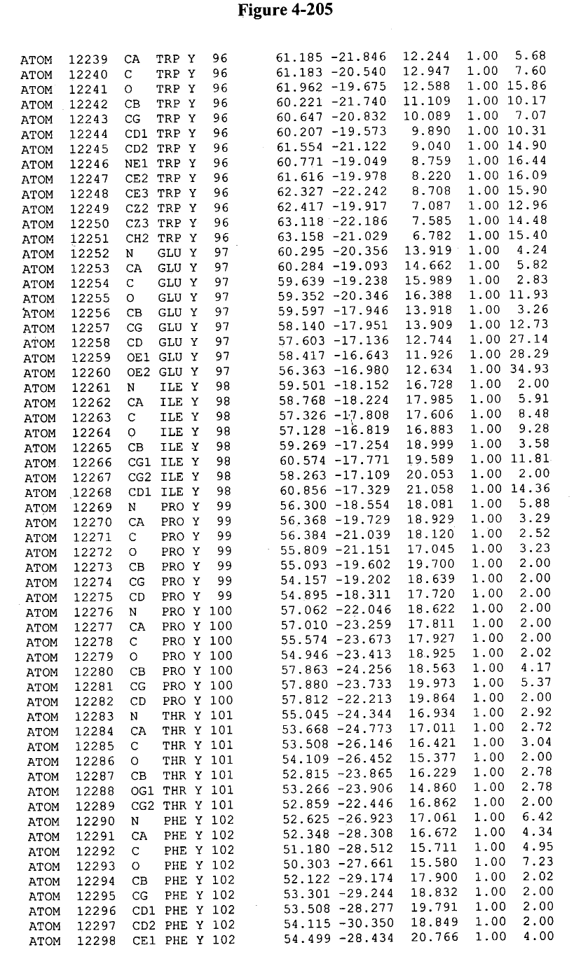

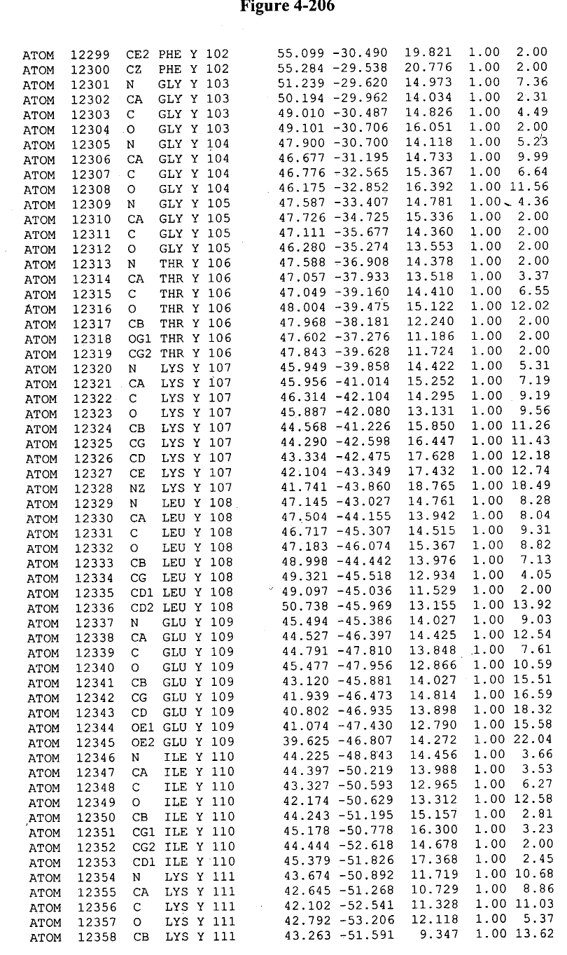

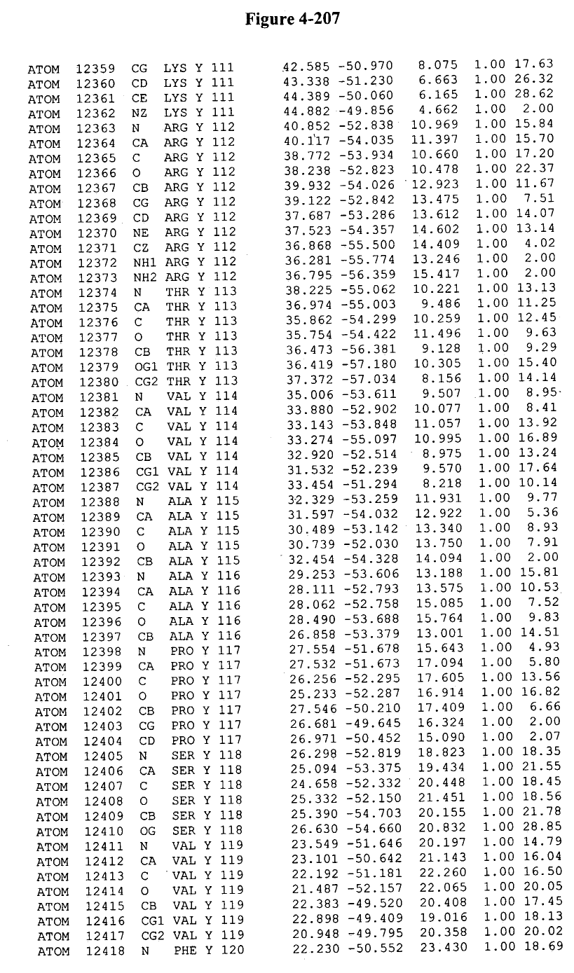

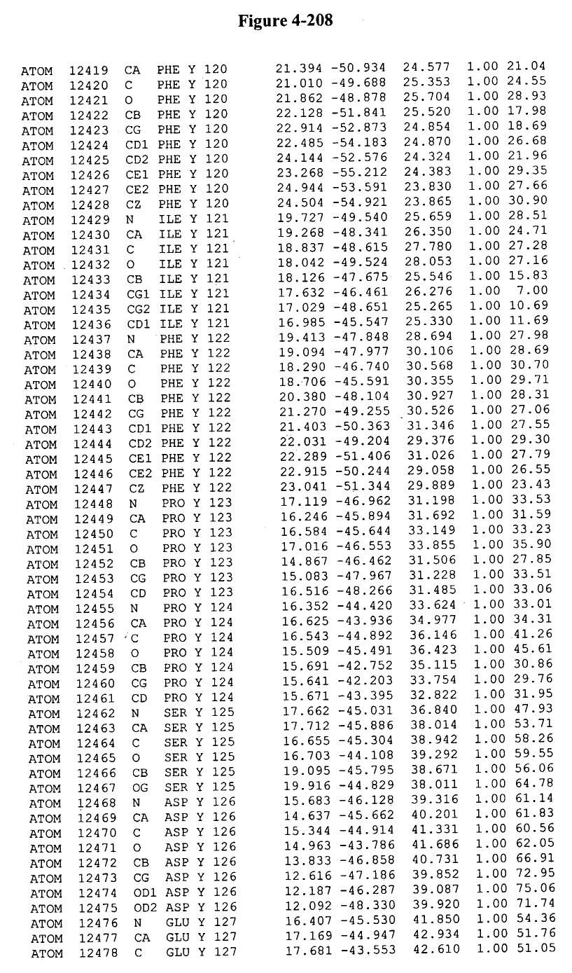

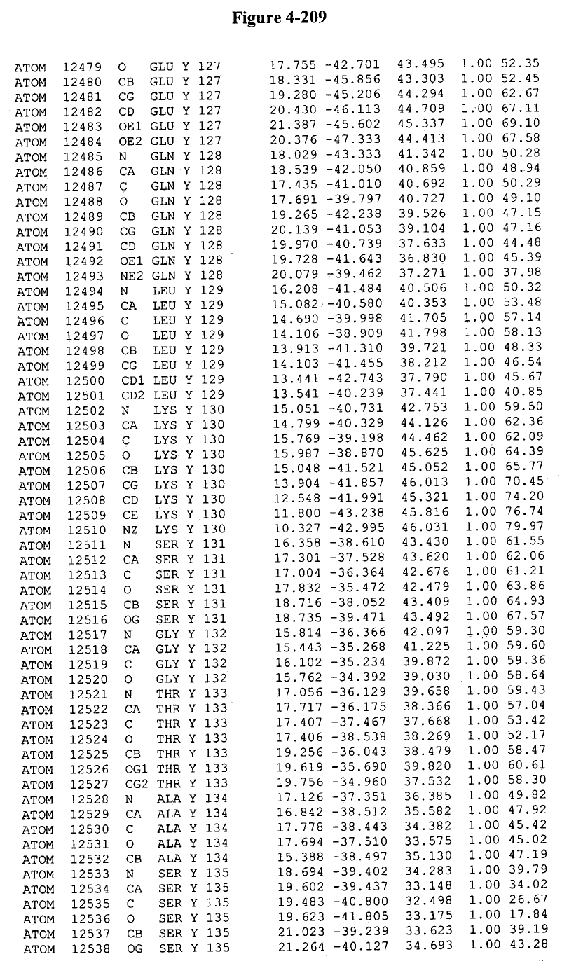

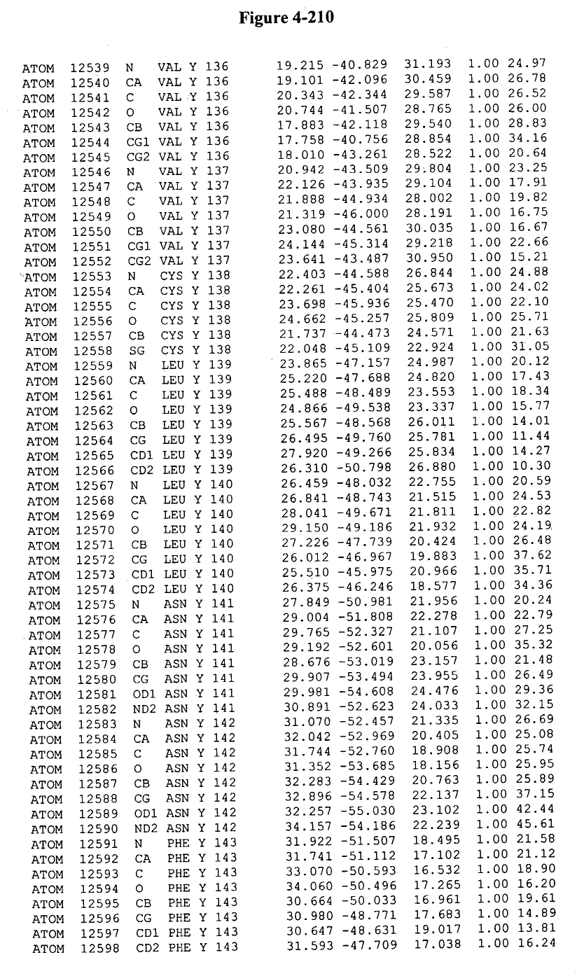

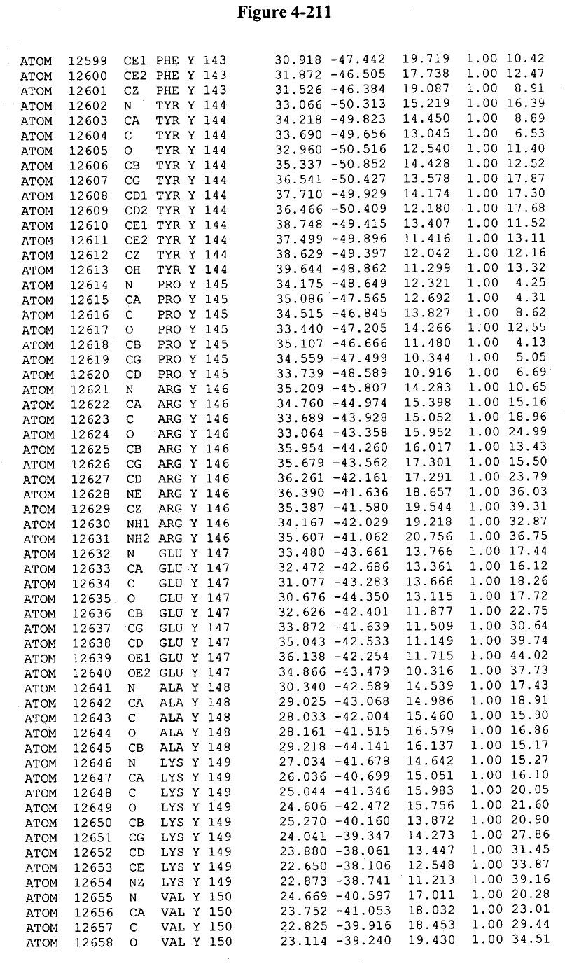

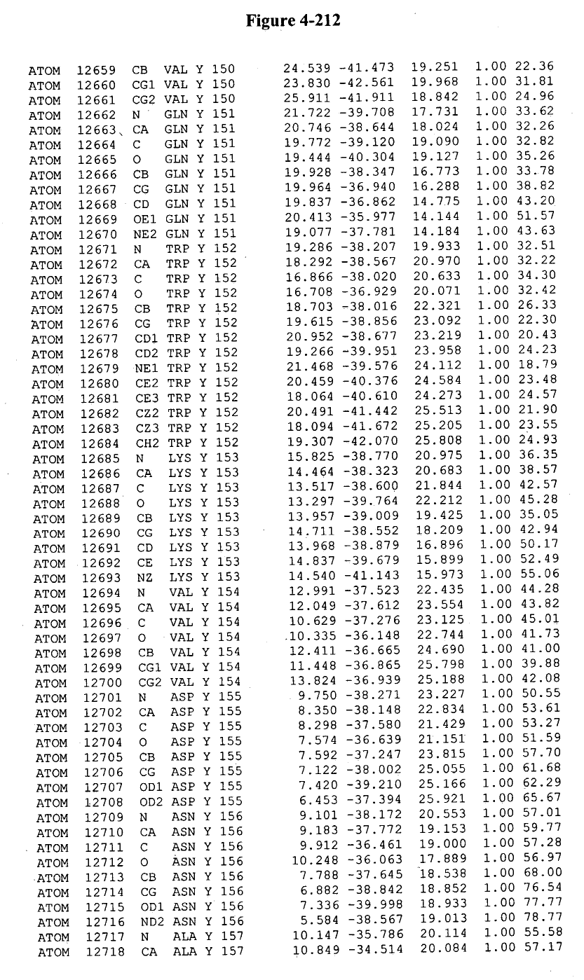

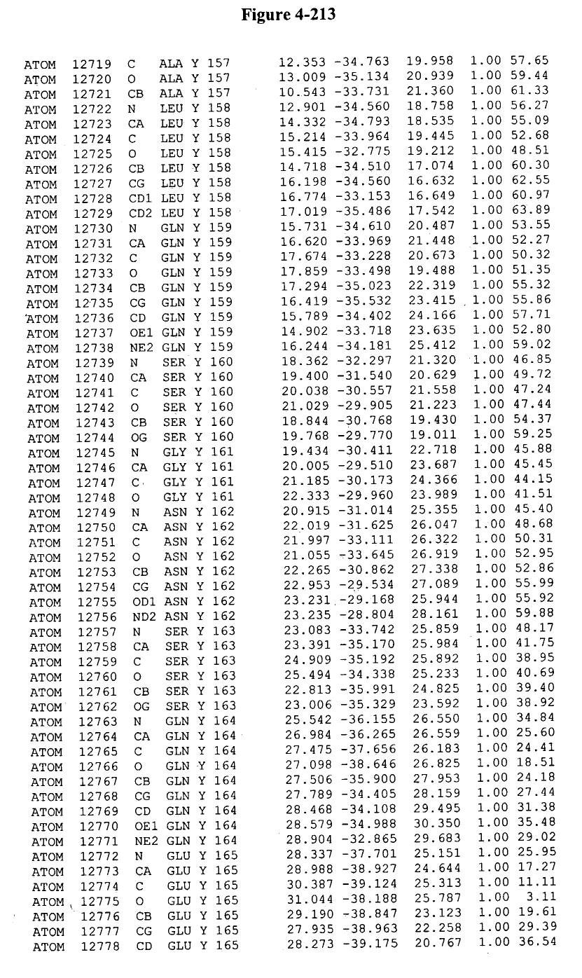

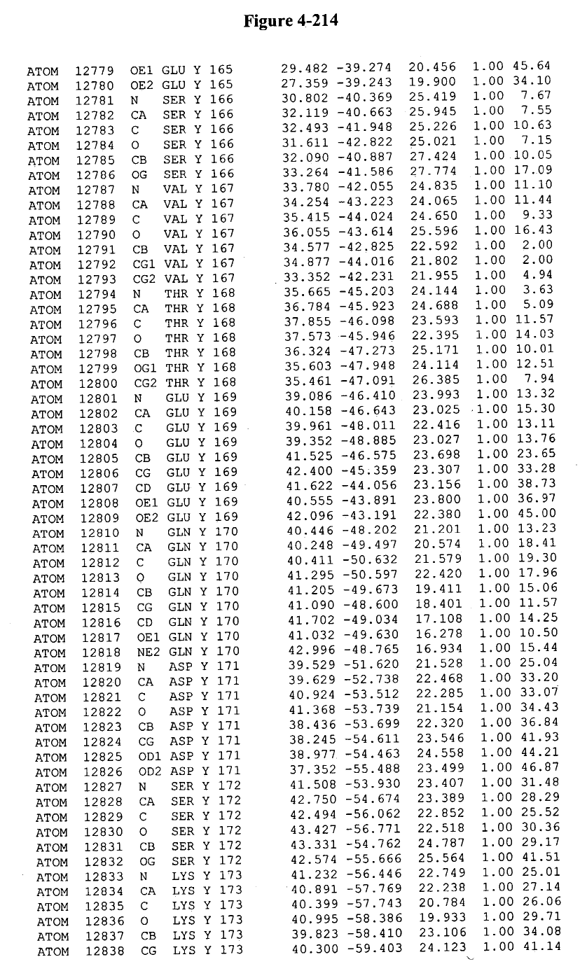

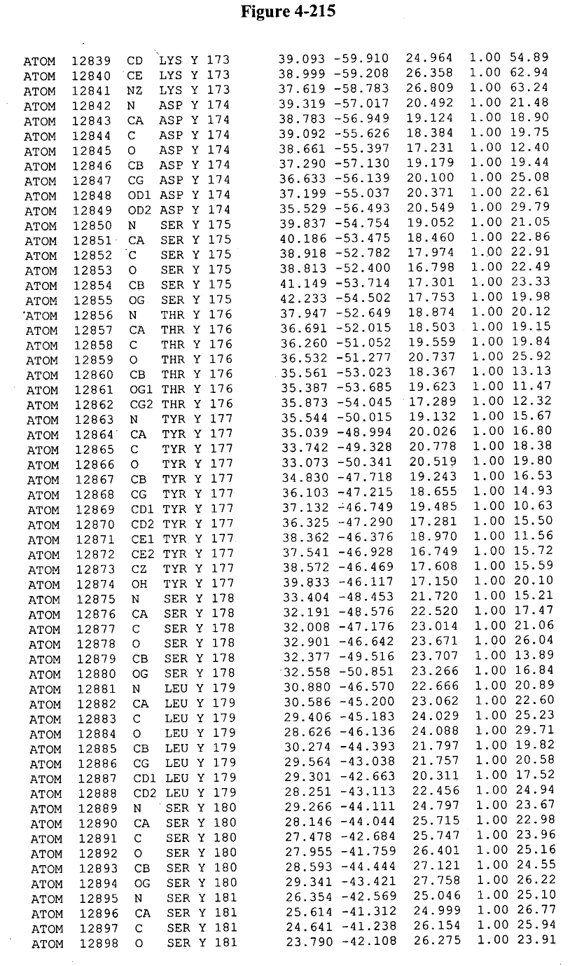

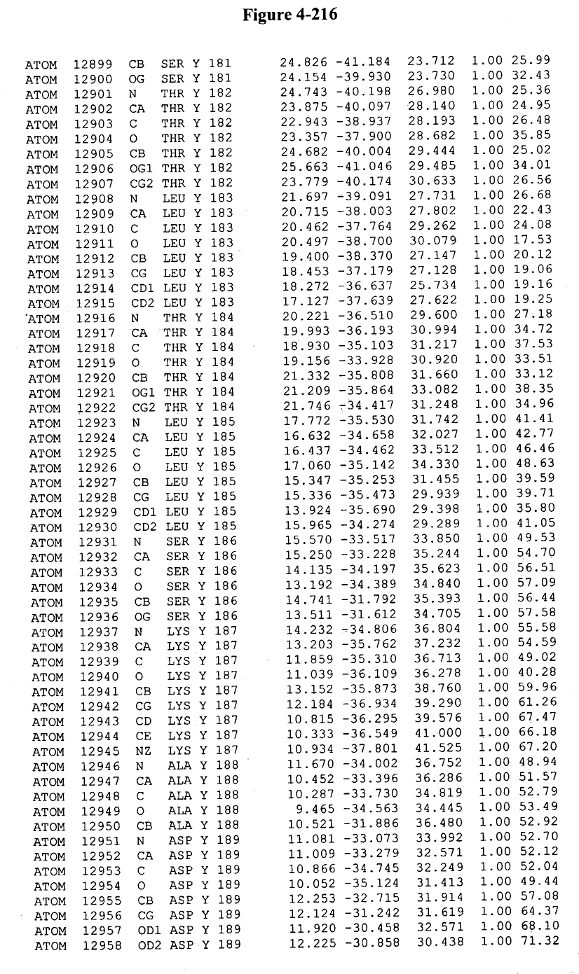

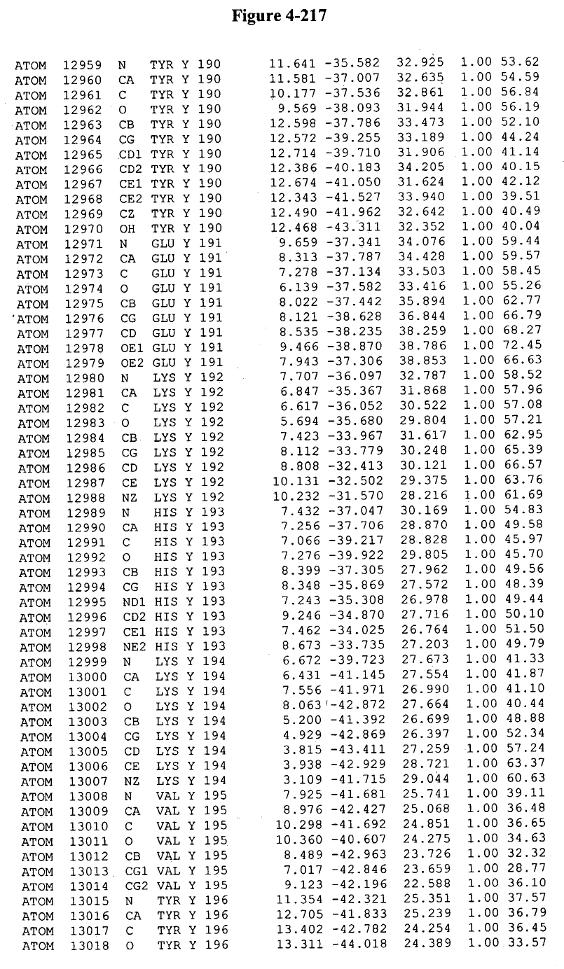

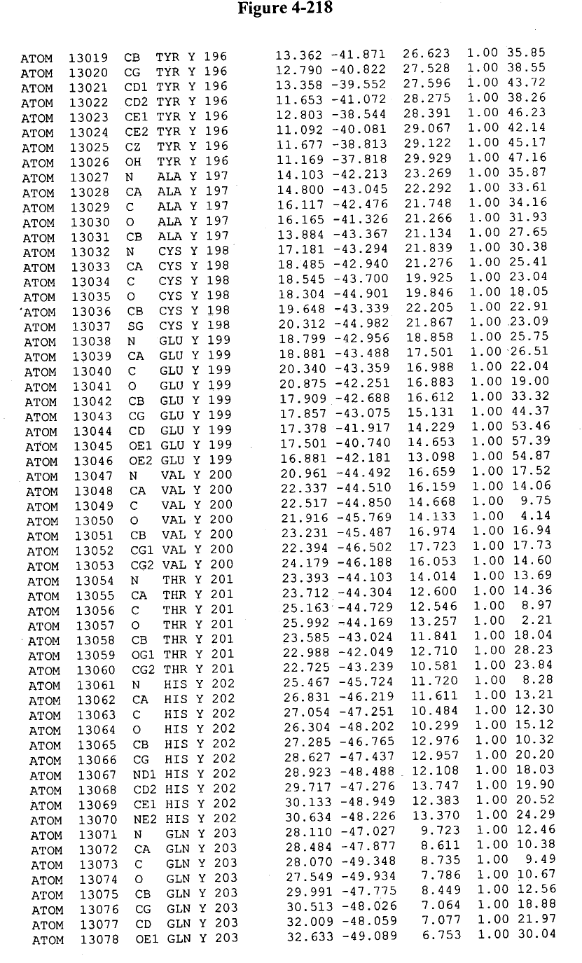

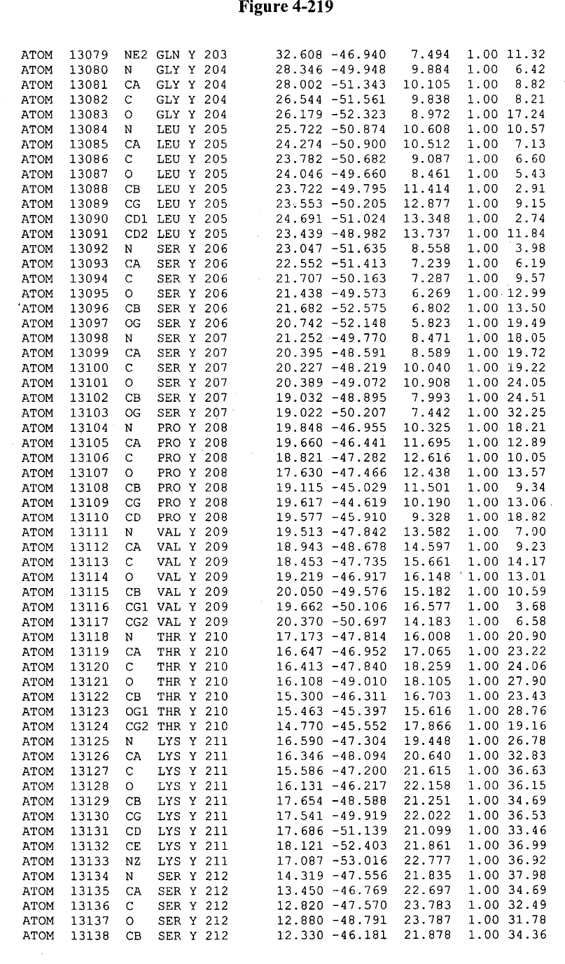

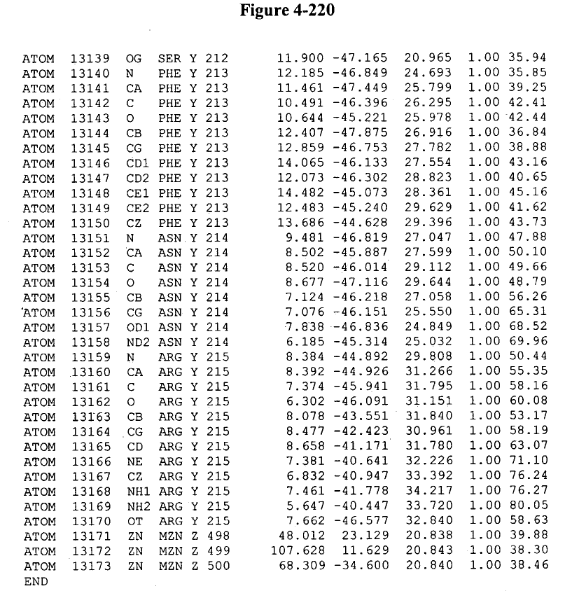

The three-dimensional structure of the extracellular domain of a CD154/Fab fragment of humanized 5c8 mAb complex is defined by a set of structure coordinates as set forth in FIG. 4. The term “structure coordinates” refers to Cartesian atomic coordinates derived from mathematical equations related to the patterns obtained on diffraction of a monochromatic beam of X-rays by the atoms (scattering centers) of an extracellular domain of a CD154/Fab fragment of humanized 5c8 mAb complex in crystal form. The diffraction data are used to calculate an electron density map of the repeating unit of the crystal. The electron density maps are then used to establish the individual atoms of the extracellular domain of a CD154/Fab fragment of humanized 5c8 mab complex.

As shown in Example 1, the epitope (also referred to as the antigenic epitope herein) on CD154 for 5c8 mAb comprises CD154 amino acids Glu129, Ala130, Ser132, Glu142, Lys143, Gly144, Tyr146, Cys178, Cys218, Ser245, Gln246, Ser248, His249 and Gly250.

A binding site defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, can bind to, inter alia, 5c8 mAb, and antigen binding fragments thereof, as well as hu5c8 mAb, and antigen binding fragments thereof.

One embodiment of the present invention provides a molecular complex comprising a first binding site defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4; or a homologue of said molecular complex, wherein said homologue comprises a second binding site that has a root mean square deviation from the backbone atoms of said amino acids between 0.00 Åand 1.50 Å, preferably between 0.00 A and 1.00 Å, more preferably between 0.00 A and 0.50 Å. The first binding site was calculated with the program CONTACT (Navaja, J. (1994) Acta Crystalloqr. A 50, 157-163) from the CCP4 program package (Collaborative Computational project No. 4. The CCP4 Suite: programs for protein crystallography Acta Cryst. D 50, 760-763). The program found all residues whose distance from contact residues of the other molecule of the complex was between 1 and 3.6 Angstroms. The first and/or the second binding site may be a binding site for 5c8 mAb, or an antigen binding fragment thereof, or hu5c8 mAb, or an antigen binding fragment thereof.

Another embodiment of the present invention provides a molecular complex comprising a first binding site, defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, that associates with one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; or a homologue of said molecular complex, wherein said homologue comprises a second binding site that has a root mean square deviation from the backbone atoms of said CD154 amino acids between 0.00 Å and 1.50 Å, preferably between 0.00 Å and 1.00 Å, more preferably between 0.00 Å and 0.50 Å. The first binding site was calculated with the program CONTACT (Navaja, J. (1994) Acta Crystalloqr. A 50, 157-163) from the CCP4 program package (Collaborative Computational project No. 4. The CCP4 Suite: programs for protein crystallography Acta Cryst. D 50, 760-763). The program found all residues whose distance from contact residues of the other molecule of the complex was between 1 and 3.6 Angstroms. The first and/or the second binding site may be a binding site for 5c8 mAb, or an antigen binding fragment thereof, or hu5c8 mAb, or an antigen binding fragment thereof.

Another embodiment of the present invention provides a molecular complex defined by structure coordinates of one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; or a homologue of said molecular complex, wherein said homologue has a root mean square deviation from the backbone atoms of said amino acids between 0.00 Å and 1.50 Å, preferably between 0.00 Å and 1.00 Å, more preferably between 0.00 Å and 0.50 Å.

Yet another embodiment of the present invention provides a molecular complex defined by at least a portion or all of the structure coordinates of all the CD154 and anti-CD154 antibody amino acids set forth in FIG. 4, or a homologue of said molecular complex, wherein said homologue has a root mean square deviation from the backbone atoms of said amino acids between 0.00 Å and 1.50 Å, preferably between 0.00 Å and 1.00 Å, more preferably between 0.00 Å and 0.50 Å. This molecular complex could have a binding site and the homologue of the molecular complex could have a binding site. Either or both of said binding sites may be a binding site for 5c8 mAb, or an antigen binding fragment thereof, or hu5c8 mAb, or an antigen binding fragment thereof.

Those of skill in the art will understand that a set of structure coordinates for a polypeptide complex is a relative set of points that define a shape in three dimensions. Thus, it is possible that an entirely different set of coordinates could define a similar or identical shape. Moreover, slight variations in the individual coordinates will have little effect on overall shape.

The variations in coordinates discussed above may be generated due to mathematical manipulations of the structure coordinates. For example, the structure coordinates set forth in FIG. 4 could be manipulated by crystallographic permutations of the structure coordinates, fractionalization of the structure coordinates, integer additions or subtractions to sets of the structure coordinates, inversion of the structure coordinates, or any combination thereof.

Alternatively, modification in the crystal structure due to mutations, additions, substitutions, and/or deletions of amino acids, or other changes in any of the components that make up the crystal could also account for variations in structure coordinates. If such variations are within an acceptable standard error as compared to the original coordinates, the resulting three dimensional shape is considered to be the same as that of the unmodified crystal.

Various computational analyses are therefore necessary to determine whether a molecular complex or a portion thereof is sufficiently similar to all or parts of the extracellular domain of a CD154/Fab fragment of humanized 5c8 mAb structure described above as to be considered the same. Such analyses may be carried out in current software applications, such as the Molecular Similarity application of QUANTA (Molecular Simulations Inc., San Diego, Calif.) version 4.1, and as described in its accompanying User's Guide.

The Molecular Similarity application permits comparisons between different structures, different conformations of the same structure, and different parts of the same structure. The procedure used in Molecular Similarity to compare structures is divided into four steps: 1) load the structures to be compared; 2) define the atom equivalences in these structures; 3) perform a fitting operation; and 4) analyze the results.

Each structure is identified by a name. One structure is identified as the target (i.e., the fixed structure); all remaining structures are working structures (i.e., moving structures). Since atom equivalency within QUANTA is defined by user input, for the purpose of this invention, equivalent atoms such as protein backbone atoms (N, Cα, C and O) will be defined for all conserved residues between the two structures being compared. Also, only rigid fitting operations will be considered.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by QUANTA.

For the purpose of this invention, any molecular complex that has a root mean square deviation of conserved residue backbone atoms (N, Cα, C, O) between 0.00 Åand 1.50 Å, preferably between 0.00 Å and 1.00 Å, more preferably between 0.00 Å and 0.50 Å, when superimposed on the relevant backbone atoms described by the structure coordinates listed in FIG. 4 are considered identical.

The term “root mean square deviation” means the square root of the arithmetic mean of the squares of the deviations from the mean. It is a way to express the deviation or variation from a trend or object. For purposes of this invention, the “root mean square deviation” defines the variation in the backbone of a protein complex from the relevant portion of the backbone of the CD154 polypeptide portion or the anti-CD154 antibody portion of the CD154/anti-CD154 antibody complex, as defined by the structure coordinates described herein.

Once the structure coordinates of a protein crystal have been determined, they are useful in solving the structures of other crystals.

In accordance with the present invention, the structure coordinates of a complex comprising the extracellular domain of CD154 and Fab fragment of, for example, humanized 5c8 mAb, and portions thereof, is stored in a machine-readable storage medium. A machine could be a computer. Such data may be used for a variety of purposes, such as drug discovery, discovery of 5c8 mAb variants with improved properties, such as improved specific binding to CD154, and X-ray crystallographic analysis of other protein crystals.

In order to use the structure coordinates generated for the CD154/anti-CD154 antibody complex or one of its binding sites or homologues thereof, it is necessary to convert them into a three-dimensional shape. This is achieved through the use of commercially available software that is capable of generating a three-dimensional graphical representation of molecular complexes, or portions thereof, from a set of structure coordinates.

Accordingly, one embodiment of this invention provides a machine-readable data storage medium comprising a data storage material encoded with machine-readable data comprising a portion of or the entire set of the structure coordinates set forth in FIG. 4. A machine could be a computer. A computer which comprises the data storage medium is also provided by this invention. This invention also provides the computer with instructions to produce three-dimensional representations of the molecular complexes of CD154/anti-CD154 antibody by processing the machine-readable data of this invention. The computer of this invention further comprises a display for displaying the structure coordinates of this invention.

A computer of this invention comprises a machine-readable data storage medium encoded with machine-readable data, wherein said data comprises one of the following four structure coordinates:

(1) the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4;

(2) the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, that associates with one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; (3) the structure coordinates of one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; or

(4) the structure coordinates of at least a portion or all of all the CD154 and anti-CD154 antibody amino acids set forth in FIG. 4;

and said computer comprises instructions for processing said machine-readable data into a three-dimensional representation of a molecular complex of this invention, or a homologue thereof. Preferably, the computer further comprises a display for displaying said structure coordinates. Such computers produce a three dimensional representation of the molecular complexes, and homologues thereof, of this invention.

This invention also provides a computer for determining at least a portion of the structure coordinates corresponding to X-ray diffraction data obtained from a molecular complex of CD154/anti-CD154 antibody, wherein said computer comprises:

a) a machine-readable data storage medium comprising a data storage material encoded with machine-readable data, wherein said data comprises at least a portion of the structure coordinates of CD154 and/or anti-CD154 antibody according to FIG. 4;

b) a machine-readable data storage medium comprising a data storage material encoded with machine-readable data, wherein said data comprises X-ray diffraction data obtained from said molecular complex; and

c) instructions for performing a Fourier transform of the machine readable data of (a) and for processing said machine readable data of (b) into structure coordinates.

Preferably, the computer further comprises a display for displaying said structure coordinates.

This invention also provides a computer for determining at least a portion of the structure coordinates corresponding to an X-ray diffraction pattern of a molecular complex, wherein said computer comprises:

a) a machine-readable data storage medium comprising a data storage material encoded with machine-readable data, wherein said data comprises at least a portion of the structure coordinates according to FIG. 4;

b) a machine-readable data storage medium comprising a data storage material encoded with machine-readable data, wherein said data comprises an X-ray diffraction pattern of said molecular complex;

c) a working memory for storing instructions for processing said machine-readable data of a) and b);

d) a central processing unit coupled to said working memory and to said machine-readable data of a) and b) for performing a Fourier transform of the machine readable data of (a) and for processing said machine readable data of (b) into structure coordinates; and

e) a display coupled to said central processing unit for displaying said structure coordinates of said molecular complex.

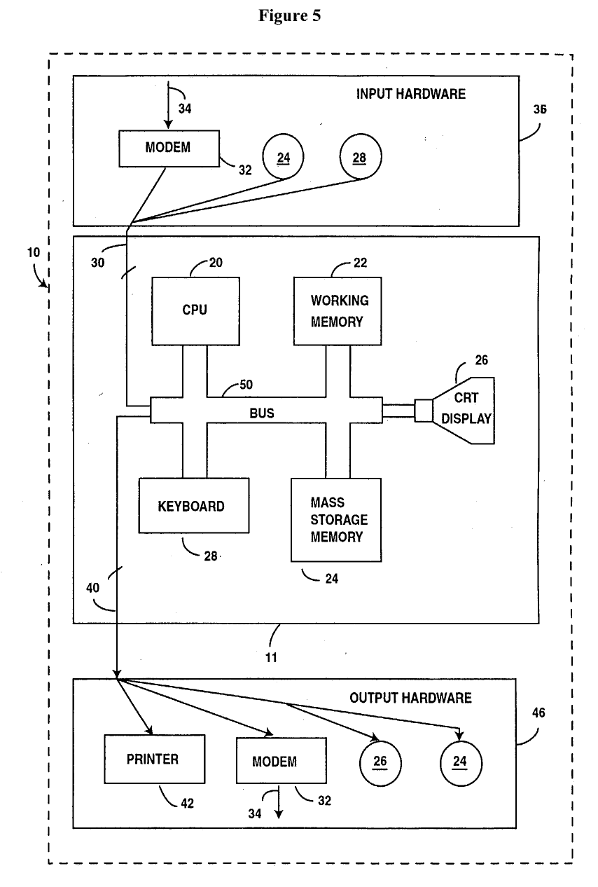

FIG. 5 demonstrates one version of these embodiments. System 10 includes a computer 11 comprising a central processing unit (“CPU”) 20, a working memory 22 which may be, e.g., RAM (random-access memory) or “core” memory, mass storage memory 24 (such as one or more disk drives or CD-ROM or DVD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. Machine-readable data of this invention may be inputted via the use of a modem or modems 32 connected by a telephone line or dedicated data line 34. Alternatively or additionally, the input hardware 36 may comprise CD-ROM or DVD-ROM drives or disk drives 24. In conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a graphical representation of a binding site of this invention using a program such as QUANTA as described herein. Output hardware might also include a printer 42, so that hard copy output may be produced, or a disk drive 24, to store system output for later use.

In operation, CPU 20 coordinates the use of the various input and output devices 36, 46, coordinates data accesses from mass storage 24 and accesses to and from working memory 22, and determines the sequence of data processing steps. A number of programs may be used to process the machine-readable data of this invention. Such programs are discussed in reference to the computational methods of drug discovery as described herein. Specific references to components of the hardware system 10 are included as appropriate throughout the following description of the data storage medium.

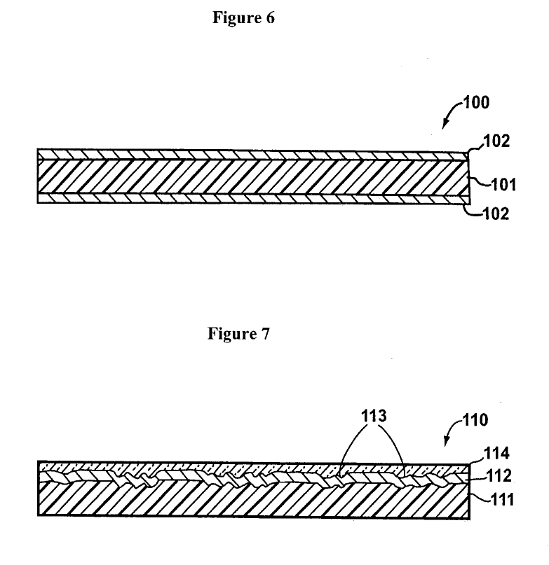

FIG. 6 shows a cross-section of a magnetic data storage medium 100 which can be encoded with a machine-readable data that can be carried out by a system such as system 10 of FIG. 5. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24.

The magnetic domains of coating 102 of medium 100 are polarized or oriented so as to encode in manner which may be conventional, machine readable data such as that described herein, for execution by a system such as system 10 of FIG. 5.

FIG. 7 shows a cross-section of an optically-readable data storage medium 110 which also can be encoded with such a machine-readable data, or set of instructions, which can be carried out by a system such as system 10 of FIG. 5. Medium 110 can be a conventional compact disk or DVD disk read only memory (CD-ROM or DVD-ROM) or a rewritable medium, such as a magneto-optical disk which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

In the case of a magneto-optical disk, as is well known, coating 112 has no pits 113, but has a plurality of magnetic domains whose polarity or orientation can be changed magnetically when heated above a certain temperature, as by a laser (not shown). The orientation of the domains can be read by measuring the polarization of laser light reflected from coating 112. The arrangement of the domains encodes the data as described above.

For the first time, the present invention permits the use of structure-based and rational drug design techniques to design, select, and synthesize chemical entities, compounds (such as agonists or antagonists of CD154), and 5c8 mAb variants with improved properties, such as higher or lower binding affinity for CD154 as compared to 5c8 mAb. Additionally, the present invention permits the use of structure-based or rational drug design techniques to make improvements of currently available CD154 antagonists, that are capable of binding to the extracellular domain of CD154/Fab fragment of humanized 5c8 mAb complex, or any portion thereof.

One particularly useful drug design technique enabled by this invention is iterative drug design. Iterative drug design is a method for optimizing associations between a protein and a compound (that compound includes an antibody) by determining and evaluating the three-dimensional structures of successive sets of protein/compound complexes.

Those of skill in the art will realize that association of natural receptors (such as CD40), or substrates with the binding sites of their corresponding ligand (such as CD154, which is also known as CD40 ligand) or enzymes is the basis of many biological mechanisms of action. Similarly, many drugs (which include mAbs) exert their biological effects through association with the binding sites of, for example, ligands (such as CD154), receptors and enzymes. Such associations may occur with all or any parts of the binding sites. For example, 5c8 mab binds to CD154 and blocks the interaction between CD154 and CD40. An understanding of such associations enables the design of drugs having more favorable associations with their target ligand, receptor or enzyme, and thus, improved biological effects. Therefore, this information is valuable in designing potential chemical entities or inhibitors (including compounds and antibodies, such as, inter alia, 5c8 mAb variants and variants of other anti-CD154 antibodies) of ligands, receptors or enzymes.

The term “binding site”, as used herein, refers to a region of a protein, that, as a result of its shape, favorably associates with another protein, a chemical entity, a compound or an antibody, and an antigen binding fragment thereof. For example, the binding site on CD154 for 5c8 mAb is the epitope of 5c8 mAb. This binding site could also be the binding site of a chemical entity, a compound or variant of 5c8 mAb, or antigen binding fragments thereof. CD154 also has a binding site for CD40.

The term “associating with” refers to a condition of proximity between two or more chemical entities, compounds and proteins, or portions thereof. The association may be non-covaient—wherein the juxtaposition is energetically favored by hydrogen bonding or van der Waals or electrostatic interactions—or it may be covalent.

In iterative drug design, crystals of a series of protein/compound or antibody complexes are obtained and then the three-dimensional structure of each new complex is solved. Such an approach provides insight into the association between the proteins and compounds or antibodies of each new complex. This is accomplished by selecting compounds or antibodies with inhibitory activity, obtaining crystals of the new protein/compound or antibody complex, solving the three-dimensional structure of the complex, and comparing the associations between the new protein/compound or antibody complex and previously solved protein/compound or antibody complexes. By observing how changes in the compound or antibody affect the protein/compound or antibody associations, these associations may be optimized.

In some cases, iterative drug design is carried out by forming successive protein-compound or antibody complexes and then crystallizing each new complex. Alternatively, a pre-formed protein crystal is soaked in the presence of an inhibitor, thereby forming a protein/compound complex and obviating the need to crystallize each individual protein/compound or antibody complex.

The structure coordinates set forth in FIG. 4 can also be used to aid in obtaining structural information about another crystallized molecular complex. This may be achieved by any of a number of well-known techniques, including molecular replacement. This method is especially useful for determining the structures of CD154 or anti-CD154 antibody mutants and homologues.

The structure coordinates set forth in FIG. 4 can also be used for determining at least a portion of the three-dimensional structure of a molecular complex which contains at least some structural features similar to at least a portion of a CD154 anti-CD154 complex. In particular, structural information about another crystallized molecular complex may be obtained. This may be achieved by any of a number of well-known techniques, including molecular replacement.

Therefore, another embodiment of this invention provides a method of utilizing molecular replacement to obtain structural information about a crystallized molecular complex whose structure is unknown comprising the steps of:

a) generating an X-ray diffraction pattern from said crystallized molecular complex; and

b) applying at least a portion of the structure coordinates set forth in FIG. 4 to the X-ray diffraction pattern to generate a three-dimensional electron density map of the molecular complex whose structure is unknown.

Preferably, the crystallized molecular complex comprises a CD154 polypeptide and an anti-CD154 antibody polypeptide.

By using molecular replacement, all or part of the structure coordinates of the extracellular domain of the CD154/Fab fragment of the humanized 5c8 mAb complex provided by this invention (and set forth in FIG. 4) can be used to determine the structure of a crystallized molecular complex whose structure is unknown more rapidly and efficiently than attempting to determine such information ab initio. This method is especially useful in determining the structure of CD154 and anti-CD154 antibody mutants and homologues.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, molecular replacement involves generating a preliminary model of a molecular complex whose structure coordinates are unknown, by orienting and positioning the relevant portion of the extracellular domain of the CD154/Fab fragment of the humanized 5c8 mAb complex according to FIG. 4 within the unit cell of the crystal of the unknown molecular complex, so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecular complex [E. Lattman, “Use of the Rotation and Translation Functions”, in Meth. Enzymol., 115, pp. 55-77 (1985); M. G. Rossmann, ed., “The Molecular Replacement Method”, Int. Sci. Rev. Ser., No. 13, Gordon & Breach, New York (1972)].

The structure of any portion of any crystallized molecular complex that is sufficiently homologous to any portion of the extracellular domain of a CD154/Fab fragment of humanized 5c8 mAb complex can be solved by this method.

In a preferred embodiment, the method of molecular replacement is utilized to obtain structural information about a molecular complex, wherein the complex comprises a CD154-like polypeptide. Preferably the CD154-like polypeptide is CD154, a mutant thereof or a homologue thereof.

The structure coordinates of the extracellular domain of a CD154/Fab fragment of a humanized 5c8 mAb complex as provided by this invention are particularly useful in solving the structure of other crystal forms of CD154-like polypeptide, preferably other crystal forms of CD154; CD154-like polypeptide/anti-CD154 antibody-like polypeptide, preferably the extracellular domain of CD154/Fab fragment of humanized 5c8 mAb; or complexes comprising any of the above.

Such structure coordinates are also particularly useful to solve the structure of crystals of CD154-like polypeptide/anti-CD154 antibody-like polypeptide complexes, particularly the extracellular domain of a CD154/Fab fragment of a humanized 5c8 mAb, co-complexed with a variety of chemical entities. This approach enables the determination of the optimal sites for interaction between chemical entities and interaction of candidate CD154 agonists or antagonists with CD154 or the extracellular domain of CD154/Fab fragment of humanized 5c8 mAb complex. For example, high resolution X-ray diffraction data collected from crystals exposed to different types of solvent allows determination of the location where each type of solvent molecule resides. Small molecules that bind tightly to these sites can then be designed and synthesized and tested for their CD154 antagonist activity.

All of the complexes referred to above may be studied using well-known X-ray diffraction techniques and may be refined versus 1.5-3.5 Åresolution X-ray data to an R value of about 0.20 or less using computer software, such as X-PLOR (Yale University, 01992, distributed by Molecular Simulations, Inc.; see, e.g., Blundell & Johnson, supra; Meth. Enzymol., vol. 114 & 115, H. W. Wyckoff et al., eds., Academic Press (1985)). This information may thus be used to optimize known CD154 antagonists, such as anti-CD154 antibodies, and more importantly, to design new or improved CD154 antagonists.

A chemical entity, a compound (including an agonist or antagonist of CD154) or a variant of the 5c8 mAb, or an antigen binding fragment thereof, or hu5c8 mAb, or an antigen binding fragment thereof, or variants of another anti-CD154 antibody, or an antigen binding fragment thereof, can be designed by computational means by performing fitting operations. A compound includes macromolecules such as proteins or polypeptides.

The present invention also encompasses methods of evaluating the potential of a chemical entity to associate with a molecular complex of this invention, or a homologue of said molecular complex.

This invention provides a method for evaluating the potential of a chemical entity to associate with a molecular complex of this invention, or a homologue of said molecular complex, comprising the steps of:

(i) employing computational means to perform a fitting operation between the chemical entity and a binding site (the binding site could be a binding site for 5c8 mAb, or an antigen binding fragment thereof, or hu5c8 mAb, or an antigen binding fragment thereof) of the molecular complex or a binding site of the homologue of the molecular complex; and

(ii) analyzing the results of said fitting operation to quantify the association between the chemical entity and either binding site.

The present invention also encompasses methods for identifying a potential agonist or antagonist of CD154 comprising the steps of:

a) using the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4±a root mean square deviation from the backbone atoms of said CD154 amino acids between 0.00 Å and 1.50 Å, preferably between 0.00 Å and 1.00 Å, more preferably between 0.00 Å and 0.50 Å; or using the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, that associate with one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4±a root mean square deviation from the backbone atoms of said CD154 amino acids between 0.00 Å and 1.50 Å, preferably between 0.00 Å and 1.00 Å, more preferably between 0.00 Å and 0.50 Å; or using at least a portion of the structure coordinates of all the amino acids of CD154 and anti-CD154 antibody according to FIG. 4±a root mean square deviation from the backbone atoms of said amino acids between 0.00 Å and 1.50 Å, preferably between 0.00 Å and 1.00 Å, more preferably between 0.00 Å and 0.50 Å; to generate a three-dimensional structure of a molecular complex comprising a binding site (the binding site could be a binding site for 5c8 mAb, or an antigen binding fragment thereof, or hu5c8 mAb, or an antigen binding fragment thereof);

b) employing said three-dimensional structure to design or select said potential agonist or antagonist;

c) synthesizing said potential agonist or antagonist; and

d) contacting said potential agonist or antagonist with CD154 to determine the ability of said potential agonist or antagonist to bind to (interact with) CD154; or contacting said potential agonist or antagonist with CD154 under conditions that permit said potential agonist or antagonist to interact with (bind to) CD154, if said potential agonist or antagonist is capable of binding to CD154.

This method could further comprise the step of:

e) determining whether said potential antagonist interrupts CD40:CD154 interaction.

A potential agonist or a potential antagonist is a compound. A compound can be a macromolecule, such as a protein or a polypeptide.

This invention also encompasses methods for evaluating the potential of a variant of 5c8 mAb, or an antigen binding fragment thereof, or humanized 5c8 mAb, or an antigen binding fragment thereof, or another anti-CD154 antibody, or an antigen binding fragment thereof, to associate with a molecular complex of this invention or a homologue of said molecular complex; comprising the steps of:

(i) employing computational means to perform a fitting operation between the variant and a binding site (the binding site could be a binding site for 5c8 mAb, or an antigen binding fragment thereof, or hu5c8 mAb, or an antigen binding fragment thereof) of a molecular complex of this invention or a binding site (the binding site could be a binding site for 5c8 mAb, or an antigen binding fragment thereof, or hu5c8 mAb, or an antigen binding fragment thereof) of a homologue of the molecular complex; and

(ii) analyzing the results of said fitting operation to quantify the association between the binding site of the molecular complex or the binding site of the homologue of the molecular complex.

Thus, the present invention provides 5c8 mAb variants (or variants of other anti-CD154 antibodies) with improved properties as compared to 5c8 mAb, such as increased or decreased binding affinity for CD154.

The present invention also encompasses the chemical entities, compounds, such as agonists or antagonists of CD154 or variants of 5c8 mAb (or other anti-CD154 antibodies), or an antigen binding fragment thereof, or hu5c8 mAb, or an antigen binding fragment thereof, identified by the methods of this invention.

For the first time, the present invention permits the use of molecular design techniques to design, select and synthesize chemical entities, compounds, including agonists or antagonists of CD154, and variants of 5c8 mAb (or another anti-CD154 antibody), and antigen binding fragments thereof, capable of binding to CD154, including CD40:CD154 binding interrupters.

The design of chemical entities, compounds including agonists or antagonists of CD154 and variants of 5c8 mAb (or another anti-CD154 antibody), and antigen binding fragments thereof, that bind to CD154 according to this invention generally involves consideration of two factors. First, the chemical entity, compound or 5c8 mAb variant must be capable of physically and structurally associating with CD154. Non-covalent molecular interactions important in the association of a protein, such as CD154, with its binding partner include hydrogen bonding, van der Waals and hydrophobic interactions.

Second, the chemical entity, compound or 5c8 mAb variant must be able to assume a conformation that allows it to associate with CD154 directly. Although certain portions of the chemical entity, compound or 5c8 mAb variant or humanities 5c8 mab variant will not directly participate in these associations, those portions of the chemical entity, 5c8 mAb variant or compound may still influence the overall conformation of the molecule. This, in turn, may have a significant impact on potency. Such conformational requirements include the overall three-dimensional structure and orientation of the chemical entity, 5c8 mAb variant or compound in relation to all or a portion of the binding site, e.g., active site or accessory binding site of CD154, or the spacing between functional groups of a compound comprising several chemical entities that directly interact with CD154.

The potential binding effect on CD154 or CD40:CD154 binding interruption of a chemical entity, compound or 5c8 mAb variant can be analyzed prior to its actual synthesis or generation and testing by the use of computer modeling techniques. If the theoretical structure of the given entity or compound or 5c8 mAb variant suggests insufficient interaction and association with CD154, synthesis and testing of the entity or compound or generation and testing of 5c8 mAb variant is obviated. However, if computer modeling indicates a strong interaction, the entity, compound or 5c8 mAb variant may then be generated and tested for its ability to bind to CD154 and interrupt its association with CD40 using the assays described below. In this manner, generation of inoperative entities, compounds or 5c8 mAb variants may be avoided.

A CD154-binding entity, compound or variant of 5c8 mAb or humanized 5c8 mAb, or antigen binding fragments of either, can be computationally evaluated and designed by means of a series of steps in which chemical entities or fragments are screened and selected for their ability to associate with the binding sites of CD154 as defined by this invention.

One skilled in the art can use one of several methods to screen chemical entities or fragments for their ability to associate with CD154 and more particularly with the binding sites of CD154. This process may begin by visual inspection of, for example, the binding sites for anti-CD154 antibody, on the computer screen based on the CD154 coordinates in FIG. 4 generated from the machine-readable storage medium. Selected fragments or chemical entities may then be positioned in a variety of orientations, or docked, within an individual binding site of CD154, as defined supra. Docking may be accomplished using software such as Quanta or Sybyl, followed by energy minimization and molecular dynamics with standard molecular mechanics forcefields, such as CHARMM and AMBER.

Specialized computer programs may also assist in the process of selecting fragments or chemical entities. These include, inter alia:

1. GRID (Goodford, P. J., “A Computational Procedure for Determining Energetically Favorable Binding Sites on Biologically Important Macromolecules”, J. Med. Chem., 28, pp. 849-857 (1985)). GRID is available from Oxford University, Oxford, UK.

2. MCSS (Miranker, A. and M. Karplus, “Functionality Maps of Binding Sites: A Multiple Copy Simultaneous Search Method.” Proteins: Structure, Function and Genetics, 11, pp. 29-34 (1991)). MCSS is available from Molecular Simulations, Burlington, Mass.

3. AUTODOCK (Goodsell, D. S. and A. J. Olsen, “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.

4. DOCK (Kuntz, I. D. et al., “A Geometric Approach to Macromolecule-Ligand Interactions”, J. Mol. Biol., 161, pp. 269-288 (1982)). DOCK is available from University of California, San Francisco, Calif.

Once suitable chemical entities or fragments have been selected, they can be assembled into a single compound. Assembly may proceed by visual inspection of the relationship of the fragments to each other on the three-dimensional image displayed on a computer screen in relation to the structure coordinates of CD154. This is followed by manual model building using software such as Quanta or Sybyl.

The above-described evaluation process for chemical entities may be performed in a similar fashion for chemical compounds and 5c8 mAb variants.

Useful programs to aid one of skill in the art in connecting the individual chemical entities or fragments include:

1. CAVEAT (Bartlett, P. A. et al, “CAVEAT: A Program to Facilitate the Structure-Derived Design of Biologically Active Molecules”. In “Molecular Recognition in Chemical and Biological Problems”, Special Pub., Royal Chem. Soc., 78, pp. 182-196 (1989)). CAVEAT is available from the University of California, Berkeley, Calif.

2. 3D Database systems such as MACCS-3D (MDL Information Systems, San Leandro, Calif.). This area is reviewed in Martin, Y. C., “3D Database Searching in Drug Design”, J. Med. Chem., 35, pp. 2145-2154 (1992)).

3. HOOK (available from Molecular Simulations, Burlington, Mass.).

Instead of proceeding to build a CD154 antagonist or a CD154 binding compound in a step-wise fashion one fragment or chemical entity at a time, as described above, CD154 antagonists or other CD154 binding compounds, including variants of 5c8 mAb or humanized 5c8 mAb, or antigen binding fragments of either, may be designed as a whole or “de novo” using either an empty binding site or optionally including some portion(s) of a known antagonist(s) of CD154 or a CD154 binding compound. These methods include:

1. LUDI (Bohm, H.-J., “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.

2. LEGEND (Nishibata, Y. and A. Itai, Tetrahedron, 47, p. 8985 (1991)). LEGEND is available from Molecular Simulations, Burlington, Mass.

3. LeapFrog (available from Tripos Associates, St. Louis, Mo.).

Other molecular modeling techniques may also be employed in accordance with this invention. See, e.g., Cohen, N. C. et al., “Molecular Modeling Software and Methods for Medicinal Chemistry, J. Med. Chem., 33, pp. 883-894 (1990). See also Navia, M. A. and M. A. Murcko, “The Use of Structural Information in Drug Design”, Current Opinions in Structural Biology, 2, pp. 202-210 (1992).

Once an entity, compound or variant of 5c8 mAb or humanized 5c8 mAb, or antigen binding fragments of either, has been designed or selected by the above methods, the efficiency with which that entity, compound or 5c8 mAb variant may bind to CD154 can be tested and optimized by computational evaluation. For example, a compound that has been designed or selected to function as a CD154 binding compound must also preferably traverse a volume not overlapping that occupied by the binding site when it is bound to the native CD154. An effective CD154 binding compound must preferably demonstrate a relatively small difference in energy between its bound and free states (i.e., a small deformation energy of binding). Thus, the most efficient CD154 binding compound should preferably be designed with a deformation energy of binding of not greater than about 10 kcal/mole, preferably, not greater than 7 kcal/mole. CD154 binding compounds may interact with the CD154 in more than one conformation that is similar in overall binding energy. In those cases, the deformation energy of binding is taken to be the difference between the energy of the free compound and the average energy of the conformations observed when the compound binds to the protein.

A compound designed or selected as binding to CD154 may be further computationally optimized so that in its bound state it would preferably lack repulsive electrostatic interaction with the target protein. Such non-complementary (e.g., electrostatic) interactions include repulsive charge-charge, dipole-dipole and charge-dipole interactions. Specifically, the sum of all electrostatic interactions between the compound and the protein when the compound is bound to CD154, preferably make a neutral or favorable contribution to the enthalpy of binding.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa. 01992); AMBER, version 4.0 (P.A. Kollman, University of California at San Francisco, 1994); QUANTA/CHARMM (Molecular Simulations, Inc., Burlington, Mass. 01994); and Insight II/Discover (Biosysm Technologies Inc., San Diego, Calif. 1994). These programs may be implemented, for instance, using a Silicon Graphics workstation, IRIS 4D/35 or IBM RISC/6000 workstation model 550. Other hardware systems and software packages will be known to those skilled in the art.

Once a CD154-binding compound has been optimally selected or designed, as described above, substitutions may then be made in some of its atoms or side groups to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to CD154 by the same computer methods described in detail above.

Another approach made possible and enabled by this invention is computational screening of small molecule data-bases for chemical entities or compounds that can bind in whole, or in part, to CD154. In this screening, the quality of fit of such entities to the binding site may be judged either by shape complementarity or by estimated interaction energy. Meng, E. C. et al., J. Comp. Chem., 13, pp. 505-524 (1992).

Compounds

The compounds of this invention can be synthetic compounds. In one embodiment, a synthetic compound designed by methods of this invention preferably has a molecular weight equal to or under about 1000 daltons. A synthetic compound designed by methods of this invention preferably is soluble under physiological conditions. A synthetic compound designed by methods of this invention preferably is bioavailable. A synthetic compound designed by methods of this invention is preferably orally administrable. A synthetic compound designed by methods of this invention preferably is able to bind its target (CD154) when the target is present at physiological concentrations. A synthetic compound designed by methods of this invention preferably is non-toxic or has a medically acceptable toxicity.

Assays for Confirming that Compounds Bind and Interrupt CD40:CD154 Interaction

A person skilled in the art is aware of conventional assays for assessing whether the entities, compounds, 5c8 mAb variants or humanized 5c8 mAb variants designed according to the methods of this invention bind specifically to CD154 and whether they interrupt CD40:CD154 interaction. These assays detect whether, or the extent to which, B cells are activated by activated T cells via the interaction between CD154 and CD40. For example, monitoring of CD23 levels on B cells, or secretion of immunoglobulins by B cells is indicative of activation of B cells by activated T cells via the interaction between CD40 and CD154. See, e.g., U.S. Pat. No. 5,474,771. Accordingly, examples of such assays are: in vitro assays for blocking CD40 and CD154 interaction, in vitro assays for T cell activation of B cells; in vitro assays for immunoglobulin production by B cells and in vivo assays for inhibition of a humoral immune response.

Conditions Associated with Inappropriate CD154 Induced Activation in a Subject

The chemical entities and compounds designed according to this invention, including agonists or antagonists of CD154, 5c8 mAb variants and humanized 5c8 mAb variants can be used to prevent or treat subjects having conditions associated with inappropriate CD154 induced activation. Treating a condition associated with inappropriate CD154 induced activation in a subject includes, inter alia, attenuating severity of the condition, suppressing effects of the condition, inhibiting the condition and reversing the condition.

Examples of conditions associated with inappropriate CD154 mediated activation in a subject, include, inter alia: an unwanted immune response, an unwanted inflammatory response, an autoimmune disease, an allergy, an inhibitor response to a therapeutic agent, rejection of a donor organ and a B cell cancer.

Examples of conditions associated with inappropriate CD154 mediated activation in a subject, include, inter alia: systemic lupus erythematosis, lupus nephritis, lupus neuritis, asthma, chronic obstructive pulmonary disease, bronchitis, emphysema, multiple sclerosis, uveitis, Alzheimer's disease, traumatic spinal cord injury, stroke, atherosclerosis, coronary restenosis, ischemic congestive heart failure, cirrhosis, hepatitis C, diabetic nephropathy, glomerulonephritis, osteoarthritis, rheumatoid arthritis, psoriasis, atopic dermatitis, systemic sclerosis, radiation-induced fibrosis, Crohn's disease, ulcerative colitis, multiple myeloma and cachexia.

Subjects

The novel CD40:CD154 binding interrupters designed according to this invention can be administered for treatment or prophylaxis to any mammalian subject suffering or about to suffer a condition associated with inappropriate CD154 activation. Preferably, the subject is a primate, more preferably a higher primate, most preferably a human. In other embodiments of this invention, the subject may be a mammal of commercial importance, or a companion animal or other animal of value, such as a member of an endangered species. Thus, a subject may be, inter alia, sheep, horses, cattle, goats, pigs, dogs, cats, rabbits, guinea pigs, hamsters, gerbils, rats and mice.

Route of Administration

The CD40:CD154 binding interrupters designed according to this invention may be administered in any manner which is medically acceptable. Depending on the specific circumstances, local or systemic administration may be desirable. Local administration may be, for example, by subconjunctival administration. Preferably, the interrupter is administered via an oral, an enteral, or a parenteral route such as by an intravenous, intraarterial, subcutaneous, intramuscular, intraorbital, intraventricular, intraperitoneal, subcapsular, intracranial, intraspinal, topical or intranasal injection, infusion or inhalation. The interrupter also may be administered by implantation of an infusion pump, or a biocompatible or bioerodiable sustained release implant, into the subject.

Dosages and Frequency of Treatment

Generally, the methods described herein involve administration of the CD40:CD154 binding interrupter at desired intervals (e.g., daily, twice weekly, weekly, biweekly, monthly or at other intervals as deemed appropriate) over at least a two- or three-week period. The administration schedule is adjusted as needed to treat the condition associated with inappropriate or abnormal CD154 activation in the subject. The present treatment regime can be repeated in the event of a subsequent episode of illness.

A CD40:CD154 binding interrupter designed using the methods of this invention may be administered in a pharmaceutically effective, prophylactically effective or therapeutically effective amount, which is an amount sufficient to produce a detectable, preferably medically beneficial effect on a subject at risk or afflicted with a condition associated with inappropriate or abnormal CD154 activation. Medically beneficial effects include preventing, inhibiting, reversing or attenuating deterioration of, or detectably improving, the subject's medical condition. The amount and frequency of dosing for any particular compound to be administered to a patient for a given immunological condition associated with inappropriate or abnormal CD154 induced activation in a subject is within the skills and clinical judgement of ordinary practitioners of the medical and pharmaceutical arts. The general dosage and administration regime may be established by preclinical and clinical trials, which involve extensive but routine studies to determine the optimal administration parameters of the compound. Even after such recommendations are made, the practitioner will often vary these dosages for different subjects based on a variety of considerations, such as the individual's age, medical status, weight, sex, and concurrent treatment with other pharmaceuticals. Determining the optimal dosage and administration regime for each CD40:CD154 binding interrupter used is a routine matter for those of skill in the medical and pharmaceutical arts.

Generally, the frequency of dosing may be determined by an attending physician or similarly skilled practitioner, and might include periods of greater dosing frequency, such as at daily or weekly intervals, alternating with periods of less frequent dosing, such as at monthly or longer intervals.

To exemplify dosing considerations for a CD40:CD154 binding interrupter, the following examples of administration strategies, for an anti-CD154 mAb, serve as a guide. The dosing amounts could easily be adjusted or adapted for other types of anti-CD154 compounds. In general, single dosages of between about 0.05 and about 50 mg/kg patient body weight are contemplated, with dosages most frequently in the 1-20 mg/kg range. For acute treatment, such as before or at the time of transplantation, or in response to any evidence that graft rejection is beginning, an effective dose of a novel CD40:CD154 binding interruptor compound of this invention may be patterned on that of a representative antibody (such as 5c8 mAb), ranges from about 1 mg/kg body weight to about 20 mg/kg body weight, administered daily for a period of about 1 to 5 days, preferably by bolus intravenous administration. The same dosage and dosing schedule may be used in the load phase of a load-maintenance regimen, with the maintenance phase involving intravenous or intramuscular administration of antibodies in a range of about 0.1 mg/kg body weight to about 20 mg/kg body weight, for a treatment period of anywhere from weekly to 3 month intervals. Chronic treatment may also be carried out by a maintenance regimen, patterned on those in which antibodies are administered by intravenous or intramuscular route, in a range of about 0.1 mg/kg body weight to about 20 mg/kg body weight, with interdose intervals ranging from about 1 week to about 3 months. In addition, chronic treatment may be effected by an intermittent bolus intravenous regimen, patterned on those in which between about 1.0 mg/kg body weight and about 100 mg/kg body weight of antibodies are administered, with the interval between successive treatments being from 1 to 6 months. For all except the intermittent bolus regimen, administration may also be by oral, pulmonary, nasal or subcutaneous routes.

For treatment, a CD40:CD154 binding interrupter can be formulated in a pharmaceutical or prophylactic composition which includes, respectively, a pharmaceutically or prophylactically effective amount of the CD40:CD154 binding interrupter dispersed in a pharmaceutically acceptable carrier. In some embodiments, the pharmaceutical or prophylactic composition can also include a pharmaceutically or prophylactically effective amount of another immunosuppressive or immunomodulatory compound, including without limitation: an agent that interrupts T cell costimulatory signaling via CD28 (e.g., CTLA4-Ig), CD80 or CD86; an agent that interrupts calcineurin signaling (e.g., cyclosporin, a macrolide such tacrolimus, formerly known as FK506); a corticosteroid; or an antiproliferative agent (e.g., azathioprine). Other therapeutically effective compounds suitable for use with the CD40:CD154 binding interruptor include rapamycin (also known as sirolimus); mycophenolate mofetil (MMF), mizoribine, deoxyspergualin, brequinar sodium, leflunomide, azaspirane and the like.

Combination therapies according to this invention for treatment of a condition associated with inappropriate or abnormal CD154 activation in a subject include the use of a CD40:CD154 binding interruptor together with agents targeted at B cells, such as anti-CD19, anti-CD28 or anti-CD20 antibody (unconjugated or radiolabeled), IL-14 antagonists, LJP394 (LaJolla Pharmaceuticals receptor blocker), IR-1116 (Takeda small molecule) and anti-Ig idiotype monoclonal antibodies. Alternatively, the combinations may include T cell/B cell targeted agents, such as CTLA41 g, IL-2 antagonists, IL-4 antagonists, IL-6 antagonists, receptor antagonists, anti-CD80/CD86 monoclonal antibodies, TNF, LFA1/ICAM antagonists, VLA4/VCAM antagonists, brequinar and IL-2 toxin conjugates (e.g., DAB), prednisone, anti-CD3 mAb (OKT3), mycophenolate mofetil (MMF), cyclophosphamide, and other immunosuppressants such as calcineurin signal blockers, including without limitation, tacrolimus (FK506). Combinations may also include T cell targeted agents, such as CD4 antagonists, CD2 antagonists and anti-IL-12 antibodies.

The immunomodulatory compound that may be co-administered with an CD40:CD154 binding interrupter to a subject with a condition associated with inappropriate or abnormal CD154 activation may be an antibody that specifically binds to a protein selected from the group consisting of CD45, CD2, IL2R, CD4, CD8 and RANK Fe.

Formulation

In general, CD40:CD154 binding interrupters of this invention are suspended, dissolved or dispersed in a pharmaceutically acceptable carrier or excipient. The resulting therapeutic composition does not adversely affect the recipient's homeostasis, particularly electrolyte balance. Thus, an exemplary carrier comprises normal physiologic saline (0.15M NaCl, pH 7.0 to 7.4). Other acceptable carriers are well known in the art and are described, for example, in Remington's Pharmaceutical Sciences, Gennaro, ed., Mack Publishing Co., 1990. Acceptable carriers can include biocompatible, inert or bioabsorbable salts, buffering agents, oligo- or polysaccharides, polymers, viscoelastic compound such as hyaluronic acid, viscosity-improving agents, preservatives, and the like.

All references cited herein are hereby incorporated by reference.

The following are examples that illustrate the methods and compositions of this invention. These examples are included for the purposes of illustration only.

### EXAMPLE 1

### Determination of the Crystal Structure of Humanized 5c8 FAB-CD154 Complex

Humanized 5c8 mAb was prepared by or for Biogen, Inc. (Cambridge, Mass.) by the following method. cDNAs encoding the variable regions of the heavy and light chains of anti-human CD154 5c8 mAb (produced by the hybridoma having ATCC Accession Number HB 10916) (as described in U.S. Pat. No. 5,474,771 and Lederman et al. J. Exp. Med. 175: 1091 (1992), the disclosures of both of which are hereby incorporated by reference) were cloned from total cellular RNA from the murine hybridoma cells by RT-PCR. For humanization, the murine CDRs were grafted onto a homologous human variable region framework, retaining murine residues deemed to be important in maintaining antigen binding, by conventional recombinant DNA technology. See sequence in FIG. 8. Using conventional recombinant DNA technology, the DNA for the variable regions were fused to human constant regions (IgG1 heavy chain and kappa light chain) and a vector for stable expression of humanized 5c8 mAb in NS0 myeloma cells was constructed. The cell line was grown and humanized 5c8 mAb was purified by conventional techniques to greater than 95% purity and shown to be biologically active by binding assay and bioassays for inhibition in vitro. The humanized 5c8 mAb maintained the binding properties of the murine 5c8 mAb.

The humanized 5c8 mAb Fab fragments were produced by cleaving whole humanized 5c8 mAb with papain and isolating the Fab fragments, as essentially described by the papain manufacturer (Pierce, Rockford, Ill.) with Pierce's Immobilized Papain (#20341) with a few modifications. The intact humanized 5c8 mAb was prepared at a concentration of 10 mg/ml in a buffer containing 20 mM phosphate, 10 mM EDTA and 25 mM cysteine, pH 7.0. Immobilized papain was added at an enzyme to substrate ratio of 1:50 and digestion was allowed to occur overnight at 37° C. with rocking. The immobilized papain was removed and the crude digest was dialyzed against 20 mM sodium acetate buffer at pH 4.5. The Fab fragments were separated from residual intact antibody, dimeric Fab fragment, and Fc fragment by cation exchange chromatography (Poros HS/M, PerSeptive Biosytems #PO42M26) with a shallow salt gradient. The humanized 5c8 mAb Fab fragments were then buffer exchanged into PBS (14.4 mM sodium phosphate dibasic, 5.6 mM sodium phosphate monobasic, 150 mM NaCl) and purified further by size exclusion chromatography (Sephacryl S300, Pharmacia Biotech). The humanized 5c8 mAb Fab fragment comprises at least amino acids 1 to 219 of the heavy chain (Gln 1 to Lys 219 in FIGS. 4 and 8) and amino acids 1 to 215 of the light chain (Asp 1 to Arg 215 in FIGS. 4 and 8). Because the humanized 5c8 mAb Fab fragments were produced by papain digestion, the exact C-termini of the heavy and light chains of hu5c8 mAb Fab fragments were not determined. Amino acids 1 to 219 of the heavy chain and amino acids 1 to 215 of the light chain were visible in the crystal structure.

The CD154 was recombinant soluble CD154 consisting of residues 116-261 of the extracellular domain of human CD154 (Karpusas et al. Structure 3, 1031-1039 (1995) and Karpusas et al. Structure 3, 1446 (1995)). See FIG. 8. Recombinant human soluble CD154 consisting of residues 116 to 261 was expressed and purified from a Pichia pastoris clone as described in Karpusas et al. Structure 3, 1031-1039 (1995) and Karpusas et al. Structure 3, 1446 (1995). The soluble CD154 was mixed with excess hu5c8 mAb Fab fragment and incubated at 37° C. for 15 minutes. The uncomplexed hu5c8 mAb Fab fragment was separated from saturated CD154-hu5c8 mab Fab complexes by size exclusion chromatography using a S200 Sephacryl column (Pharmacia, Gibco). The CD154-hu5c8 mAb Fab complexes were further concentrated to 10-15 mg/ml in PBS buffer using Centricon Plus-20 (Amicon Bioseparations, Millipore). The stoichiometry of CD154 and hu5c8 Fab fragment in the saturated complexes was verified by SDS-PAGE analysis of the complexes with and without crosslinking reagent.

In order to determine conditions of crystallization, an incomplete factorial screen (Jancarick & Kim (1991) J. Appl. Crystalloqr. 24, 409-411) was set up using the Crystal Screen kits from Hampton Research (Riverside, Calif.). In a typical experiment, protein solution was mixed with an equal volume of reservoir solution and a drop of the mixture was suspended under a glass cover slip over the reservoir solution. Crystals were grown by vapor diffusion at room temperature by mixing a reservoir solution of 20% (w/v) PEG MME 550, 0.1 M MES pH 6.5, 0.01 zinc sulfate with equal volume of CD154-5c8 Fab complex solution. The crystals were thin and extremely fragile plates with dimensions 0.7×0.7 x 0.02 mm. They grew within a few days and were easy to reproduce. Some crystals were washed and dissolved and the sample was subjected to SDS-PAGE confirming that the crystals consisted of CD154-hu5c8 mAb Fab complex.

The crystals were cryoprotected by soaking them in a solution containing 25% PEG 400, 20% PEG MME 550, 0.1 M MES pH 6.5, 0.01 zinc sulfate and frozen in the liquid nitrogen gas stream (−175° C.). The procedure of crystal annealing was performed (Harp et al. (1998), Acta Cryst D54, 622-628). Crystals were transferred quickly after freezing in a 0.3 ml solution of 25% PEG 400, 20% PEG MME 550, 0.1 M MES pH 6.5, 0.01 zinc sulfate at room temperature for 3 minutes and then were frozen again in the liquid nitrogen gas stream.

A native X-ray data set up to 3.1 Å resolution was collected from one crystal by using an R-AXIS II image plate detector system (Molecular Structure Corporation, Woodlands, Tex.). The data were integrated and reduced using the HKL program package (Otwinowski, Z. (1993) Oscillation data reduction program., 56-62, Proceeding of the CCP4 study weekend: data collection and processing, Sawer, L., Issacs, N. & Bailey S. eds, Daresbury Laboratory, Warrington, UK). The data collection required about 4 days. The data set was 96.1% complete and had an R-merge of 7.6%. See Table 1 for additional data statistics.

Data processing suggested a monoclinic unit cell with approximate cell dimensions a=224.48 Å, b=129.91 Å, c=96.49 Åand β=109.6B. The space group was identified as C2. The Matthews volume (Matthews, B. W. (1968), J.Mol.Biol. 33, 491-497) was 3.1 Å3 Da⁻¹, assuming a complex of a CD154 trimer and 3 Fab fragments in the asymmetric unit, with a solvent content of 60.7%. The self rotation function calculated with XPLOR (Brunger, A. T. (1992) X-PLOR Version 3.1: A system for X-ray Crystallography and NMR, Yale University Press, New Haven, Conn., USA) exhibited a strong peak of 6.9763 at K=120° which indicated that there was a 3-fold axis that was perpendicular to the ab plane of the unit cell.

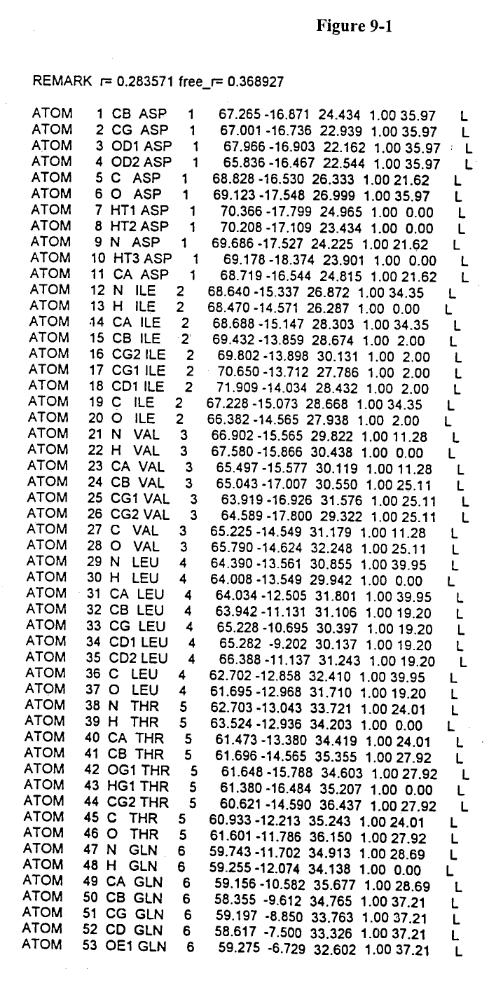

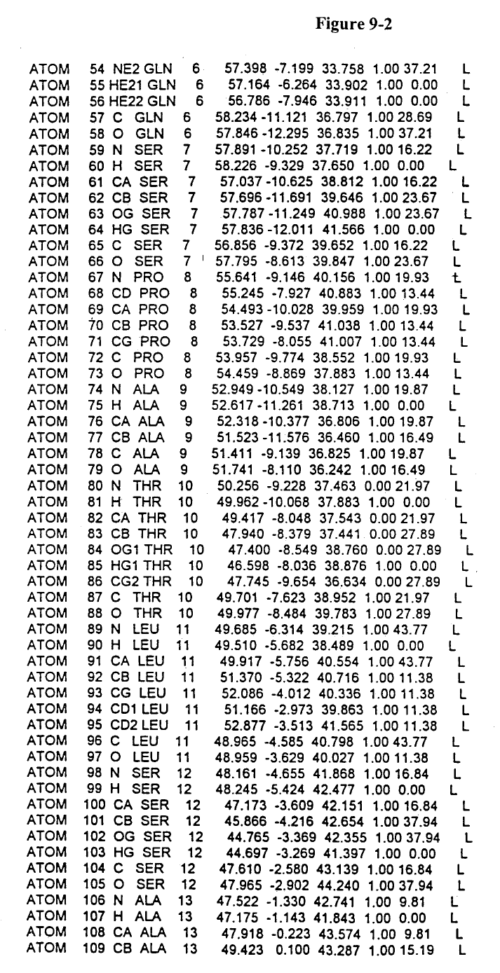

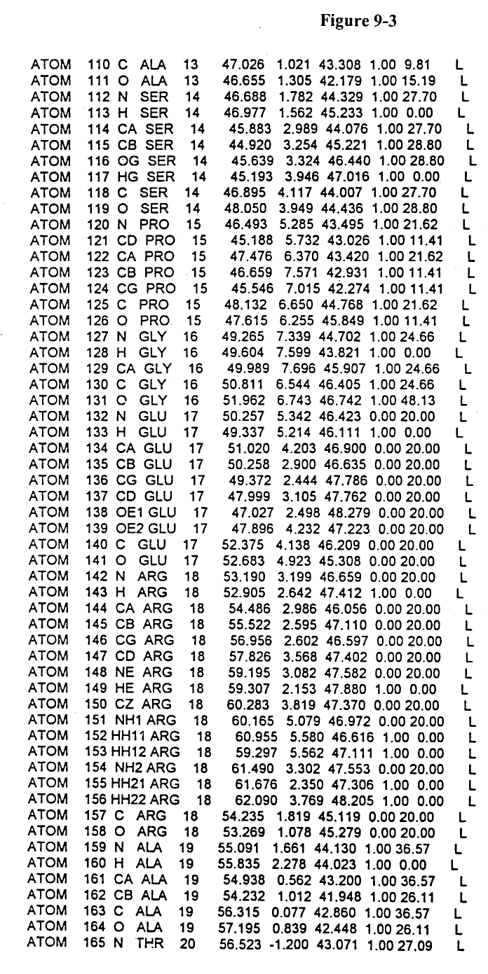

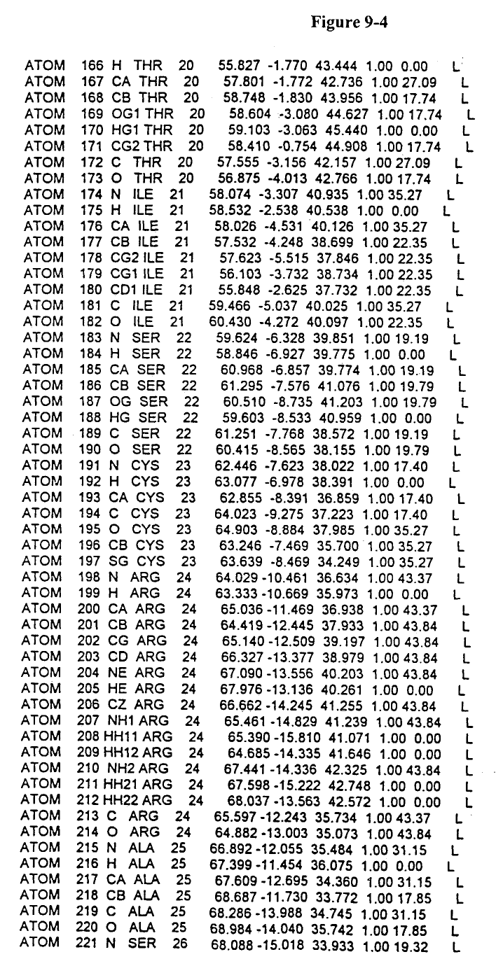

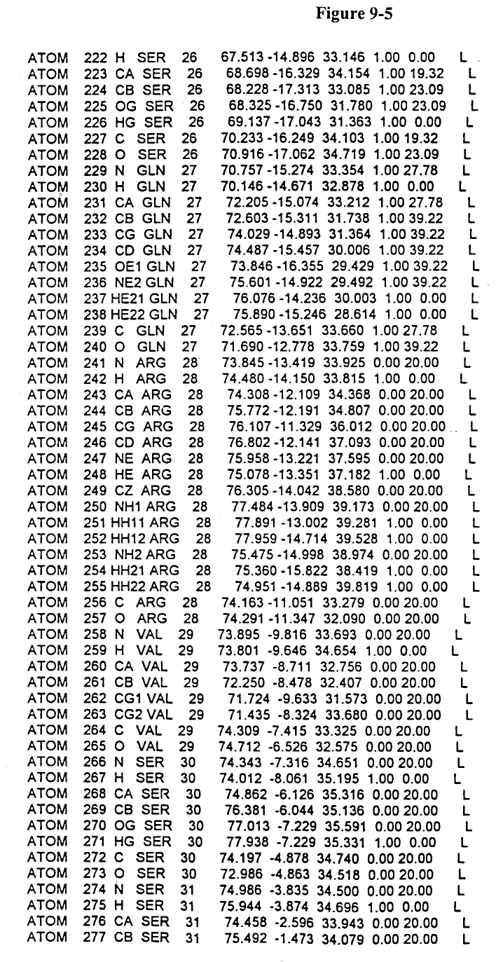

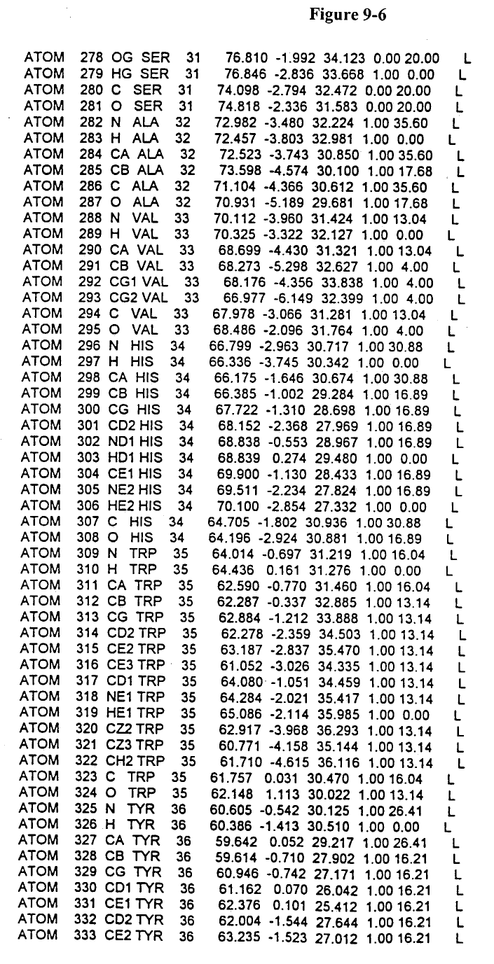

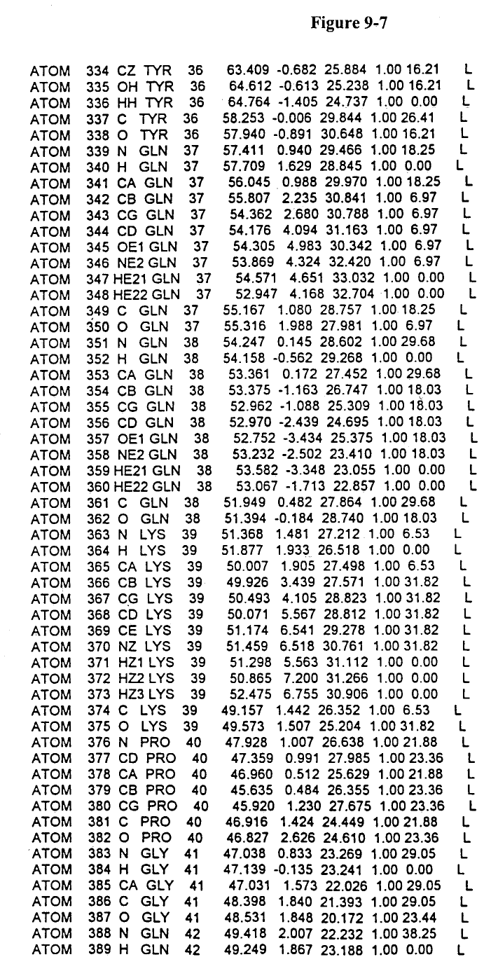

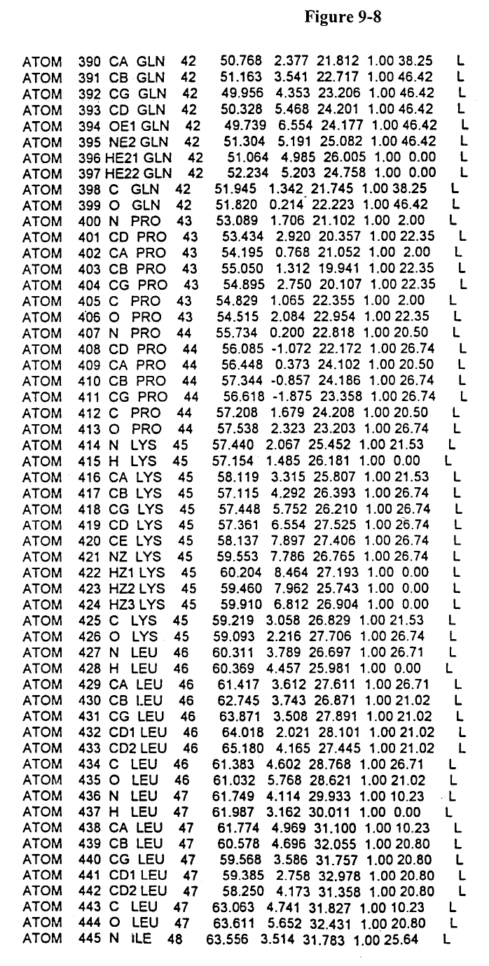

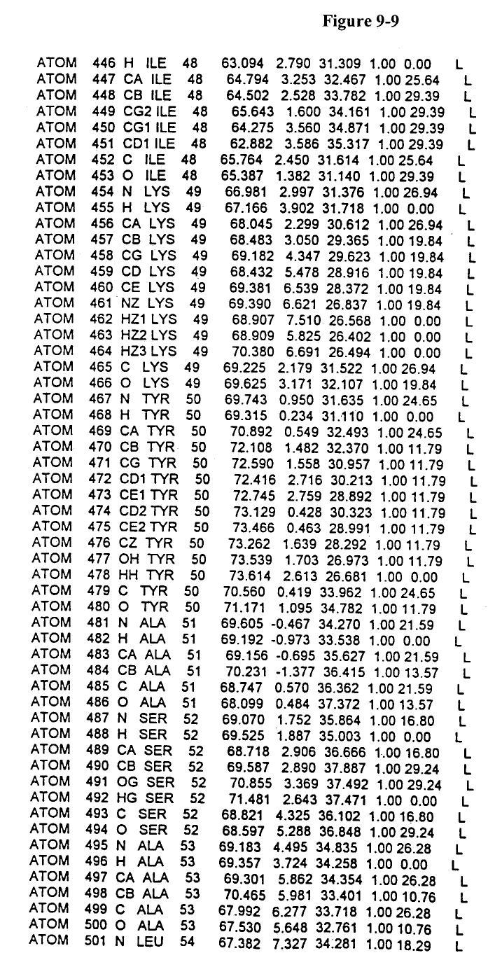

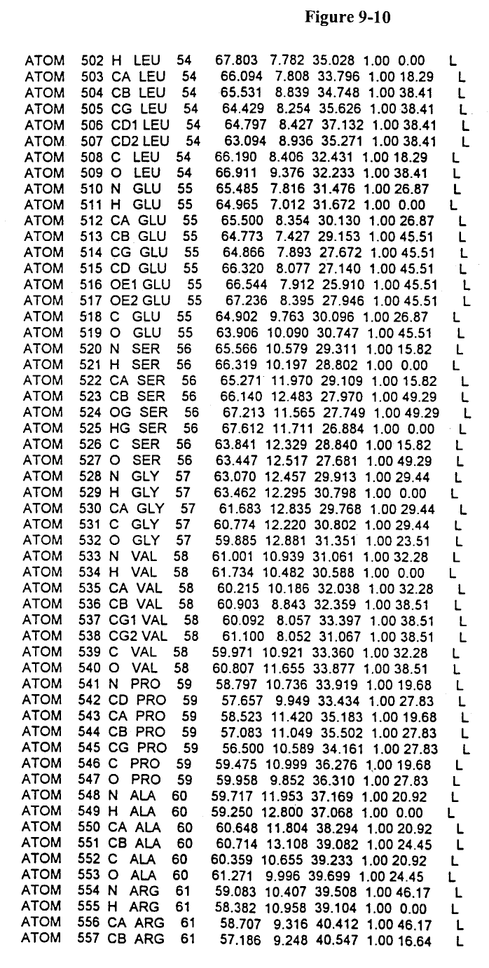

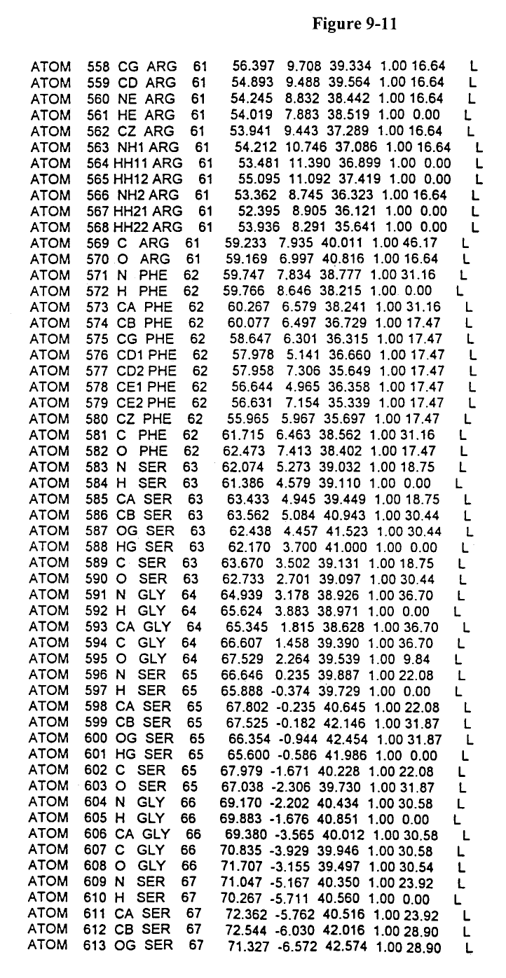

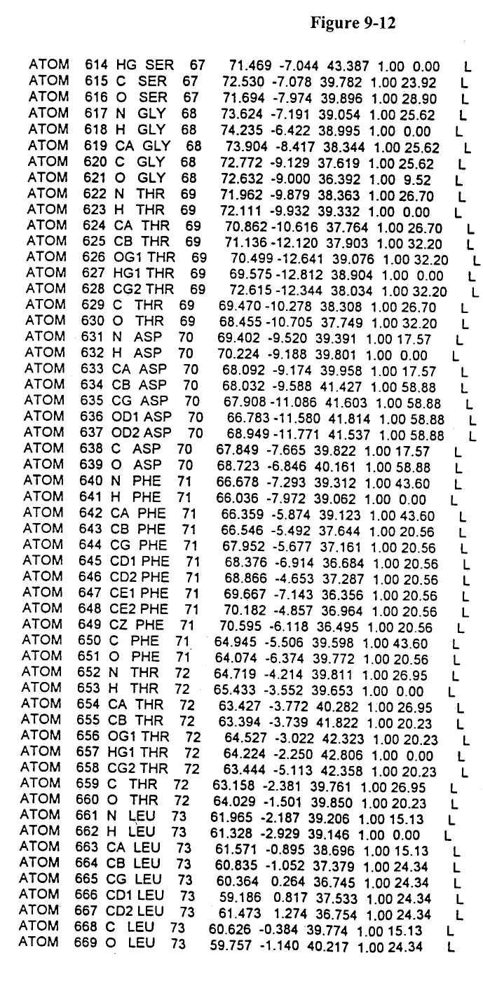

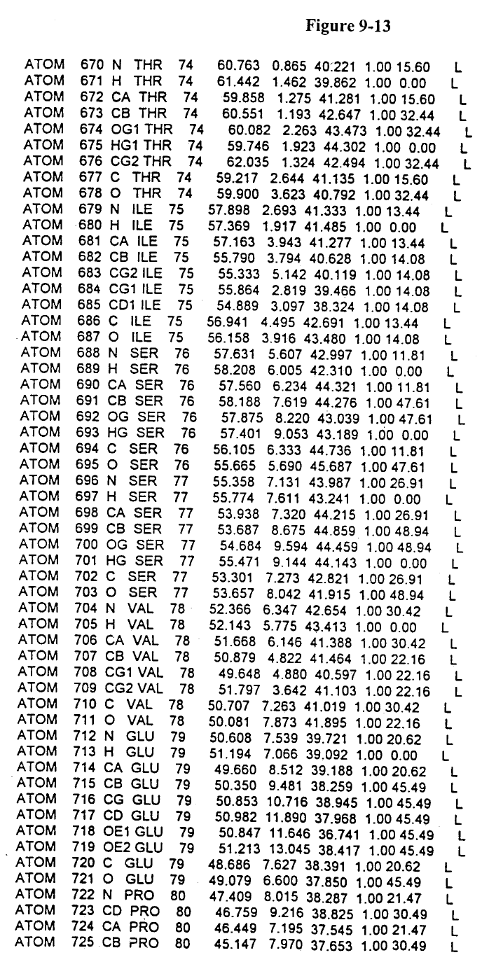

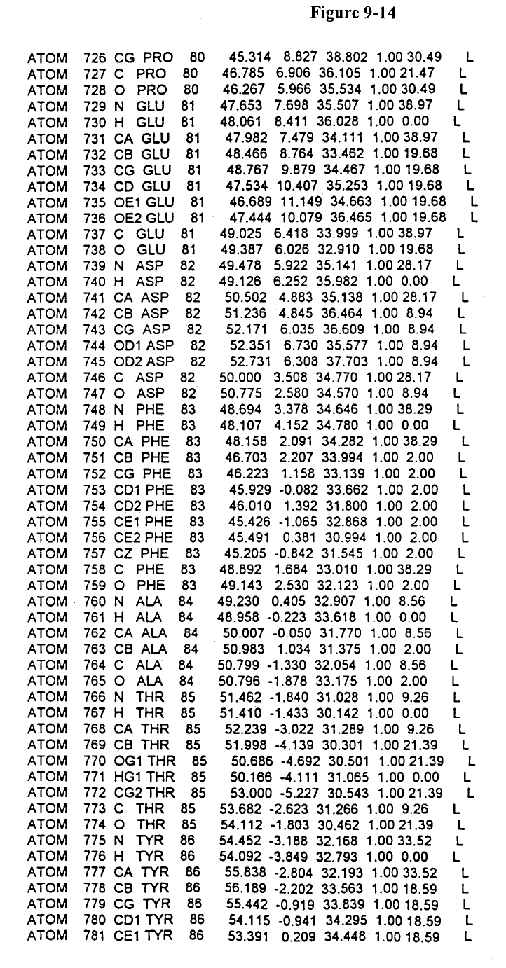

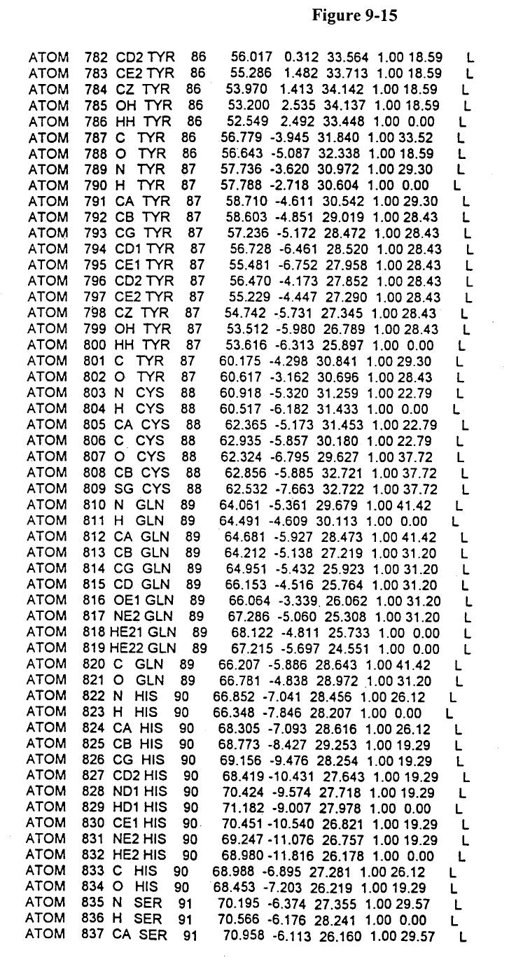

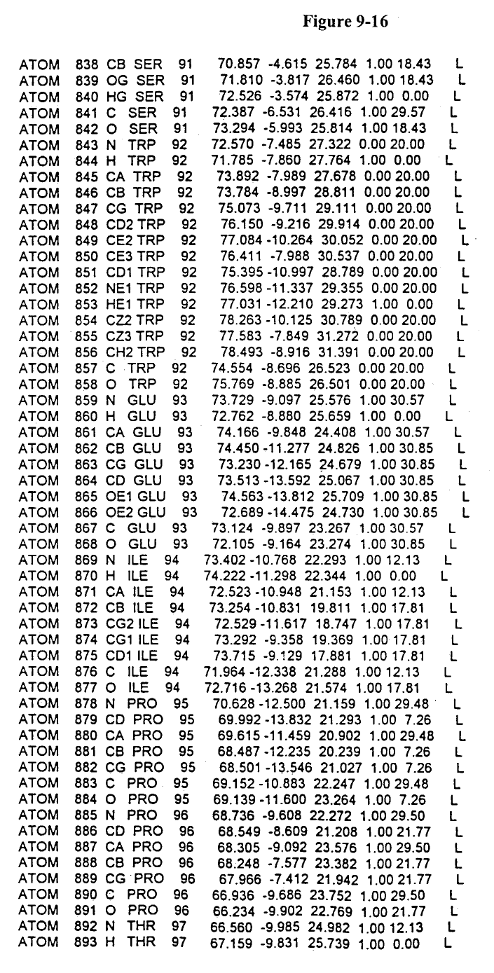

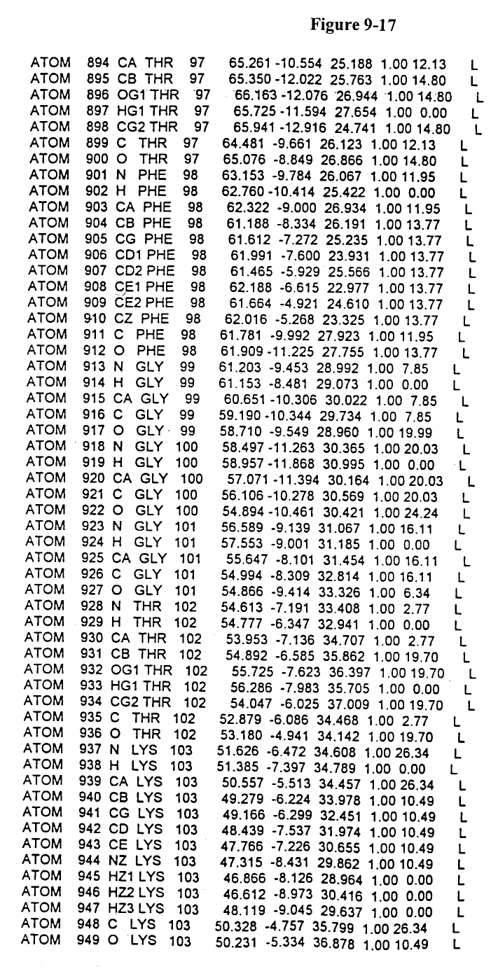

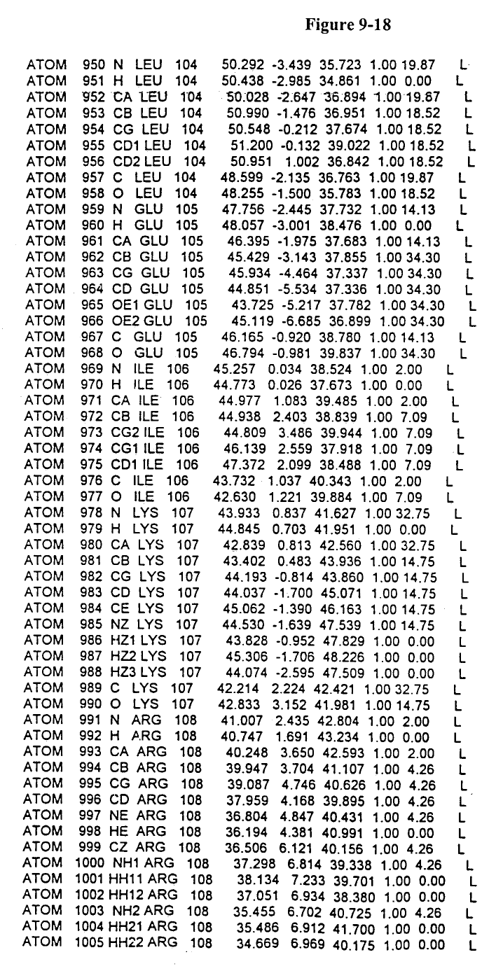

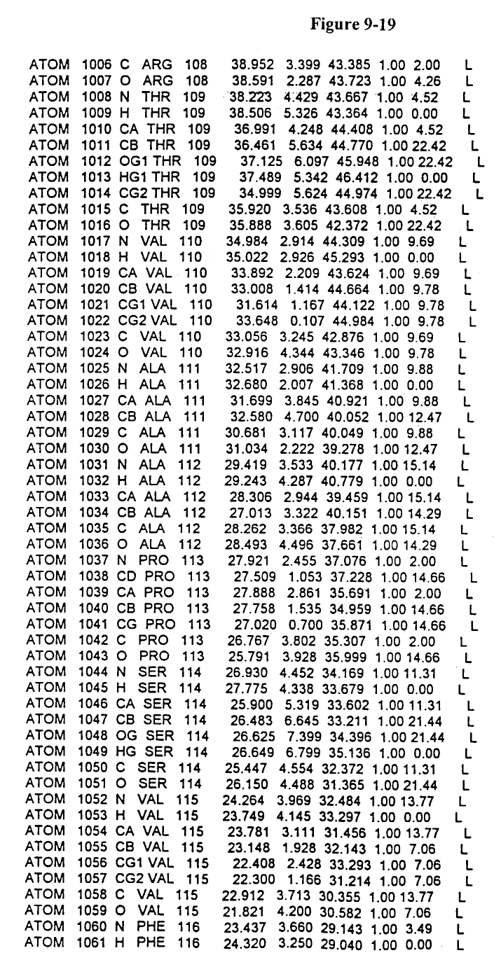

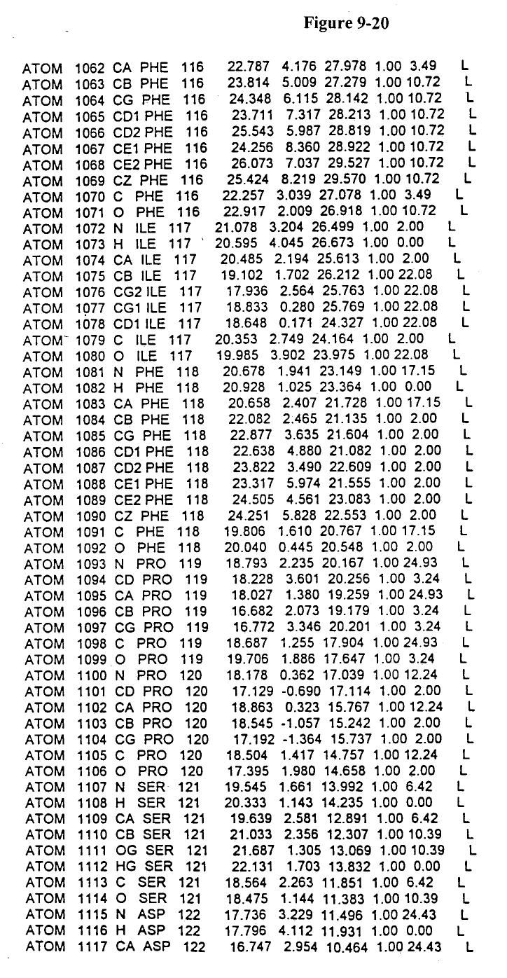

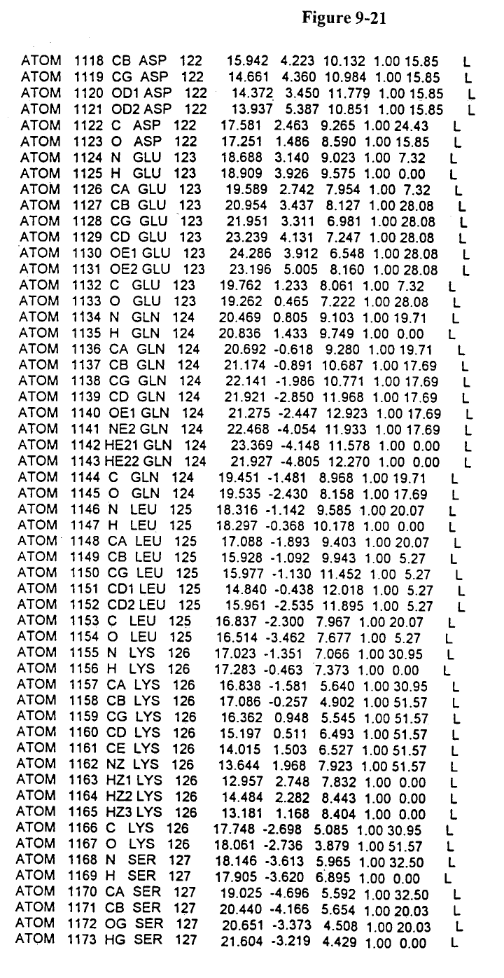

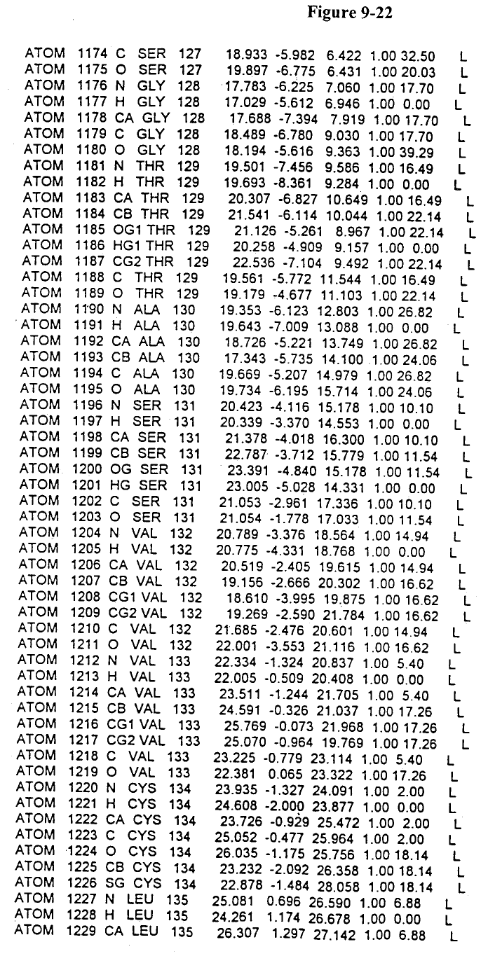

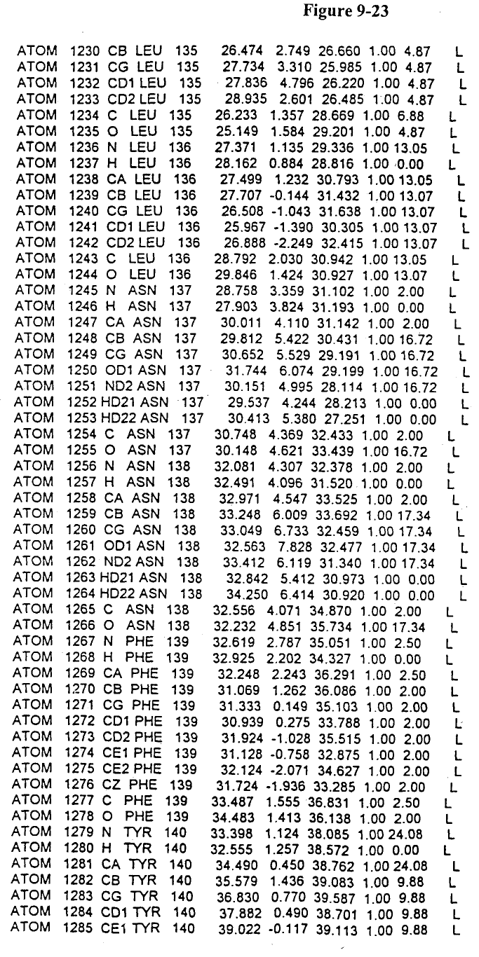

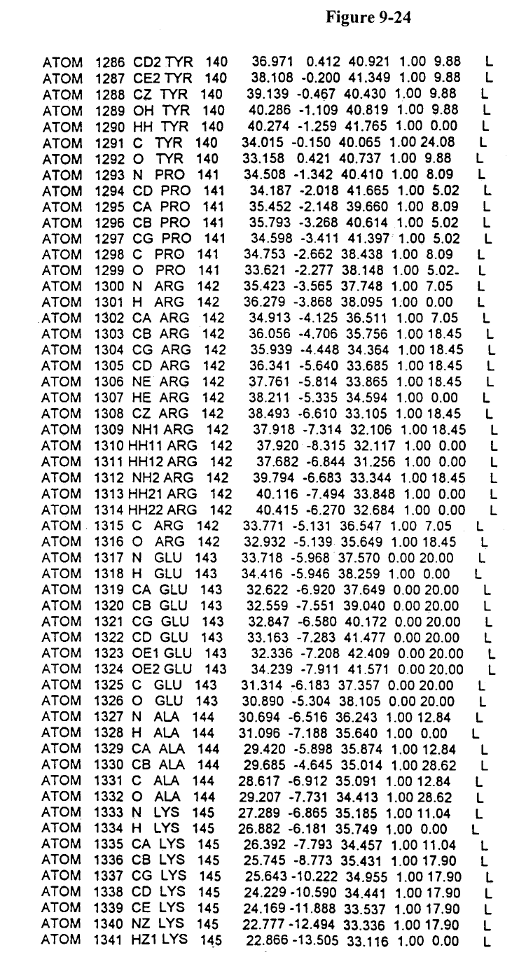

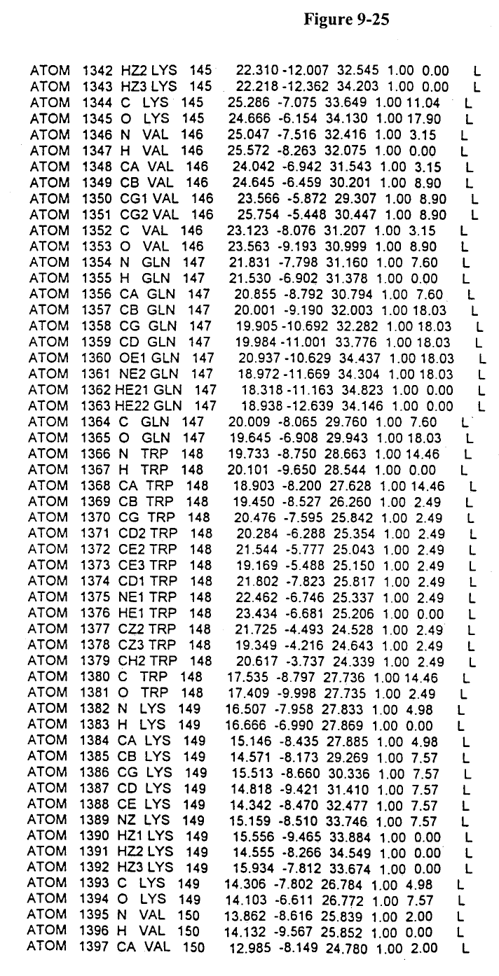

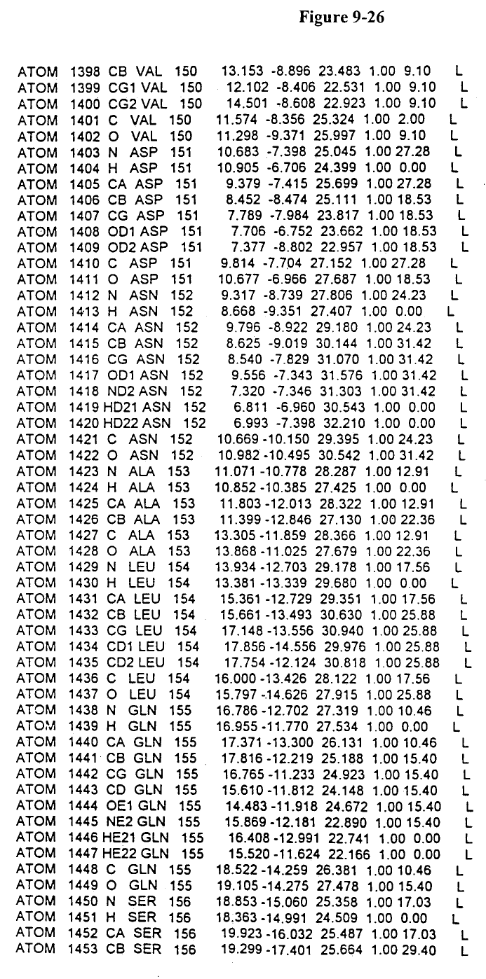

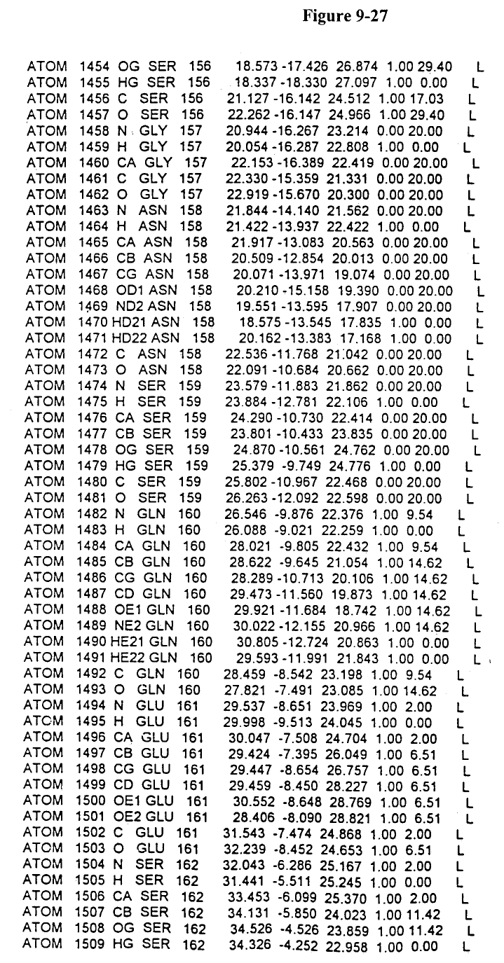

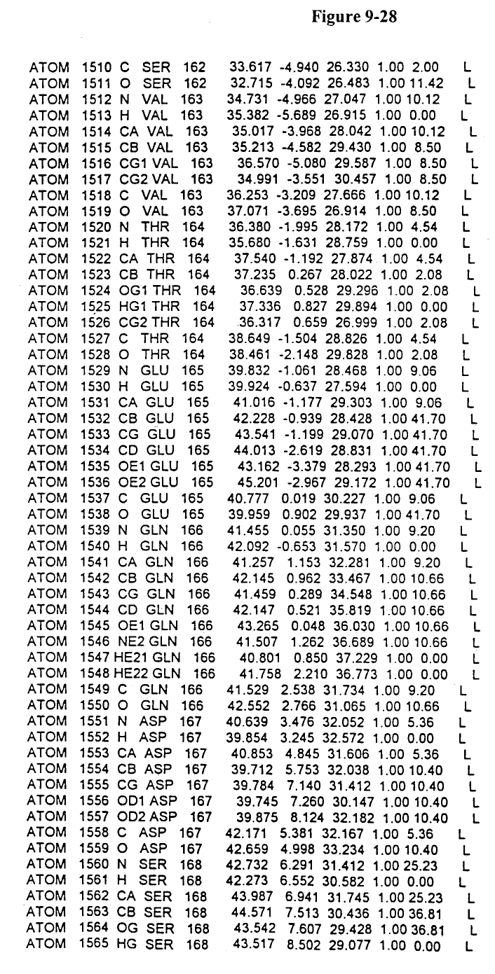

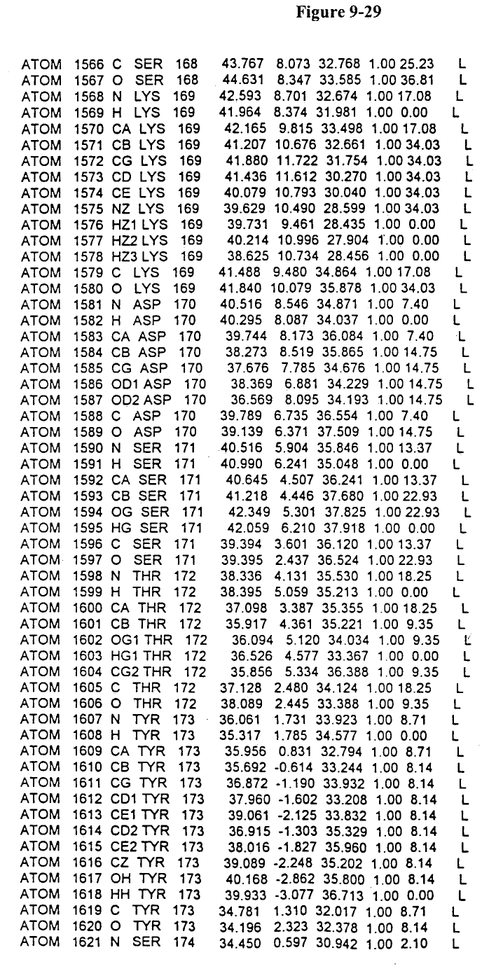

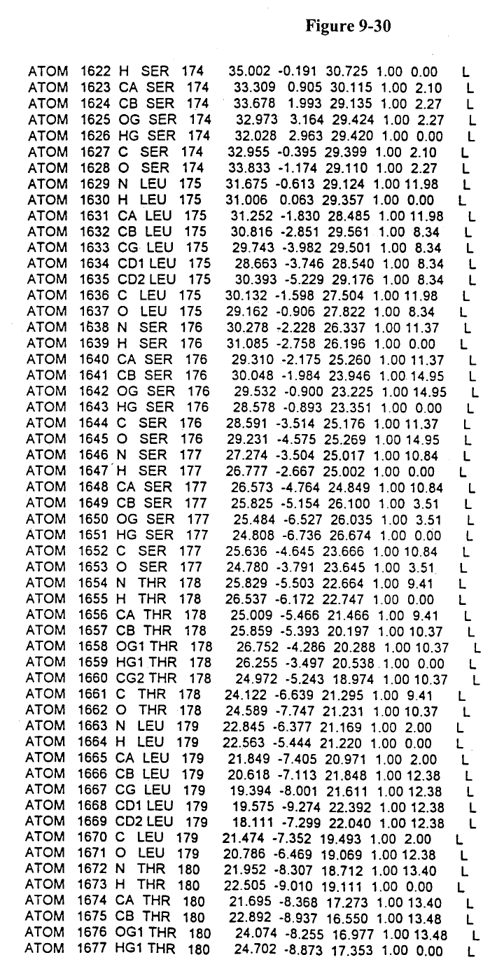

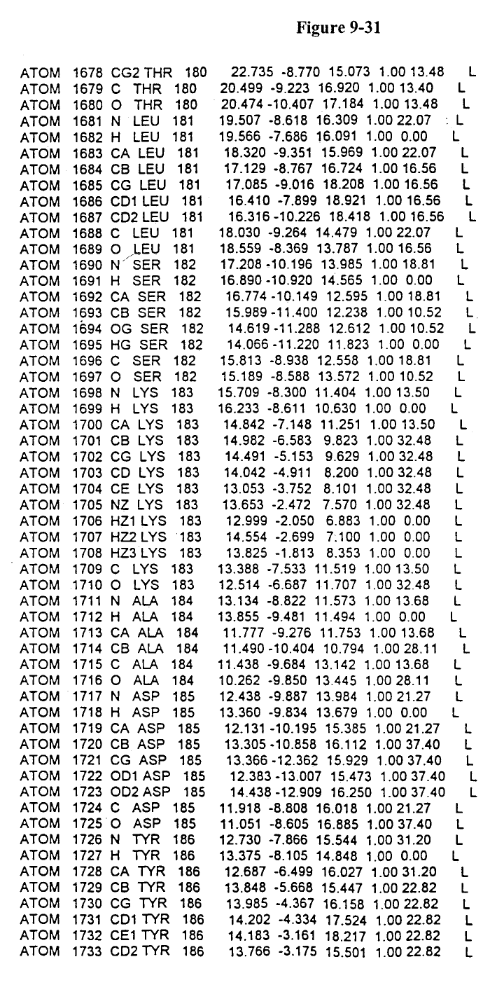

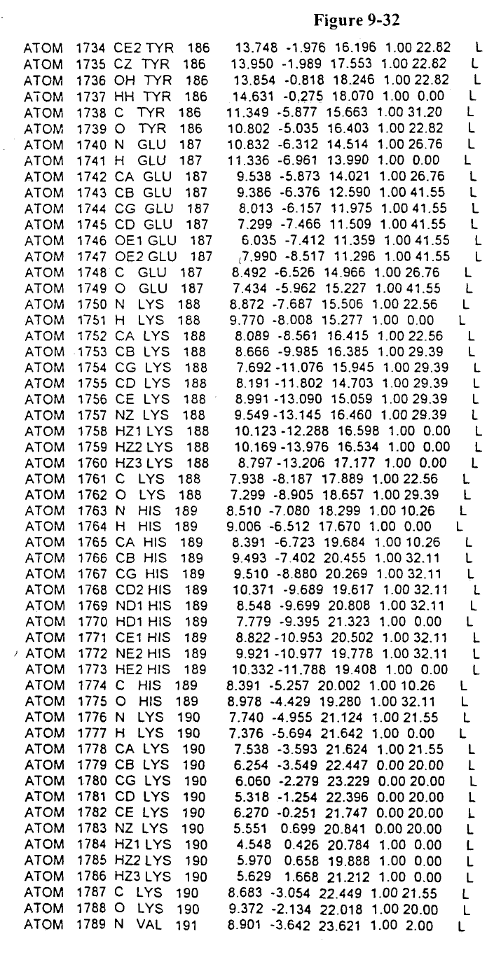

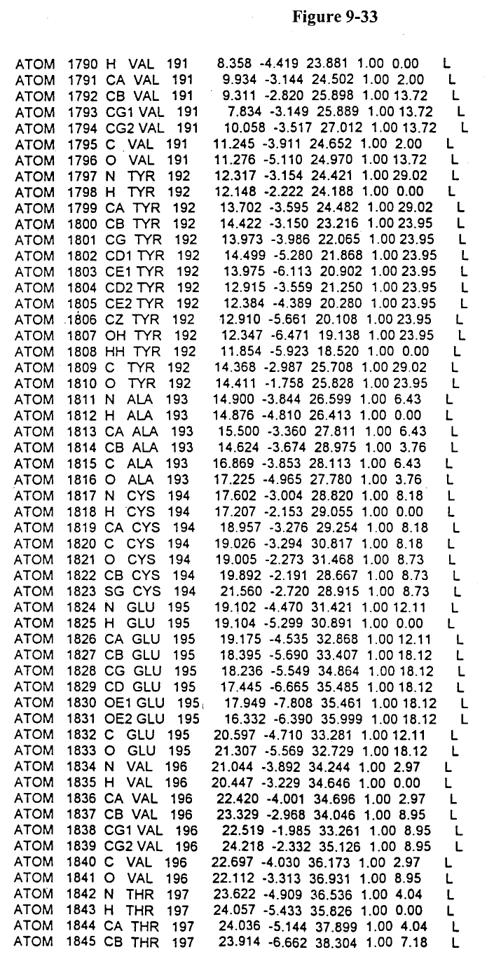

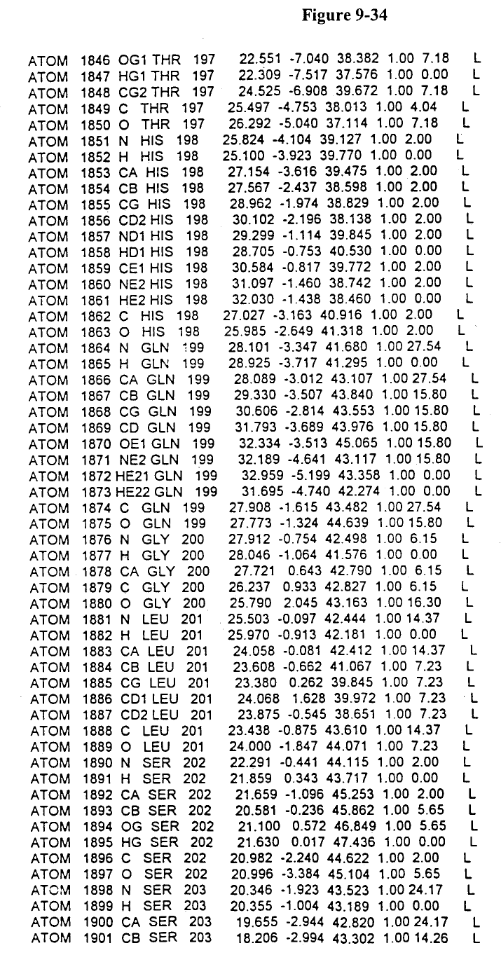

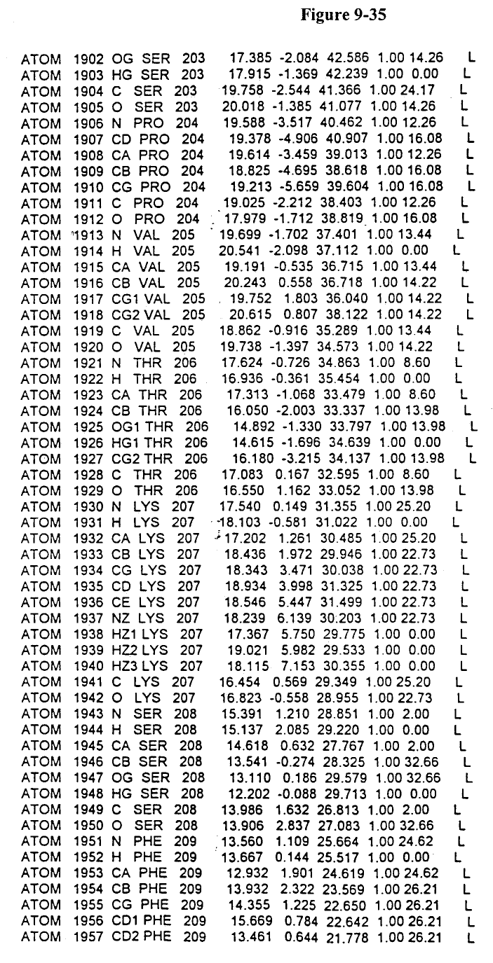

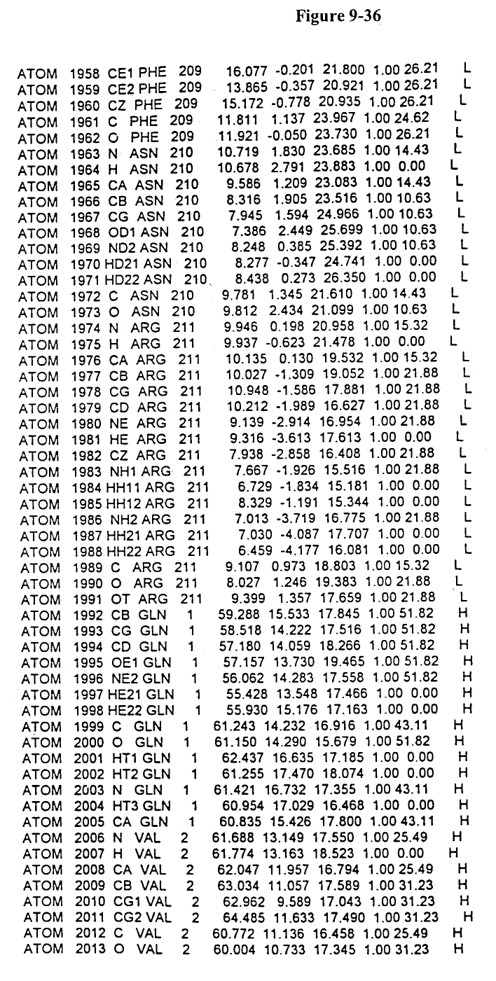

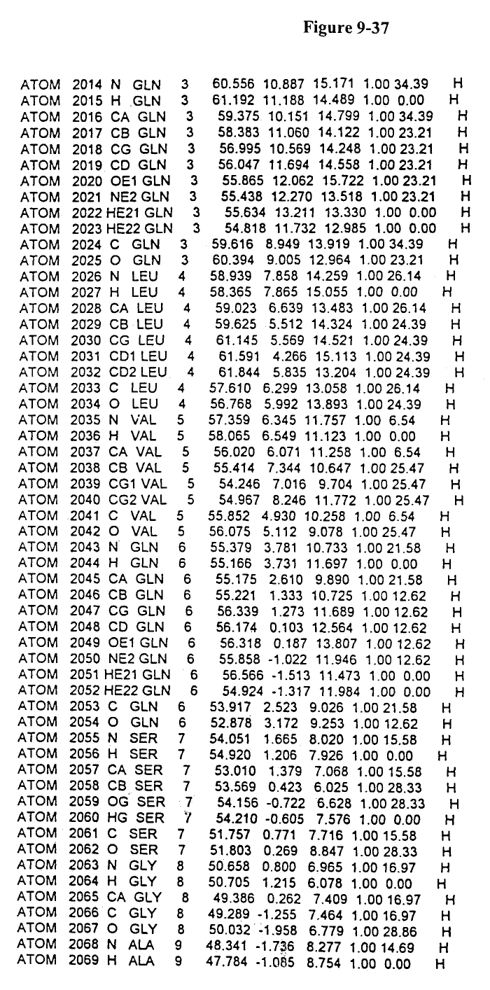

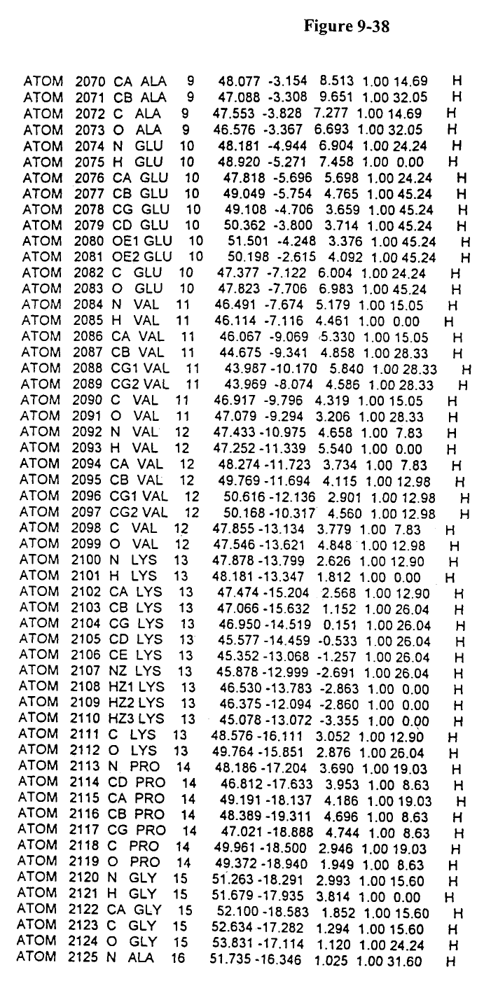

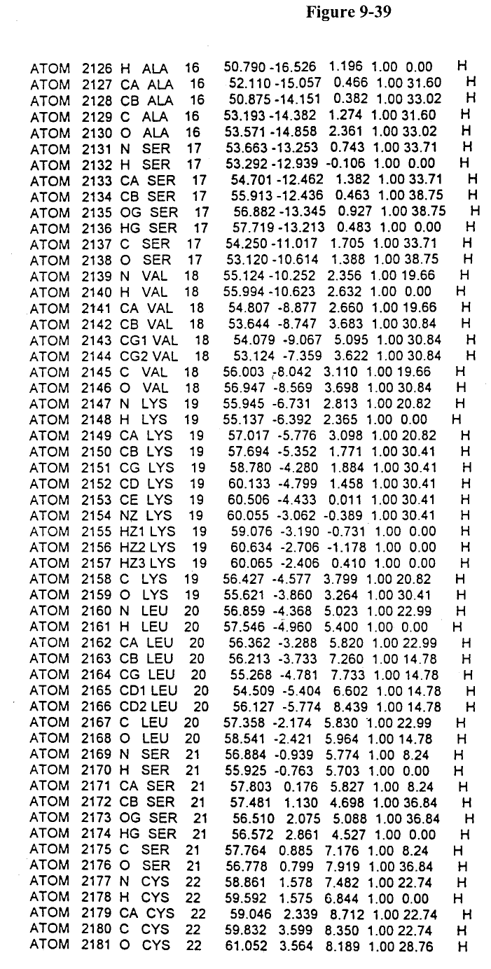

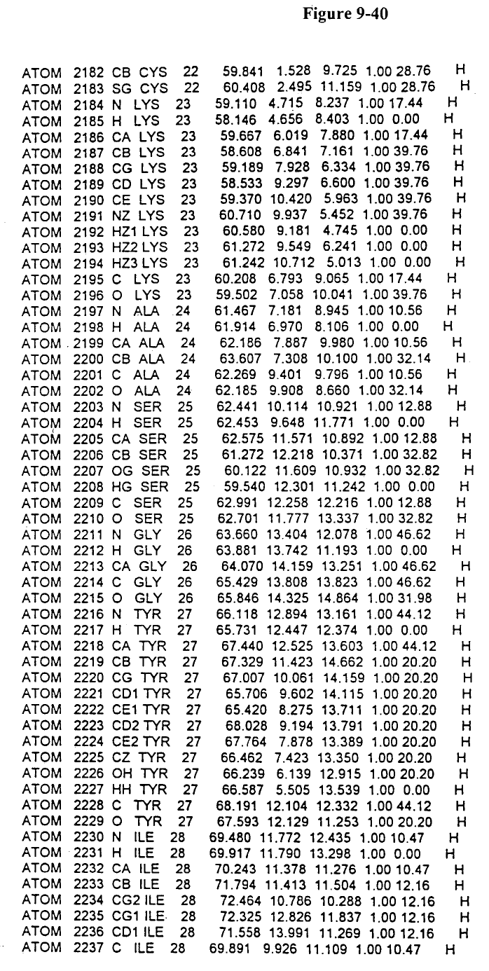

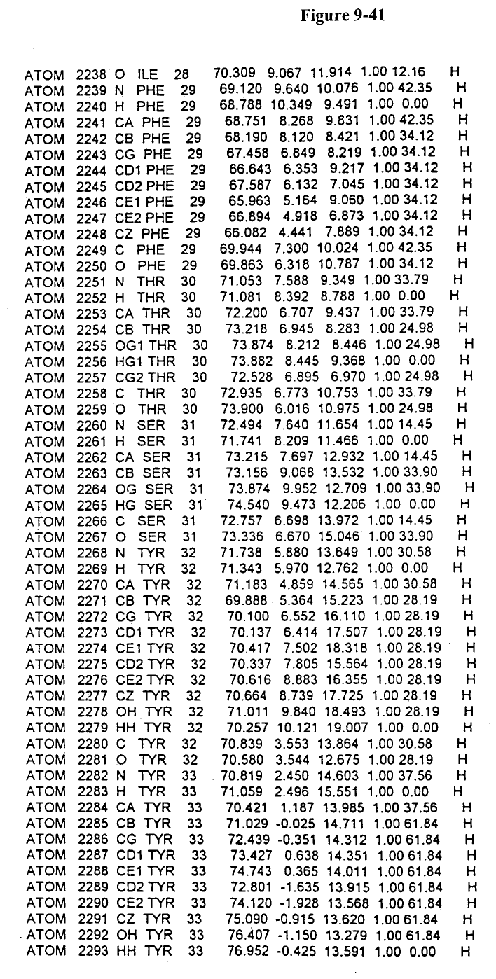

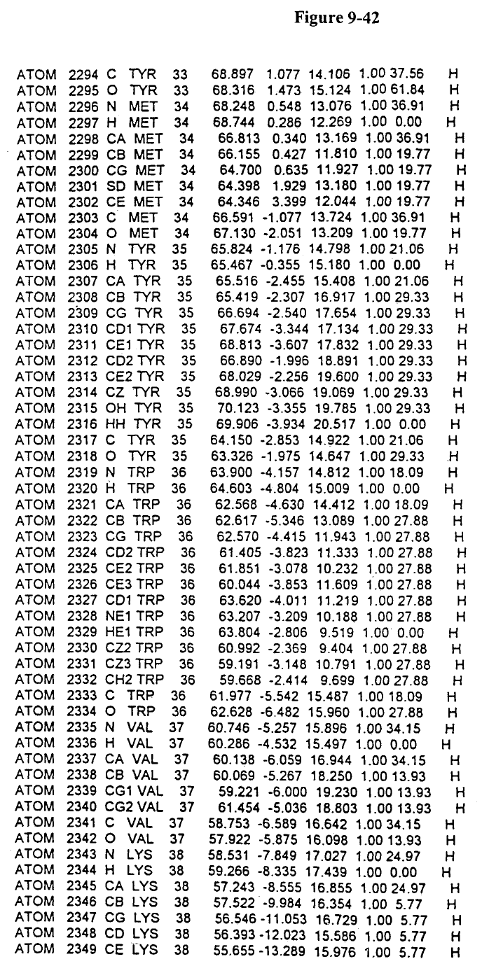

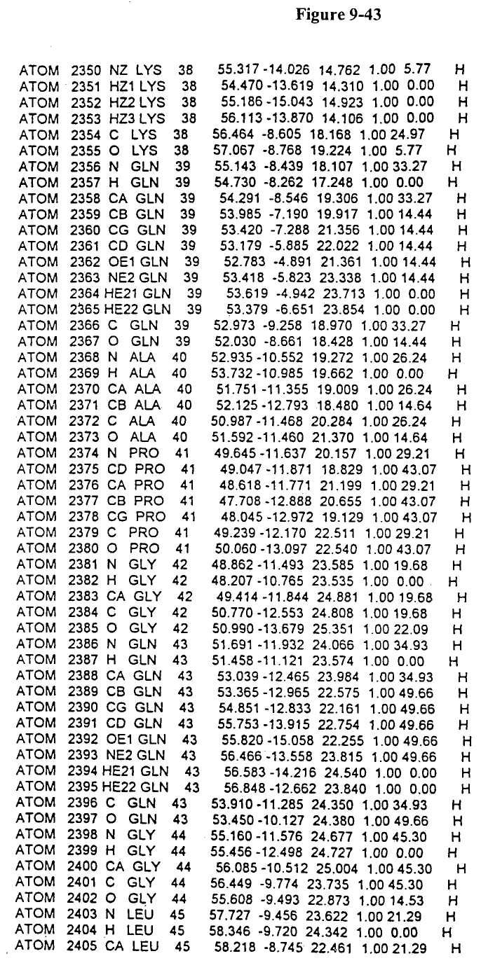

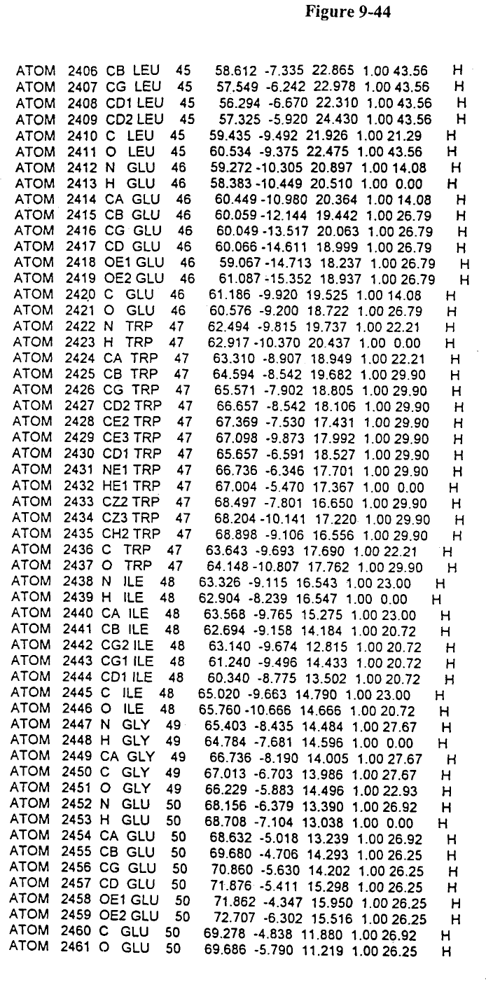

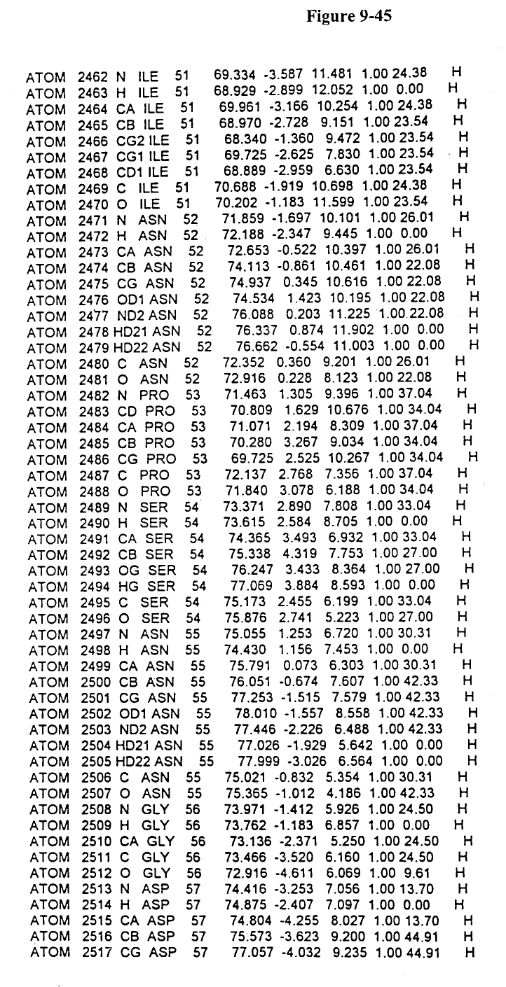

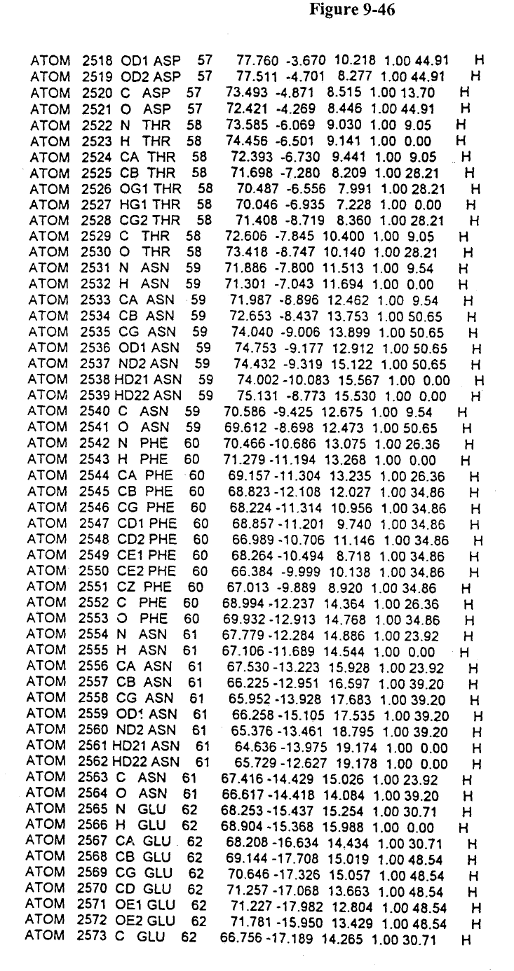

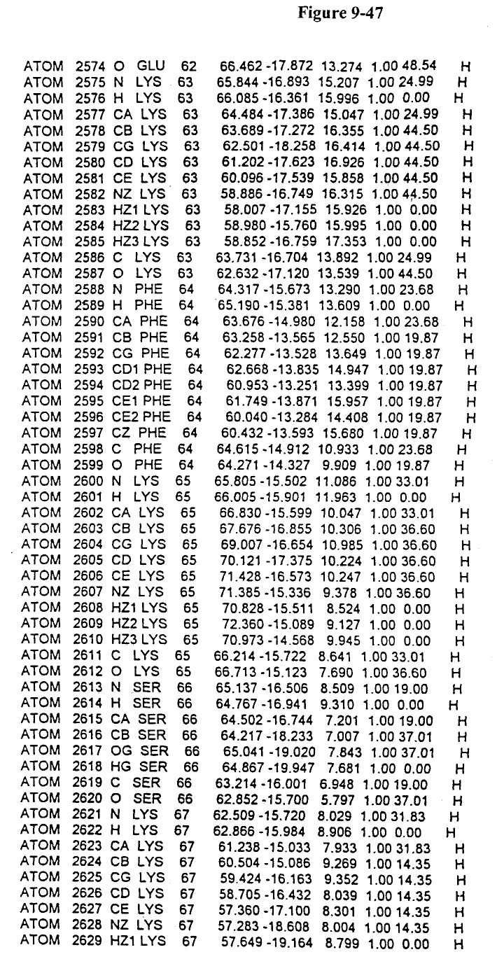

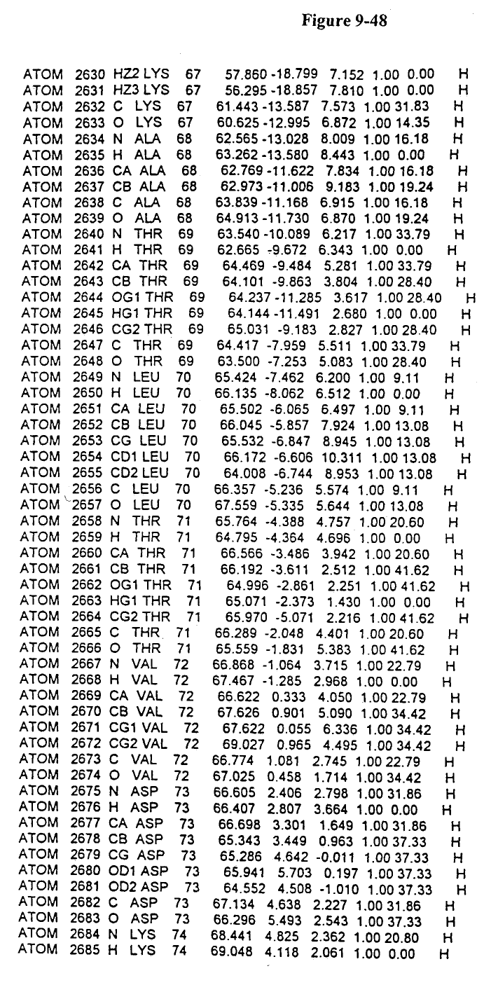

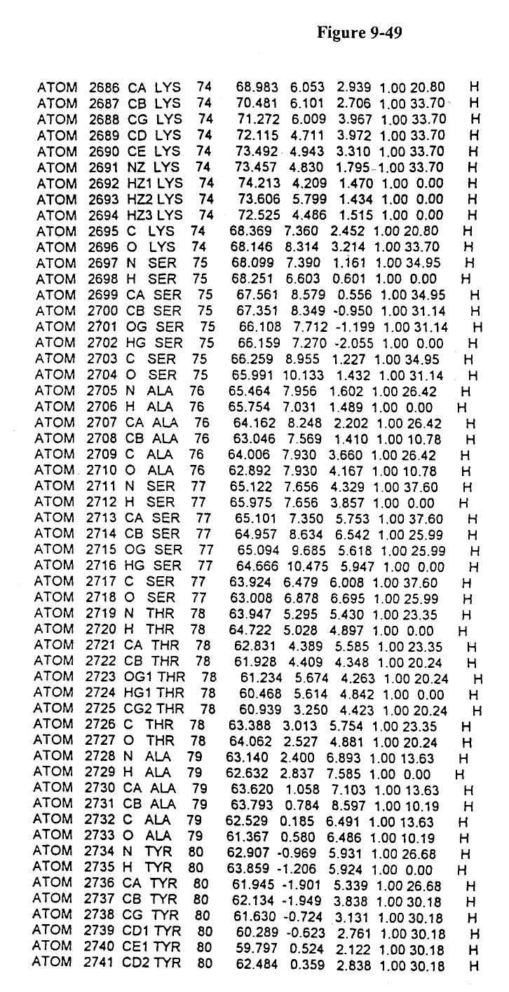

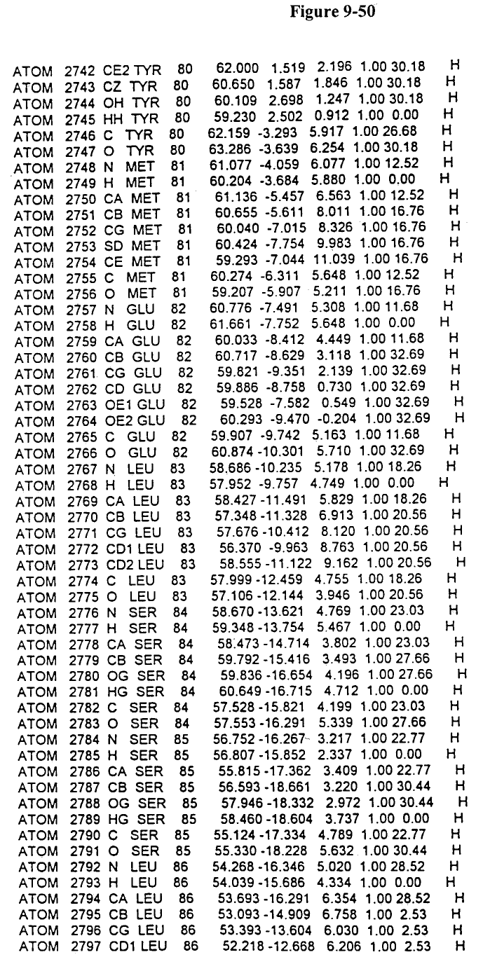

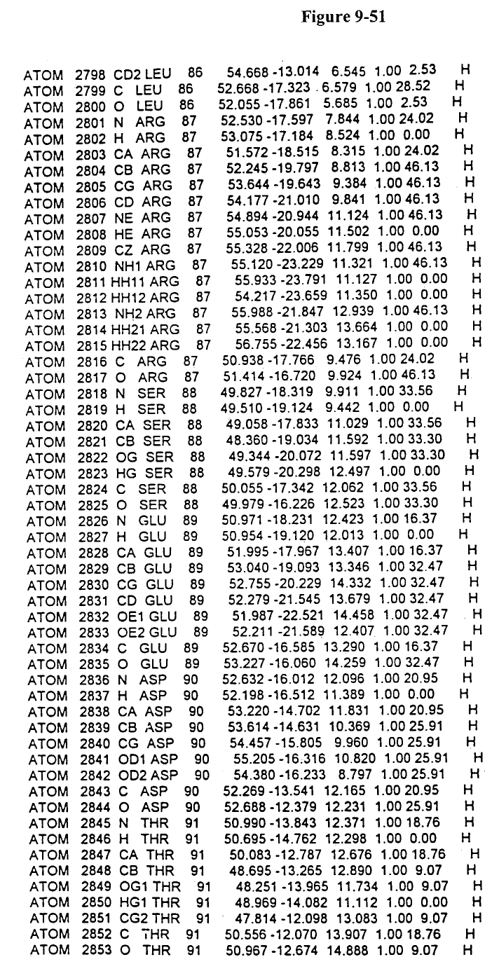

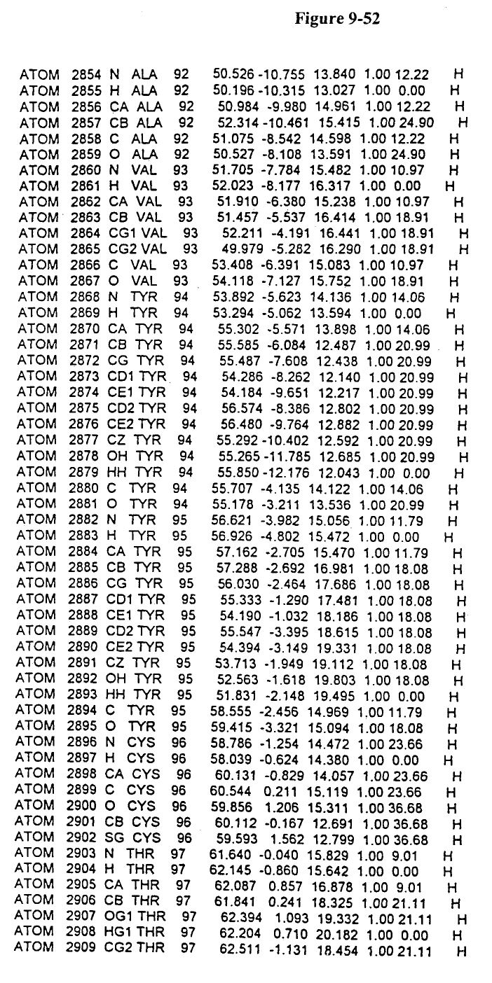

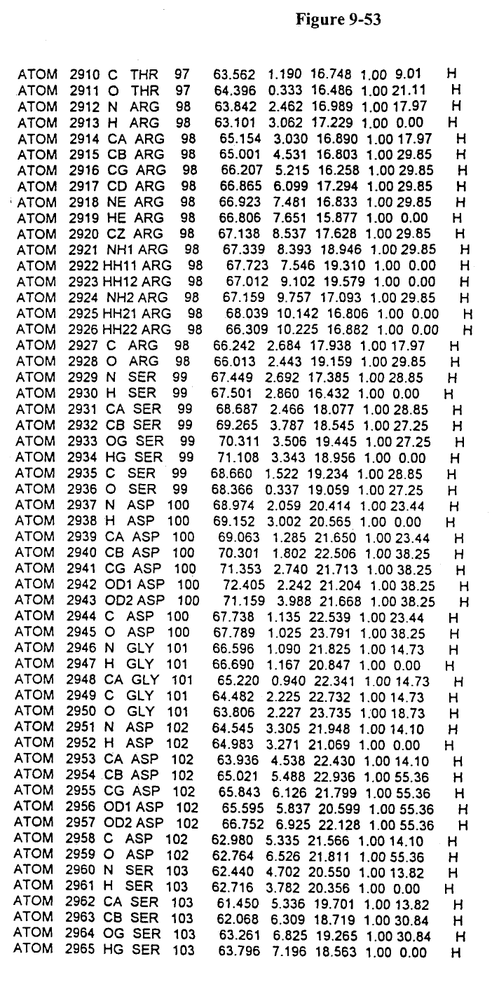

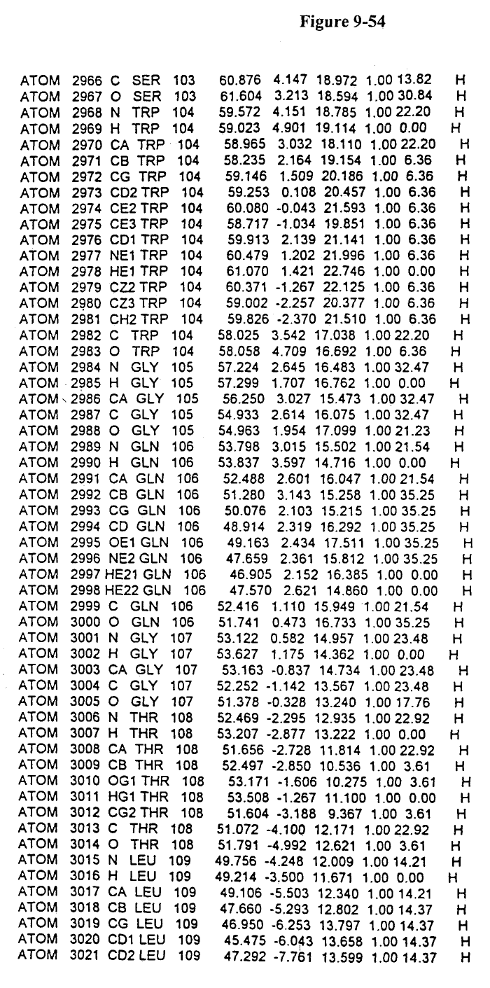

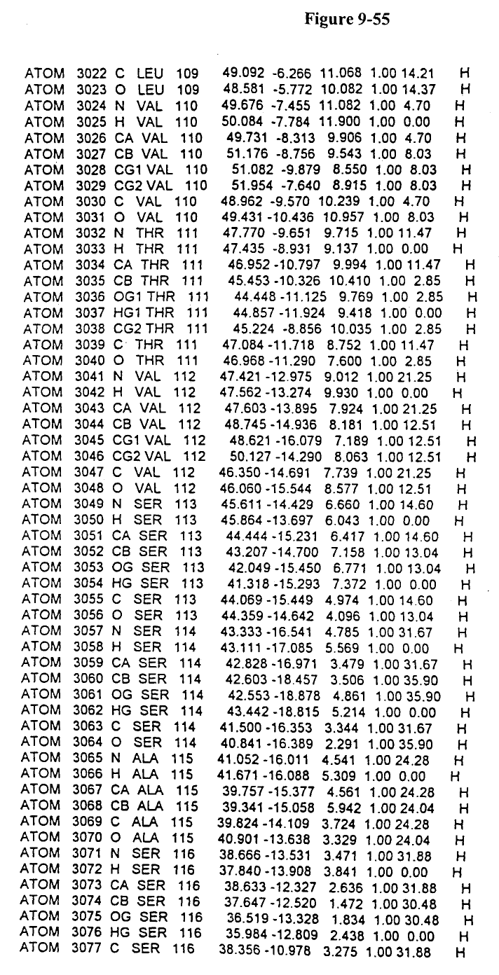

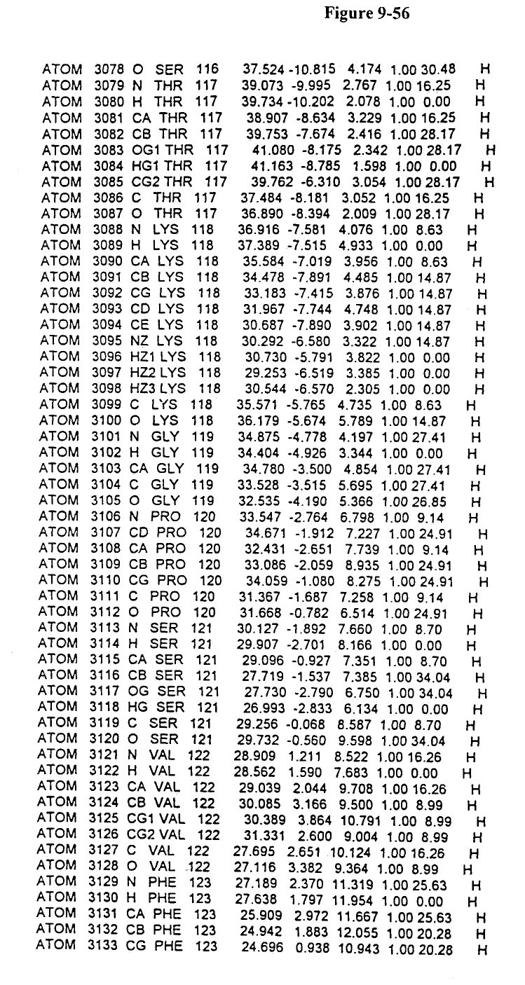

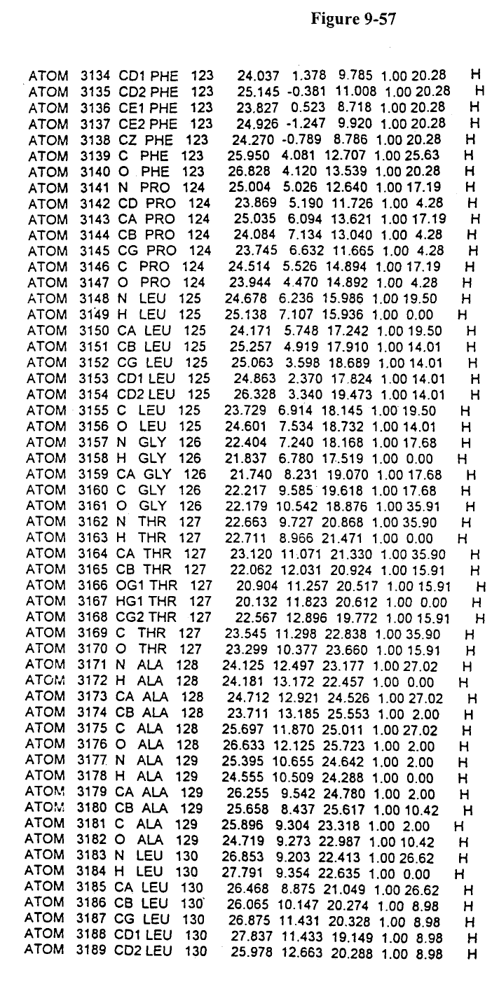

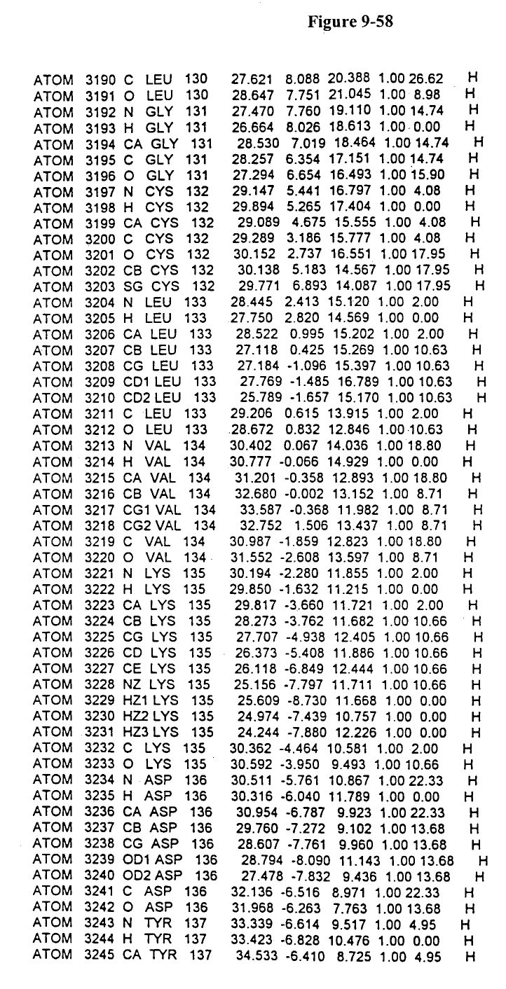

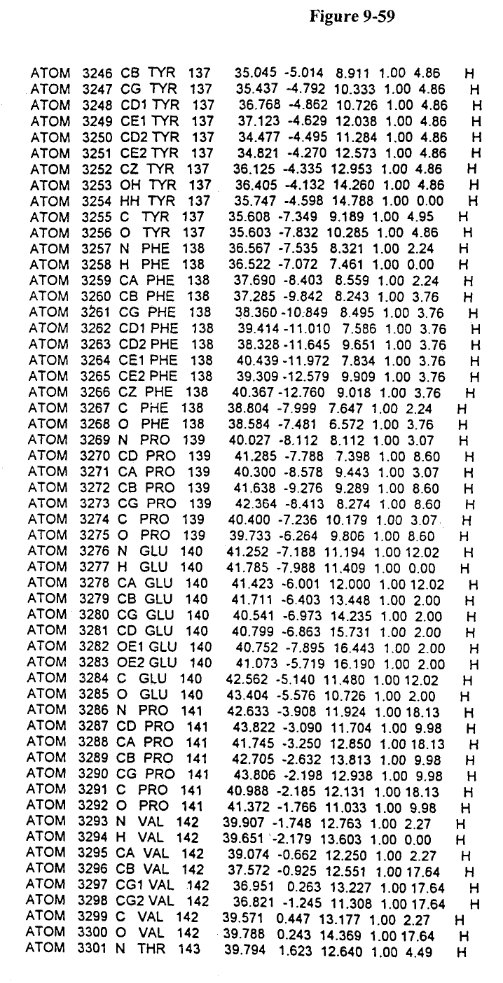

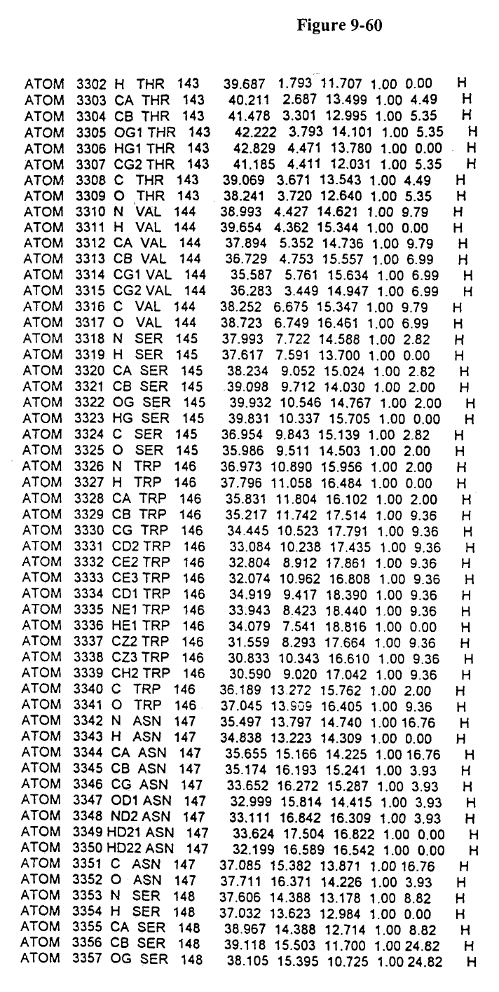

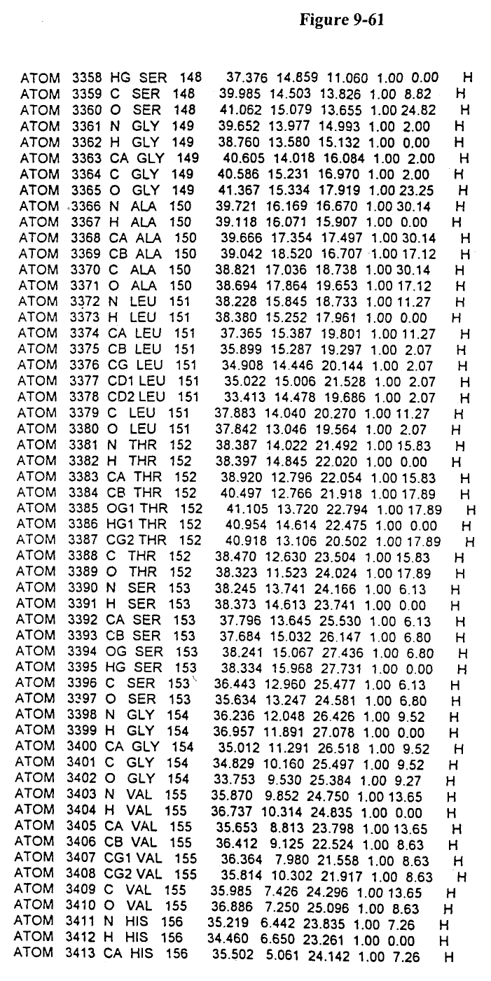

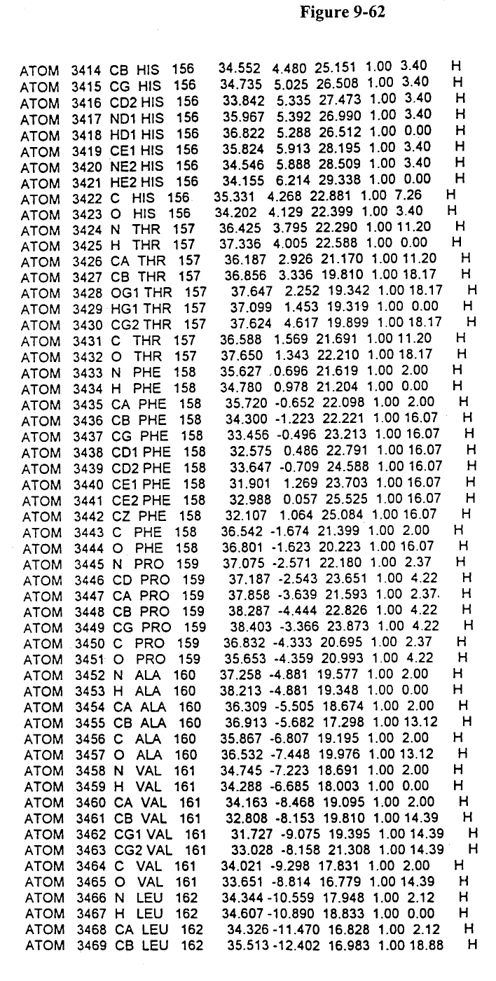

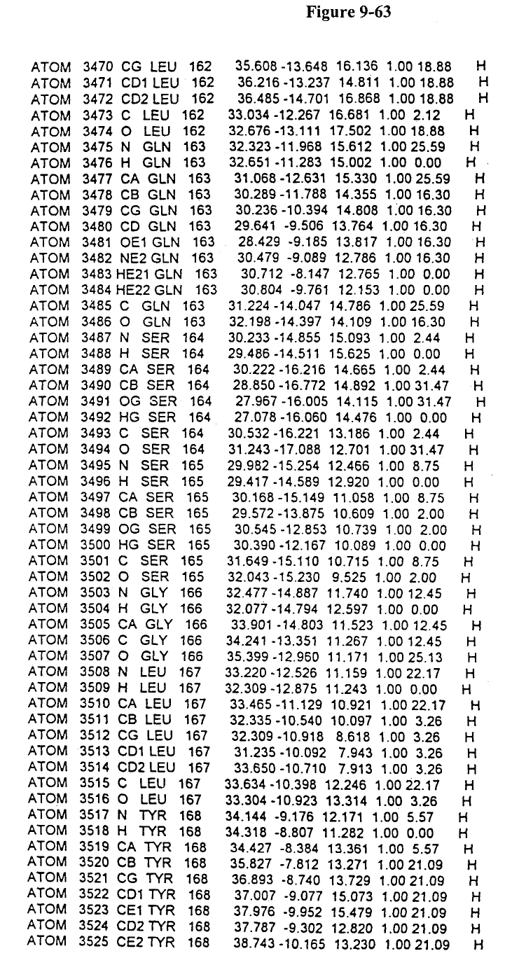

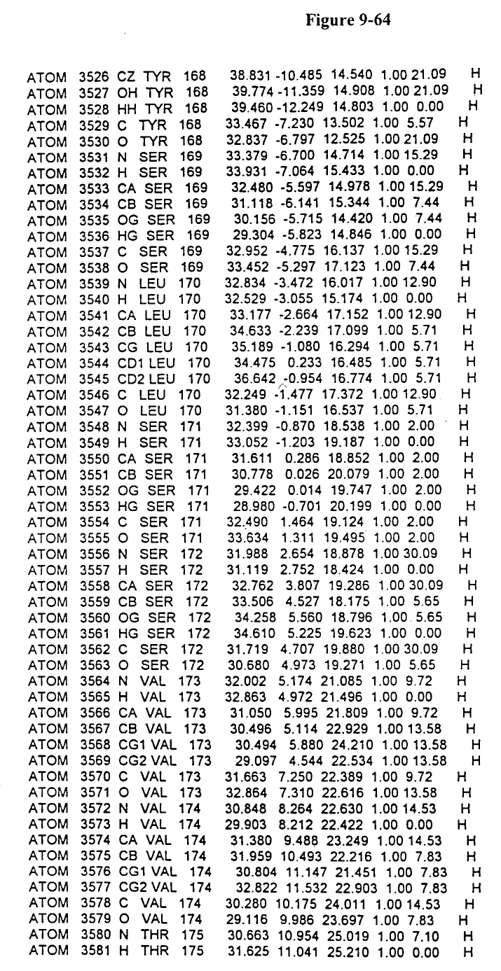

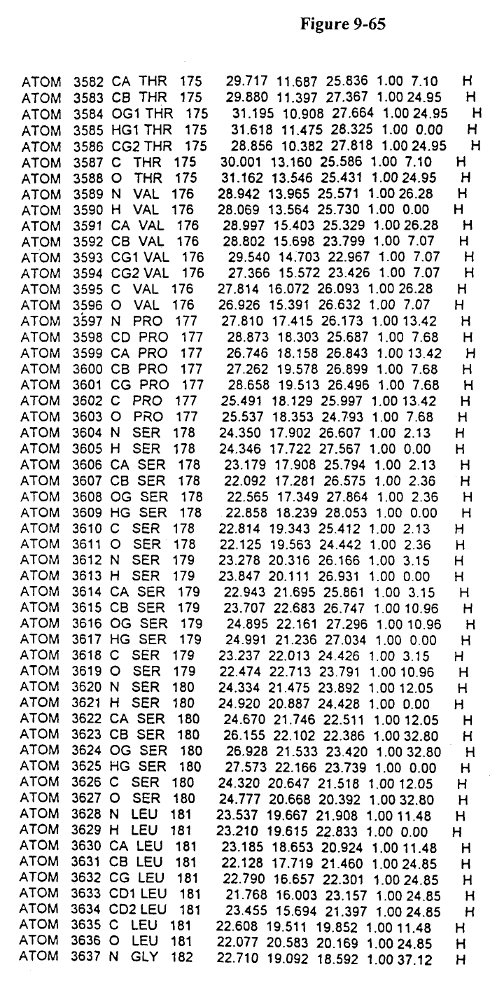

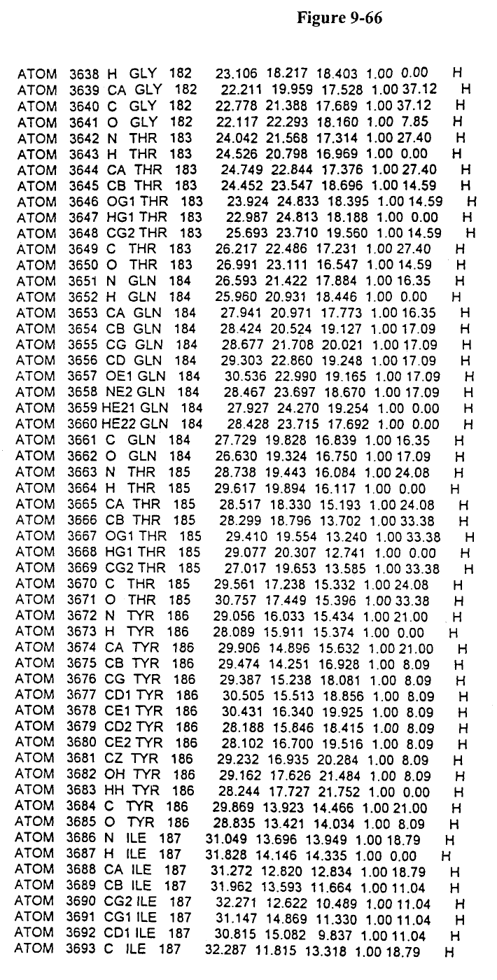

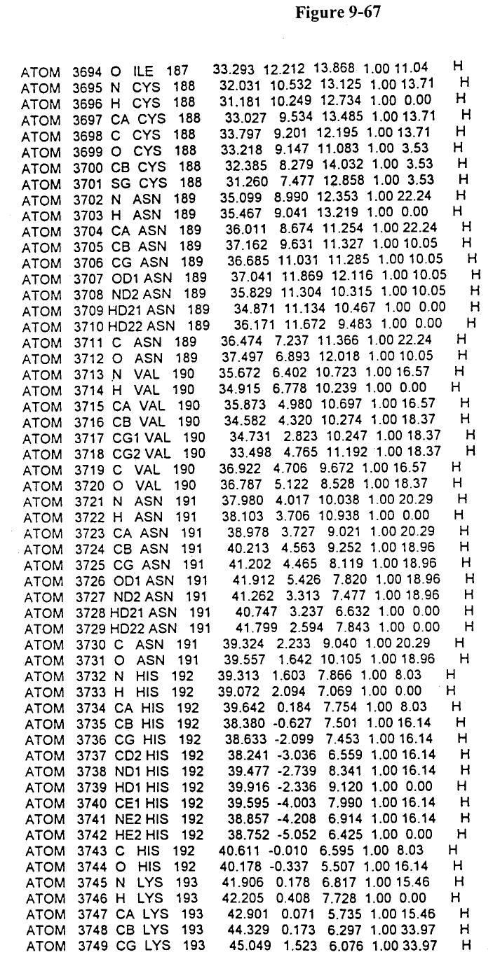

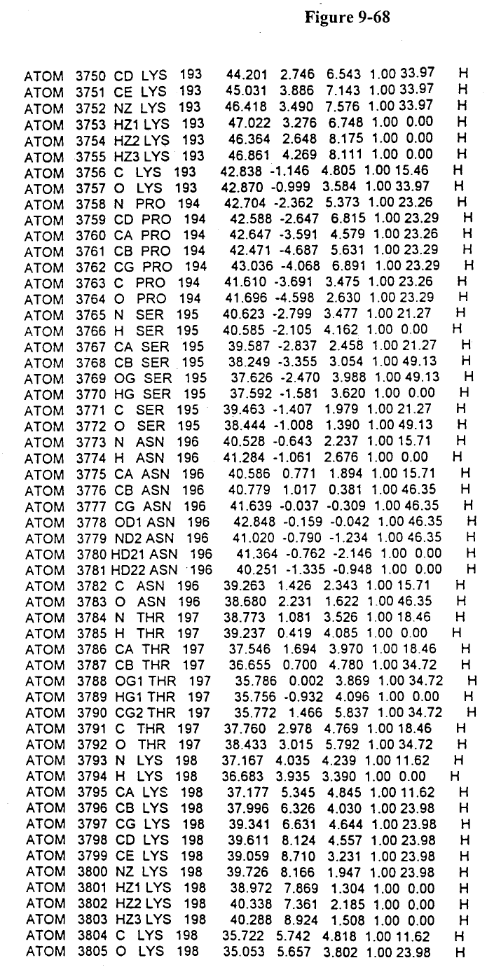

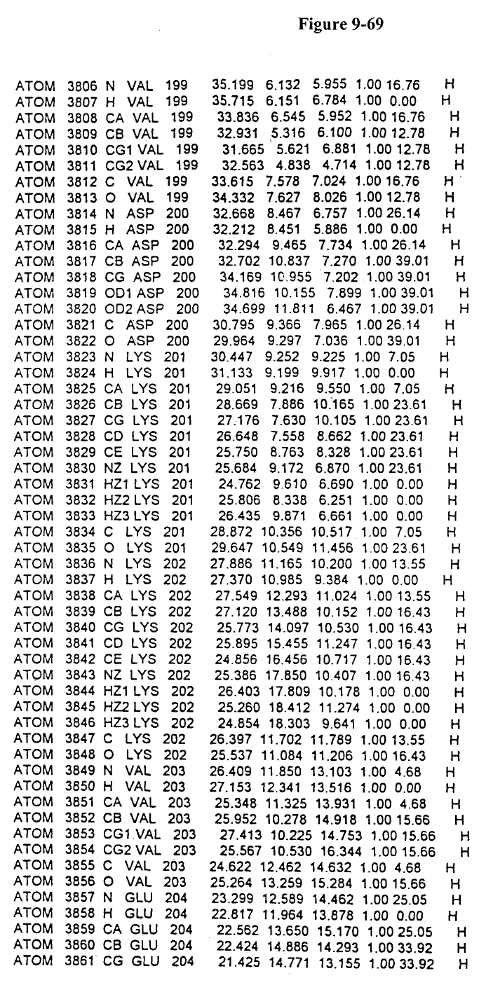

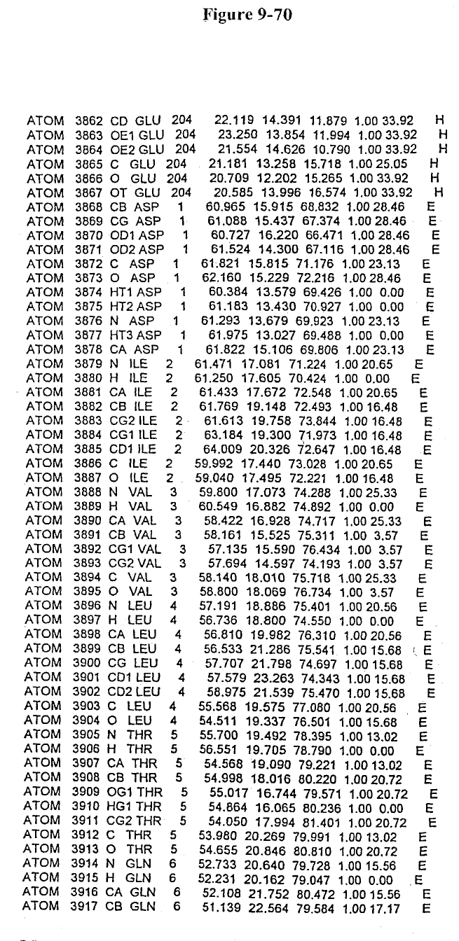

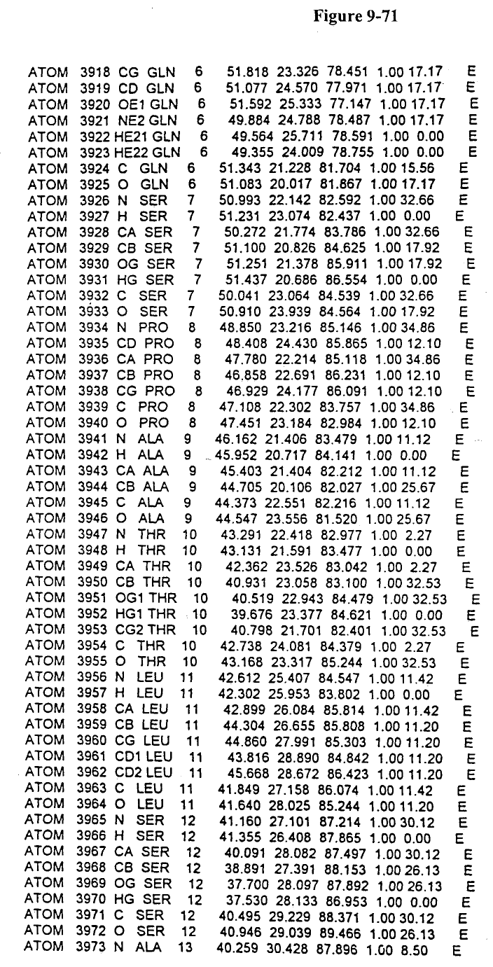

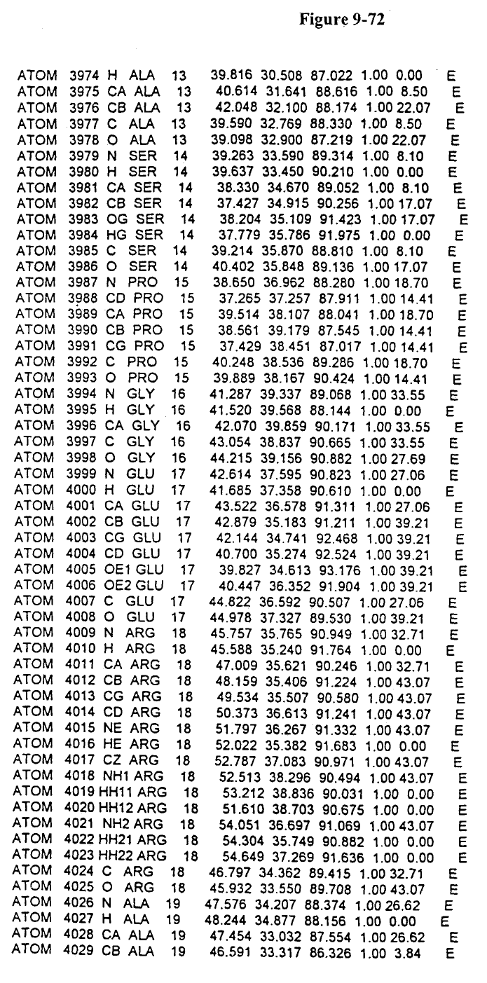

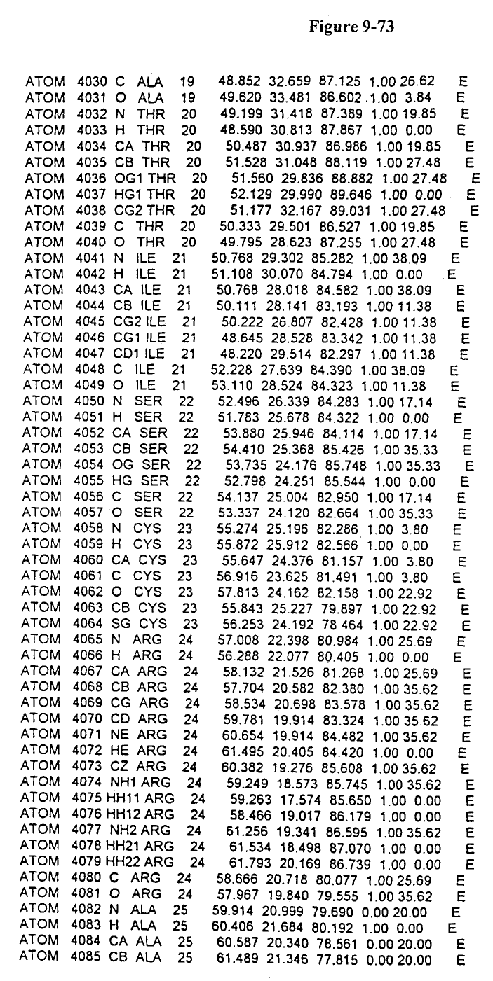

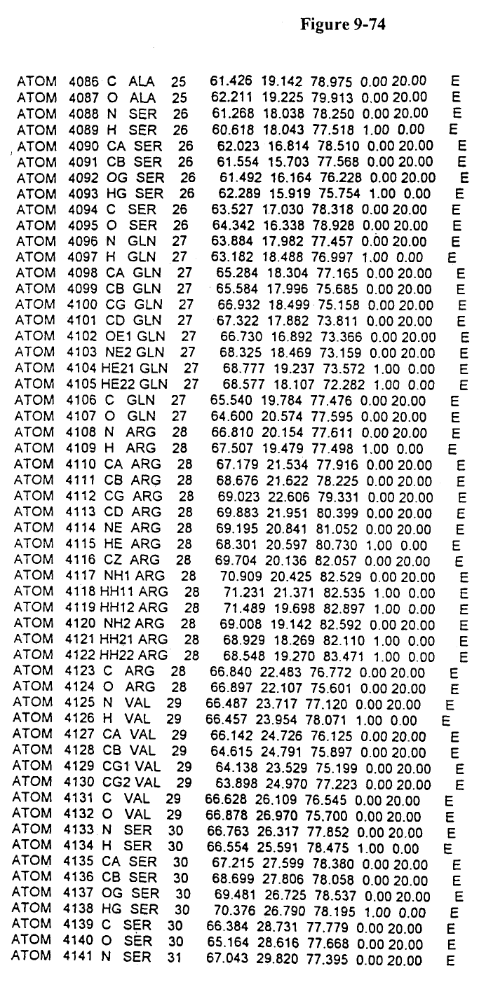

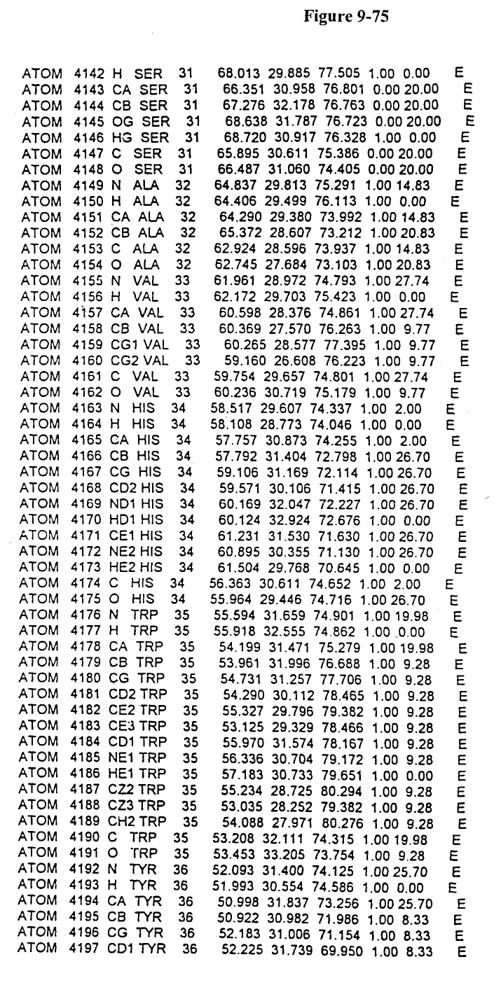

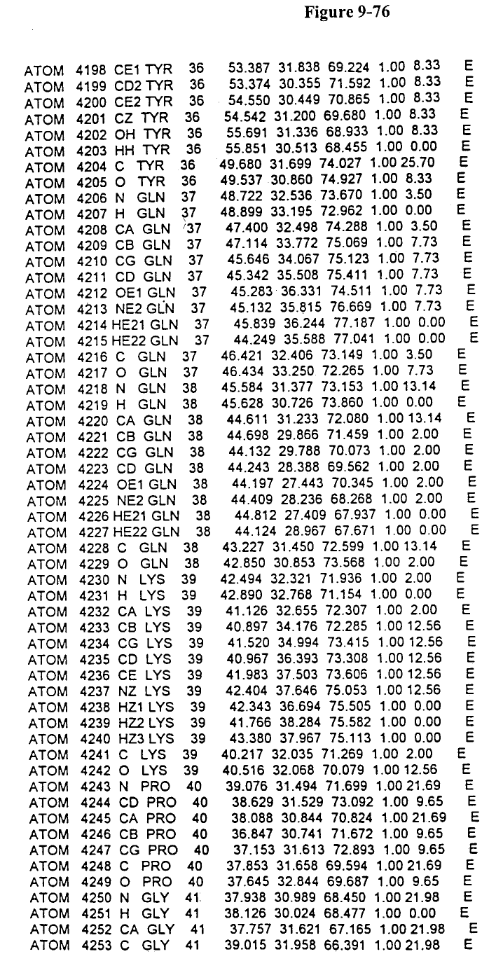

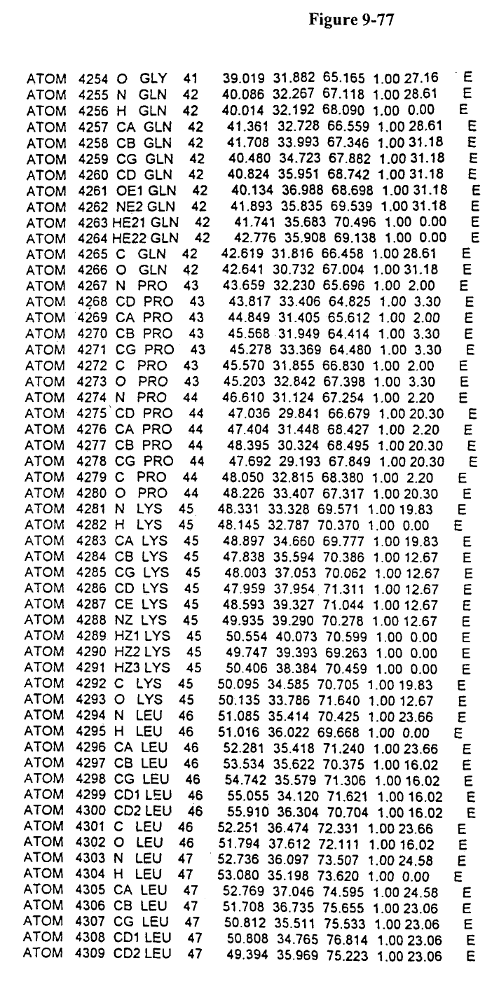

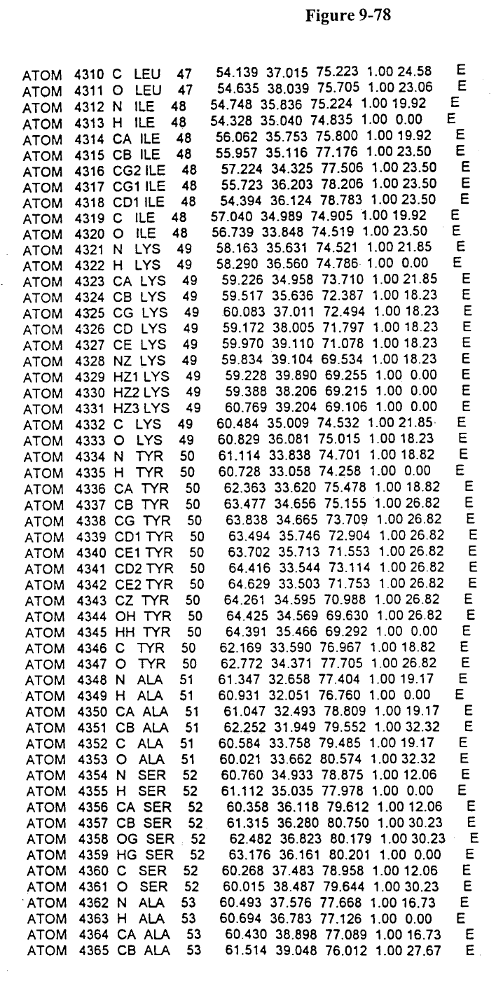

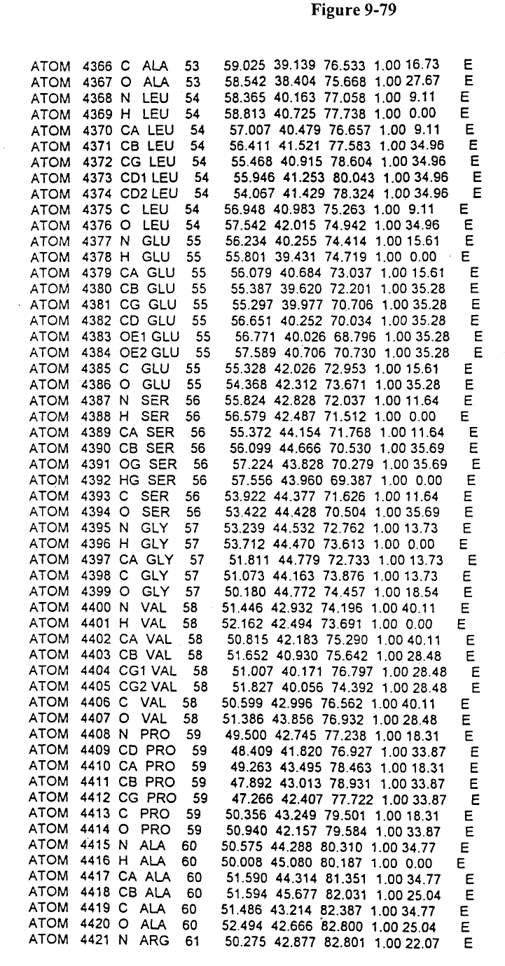

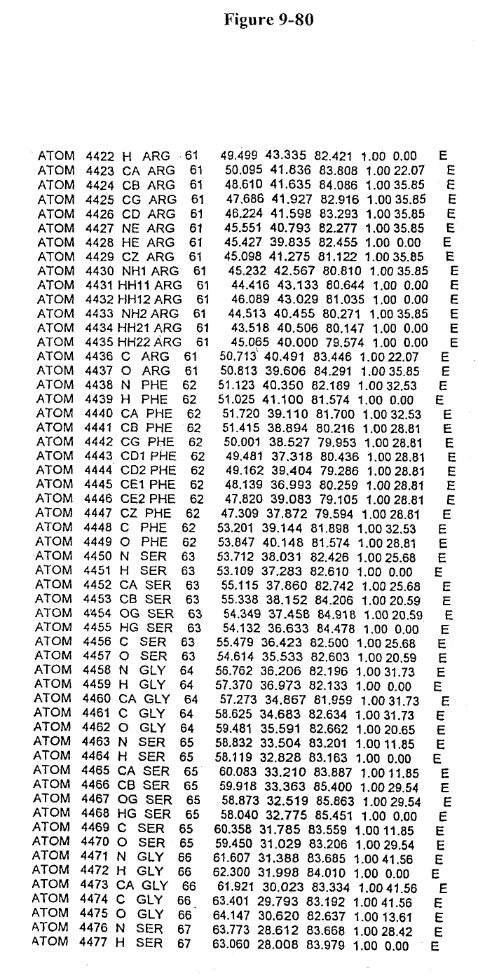

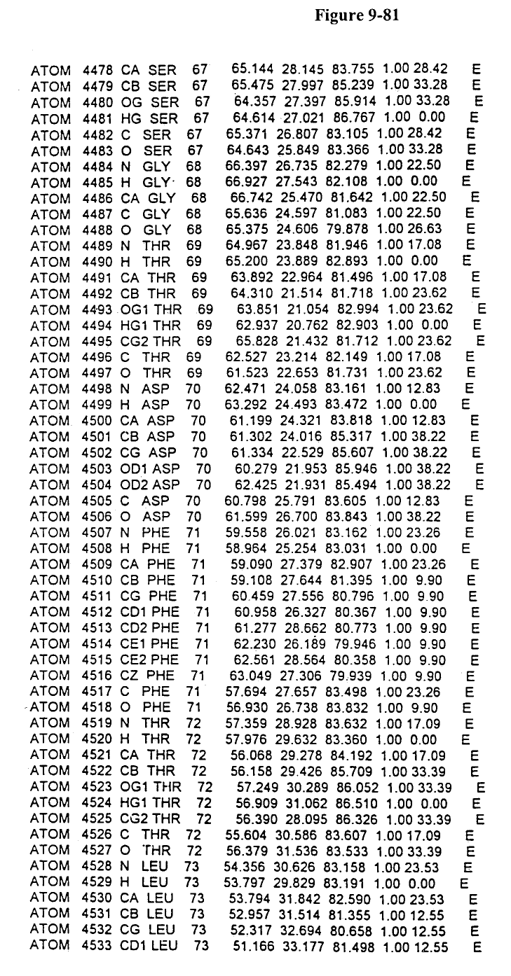

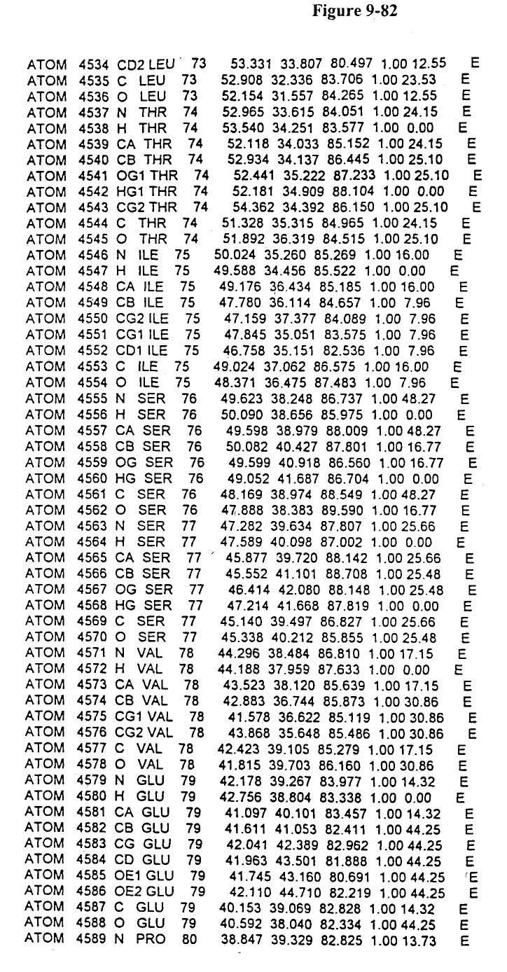

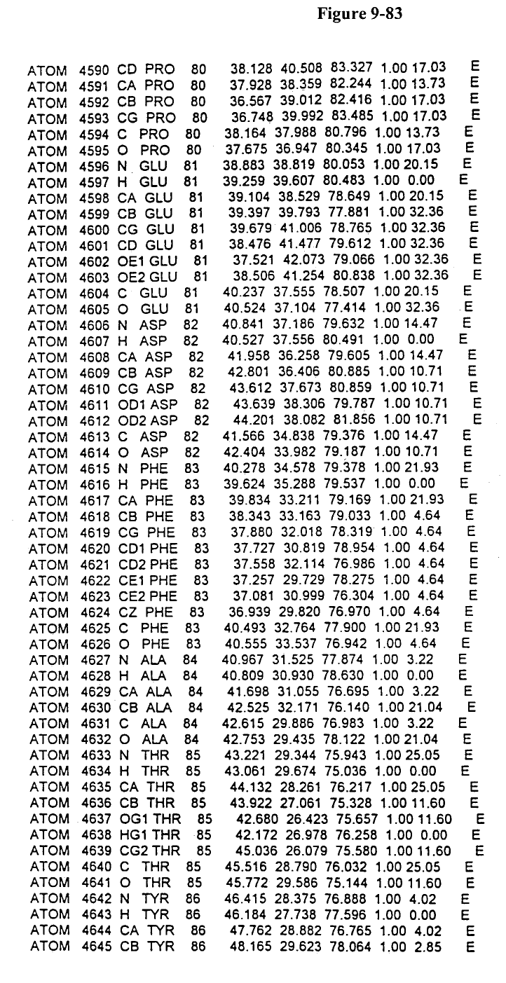

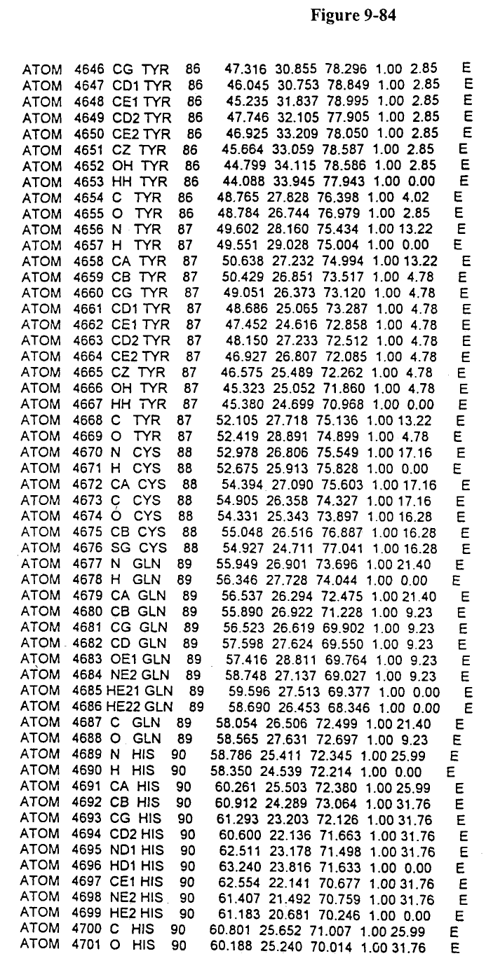

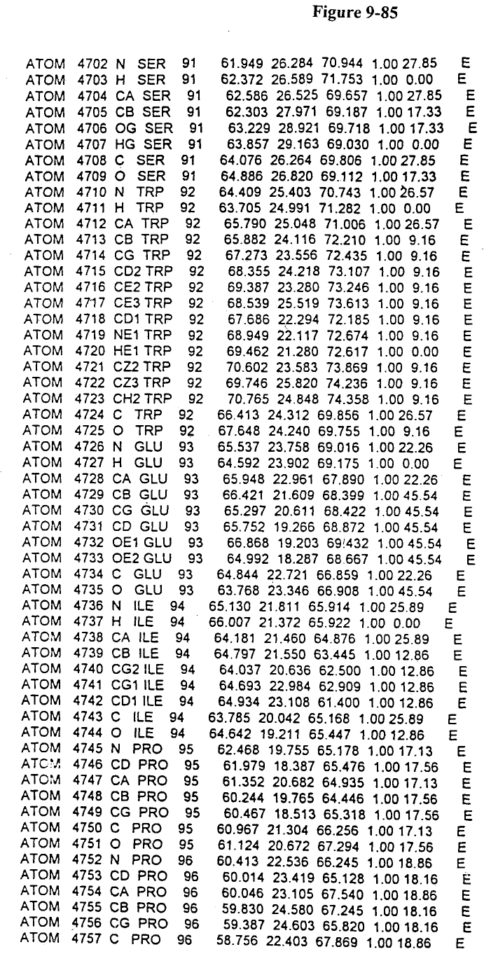

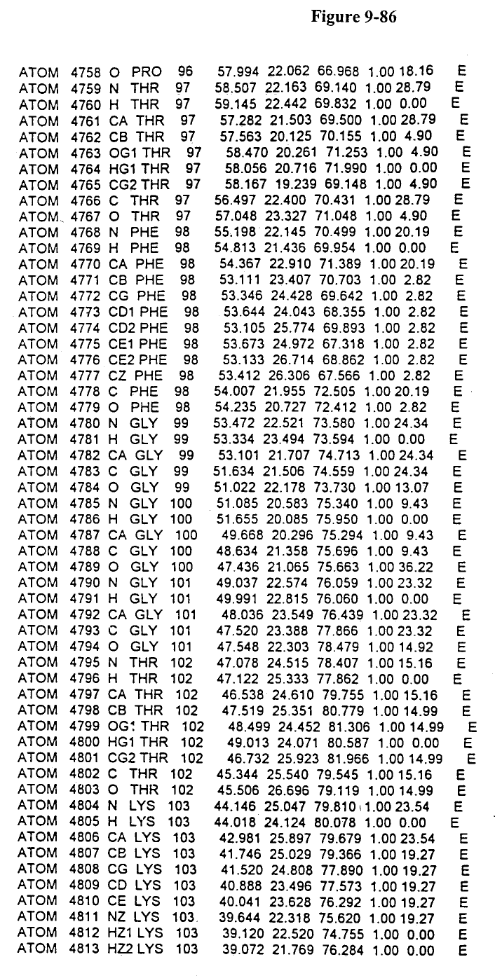

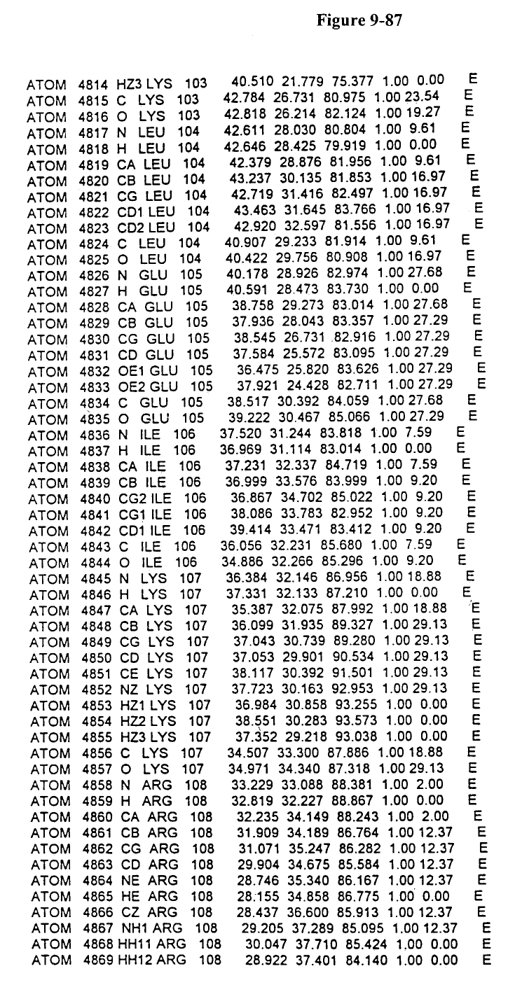

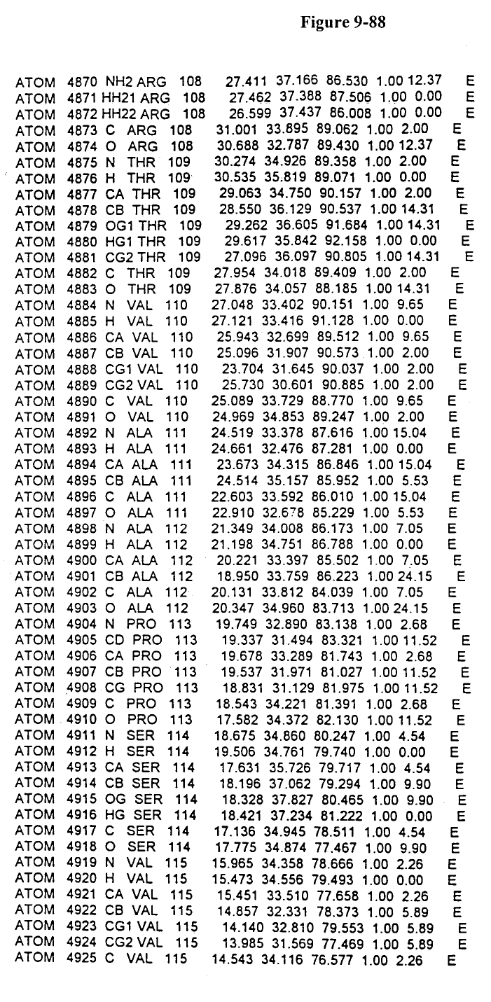

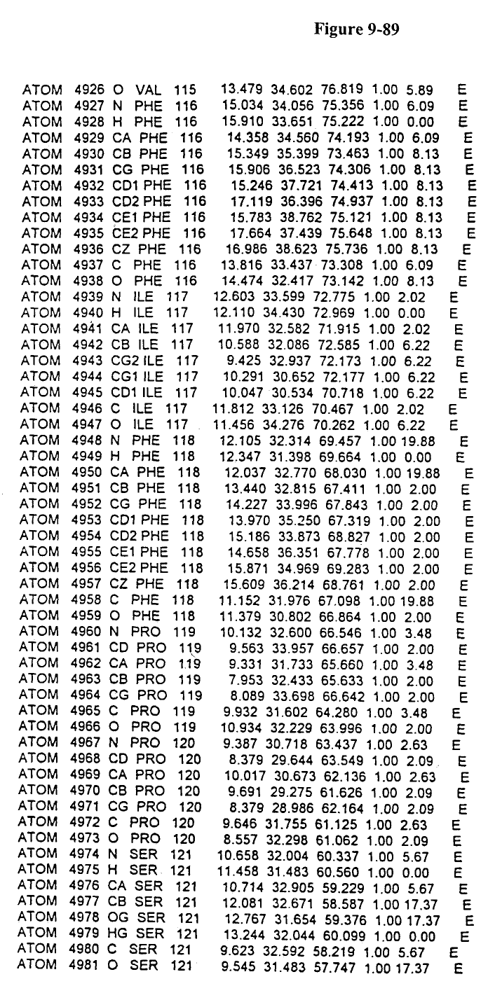

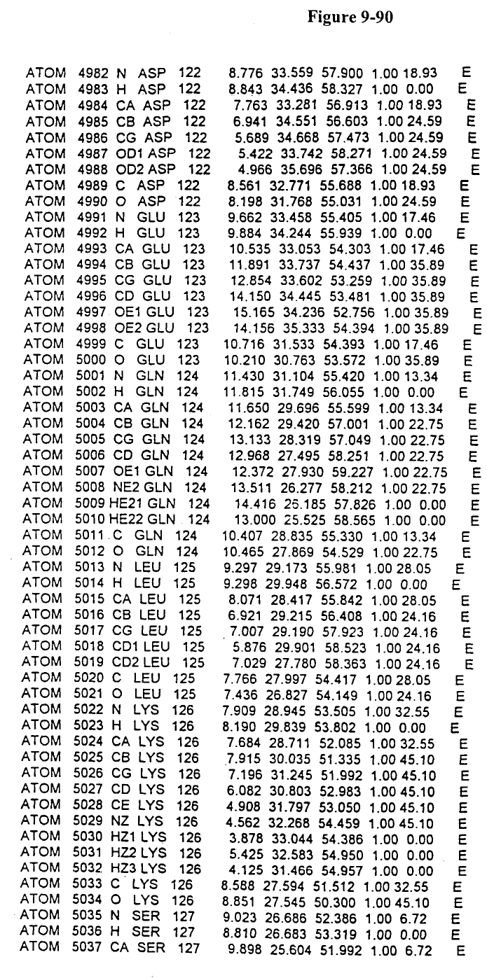

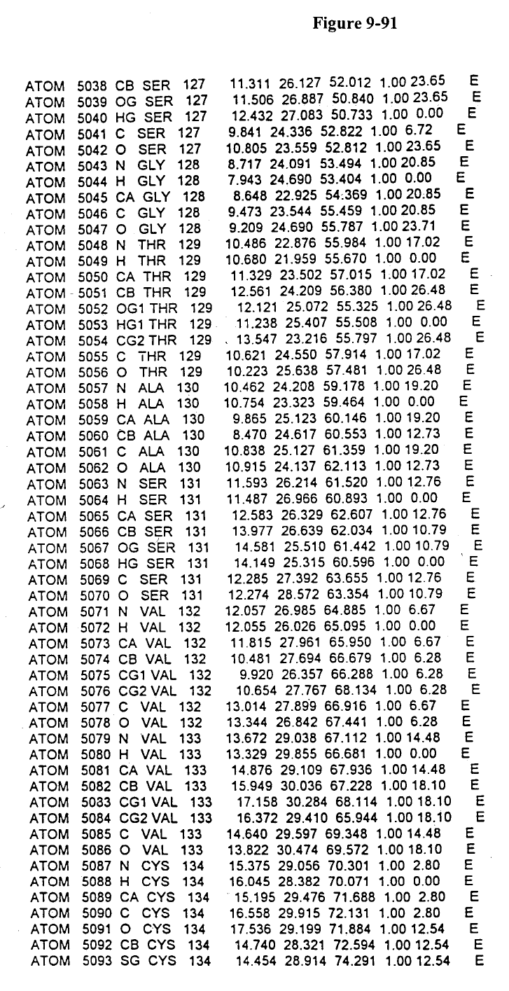

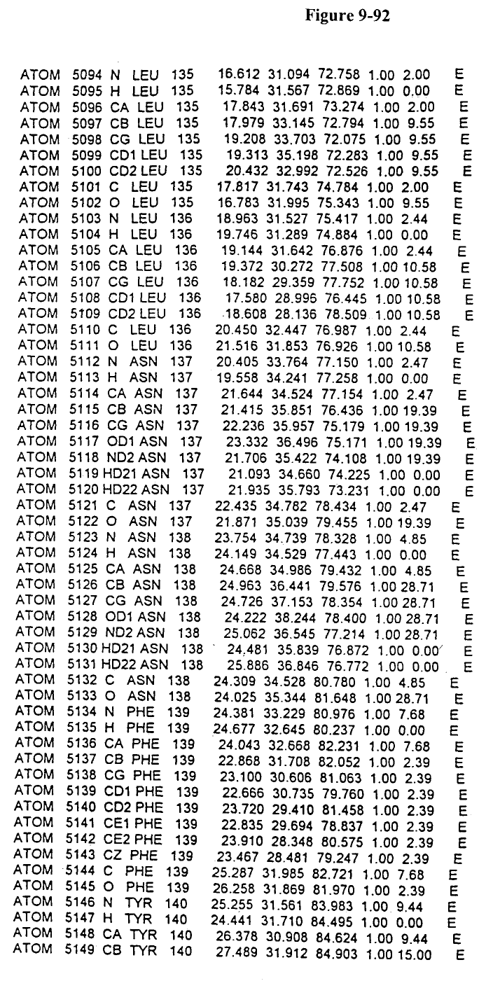

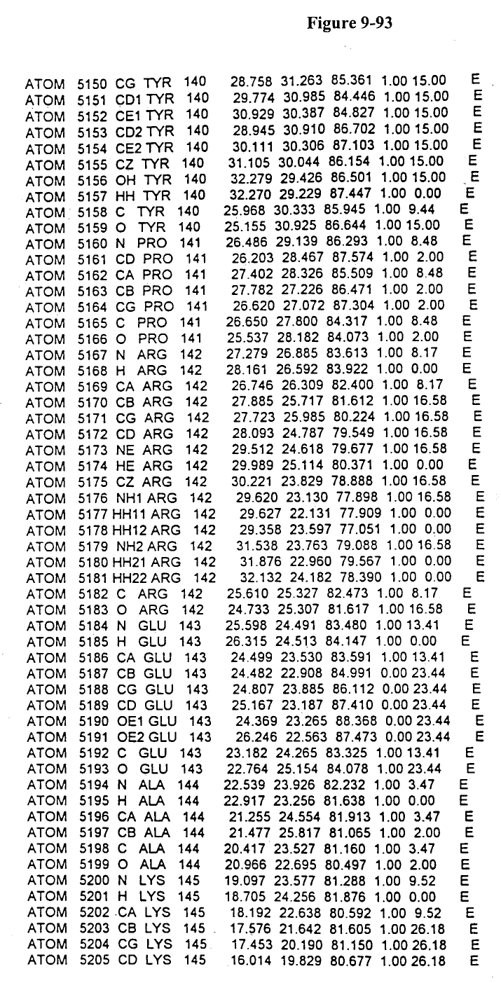

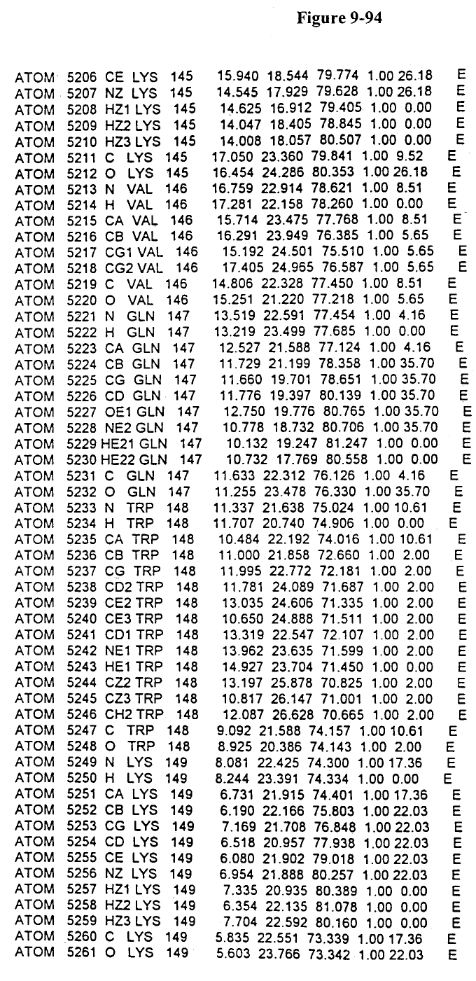

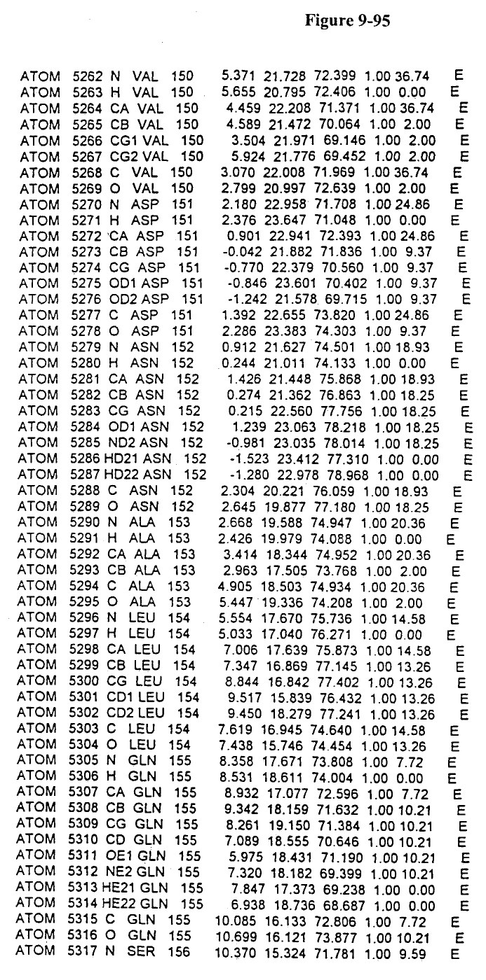

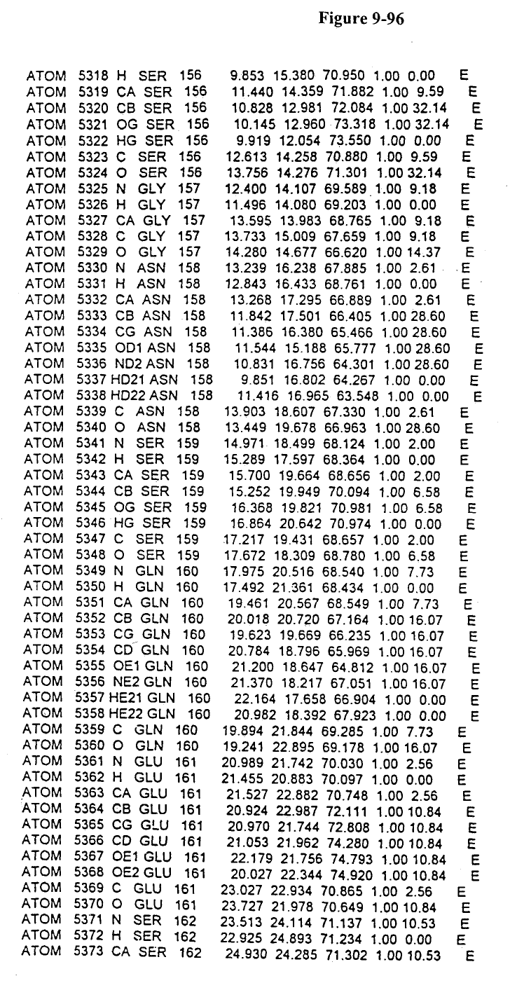

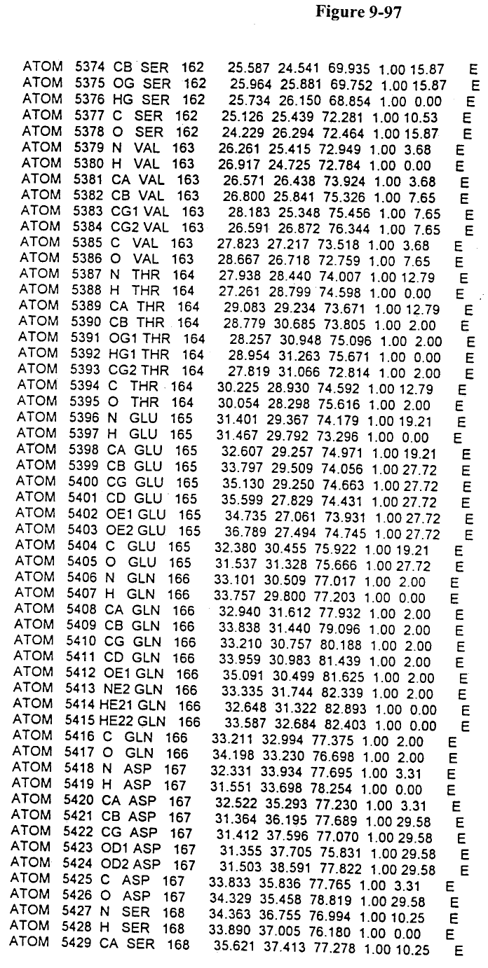

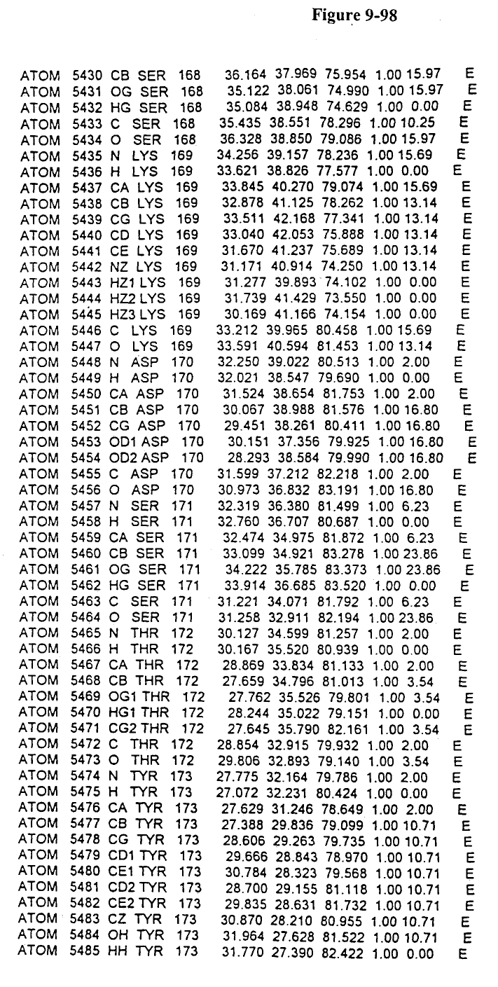

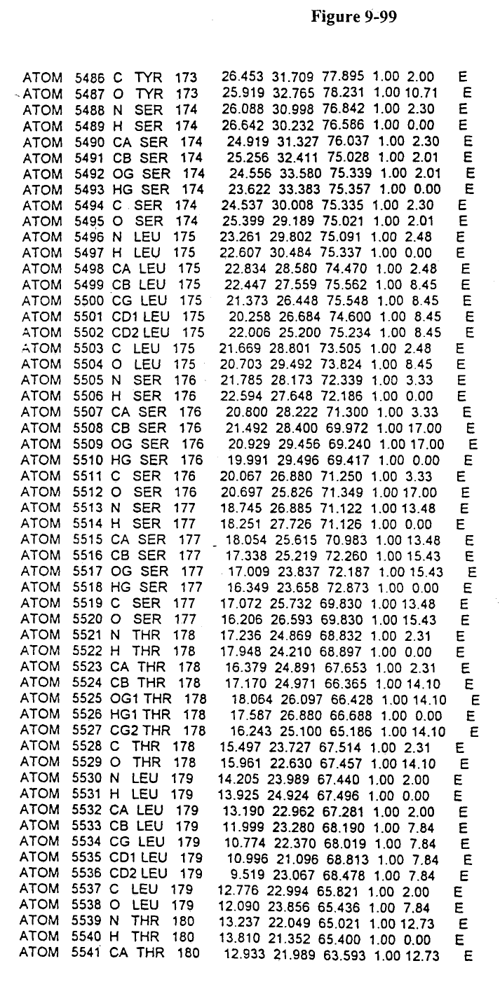

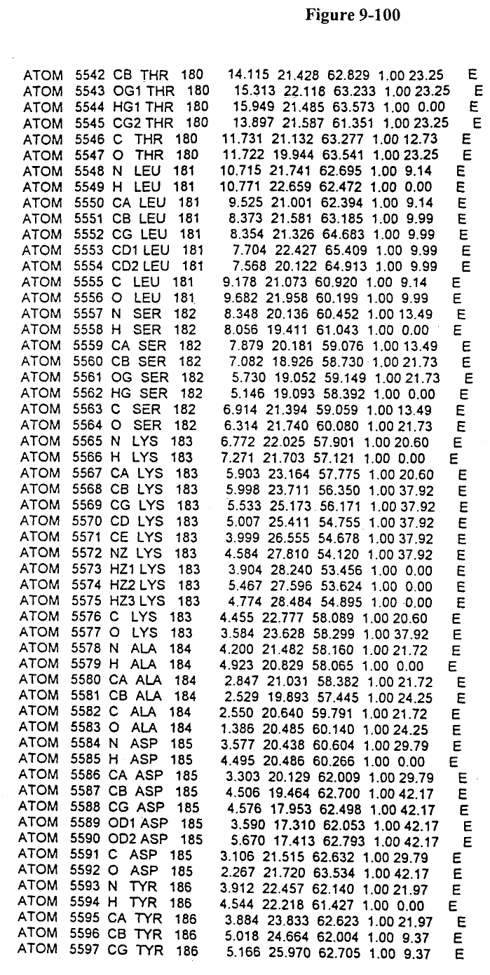

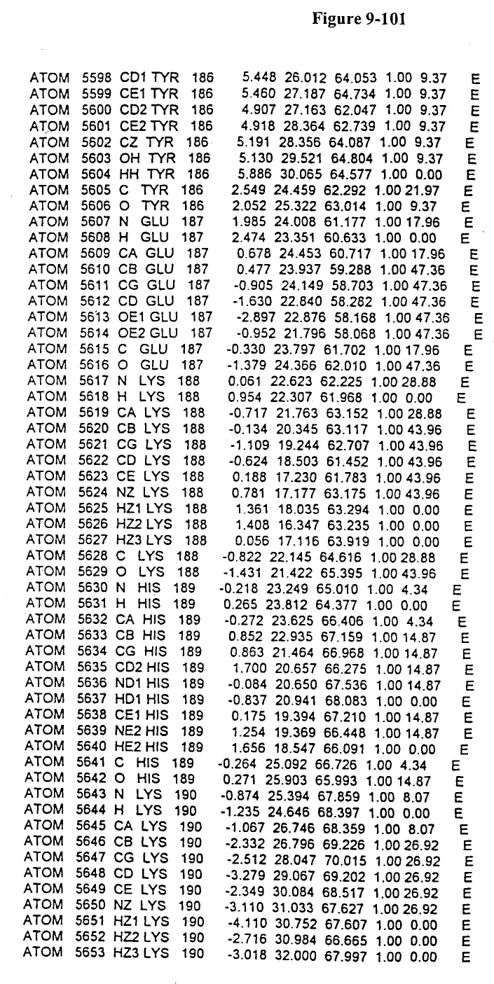

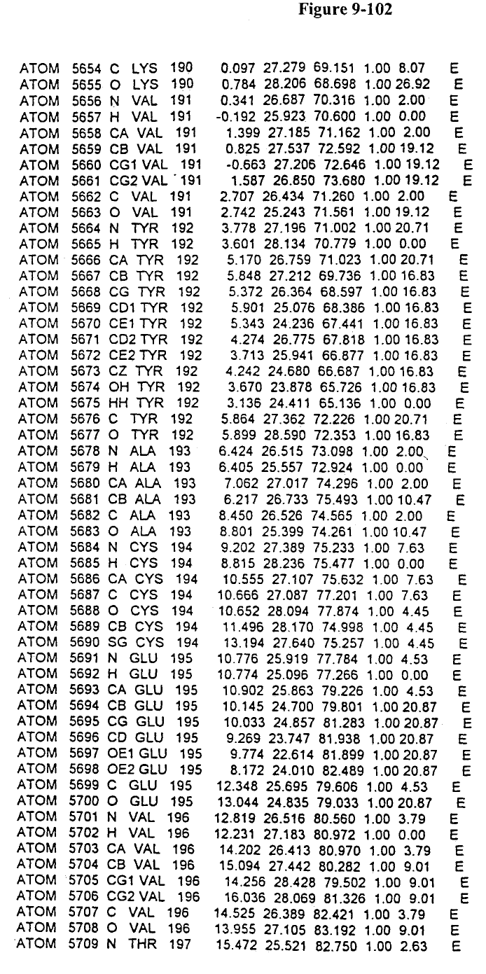

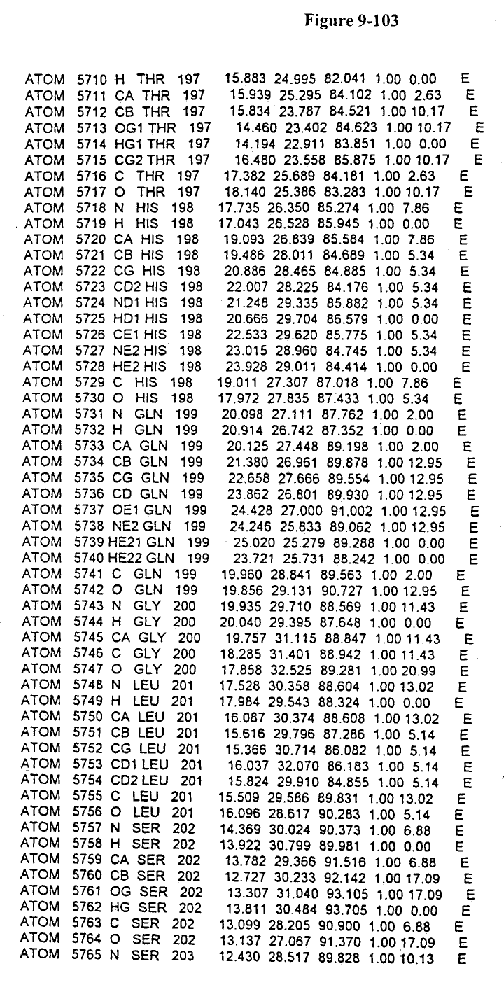

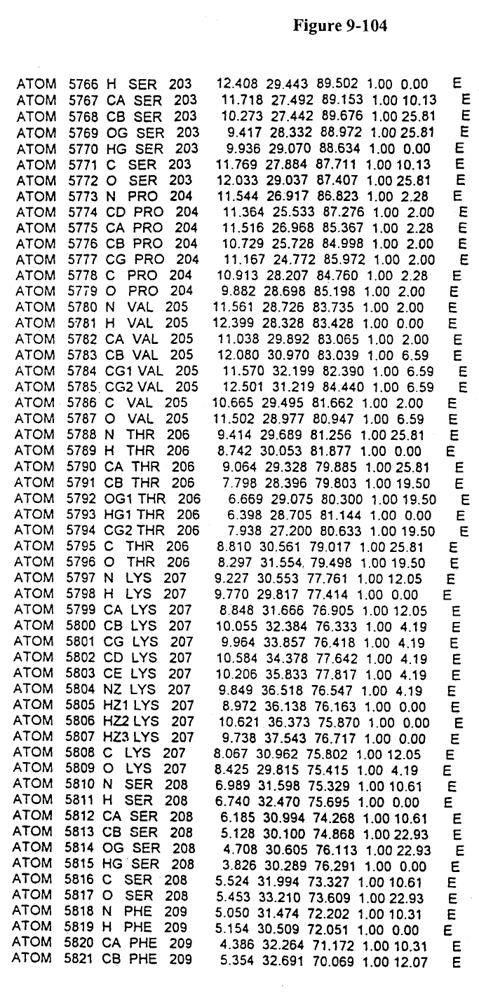

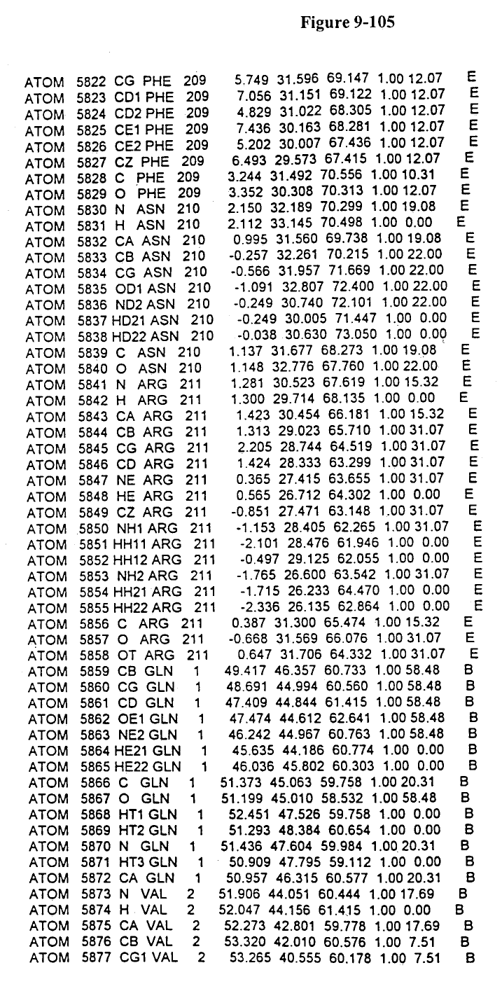

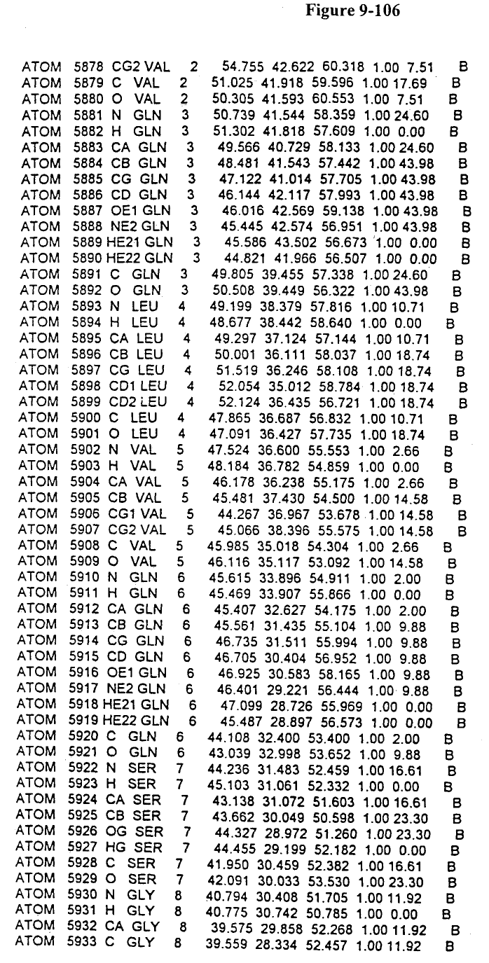

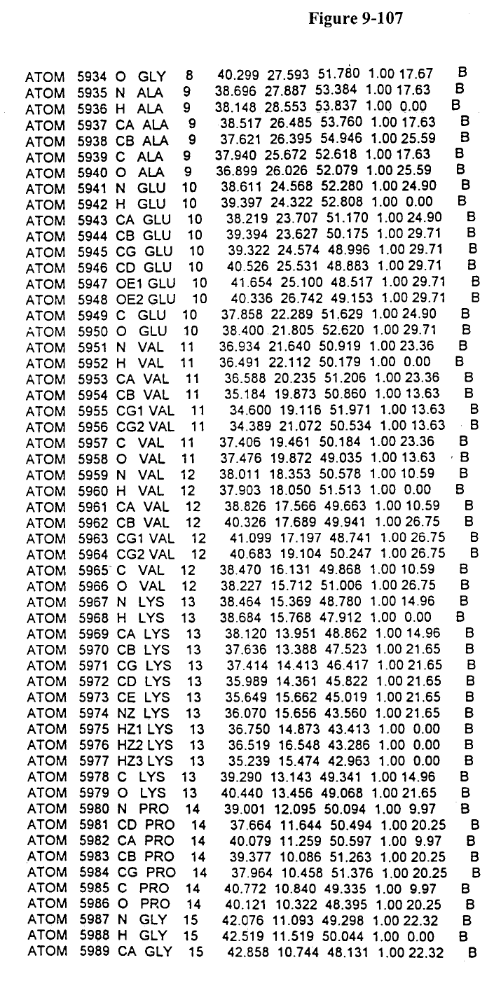

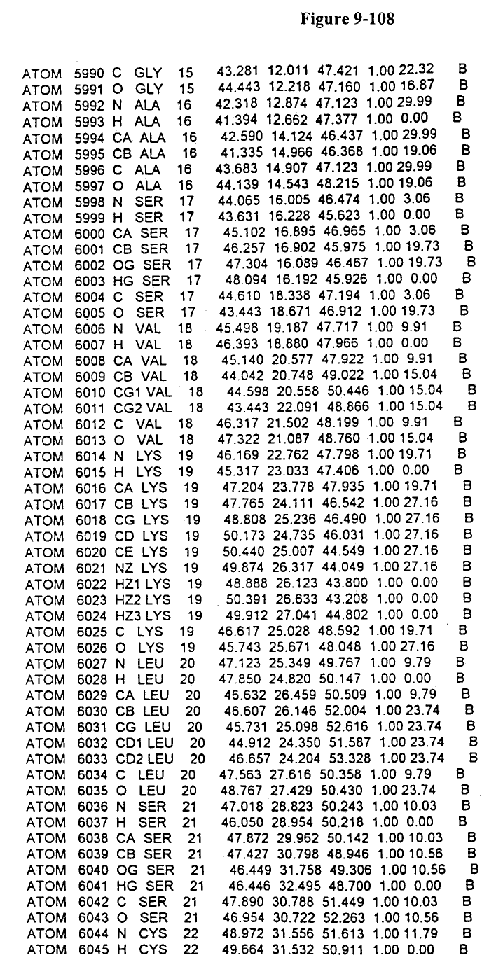

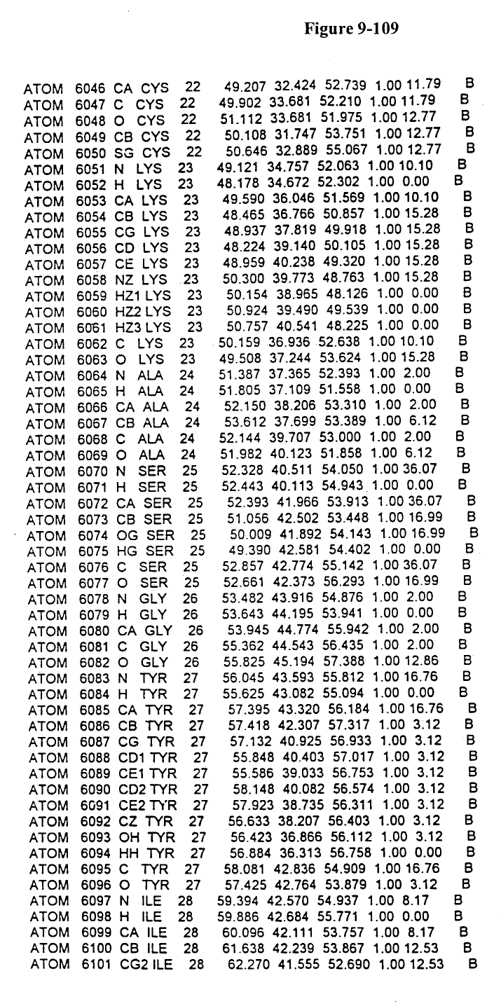

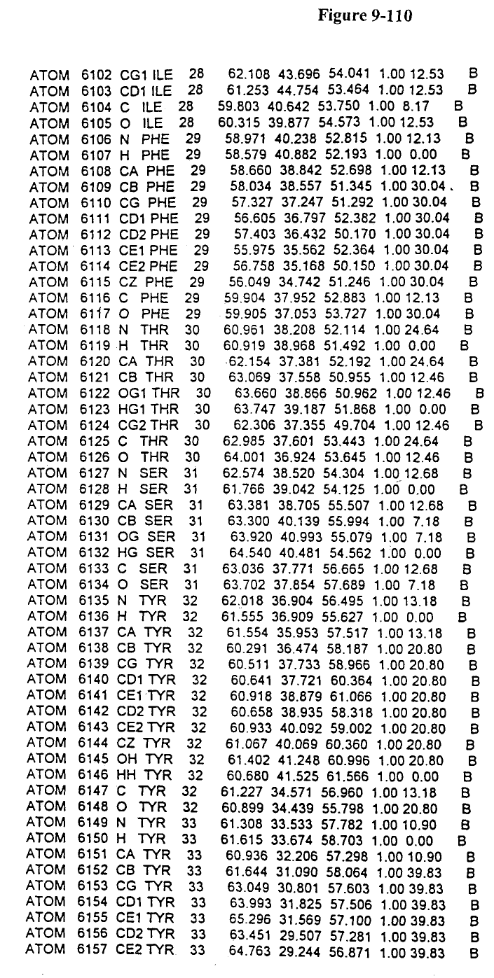

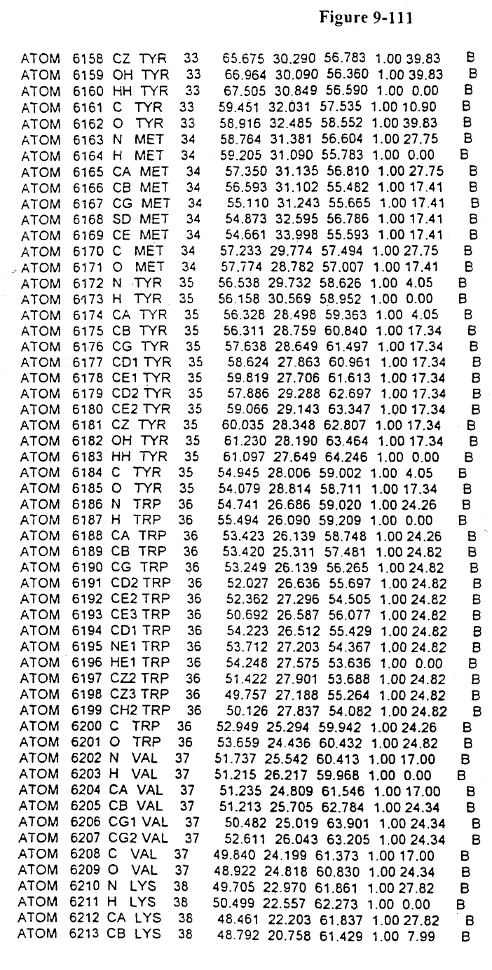

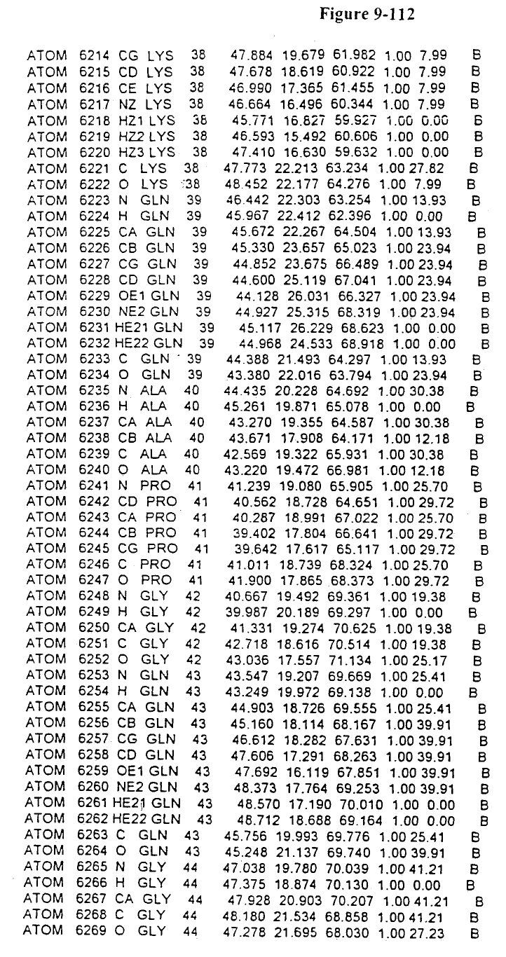

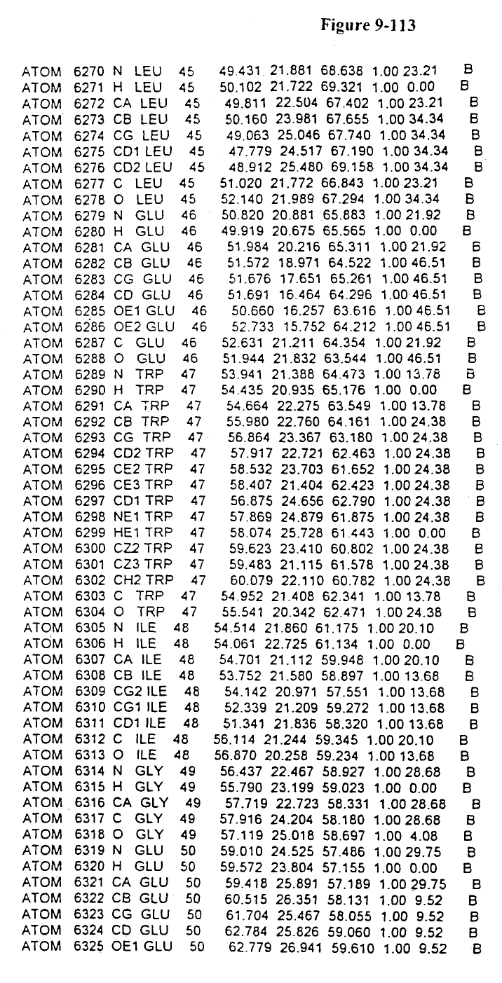

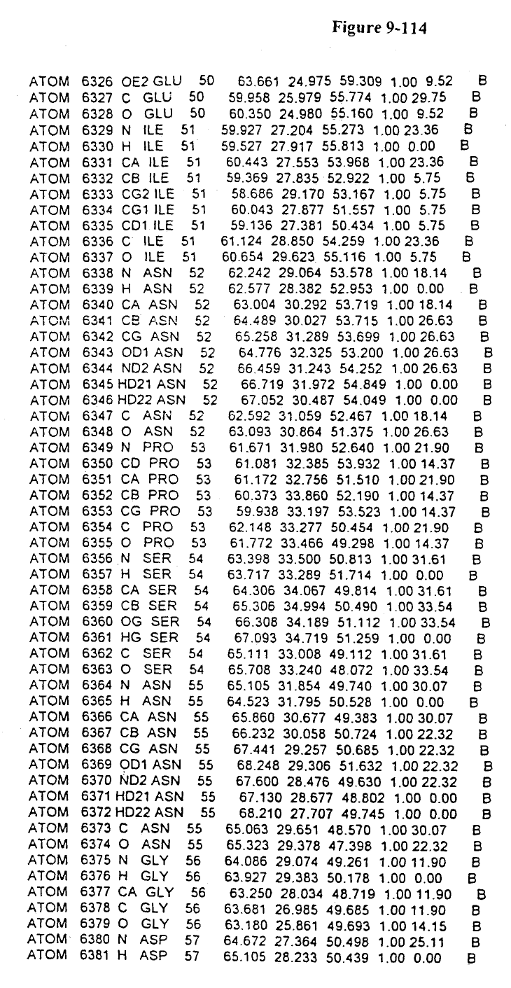

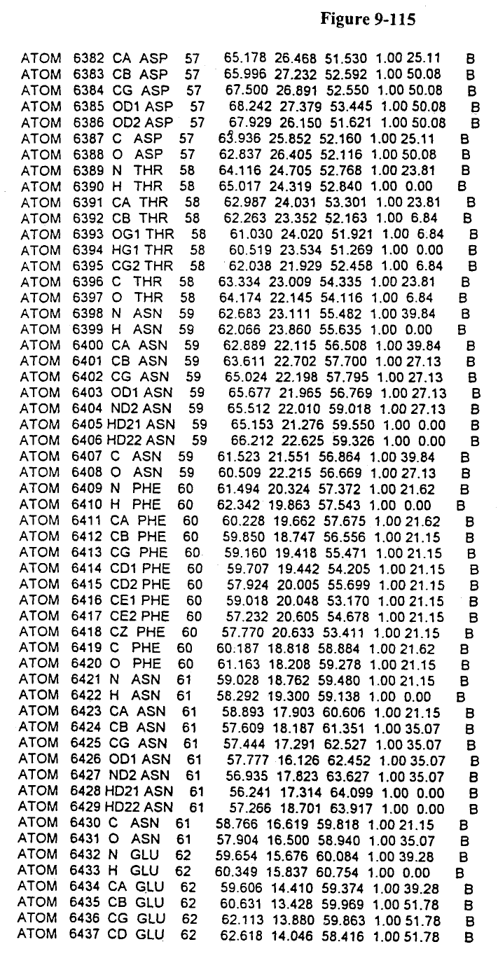

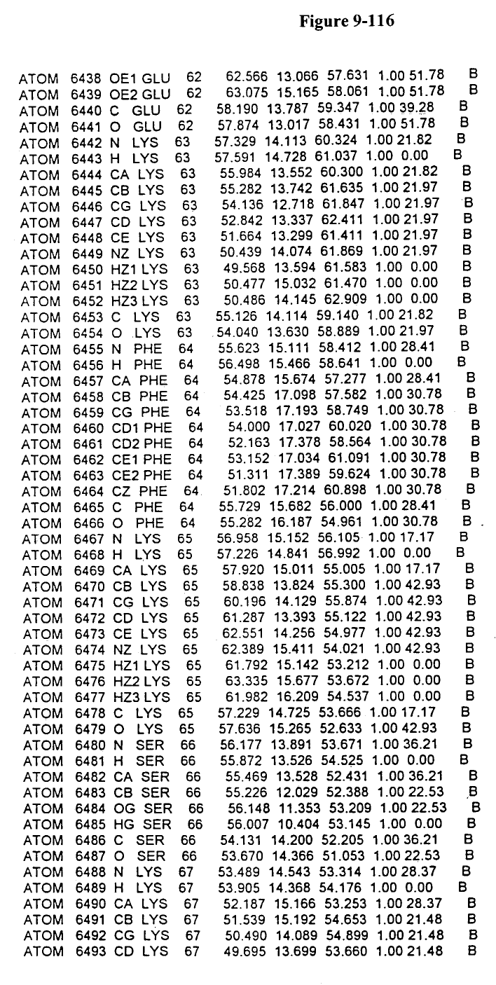

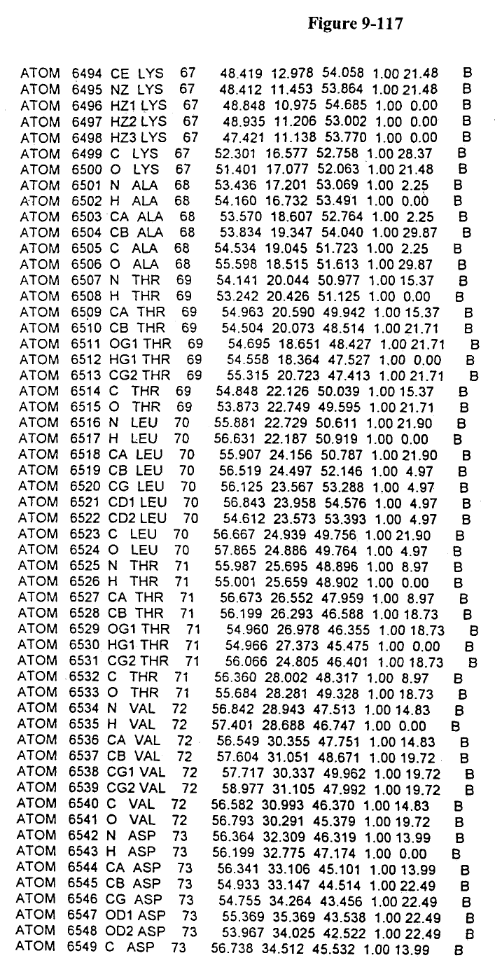

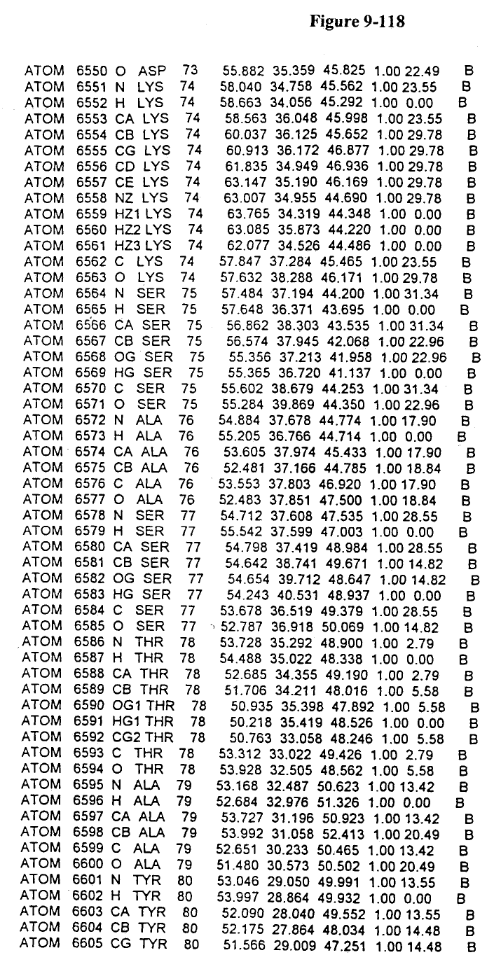

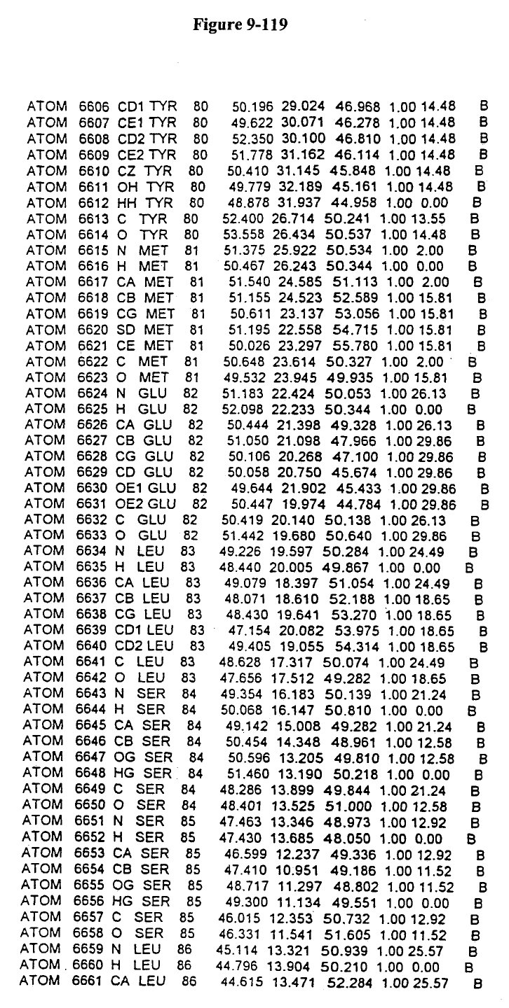

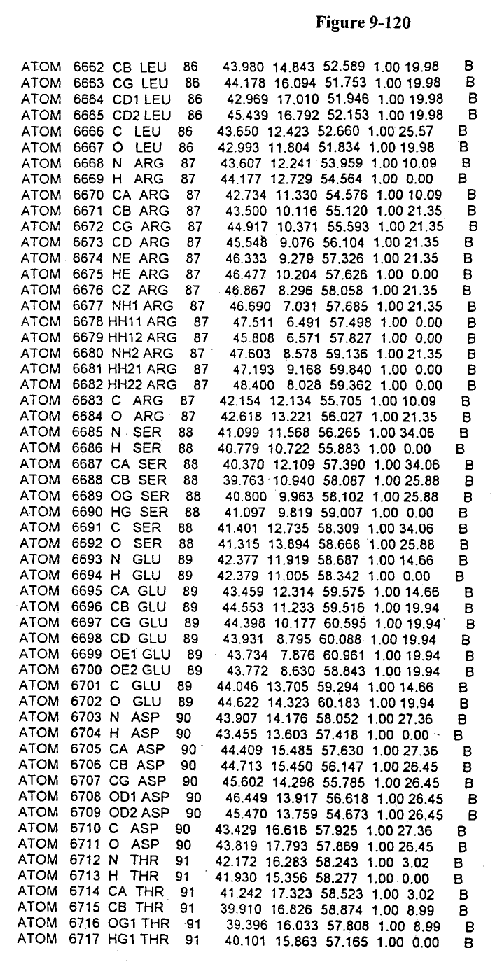

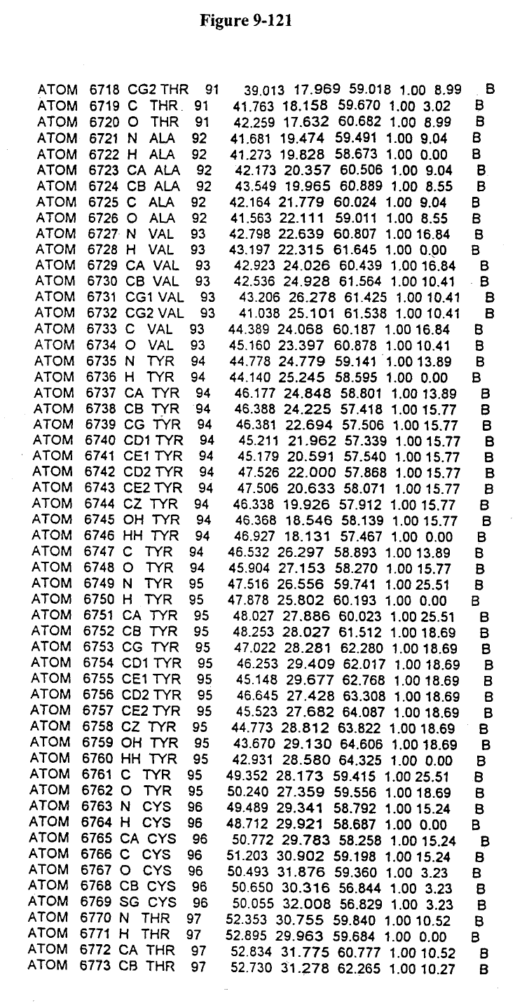

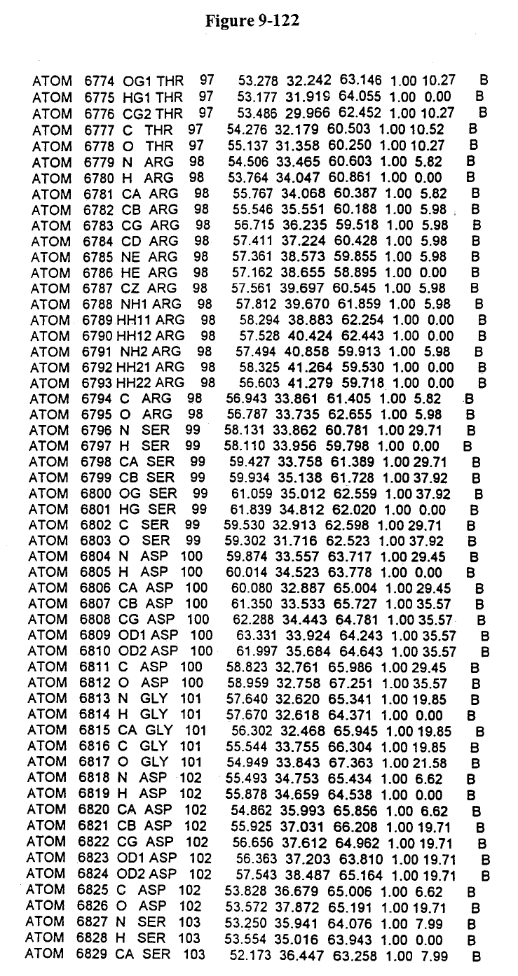

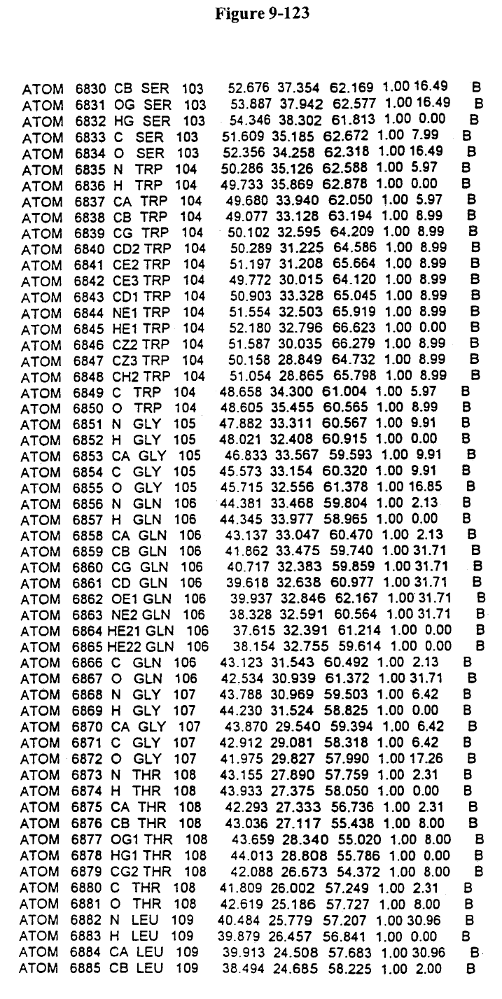

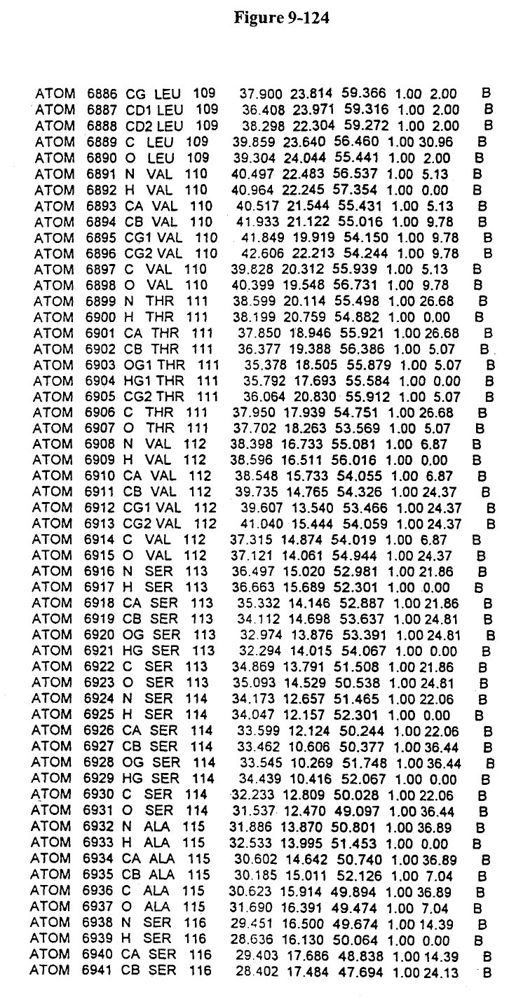

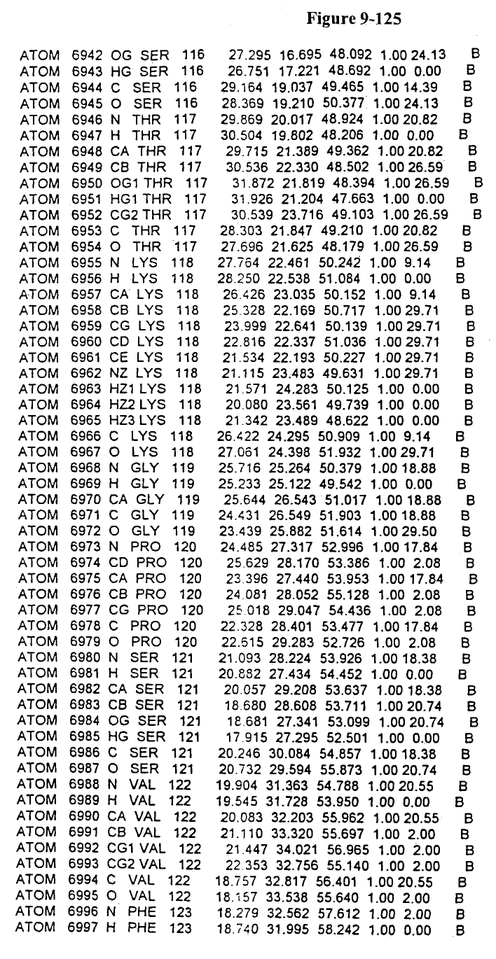

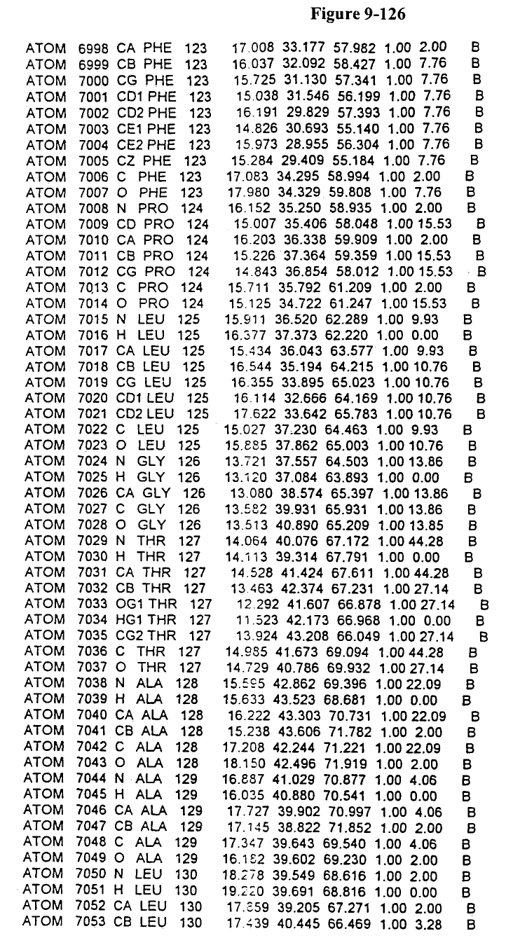

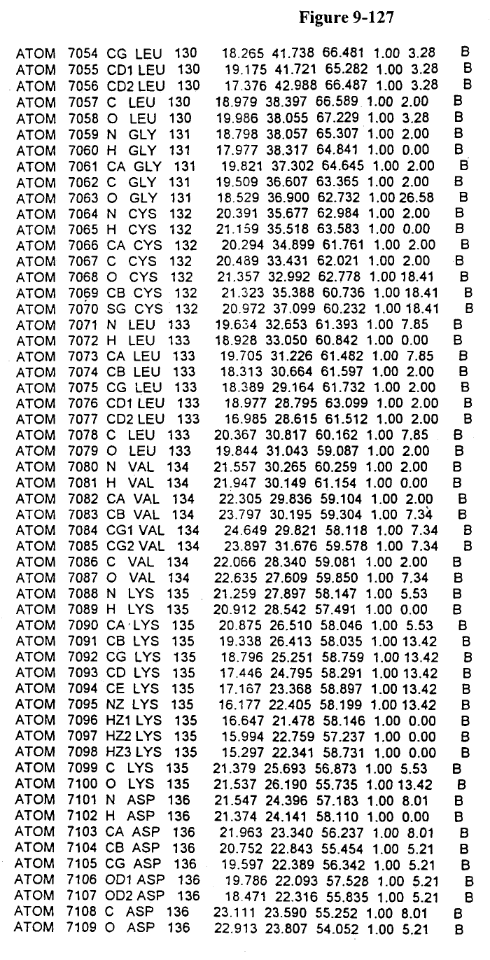

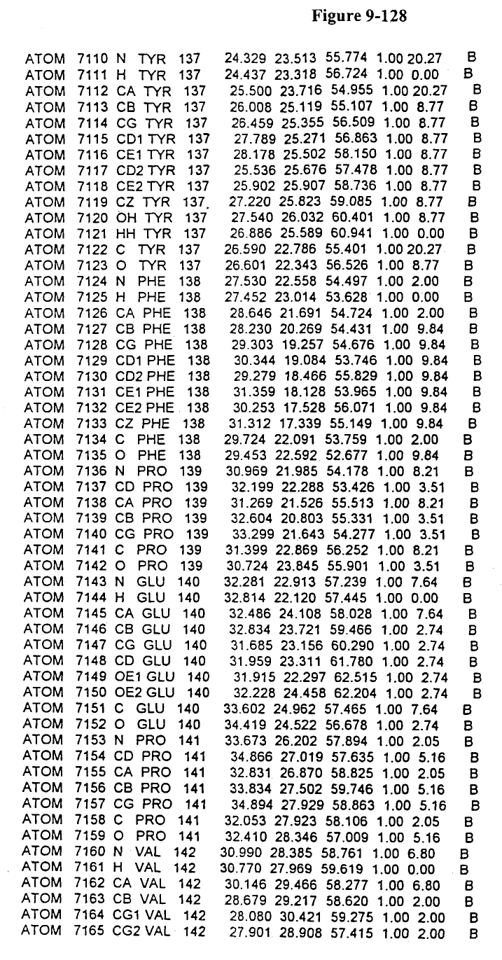

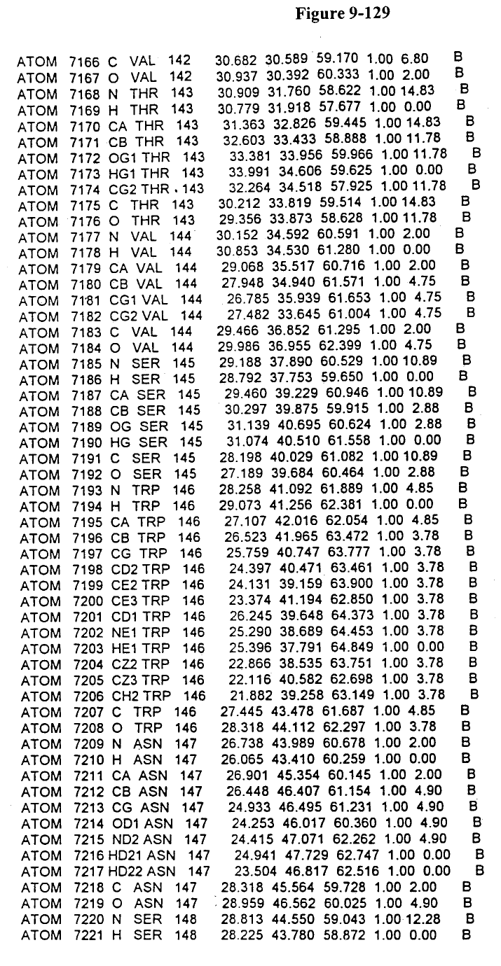

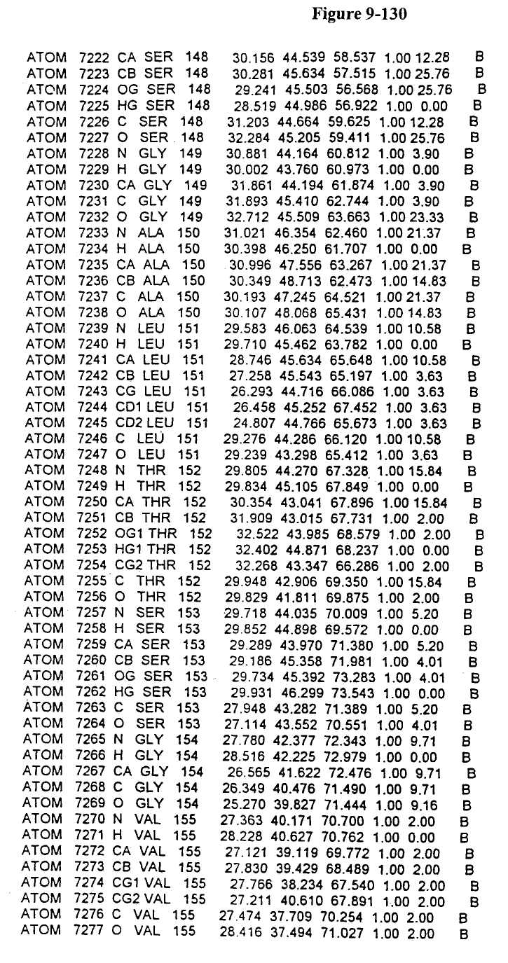

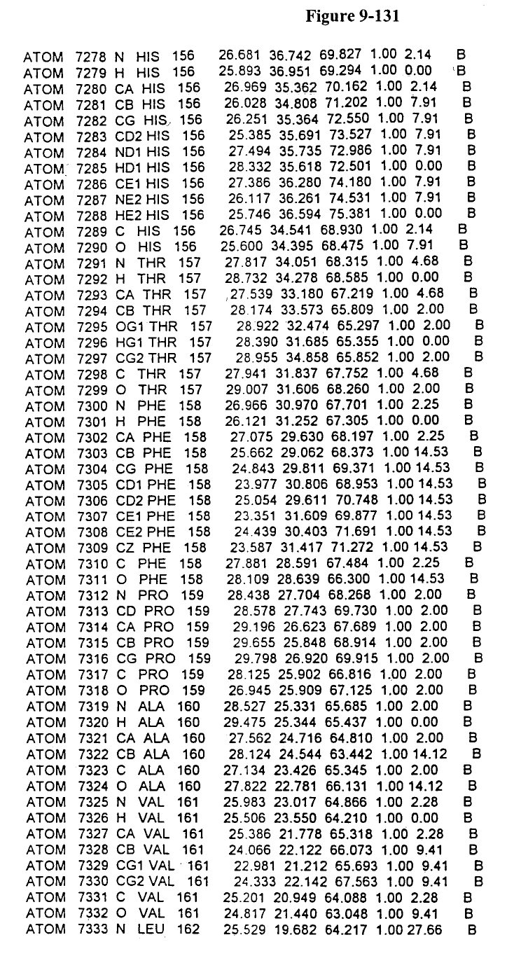

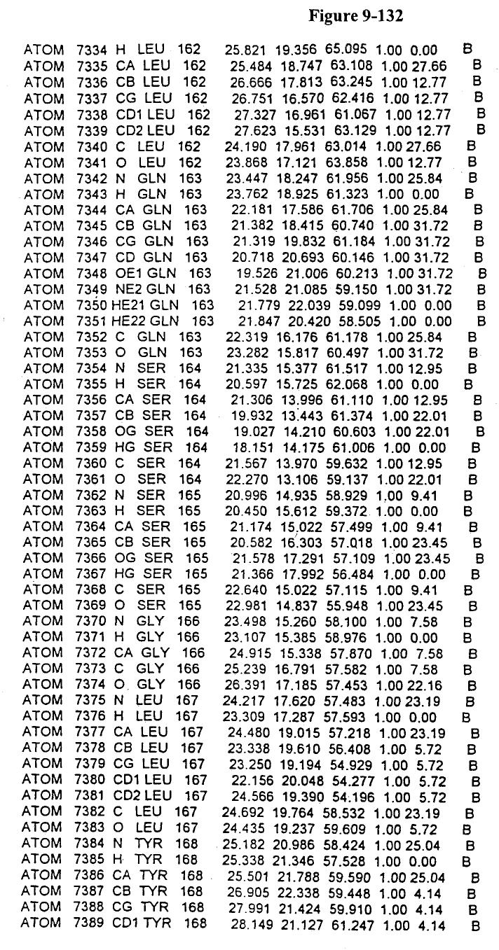

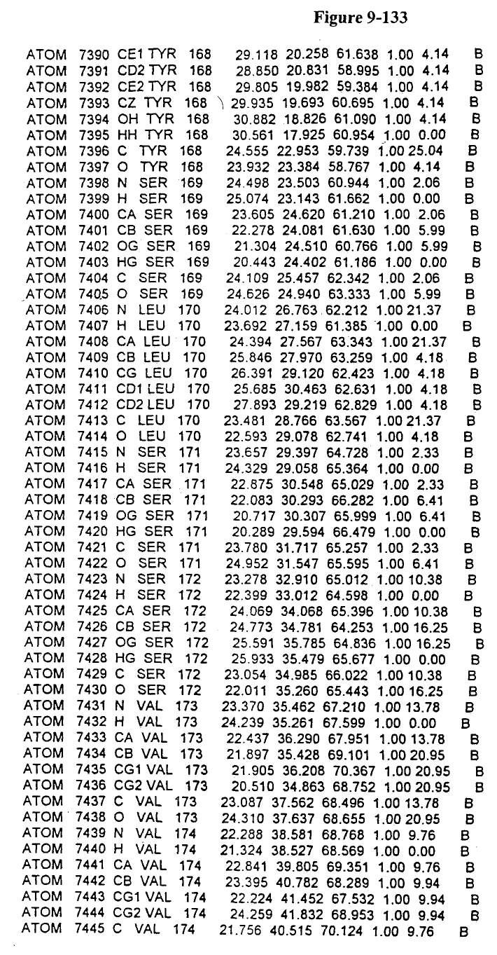

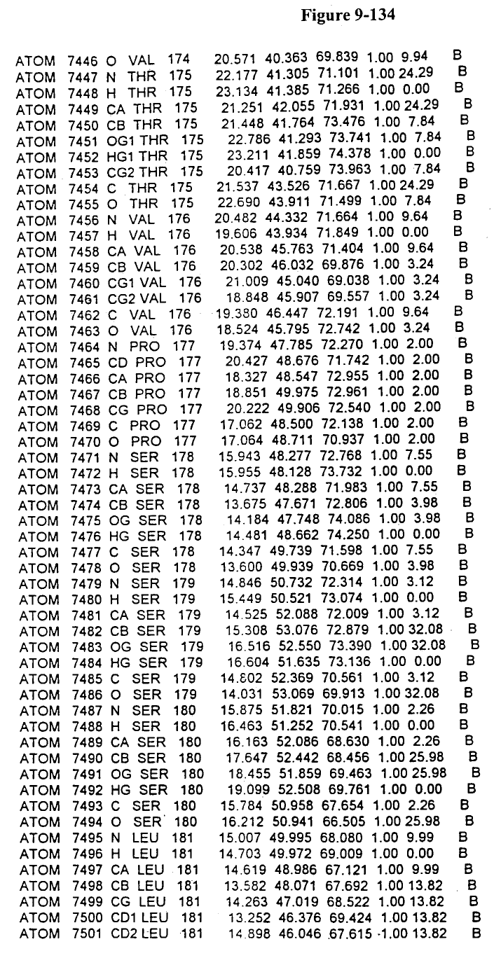

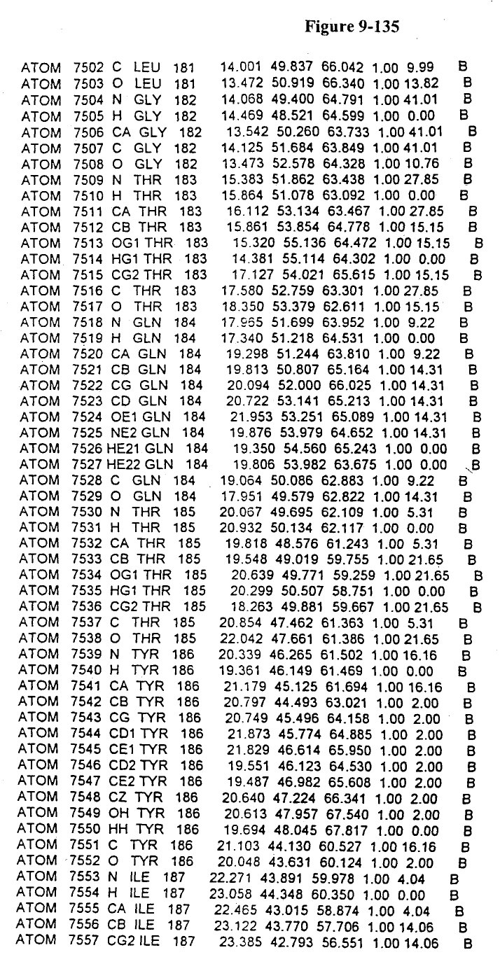

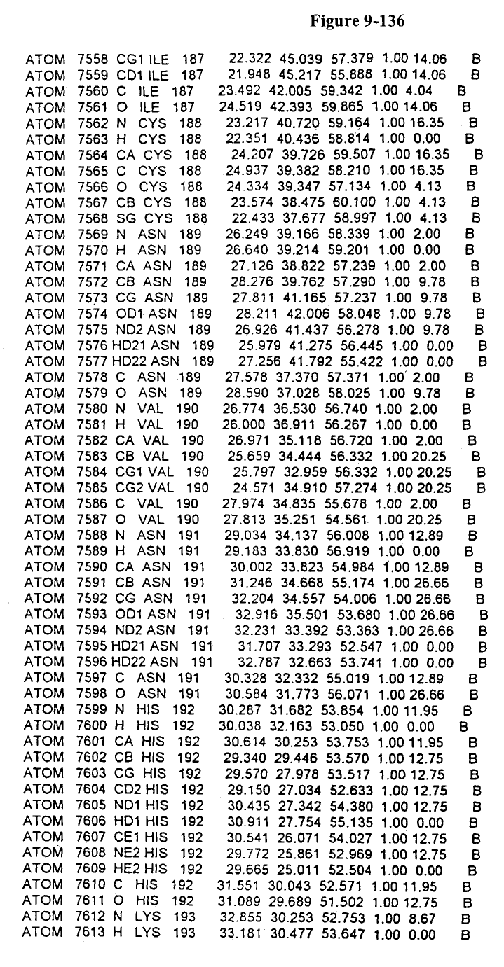

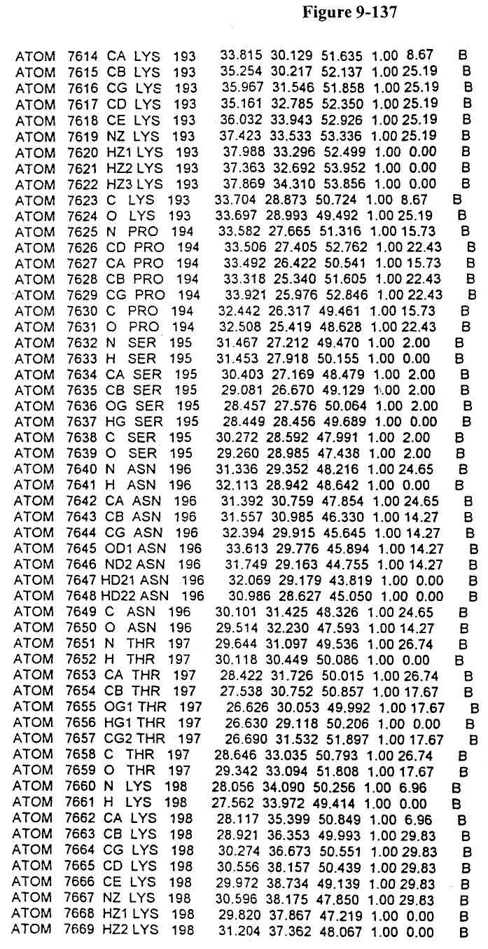

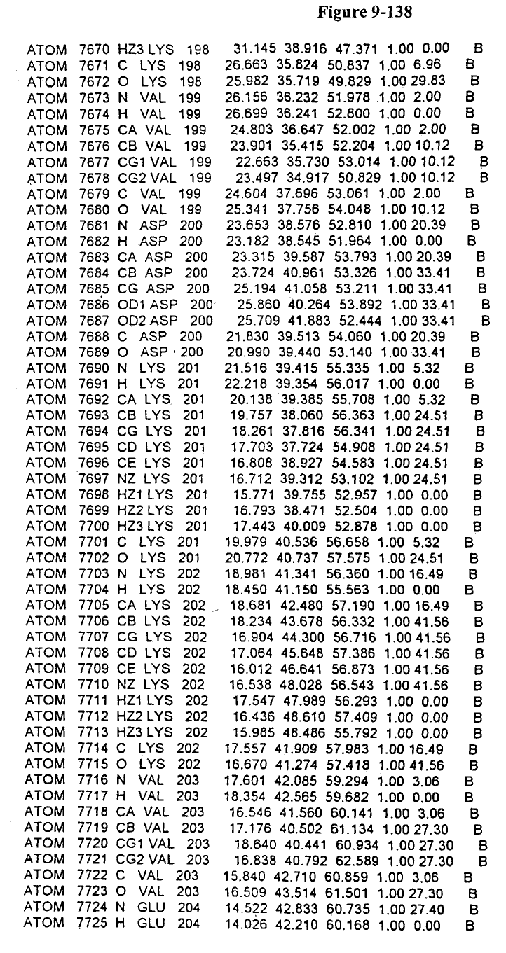

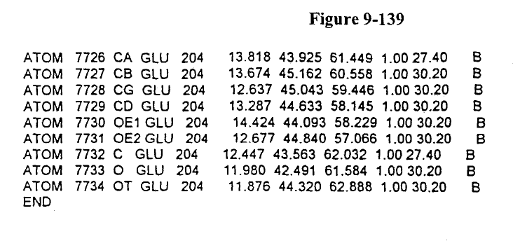

Subsequent molecular replacement computing was done with the program AMoRe (Navaja, J. (1994) Acta Crystalloqr. A 50, 157-163) from the CCP4 program package (Collaborative Computational Project No. 4. The CCP4 Suite: programs for protein crystallography. Acta Cryst. D50, 760-763). The CCP4 Suite: programs for protein crystallography Acta Cryst. D 50, 760-763). Molecular graphics manipulations were done with the program QUANTA (Molecular Simulations, Inc., San Diego, Calif.). The coordinates for a trimer of the extracellular domain of CD154 (chains A, B and C) from the crystal structure of human CD154 (Karpusas et al. Structure 3, 1031-1039 (1995), Karpusas et al. Structure 3, 1446 (1995), U.S. patent application Ser. No. 09/180,209 and PCT patent application WO97/00895, the disclosures of all of which are hereby incorporated by reference) (PDB entry code laly) was used as a probe for rotation and translation searches. The coordinates of all atoms, including side chains, were included in the search model. The rotation search gave a single solution with a correlation coefficient (cc) of 24.4 that was consistent with the 3-fold axis predicted by the self-rotation search. This solution was used for a translation search that yielded a single peak with a cc of 19.0 and an R-factor of 50.7%. Using rigid body refinement, these values refined to cc of 20.0 and an R-factor of 50.3%. Subsequently searches for the humanized 5c8 mAb Fab fragment were carried out, keeping the CD154 solution fixed. A partially refined crystal structure of the uncomplexed human humanized 5c8 mAb Fab was used as a search probe. FIG. 9 shows the structure coordinates of that crystal structure. The rotation search produced several non-prominent peaks including some related by a 3-fold axis. Translation searches for each of these peaks confirmed that the peaks related by the 3-fold axis correspond to the correct solutions and allowed the 3 humanized 5c8 mAb Fab fragments (cc of 20.6 and an R-factor of 50.2%) to be located. Rigid body refinement of the CD154 trimer and the 3 Fab fragments resulted in cc of 35.1 and an R-factor of 48.7%.

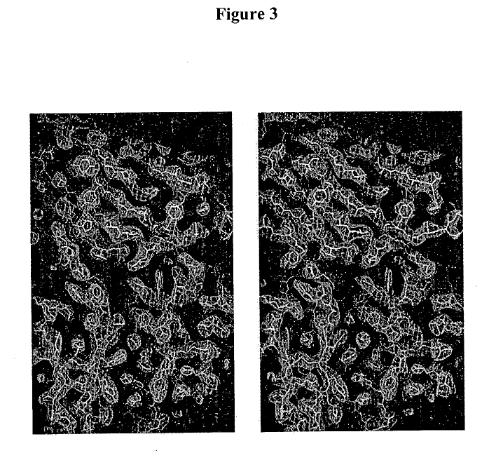

Calculation of a 2Fo-Fc electron density map (FIG. 3) showed continuous electron density for the CD154 and Fv domains of the Fab fragments but weak or no density for the constant domains of the Fab fragments. This indicated that the constant domains of the Fab were not correctly located, apparently because the elbow angle of the Fab differed from that of the search probe. To locate the constant domain, the elbow angle of the Fab (keeping the Fv fixed) was modified in increments of 10° by using a script from the XPLOR package and the correlation coefficient was monitored. The correlation coefficient had its highest value for an elbow angle of −50°, corresponding to the approximate position of the constant domain. Subsequent rigid body refinement with XPLOR, using data in the 20-4 Åresolution range, optimized the position of the constant domain, reducing the R-factor from 49.4% to 40.0% (R-free=40.5%).

All subsequent refinement computing was carried out with the XPLOR program. Five percent of the data were allocated for the calculation of R-free factor. To reduce model bias, partial models were used for 2Fo-Fc map calculation and model refinement. The initial partial model was subjected to simulated annealing and grouped B-factor refinement with non-crystallographic symmetry restraints. The R-work and R-free factors dropped to 27.0% and 32.0% respectively. Several cycles consisting of iterative model building, maximum likelihood positional refinement (Adams, P. D. et al. (1997) Proc. Natl. Acad. Sci. USA 94, 5018-5023) and B-factor refinement followed. Simulated annealing omit maps were calculated to confirm modeling of certain regions of the structure. Only model adjustments that resulted to a drop in the R-free factor were accepted. No bulk solvent correction was applied. The non-crystallographic symmetry restraints were removed in the final steps of refinement. The R-work and R-free factors of the final model were 23.3% and 28.5% respectively for the data (F>2σ) in the 35-3.1 Åresolution range. Stereochemistry statistics were calculated with PROCHECK (Laskowski, R. A., MacArthur, M. W., Moss, D. S., and Thornton, J. M. (1993) J. Appl. Crystallogr. 26, 283-290). Hydrogen bonds (<3.6 Å) were found with the program CONTACT (Collaborative Computational Project No. 4. The CCP4 Suite: programs for protein crystallography. Acta Cryst. D50, 760-763). The final model consisted of 13,173 atoms constituting 9 polypeptide chains (chain names are A, B, C for the 3 CD154 monomers, H, K, X for the 3 Fab heavy chains and L, M, Y for the 3 Fab light chains). Table 1 shows the details and summary of the crystallographic analysis.

TABLE 1
Summary of crystallographic analysis

| Data collection | |
|---|---|
| Cell dimensions | 224.48, |
| a, b, c (Å) | 129.91, 96.49 |
| β (°) | 109.62 |
| Space group | C2 |
| Resolution (Å) | 35-3.1 |
| | (3.21-3.1)† |
| Unique reflections | 46508 |
| Completeness (%) | 96.1 (87.7)† |
| Average I/σ | 7.52 (1.97)† |
| R_{merge}* (%) | 7.6 (18.8)† |
| Model | |
| Number of non-H atoms | 16,203 |
| Number of protein residues | 1731 |
| Contents of asymmetric unit | 3 Fab |
| | fragments, |
| | 1 CD154 trimer |
| Average B-factor (Å²) | 18.8 |
| Refinement | |
| Resolution range used (F > 2δ) | 35-3.1 |
| R-factor (%) | 23.3 |
| R-free (%) | 28.5 |
| Stereochemistry | |
| RMS deviations | |
| Bond lengths (Å) | 0.014 |
| Angles (°) | 1.89 |

*R_{merge} = ΣₕΣᵢ | Iₕᵢ − Iₕ |/ΣₕᵢIₕᵢ
†Values for the highest resolution shell given in parenthesis.

The structure of the globular part of the CD154 extracellular domain (residues 116-261) complexed with the Fab fragment of the humanized 5c8 mAb was determined at 3.1 Åresolution by molecular replacement and refined to a crystallographic R value of 23.3% (R-free 28.5%). The residues of CD154 visible in the crystal structure were amino acids 119 to 261 (Asn 119 to Leu 261 in FIGS. 4 and 8). The asymmetric unit of the crystal contained a single complex consisting of a CD154 homotrimer and three Fab fragments. Almost all residues except N-terminal residues 116-118 of CD154 were well-defined in the final 2Fo-Fc electron density map. The final model consisted of 1731 amino acid residues constituting 9 polypeptide chains and 3 zinc ions. No water molecules have been included in the model. Some electron density was observed for the carbohydrate of CD154 but was not of sufficient quality to allow modeling of carbohydrate residues. The stereochemistry was good (root mean square (r.m.s.) deviations on bond lengths is 0.014 Åand on bond angles is 1.89°). The r.m.s. positional deviation between equivalent residues from different CD154 monomers or Fab fragments was small (0.18 Åfor main chain atoms) due to using non-crystallographic symmetry restraints during most of the refinement process. All non-glycine residues, except residue 183 of CD154, were in the allowed regions of the Ramachandran diagram. The average B-factor of the main chain atoms was 18.8 Å². The constant domains of the Fab fragments have much higher B-factors (average B-factor ˜29.5 Å²) compared to the variable domains (average B-factor 14.1 Å²). This appears to be the consequence of fewer crystal contacts for the constant domain of the Fab fragment compared to the variable domain.

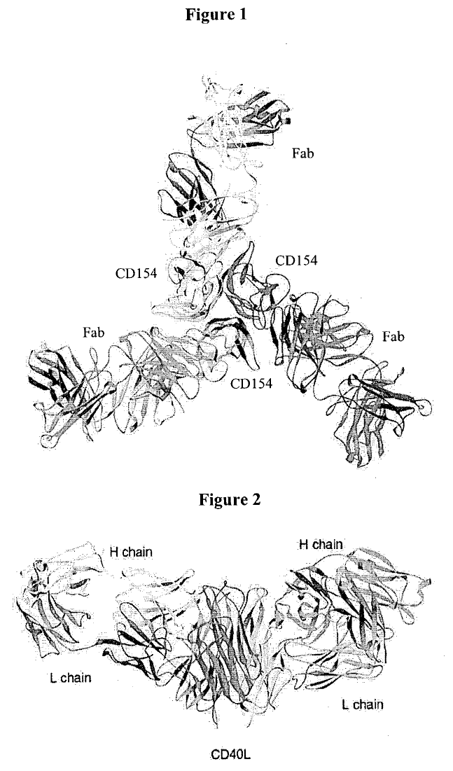

The complex had the shape of a 3-blade propeller and consisted of three hu5c8 mAb Fab molecules radially bound on a single CD154 homotrimer (FIGS. 1 and 2). The dimensions of the complex were 140×140×60 Å. The 3-fold axis of the CD154 trimer coincided with the non-crystallographic 3-fold axis of the complex. The approximate pseudo 2-fold axes of the Fab fragments, which related the heavy and light chains, intersected the 3-fold symmetry axis of the complex and had an approximate angle of 30° upward to a plane perpendicular to the 3-fold axis. When the fact that CD154 is on the cell surface is taken into consideration, this plane is expected to coincide with the cell surface.

The crystallized CD154 fragment is a homotrimeric protein and each monomer folded as β-sheet sandwich with Greek key topology. The overall shape of the trimer resembled that of a truncated pyramid. The structure of CD154 in the complex with the Fab was very similar to the structure of the uncomplexed human CD154 (Karpusas et al. Structure 3, 1031-1039 (1995) and Karpusas et al. Structure 3, 1446 (1995), U.S. patent application Ser. No. 09/180,209 and PCT patent application WO 97/00895). The A-A″ loop of CD154 maintained the extended conformation that was observed originally in the uncomplexed CD154 crystal structure and was not typical of other members of the TNF family. It further suggests that this conformation is real and not a consequence of crystal contacts. Superimposition of uncomplexed human CD154 trimer (Karpusas et al. Structure 3, 1031-1039 (1995) and Karpusas et al. Structure 3, 1446 (1995), U.S. patent application Ser. No. 09/180,209 and PCT patent application WO 97/00895)(PDB entry laly) on the complexed CD154 trimer, showed that there were no significant conformational changes of CD154 upon hu5c8 mAb Fab binding (r.m.s. deviation is 0.76 Åfor main chain atoms). The biggest differences (up to 4 Åshifts) were observed in the CD and EF loops of CD154, which are located to the “top” of the truncated pyramid, away from the hu5c8 Fab epitope. These loops are known to be the most mobile regions of the CD154 moiety (Karpusas et al. Structure 3, 1031-1039 (1995) and Karpusas et al. Structure 3, 1446 (1995), U.S. patent application Ser. No. 09/180,209 and PCT patent application WO 97/00895) and therefore the observed differences were not likely to be a consequence of hu5c8 Fab binding. Some significant differences, particularly compensatory rotamer shifts, were observed for the side chains of a few residues of the binding epitope, including Y145 and R203 residues of CD154 that were shown to be important for CD40 binding.

The Fab fragment was obtained from a humanized version of the original murine 5c8 mAb (FIG. 8). The humanized L chain construct was based on the human subgroup III k chain from hybridoma AE6-5 (Spatz, L. A. et al. (1990), J Immunol 144, 2821-8). The H chain construct was based on the subgroup I 21/28CL gene (Dersimonian, H. et al. (1987), J Immunol 139, 2496-501). The modeled Fab structure consisted of residues 1-219 of the heavy chain and 1-215 of the light chain. The variable domain of hu5c8 mab Fab can be superimposed to the variable domain of the anti-pl85HER2 antibody (PDB entry 1fvd) with an r.m.s. positional deviation of 1.36 Åfor 1040 equivalent atoms. The elbow angle of the complexed hu5c8 Fab differed by 49.9° from that of the uncomplexed hu5c8 Fab. The complementarity determining region CDR L1, CDR L2 and CDR L3 loops of the light chain have canonical structures 3, 1 and 1 respectively (Chothia et al. (1989) Nature 342, 877-883). The CDR H1 and CDR H2 loops of the heavy chain have canonical structures 1 and 2.

The interaction of a single Fab fragment with CD154 resulted in a total solvent accessible area of 771 Å² for CD154 and 765 Å² for the hu5c8 mAb Fab being buried, assuming a 1.4 A solvent probe. The antigenic epitope of 5c8 mAb is located on the right-hand side of the intersubunit cleft of CD154 and is elongated and continuous. The long axis of the epitope footprint is parallel to the long axis of the CD40 binding site. Interestingly, although CD154 only exists as a trimer on the cell surface, the epitope is composed only of residues from a single monomer of CD154.

The epitope of hu5c8 mAb Fab on CD154 consisted of residues E129, A130, S132, E142, K143, G144, Y146 of the A-A” loop; C178 of the C strand; and C218, S245, Q246, S248, H249, G250 of the G-H loop of CD154.

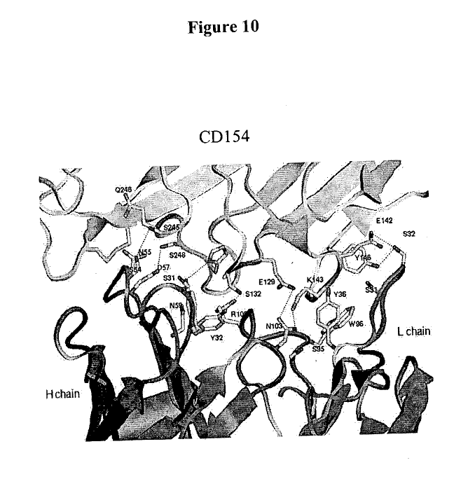

The hu5c8 mAb Fab was observed to use CDR H1, CDR H2 and CDR H3 hypervariable regions as well as CDR L1 and CDR L3 to form contacts with CD154. Most of the buried surface area was contributed by the heavy chain (61%). The residues of hu5c8 Fab involved in contacts with CD154 were S31(H), Y32(H), Y33(H) of CDR H1; N52(H), S54(H), D57(H), N59(H) of CDR H2; R102(H), N103(H) of CDR H3 and S31(L), S32(L), Y36(L) of CDR L1; S95(L), W96(L) of CDR L3. The contacts were mixed in character. There were several polar interactions, some of which involved several main chain atoms while others involved side chain atoms (FIG. 10). For example, the side chain of Y32(H) was observed to interact with the side chain of S132 of CD154 and the side chain of D57(H) was observed to interact with S248 of CD154. Also, the O1 atom of the N55 side chain was observed to form an H-bond with the carbonyl oxygen of Cys178 of CD154. No salt bridge interactions were found in the interface. In addition, several aromatic residues (Y146, H249 of CD154, Y32, Y33 of the heavy chain and Y36, W96 of the light chain) contribute to van der Waals contacts between CD154 and the antibody.

Based on the co-crystal structure as described in Example 1, the epitope for hu5c8 mAb on CD154 overlaps but is not identical with the putative CD40 binding site. This is in agreement with previous conclusions based on mutagenesis data (Garber, E. et al. (1999), J Biol Chem 274, 33545-50). For example, residues K143, and Y146, which have been identified by mutagenesis to be important for the interaction of CD154 with CD40 (Bajorath, J. et al., (1995), Biochemistry 34, 1833-44 and Singh, J. et al.(1998) Protein Sci 7, 1124-35) are also involved in interactions with hu5c8 mAb. In particular, the K143 side chain was observed to interact with the side chain of N103(H) of hu5c8 mAb as well as the main chain carbonyl of S95(L) of hu5c8 mAb. Y146 was observed to interact with the S32(L) of the hu5c8 mAb. This interaction occurred at the bottom of the cleft formed between the heavy and light chain and appeared to be the most prominent feature of the antigen-antibody interaction. The overall structure of CD154 was very similar to that of the uncomplexed CD154. Thus, the neutralizing effect of the hu5c8 mAb appears to be a consequence of steric blocking of CD154-CD40 interactions and not of any antibody-induced conformational changes. The solvent accessible surface buried upon complexation of CD154 with CD40 has been predicted to be in the range of 834 to 1123 Å² (Bajorath, J. et al. (1995), Biochemistry 34, 9884-92 and Singh, J. et al.(1998) Protein Sci 7, 1124-35). This is larger than the surface area of 765 Å² buried in the CD154-hu5c8 Fab complex. The epitope is a relatively flat region of the surface of CD154.

Significant electron density was observed for the biantennary complex-type carbohydrate attached to residue N240 of CD154. The carbohydrate chain was accommodated within a large solvent channel of the crystal lattice, about 100 Åwide. The electron density of the carbohydrate was not of sufficient quality to allow model building of the its residues, presumably due to disorder. However, it was apparent that the carbohydrate forms extensive non-covalent interactions with the heavy chain of the hu5c8 mAb. Residues of the antibody that were observed to interact within contact distance include Q43(H), E62(H), K63(H) and S66(H). These contacts may contribute to the energy of the interaction of hu5c8 mAb with CD154. CD154 mutant N240Q, which lacks a carbohydrate, exhibited a reduced level of immunoprecipitation with hu5c8 mAb (Table 2). However the level of expression of the mutant is lower than wild-type (WT) which makes it difficult to ascertain whether or not the loss of the carbohydrate has a negative effect on hu5c8 mAb binding. Additionally, the electron density map revealed that the carbohydrate interacts with R207 of CD154, a residue that has an important contribution to the positive electrostatic potential in its immediate region of CD154 molecule (Singh, J. et al. (1998), Protein Sci 7, 1124-35).

A zinc ion was found to be located near the binding site. It was coordinated by D100(H), D106(H) and E59(L) and there were no direct contacts of the ion to CD154. This ion is unlikely to play a functional role and its presence is probably a crystallization artifact.

In summary, the crystal structure of CD154 in complex with the humanized 5c8 mab Fab according to this invention constitutes the first available structure of a TNF family member in complex with a neutralizing antibody Fab fragment. The structure showed that the antibody inhibits CD154 function by sterically blocking the binding site of CD40 receptor. The possibility that antibody binding may prevent conformational changes of CD154, which may be necessary for CD40 binding, can not be discounted. However, comparison of available TNF ligand structures and their complexes with receptors does not show evidence of significant conformational changes, upon receptor binding, that are distant from the binding site (Banner, K. H. et al. (1996), Br J Pharmacol 119, 1255-61 and Hymowitz, S. G. et al. (1999), Mol Cell 4, 563-71).

The epitope of the antibody was located just above a cluster of hydrophobic core residues whose mutation has been associated with HIGMS. It has been proposed that these mutations may cause structural perturbations of a region of the surface that is important for receptor binding (Karpusas, M. et al. (1995), Structure 3, 1426). It is interesting that the CD154 antigenic epitope for hu5c8 mAb coincides with the region of the surface that is most likely to be perturbed by the mutations.

### EXAMPLE 2

### Assessment of the Nature of the 5C8 Monoclonal Antibody and CD154 Interface by Examining the Ability of Site-Directed Mutants of CD154 to Bind to hu5c8 mAb and CD40

The location and nature of the CD154 antigenic epitope of 5c8 mAb was studied by site-directed mutagenesis of human CD154. Mutation sites selected included surface residues in the vicinity of the putative CD40 binding site, residues of the interface of two CD154 monomers as well as residues involved in mutations associated with Hyper-IgM syndrome (HIGMS). Residue substitutions included changes to alanine, charge-reversal mutations or changes to other residues.

Construction and expression of CD154 mutants has been described previously (Singh, J. et al. (1998), Protein Sci 7, 1124-35, the disclosure of which is hereby incorporated by reference). Briefly, mutants of human CD154 were made by unique site elimination mutagenesis using a Pharmacia kit (Pharmacia, N.J.). COS cells were transfected with an expression vector containing the mutant gene and an SV40 origin site for amplification. Transfected cells were metabolically labeled and harvested. Cell lysates were pre-cleared with protein A sepharose beads and anti-CD154 monoclonal antibodies. Immune complexes collected on beads were washed and subjected to SDS electrophoresis. Immunoprecipitation of each mutant human CD154 protein was compared to that of wild-type human CD154 protein.

Mutated full-length CD154 was transiently expressed in its full-length membrane-bound form. Expression of mutant CD154s was confirmed with immunoprecipitation of detergent extracts from metabolically labeled cells with polyclonal antibodies directed against synthetic peptides from the N and C-termini of CD154 (Singh, J. et al. (1998), Protein Sci 7, 1124-35 and Garber, E. et al. (1999), J Biol Chem 274, 33545-50). 5c8 mAb binding to CD154 mutants was assessed by assaying the ability of CD154 mutants from detergent extracts to be immunoprecipitated by 5c8 mAb. Similarly, CD40 binding to CD154 mutants was assessed by immunoprecipitation with CD40-Fc. CD40-Fc is a fusion protein of the extracellular domain of CD40 and a human IgG Fc fragment (Hsu et al. (1997) J. Biol. Chem., 272: 911-915, the disclosure of which is hereby incorporated by reference). Table 2 summarizes data for 23 single residue mutations of human CD154.

TABLE 2
Summary of mutagenesis data*

| Mutation | 5c8 mAb | CD40-Fc | Type |
|---|---|---|---|
| A123E | − | − | HIGMS |
| V126A | − | − | HIGMS |
| S128R | − | − | HIGMS (S128R/E129G) |
| E129G | − | − | HIGMS (S128R/E129G) |
| K133A | + | + | surface charge |
| W140G | − | − | HIGMS |
| E142K | + | + | murine residue |
| K143A | +/− | +/− | surface charge |
| G144E | + | − | HIGMS |
| Y145A | +/− | − | surface residue |
| L155P | − | − | HIGMS |
| Y170C | − | − | HIGMS |
| R203A | + | +/− | surface charge |
| I204A | + | + | monomer interface |
| R207A | + | +/− | surface charge |
| T211D | + | + | HIGMS |
| G227V | − | − | HIGMS |
| A235P | − | − | HIGMS |
| H249A | − | − | surface charge |
| T251A | + | + | monomer interface |
| T254M | − | − | HIGMS |
| F253A | − | − | monomer interface |
| G257S | − | − | HIGMS |
| K216 | + | | |

*“+, +/−, −” symbols indicate immunoprecipitation levels in comparison to WT: “+” comparable signal; “+/−” reduced but detectable signal; “−” undetectable signal.

The effects for some of these mutations were previously described in the context of the crystal structure of CD154 and homology model of CD40 (Singh, J. et al. (1998), Protein Sci 7, 1124-35 and Garber, E. et al. (1999), J Biol Chem 274, 33545-50). Here, the effects of these and additional mutations on 5c8 mAb binding were interpreted in the context of the crystal structure of CD154-5c8 mAb Fab.

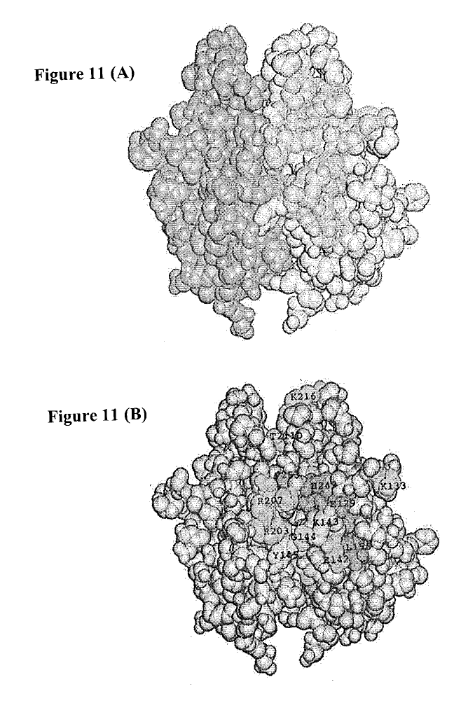

Mutation of surface residues of CD154 had an effect on immunoprecipitation that, in general, correlated with the location of the antigenic epitope of 5c8 mAb as determined from the crystal structure (Table 2 and FIG. 11). The complete loss of immunoprecipitation due to mutation H249A suggests that surface residue H249 may play important role in the energetics of the CD154-5c8 mAb interaction. This conclusion relies on the observation that the CD154-hu5c8 mAb Fab interaction surface in the co-crystal structure described in Example 1 (as well as the CD154-CD40 interaction surface) was very extensive and therefore the loss of single residue side chains in most cases is not likely to result in complete loss of the interaction between CD154 and 5c8 mAb. The crystal structure showed that residue H249 lies in the middle of the epitope and interacts with Y33(H) of 5c8 mAb (Example 1). Mutation of residue E129 to glycine also resulted in complete loss of immunoprecipitation. This residue was observed to interact with N103(H) of hu5c8 mAb Fab and its substitution with glycine resulted in loss of this interaction. As discussed further below, mutation E129G also resulted in structural perturbations that may contribute to the loss of immunoprecipitation. The substitution of other surface residues, such as K143, had a more intermediate effect. Of interest is surface residue Y145, which was not observed to form direct interactions with 5c8 mAb. However, mutation Y145A had a intermediate effect on immunoprecipitation with 5c8 mAb. The OH group of the side chain of Y145 was shown to interact with the carbonyl oxygen of E230 of the adjacent CD154 monomer in the co-crystal structure described in Example 1. This suggests that the residue may also play a structure-stabilizing role that could explain the observed effect in immunoprecipitation.

Most of the HIGMS mutations resulted in complete loss of ability of CD154 to be immunoprecipitated by 5c8 mAb (Garber, E. et al. (1999) J Biol Chem 274, 33545-50 and Bajorath, J., et al.(1995), Biochemistry 34, 1833-44). Inspection of the co-crystal structure showed that most of the HIGMS mutations involve residues that are not directly involved in 5c8 mab interactions and are more likely to play a structural role. For example, residues A123, V126, W140, L155, Y170, A235, T254, G257 are buried residues and are likely to be important for protein folding and stability. Consistent with that view, all Hyper-IgM mutations that affected 5c8 mAb binding also affected CD40-Fc binding. It appears that most HIGMS mutations affect the structure locally, since it has been shown that these mutations do not cause an alteration in structure that is sufficient to completely prevent homotrimerization (Garber, E. et al. (1999) J Biol Chem 274, 33545-50). Interestingly, most of the residues involved in known HIGMS mutations form a cluster buried underneath the surface area of the 5c8 mAb epitope on CD154 (FIG. 11). This fact makes it more likely that the structural perturbations induced by the mutations propagate to the surface area of the epitope and result in loss of 5c8 mAb binding.

HIGMS double mutation S128R/E129G and single mutation T211D are the only HIGMS mutations that involve essentially exposed surface residues. To dissect the contribution of each mutated residue of HIGMS mutation S128R/E129G, single mutants S128R and E129G were generated in addition to the double mutant protein. The double mutation S128R/E129G and the single mutations S128R and E129G resulted in complete loss of 5c8 mAb and CD40-Fc binding (Table 2). Inspection of the crystal structure showed that residue S128 does not interact at all with 5c8 mAb, however it stabilizes essential residue H249 which interacts with Y33(H) of 5c8 mAb. Its substitution with arginine may disrupt this interaction and the introduction of the positive charge may alter the local electrostatic potential. The other residue involved in the mutation, E129, was observed to interact with N103(H) of 5c8 mAb and also stabilizes the conformation of K143 of CD154 which interacts with N103(H) of 5c8 mAb (FIGS. 10 and 11). Its substitution with glycine resulted in loss of these interactions. Previous studies have also shown that while mutant E129G binds weakly to CD40-Fc, mutant E129A binds like wild-type to CD40-Fc (Bajorath, J., et al.(1995), Biochemistry 34, 1833-44). This suggested that the substitution to glycine introduces additional flexibility to the loop and may perturb the structure of CD154. Thus the effect of the E129G mutation on 5c8 mAb binding is a combination of loss of interactions and local perturbation of the structure which may result in loss of additional interactions. T211D mutant behaved like wild-type in terms of 5c8 mAb and CD40-Fc binding and it has been concluded that the mutation is a result of polymorphism of the CD154 gene (Garber, E. et al. (1999) J Biol Chem 274, 33545-50). This residue is surface-exposed and lies near the top of the pyramid, away from the epitope (FIG. 11). Previous crystallographic analysis has confirmed that the T211D mutant protein folds like wild-type (Garber, E. et al. (1999) J Biol Chem 274, 33545-50).

Equivalents

The invention may be embodied in other specific forms without departing from the spirit or essential characteristics thereof. The foregoing embodiments are therefore to be considered in all respects illustrative of, rather than limiting on, the invention disclosed herein. All changes which come within the meaning and range of equivalency of the claims are intended to be embraced therein.

We claim:

1. A crystallizable composition comprising a CD154 polypeptide complexed with an anti-CD154 antibody or an antigen binding fragment of said antibody.

2. The crystallizable composition according to claim 1, wherein said anti-CD154 antibody is a monoclonal antibody.

3. The crystallizable composition according to claim 1, wherein said CD154 polypeptide is a polypeptide comprising the extra-cellular domain of CD154.

4. The crystallizable composition according to claim 1, wherein said CD154 polypeptide comprises a polypeptide consisting of amino acid 116 to amino acid 261 of CD154.

5. The crystallizable composition according to claim 1, wherein said anti-CD154 antibody is a monoclonal antibody which specifically binds the 5c8 antigen, which is specifically bound by monoclonal antibody 5c8 (produced by the hybridoma having ATCC Accession No. HB 10916).

6. The crystallizable composition according to claim 1, wherein said fragment is a Fab fragment.

7. The crystallizable composition according to claim 6, wherein said Fab fragment is a Fab fragment of monoclonal antibody 5c8 (produced by the hybridoma having ATCC Accession No. HB 10916), or of humanized 5c8 mAb.

8. A crystallizable composition comprising a trimer of CD154 polypeptides and three anti-CD154 monoclonal antibodies, or antigen binding fragments thereof, wherein each of said polypeptides comprises the extra-cellular domain of CD154.

9. A crystal comprising a CD154 polypeptide complexed with an anti-CD154 antibody, or an antigen binding fragment thereof.

10. The crystal according to claim 9, wherein said CD154 polypeptide comprises the extra-cellular domain of CD154 polypeptide.

11. The crystal according to claim 9, wherein said CD154 polypeptide comprises a polypeptide consisting of amino acid 116 to amino acid 261 of CD154.

12. The crystal according to claim 9, wherein said anti-CD154 antibody is a monoclonal antibody.

13. The crystal according to claim 9, wherein said anti-CD154 antibody is a monoclonal antibody which specifically binds the 5c8 antigen, which is specifically bound by monoclonal antibody 5c8 (produced by the hybridoma having ATCC Accession No. HB 10916).

14. The crystal according to claim 9, wherein said fragment is a Fab fragment.

15. The crystal according to claim 14, wherein said Fab fragment is a Fab fragment of monoclonal antibody 5c8 (produced by the hybridoma having ATCC Accession No. HB 10916) or of humanized 5c8 monoclonal antibody.

16. A crystal comprising a trimer of CD154 polypeptides and three anti-CD154 antibodies, or antigen binding fragments thereof, wherein each of said polypeptides comprises the extra-cellular domain of CD154.

17. A computer for producing a three-dimensional representation of:
a) a molecular complex comprising a first binding site defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4; or
b) a homologue of said molecular complex, wherein said homologue comprises a second binding site that has a root mean square deviation from the backbone atoms of said amino acids between 0.00 Å and 1.50A;
wherein said computer comprises:
(i) a machine-readable data storage medium comprising a data storage material encoded with machine-readable data, wherein said data comprises the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4; and
(ii) instructions for processing said machine-readable data into said three-dimensional representation.

18. The computer for producing a three-dimensional representation according to claim 17, wherein said homologue comprises a second binding site that has a root mean square deviation from the backbone atoms of said amino acids of between 0.00 Å and 1.0OA.

19. The computer for producing a three-dimensional representation according to claim 17, wherein said homologue comprises a second binding site that has a root mean square deviation from the backbone atoms of said amino acids of between 0.00 Å and 0.50 Å.

20. The computer according to any one of claims 17-19, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

21. The computer according to any one of claims 17-19, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

22. A computer for producing a three-dimensional representation of:
a) a molecular complex comprising a first binding site, defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, that associates with one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; or
b) a homologue of said molecular complex, wherein said homologue comprises a second binding site that has a root mean square deviation from the backbone atoms of said CD154 amino acids and said one or more anti-CD154 amino acids between 0.00 Å and 1.50 Å;
wherein said computer comprises:
(i) a machine-readable data storage medium comprising a data storage material encoded with machine-readable data, wherein said data comprises the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4 and the structure coordinates of one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; and
(ii) instructions for processing said machine-readable data into said three-dimensional representation.

23. The computer for producing a three-dimensional representation according to claim 22, wherein said homologue comprises a second binding site that has a root mean square deviation from the backbone atoms of said CD154 amino acids of between 0.00 Å and 1.00 Å.

24. The computer for producing a three-dimensional representation according to claim 22, wherein said homologue comprises a second binding site that has a root mean square deviation from the backbone atoms of said CD154 amino acids of between 0.00 Å and 0.50 Å.

25. The computer according to any one of claims 22-24, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

26. The computer according to any one of claims 22-24, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

27. A computer for producing a three-dimensional representation of:
a) a molecular complex defined by structure coordinates of one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; or
b) a homologue of said molecular complex, wherein said homologue has a root mean square deviation from the backbone atoms of said amino acids between 0.00 Å and 1.50 Å;
wherein said computer comprises:
(i) a machine-readable data storage medium comprising a data storage material encoded with machine-readable data, wherein said data comprises the structure coordinates of anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; and
(ii) instructions for processing said machine-readable data into said three-dimensional representation.

28. The computer for producing a three-dimensional representation according to claim 27, wherein said homologue has a root mean square deviation from the backbone atoms of said amino acids of between 0.00 Å and 1.00 Å.

29. The computer for producing a three-dimensional representation according to claim 27, wherein said homologue has a root mean square deviation from the backbone atoms of said amino acids of between 0.00 Å and 0.50 Å.

30. A computer for producing a three-dimensional representation of:
a) a molecular complex defined by at least a portion of the structure coordinates of all the CD154 and anti-CD154 antibody amino acids set forth in FIG. 4, or
b) a homologue of said molecular complex, wherein said homologue has a root mean square deviation from the backbone atoms of said amino acids between 0.00 Å than 1.50 Å; and
wherein said computer comprises:
(i) a machine-readable data storage medium comprising a data storage material encoded with machine-readable data, wherein said data comprises at least a portion of the structure coordinates of all of the CD154 and anti-CD154 antibody amino acids set forth in FIG. 4; and
(ii) instructions for processing said machine-readable data into said three-dimensional representation.

31. The computer for producing a three-dimensional representation according to claim 30, wherein said homologue has a root mean square deviation from the backbone atoms of said amino acids of between 0.00 Å and 1.00 Å.

32. The computer for producing a three-dimensional representation according to claim 30, wherein said homologue has a root mean square deviation from the backbone atoms of said amino acids of between 0.00 Å and 0.50 Å.

33. A computer for determining at least a portion of the structure coordinates corresponding to X-ray diffraction data obtained from a molecular complex, wherein said computer comprises:
a) a machine-readable data storage medium comprising a data storage material encoded with machine-readable data, wherein said data comprises at least a portion of the structure coordinates of CD154 or anti-CD154 antibody according to FIG. 4;
b) a machine-readable data storage medium comprising a data storage material encoded with machine-readable data, wherein said data comprises X-ray diffraction data obtained from said molecular complex; and
c) instructions for performing a Fourier transform of the machine readable data of (a) and for processing said machine readable data of (b) into structure coordinates.

34. The computer according to any one of claims 17-19, 22-24 or 27-33, further comprising a display for displaying said structure coordinates.

35. The computer according to claim 20, further comprising a display for displaying said structure coordinates.

36. The computer according to claim 21, further comprising a display for displaying said structure coordinates.

37. The computer according to claim 25, further comprising a display for displaying said structure coordinates.

38. The computer according to claim 26, further comprising a display for displaying said structure coordinates.

39. A method for evaluating the potential of a chemical entity to associate with:
a) a molecular complex comprising a first binding site defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4; or
b) a homologue of said molecular complex, wherein said homologue comprises a second binding site that has a root mean square deviation from the backbone atoms of said amino acids between 0.00 Å and 1.50 Å;
comprising the steps of:
(i) employing computational means to perform a fitting operation between the chemical entity and said first binding site of the molecular complex or said second binding site of said homologue of said molecular complex; and
(ii) analyzing the results of said fitting operation to quantify the association between the chemical entity and said first binding site or said second binding site.

40. The method according to claim 39, wherein said homologue comprises a second binding site that has a root mean square deviation from the backbone atoms of said amino acids of between 0.00 Å and 1.00 Å.

41. The method according to claim 39, wherein said homologue has a second binding site that has a root mean square deviation from the backbone atoms of said amino acids of between 0.00 Å and 0.50 Å.

42. The method according to any one of claims 39-41, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

43. The method according to any one of claims 39-41, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

44. A method for evaluating the potential of a chemical entity to associate with:
a) a molecular complex comprising a first binding site, defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, that associates with one or more anti-CD154 antibody amino acids Ser31, Tyr32, Tyr33, Asn52, Ser54, Asp57, Asn59, Arg102, Asn103 of the heavy chain and amino acids Ser31; Ser32, Tyr36, Ser95 and Trp96 of the light chain according to FIG. 4; or
b) a homologue of said molecular complex, wherein said homologue comprises a second binding site that has a root mean square deviation from the backbone atoms of said CD154 amino acids between 0.00 Å and 1.50 Å;
comprising the steps of:
(i) employing computational means to perform a fitting operation between the chemical entity and said first binding site or said second binding site; and
(ii) analyzing the results of said fitting operation to quantify the association between the chemical entity and said first binding site or said second binding site.

45. The method according to claim 44, wherein said homologue comprises a second binding site that has a root mean square deviation from the backbone atoms of said CD154 amino acids of between 0.00 Å and 1.00 Å.

46. The method according to claim 44, wherein said homologue comprises a second binding site that has a root mean square deviation from the backbone atoms of said CD154 amino acids of between 0.00 Å and 0.50 Å.

47. The method according to any one of claims 44-46, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

48. The method according to any one of claims 44-46, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

49. A method for evaluating the potential of a chemical entity to associate with:
a) a molecular complex defined by at least a portion of the structure coordinates of all the CD154 and anti-CD154 antibody amino acids, as set forth in FIG. 4; or
b) a homologue of said molecular complex having a root mean square deviation from the backbone atoms of said amino acids between 0.00 Å and 1.50 Å;
comprising the steps of:
(i) employing computational means to perform a fitting operation between the chemical entity and a first binding site of said molecular complex or a second binding site of said homologue of said molecular complex; and
(ii) analyzing the results of said fitting operation to quantify the association between the chemical entity and said first binding site or said second binding site.

50. The method according to claim 49, wherein said homologue has a root mean square deviation from the backbone atoms of said amino acids between 0.00 Å and 1.00 Å.

51. The method according to claim 49, wherein said homologue has a root mean square deviation from the backbone atoms of said amino acids between 0.00 Å and 0.50 Å.

52. The method according to any one of claims 49-51, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

53. The method according to any one of claims 49-51, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

54. A chemical entity identified by the method according to any one of claims 39-53.

55. A compound assembled from one or more chemical entities according to claim 54.

56. A method for identifying a potential agonist or antagonist of CD154 comprising the steps of:
a) using the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4±a root mean square deviation from the backbone atoms of said amino acids between 0.00 Å and 1.50 Å, to generate a three-dimensional structure of a molecular complex comprising a binding site;
b) employing said three-dimensional structure to design or select said potential agonist or antagonist;
c) synthesizing said potential agonist or antagonist; and
d) contacting said potential agonist or antagonist with CD154 to determine the ability of said potential agonist or antagonist to bind to CD154.

57. The method according to claim 56, wherein said root mean square deviation from the backbone atoms of said amino acids is between 0.00 Å and 1.00A.

58. The method according to claim 56, wherein said root mean square deviation from the backbone atoms of said amino acids is between 0.00 Å and 0.50 Å.

59. The method according to any one of claims 56-58, wherein said binding site is a binding site for 5c8 mAb (produced by the hybridoma having ATCC Accession No. HB 10916), or an antigen binding fragment thereof, or humanized 5c8 mAb, or an antigen binding fragment thereof.

60. A method for identifying a potential agonist or antagonist of CD154 comprising the steps of:
a) using the structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, wherein said CD154 amino acids associate with one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4±a root mean square deviation from the backbone atoms of said CD154 amino acids between 0.00 Å and 1.50 Å, to generate a three-dimensional structure of a molecular complex comprising a binding site;
b) employing said three-dimensional structure to design or select said potential agonist or antagonist;
c) synthesizing said potential agonist or antagonist; and
d) contacting said potential agonist or antagonist with CD154 to determine the ability of said potential agonist or antagonist to bind to CD154.

61. The method according to claim 60, wherein said root mean square deviation from the backbone atoms of said CD154 amino acids is between 0.00 Å and 1.00 Å.

62. The method according to claim 60, wherein said root mean square deviation from the backbone atoms of said CD154 amino acids is between 0.00 Å and 0.50 Å.

63. The method according to any one of claims 60-62, wherein said binding site is a binding site for 5c8 mAb (produced by the hybridoma having ATCC Accession No. HB 10916), or a variant of an antigen binding fragment thereof, or humanized 5c8 mAb, or an antigen binding fragment thereof.

64. A method for identifying a potential agonist or antagonist of CD154 comprising the steps of:
a) using at least a portion of the structure coordinates of all the amino acids of CD154 and anti-CD154 antibody according to FIG. 4±a root mean square deviation from the backbone atoms of said CD154 amino acids between 0.00 Å and 1.50 Å, to generate a three-dimensional structure of a molecular complex comprising a binding site;
b) employing said three-dimensional structure to design or select said potential agonist or antagonist;
c) synthesizing said potential agonist or antagonist; and
d) contacting said potential agonist or antagonist with CD154 to determine the ability of said potential agonist or antagonist to bind to CD154.

65. The method according to claim 64, wherein said root mean square deviation from the backbone atoms of said amino acids is between 0.00 Å and 1.0OA.

66. The method according to claim 64, wherein said root mean square deviation from the backbone atoms of said amino acids is between 0.00 Å and 0.50 Å.

67. The method according to any one of claims 64-66, wherein said binding site is a binding site for 5c8 mAb (produced by the hybridoma having ATCC Accession No. HB 10916), or a variant of an antigen binding fragment thereof, or humanized 5c8 mAb, or an antigen binding fragment thereof.

68. The method according to any one of claims 56-58, 60-62 or 64-66, further comprising the step of:
(e) determining whether said potential antagonist interrupts CD40:CD154 interaction.

69. The method according to claim 59, further comprising the step of:
(e) determining whether said potential antagonist interrupts CD40:CD154 interaction.

70. The method according to claim 63, further comprising the step of:
(e) determining whether said potential antagonist interrupts CD40:CD154 interaction.

71. The method according to claim 6-7, further comprising the step of:
(e) determining whether said potential antagonist interrupts CD40:CD154 interaction.

72. A potential agonist or antagonist of CD154 identified by the method according to any one of claims 57-71.

73. A method for evaluating the potential of a variant of monoclonal antibody 5c8, or a variant of an antigen binding fragment thereof, or a variant of humanized 5c8 mAb, or a variant of an antigen binding fragment thereof, to associate with:
a) a molecular complex comprising a first binding site defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4; or
b) a homologue of said molecular complex, wherein said homologue comprises a second binding site that has a root mean square deviation from the backbone atoms of said amino acids between 0.00 Å and 1.50 Å;
comprising the steps of:
(i) employing computational means to perform a fitting operation between the variant and said first binding site or said second binding site; and
(ii) analyzing the results of said fitting operation to quantify the association between the variant and said first binding site or said second binding site.

74. The method according to claim 73, wherein said homologue has a root mean square deviation from the backbone atoms of said amino acids of between 0.00 Å and 1.00 Å.

75. The method according to claim 73, wherein said homologue has a root mean square deviation from the backbone atoms of said amino acids of between 0.00 Å and 0.50 Å.

76. The method according to any one of claims 73-75, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

77. The method according to any one of claims 73-75, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

78. A method for evaluating the potential of a variant of monoclonal antibody 5c8, or a variant of an antigen binding fragment thereof, or a variant of humanized 5c8 mAb, or a variant of an antigen binding fragment thereof, to associate with:
a) a molecular complex comprising a first binding site, defined by structure coordinates of CD154 amino acids Glu129, Ala130, Ser132, Glu142, Lys143, Gly144, Tyr146, Cys178, Cys218, Ser245, Gln246, Ser248, His249 and Gly250 according to FIG. 4, that associates with one or more anti-CD154 antibody amino acids Ser31, Tyr32, Tyr33, Asn52, Ser54, Asp57, Asn59, Arg102, Asn103 of the heavy chain and amino acids Ser31, Ser32, Tyr36, Ser95 and Trp96 of the light chain according to FIG. 4; or
b) a homologue of said molecular complex, wherein said homologue comprises a second binding site that has a root mean square deviation from the backbone atoms of said CD154 amino acids between 0.00 Å and 1.50 Å;
comprising the steps of:
(i) employing computational means to perform a fitting operation between the variant and said first binding site or said second binding site; and
(ii) analyzing the results of said fitting operation to quantify the association between the variant and said first binding site or said second binding site.

79. The method according to claim 78, wherein said homologue has a root mean square deviation from the backbone atoms of said CD154 amino acids of between 0.00 Å and 1.00 Å.

80. The method according to claim 78, wherein said homologue has a root mean square deviation from the backbone atoms of said CD154 amino acids of between 0.00 Å and 0.50 Å.

81. The method according to any one of claims 78-80, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

82. The method according to any one of claims 78-80, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

83. A method for evaluating the potential of a variant of monoclonal antibody 5c8, or a variant of an antigen binding fragment thereof, or a variant of humanized 5c8 mAb, or a variant of an antigen binding fragment thereof, to associate with:
a) a molecular complex defined by at least a portion of the structure coordinates of all the CD154 and anti-CD154 antibody amino acids, as set forth in FIG. 4; or
b) a homologue of said molecular complex having a root mean square deviation from the backbone atoms of said amino acids between 0.00 Å and 1.50 Å;
comprising the steps of:
(i) employing computational means to perform a fitting operation between the variant and a first binding site of the molecular complex or a second binding site of the homologue of the molecular complex; and
(ii) analyzing the results of said fitting operation to quantify the association between the variant and said first binding site or said second binding site.

84. The method according to claim 83, wherein said homologue has a root mean square deviation from the backbone atoms of said amino acids of between 0.00 Å and 1.00 Å.

85. The method according to claim 83, wherein said homologue has a root mean square deviation from the backbone atoms of said amino acids of between 0.00 Å and 0.50 Å.

86. The method according to any one of claims 83-85, wherein said first binding site is a binding site for 5c8 mAb (produced by the hybridoma having ATCC Accession No. HB 10916), or an antigen binding fragment thereof, or humanized 5c8 mAb, or an antigen binding fragment thereof.

87. The method according to any one of claims 83-85, wherein said second binding site is a binding site for 5c8 mAb (produced by the hybridoma having ATCC Accession No. HB 10916), or an antigen binding fragment thereof, or humanized 5c8 mAb, or an antigen binding fragment thereof.

88. A variant of monoclonal antibody 5c8 or a variant of humanized 5c8 mAb, or a variant of an antigen binding fragment thereof, identified by the method according to any one of claims 73-87.

89. A pharmaceutical composition comprising a pharmaceutically acceptable carrier and a variant of monoclonal antibody 5c8 or a variant humanized 5c8 mAb, or a variant of an antigen binding fragment thereof, according to claim 88, a potential agonist or antagonist of CD154 according to claim 72, a chemical entity according to claim 54 or a compound according to claim 55.

90. A method of treating a condition associated with inappropriate CD154 induced activation in a subject, comprising the step of administering an effective amount of a pharmaceutical composition according to claim 89 to the subject.

91. A method of attenuating severity of a condition associated with inappropriate CD154 mediated activation in a subject, comprising the step of administering an effective amount of a pharmaceutical composition according to claim 89 to the subject.

92. A method of suppressing effects of a condition associated with inappropriate CD154 mediated activation in a subject, comprising the step of administering an effective amount of a pharmaceutical composition according to claim 89 to the subject.

93. A method of preventing development of a condition associated with inappropriate CD154 mediated activation in a subject, comprising the step of administering an effective amount of a pharmaceutical composition according to claim 89 to the subject.

94. A method of delaying onset of a condition associated with inappropriate CD154 mediated activation in a subject, comprising the step of administering an effective amount of a pharmaceutical composition according to claim 89 to the subject.

95. A method of inhibiting a condition associated with inappropriate CD154 mediated activation in a subject, comprising the step of administering an effective amount of a pharmaceutical composition according to claim 89 to the subject.

96. A method of reversing a condition associated with inappropriate CD154 mediated activation in a subject, comprising the step of administering an effective amount of a pharmaceutical composition according to claim 89 to the subject.

97. A method of treating a condition associated with inappropriate CD154 mediated activation in a subject, comprising the step of administering an effective amount of a pharmaceutical composition according to claim 89 to the subject.

98. A method of preventing a condition associated with inappropriate CD154 mediated activation in a subject, comprising the step of administering an effective amount of a pharmaceutical composition according to claim 89 to the subject.

99. The method according to any one of claims 88-98, wherein the subject is a primate.

100. The method according claim 99, wherein said primate is a human.

101. The method according to any one of claims 88-98, wherein the condition is an unwanted immune response.

102. The method according to any one of claims 88-98, wherein the condition is an unwanted inflammatory response.

103. The method according to any one of claims 88-98, wherein the condition is an autoimmune disease.

104. The method according to any one of claims 88-98, wherein the condition is an allergy.

105. The method according to any one of claims 88-98, wherein the condition is an inhibitor response to a therapeutic agent.

106. The method according to any one of claims 88-98, wherein the condition is rejection of a donor organ.

107. The method according to any one of claims 88-98, wherein the condition is a B cell cancer.

108. The method according to any one of claims 88-98, wherein the condition is selected from the group consisting of: systemic lupus erythematosis, lupus nephritis, lupus neuritis, asthma, chronic obstructive pulmonary disease, bronchitis, emphysema, multiple sclerosis, uveitis, Alzheimer's disease, traumatic spinal cord injury, stroke, atherosclerosis, coronary restenosis, ischemic congestive heart failure, cirrhosis, hepatitis C, diabetic nephropathy, glomerulonephritis, osteoarthritis, rheumatoid arthritis, psoriasis, atopic dermatitis, systemic sclerosis, radiation-induced fibrosis, Crohn's disease, ulcerative colitis, multiple myeloma and cachexia.

109. A computer for determining at least a portion of the structure coordinates corresponding to an X-ray diffraction pattern of a molecular complex, wherein said computer comprises:
a) a machine-readable data storage medium comprising a data storage material encoded with machine-readable data, wherein said data comprises at least a portion of the structure coordinates according to FIG. 4;
b) a machine-readable data storage medium comprising a data storage material encoded with machine-readable data, wherein said data comprises an X-ray diffraction pattern of said molecular complex;
c) a working memory for storing instructions for processing said machine-readable data of a) and b);
d) a central processing unit coupled to said working memory and to said machine-readable data of a) and b) for performing a Fourier transform of the machine readable data of (a) and for processing said machine readable data of (b) into structure coordinates; and
e) a display coupled to said central processing unit for displaying said structure coordinates of said molecular complex.
